(19) United States
(12) Patent Application Publication
(10) Pub. No.: US 2024/0343726 A1
(43) Pub. Date: Oct. 17, 2024

(54) PYRROLOPYRIDINE-3- AND 4-CARBOXAMIDE COMPOSITIONS AND METHODS FOR CELLULAR PROLIFERATION

(71) Applicant: The Rockefeller University, New York, NY (US)

(72) Inventors: A. James HUDSPETH, New York, NY (US); Nathaniel KASTAN, New York, NY (US); Rui LIANG, East Brunswick, NJ (US); David John HUGGINS, New York, NY (US); Nigel John LIVERTON, Harleysville, PA (US); John David GINN, New Milford, CT (US); Ksenia GNEDEVA, New York, NY (US)

(73) Assignee: The Rockefeller University, New York, NY (US)

(21) Appl. No.: 18/294,336

(22) PCT Filed: Aug. 1, 2022
(86) PCT No.: PCT/US2022/074373

§ 371 Date: Feb. 1, 2024

Related U.S. Application Data
(60) Provisional application No. 63/228,348, filed on Aug. 2, 2021.

(51) Int. Cl. C07D 471/04; A61K 31/437; A61K 31/444; A61K 31/4985; A61K 31/5377; A61K 31/541; A61K 31/554; C07D 519/00

(52) U.S. Cl. CPC C07D 471/04; A61K 31/437; A61K 31/444; A61K 31/4985; A61K 31/5377; A61K 31/541; A61K 31/554; C07D 519/00

(57) ABSTRACT

N-(3-substituted thiazaheterocyclylidene)-1H-pyrrolo[2,3-b]pyridine-3-carboxamides, N-(3-substituted thiazaheterocyclylidene)-1H-pyrrolo[2,3-b]pyridine-4-carboxamides and N-(3-substituted thiazaheterocyclylidene)-1H-pyrrolo[3,2-b]pyridine-1-carboxamides

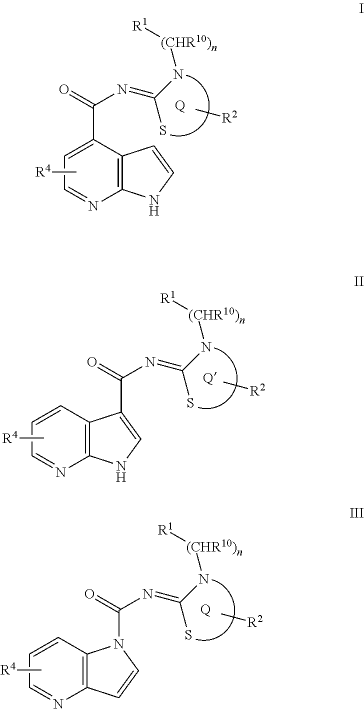

wherein the ring designated Q or Q′ is a five-, six-, or seven-membered heterocycle containing one sulfur and one nitrogen are disclosed. The compounds activate Yap and inhibit Lats kinases. They are therefore useful for treating hearing loss.

### GOVERNMENT RIGHTS STATEMENT

This invention was made with government support under grant number T32GM007739 awarded by National Institutes of Health. The government has certain rights in the invention.

### BACKGROUND OF THE INVENTION

### Technical Field

The present application relates generally to N-(3-substituted thiazaheterocyclylidene)-1H-pyrrolo[2,3-b]pyridine-3-carboxamides, N-(3-substituted thiazaheterocyclylidene)-1H-pyrrolo[2,3-b]pyridine-4-carboxamides and N-(3-substituted thiazaheterocyclylidene)-1H-pyrrolo[3,2-b]pyridine-1-carboxamides that inhibit Lats kinases and thus increase Yap activity. The compounds are useful for inducing the proliferation of supporting cells in the inner ear, and thus potentially for treating hearing loss.

### Background Information

Initiated in response to injury, regeneration is a complex process that can restore the structure and function of damaged tissue. Some adult mammalian tissues retain a gradually declining regenerative capability beyond development. Regeneration occurs either by activation and amplification of resident stem cells, as in the epithelia of the skin and intestine, or through cellular dedifferentiation and proliferation, as in the liver. In other instances, such as central nervous and cardiac-muscle tissues, cells exhibit little or no potential for regeneration after injury.

In view of its fundamental roles in development, proliferation, stem-cell maintenance, and dedifferentiation, Hippo signaling is an inviting target for driving regeneration. The regenerative potential of the Hippo pathway has become abundantly clear in numerous organs, including the heart, retina, liver, and intestine. Hippo signaling limits the size of the developing murine utricle, a sensory organ in the vestibular portion of the inner ear, and the Yap-Tead complex is active during—and necessary for—proliferative regeneration in the neonatal utricle. These observations suggest that chemical activation of Yap signaling might engender supporting-cell proliferation in adult tissue, a key missing step in the regeneration of the mammalian inner ear.

In an effort to identify activators of Yap, we conducted a small-molecule screen on cultured cells. We identified the compound, which we found to function as an inhibitor of Lats kinases. To test our original hypothesis, we treated utricles explanted from adult mice with the substance and found that a few days' exposure caused supporting cells to reenter the cell cycle, a critical step towards robust hair-cell regeneration.

### SUMMARY OF THE INVENTION

The invention is directed to to N-(3-substituted thiazaheterocyclylidene)-1H-pyrrolo[2,3-b]pyridine-3-carboxamides, N-(3-substituted thiazaheterocyclylidene)-1H-pyrrolo[2,3-b]pyridine-4-carboxamides and N-(3-substituted thiazaheterocyclylidene)-1H-pyrrolo[3,2-b]pyridine-1-carboxamides, pharmaceutical compositions and methods for inhibiting Lats or activating Yap, and thereby stimulating regeneration of target cells, particularly hair-cells.

The present invention relates, in a first aspect, to compounds of formula formula I, II, or III:

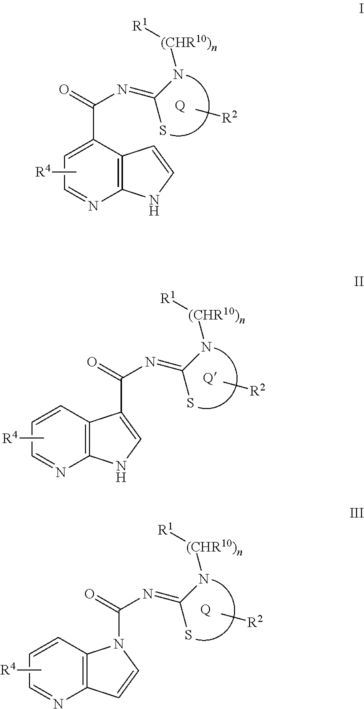

wherein:
the ring designated Q is a five-, six-, or seven-membered heterocycle containing one sulfur and one nitrogen,
the ring designated Q′ is a thiazolidine, a six-, or seven-membered heterocycle containing one sulfur and one nitrogen, or, when R² is —(CH₂)ₘNR³⁰R³¹ or a divalent three or four carbon residue that forms a fused ring, Q′ may additionally be a thiazole;
R¹ is selected from the group consisting of (C₁-C₆)alkyl, —COOH, (C₃-C₇)carbomonocyclyl, (C₉-C₁₁)carbobicyclyl, heteromonocyclyl other than 3-piperidinyl, and heterobicyclyl, wherein said (C₁-C₆)alkyl, (C₃-C₇)carbomonocyclyl, (C₉-C₁₁)carbobicyclyl, heteromonocyclyl, and heterobicyclyl may be optionally substituted with from one to three substituents selected independently from the group consisting of halogen, cyano, hydroxy, nitro, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, heteroaryl, benzenesulfonyl, (C₁-C₃)alkoxycarbonyl, aminocarbonyl, (C₁-C₃)alkylamino, di(C₁-C₃)alkylamino, amino(C₁-C₃)alkyl, (C₁-C₃)alkylamino(C₁-C₃)alkyl (C₁-C₃)dialkylamino(C₁-C₃)alkyl, (C₁-C₃)alkylthio, (C₁-C₃)alkylsulfonylamino, (C₁-C₃)alkylsulfinyl, (C₁-C₃)alkylsulfonyl, phenoxy, and benzyloxy; or, when R² is a divalent three or four carbon residue that forms a fused ring,
R¹ may provide a point of attachment for the ring;

### R² is

(a) one or two monovalent substituents selected independently from the group consisting of hydrogen, halogen, (C₁-C₇)hydrocarbyl, halo(C₁-C₆)alkyl, (C₁-C₆)acyl, hydroxy(C₁-C₃)alkyl, —C(═O)O(C₁-C₆)alkyl, —C(═O)NR²⁰R²¹, (C₁-C₆)oxaalkyl, and —(CH₂)ₘNR³⁰R³¹, or
(b) divalent ═O, or
(c) a divalent three or four carbon residue that forms a fused ring,
   with the proviso that, when R² is methyl, it is not at the 5-position of a thiazole;
   R⁴ is selected from the group consisting of hydrogen, halogen, (C₁-C₆)hydrocarbyl, halo(C₁-C₆)alkyl, (C₁-C₆)acyl, and (C₁-C₃)alkoxy;
   R¹⁰ is selected independently in each instance from the group consisting of hydrogen and methyl;
   R²⁰ is selected from the group consisting of hydrogen and (C₁-C₆)hydrocarbyl;
   R²¹ is selected from the group consisting of hydrogen, (C₁-C₆)hydrocarbyl, (C₁-C₆)oxaalkyl, amino(C₁-C₆)alkyl, (C₁-C₃)alkylamino(C₁-C₆)alkyl, di(C₁-C₃)alkylamino(C₁-C₆)alkyl, and —(CH₂)ₘ-Het, wherein Het is an aliphatic mono- or bicyclic heterocycle, optionally substituted with a substituent selected from the group consisting hydroxy, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, aminocarbonyl, (C₁-C₃)alkylaminocarbonyl, di(C₁-C₃)alkylaminocarbonyl, (C₁-C₃)alkylamino, and di(C₁-C₃)alkylamino;
   or, taken together with the nitrogen to which they are attached, R²⁰ and R²¹ form an aliphatic heterocyle;
   R³⁰ is selected from the group consisting of hydrogen and (C₁-C₆)hydrocarbyl;
   R³¹ is selected from the group consisting of hydrogen, (C₁-C₆)hydrocarbyl, (C₁-C₆)oxaalkyl, amino(C₁-C₆)alkyl, (C₁-C₃)alkylamino(C₁-C₆)alkyl, di(C₁-C₃)alkylamino(C₁-C₆)alkyl, and (C₁-C₆)acyl;
   or, taken together with the nitrogen to which they are attached, R³⁰ and R^{3′} form an aliphatic heterocyle;
   n is zero, one or two; and
   m is zero, one or two.

In another aspect, the invention relates to pharmaceutical compositions comprising a pharmaceutically acceptable carrier and a compound as described herein.

In another aspect, the invention relates to a method of for activating YAP in a cell expressing YAP comprising exposing the cell to a compound as described herein.

In another aspect, the invention relates to a method for LATS inhibition in a cell population expressing LATS comprising exposing the cell population to a compound as described herein.

In another aspect, the invention relates to a method for stimulating hair cell regeneration comprising exposing a supporting-cell population to a compound as described herein.

In another aspect, the invention relates to a method of treating a subject having, or at risk of developing, hearing loss, comprising administering to the subject an effective amount of a compound as described herein.

### DETAILED DESCRIPTION OF THE INVENTION

It has been found that compounds of formula I, II, or III.

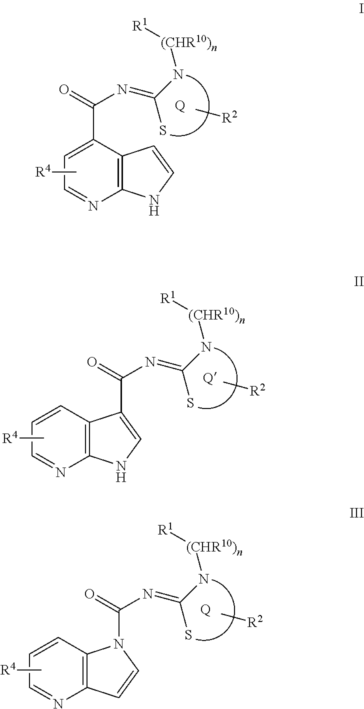

are useful for inhibiting Lats or activating Yap and are therefore potential therapeutic agents for stimulating regeneration of target cells, particularly hair-cells. Such compounds would be useful for treating hearing loss. The invention can be broken down into three subgenera.

In a first subgenus, the compounds are N-(3-substituted thiazaheterocyclylidene)-1H-pyrrolo[2,3-b]pyridine-3-carboxamides of formula I:

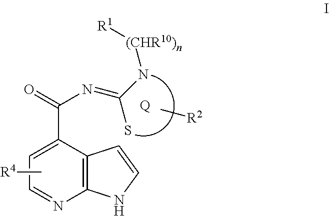

In a second subgenus, the compounds are N-(3-substituted thiazaheterocyclylidene)-1H-pyrrolo[2,3-b]pyridine-4-carboxamides of formula I:

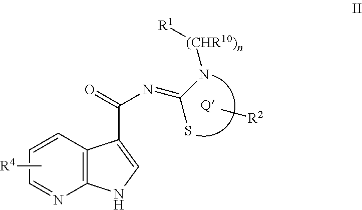

In a third subgenus, the compounds are N-(3-substituted thiazaheterocyclylidene)-1H-pyrrolo[3,2-b]pyridine-1-carboxamides of formula I:

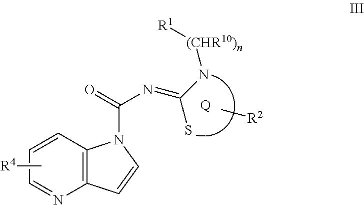

In some embodiments of subgenera I, II and III, Q or Q′ is thiazolidine:

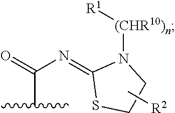

thiazine or dihydrothiazine:

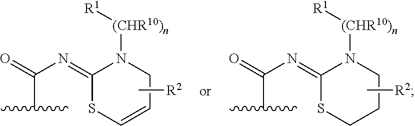

or thiazepane:

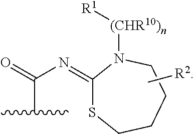

In some embodiments of subgenera I and III, Q is thiazole:

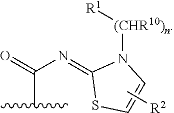

In some embodiments of subgenus II-specifically when when R² is —(CH₂)ₘNR³⁰R³¹ or when R² is a divalent three or four carbon residue that forms a fused ring -Q′ may additionally be a thiazole:

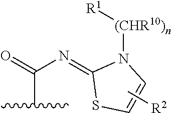

In some embodiments of the formulae I and II, n may be zero. In other embodiments of the formulae I-III, n may be one. In these embodiments, R¹⁰ may be hydrogen.

In some embodiments R¹ is selected from the group consisting of carboxy, —C(═O)NR²⁰R²¹, and optionally substituted (C₁-C₄)alkyl, phenyl, cyclohexyl, 5-membered heterocyclyl, 6-membered heterocyclyl and heterobicyclyl. In some of these R¹ is selected from the group consisting of methyl, ethyl, hydroxyethyl, hydroxypropyl, —C(═O)NMe₂, aminobutyl, and carboxyethyl. In others R¹ is optionally substituted cyclohexyl. In some of these R¹ is optionally substituted heterocyclyl. The heterocycle may be pyridinyl, pyrazolyl, piperidinyl, tetrahydropyranyl, tetrahydrofuranyl, and tetrahydroisoquinolinyl, each optionally substituted. Optional substituents may include one or two substituents selected independently from the group consisting of amino, hydroxy, (C₁-C₃)acyl, and (C₁-C₆)hydrocarbyl.

In some embodiments, R¹ is optionally substituted phenyl. The phenyl may be substituted with one or two substituents selected independently from the group consisting of halogen, cyano, hydroxy, amino, carboxy, (C₁-C₆)hydrocarbyl, trifluoromethyl, methoxy, acetyl, formyl, hydroxy(C₁-C₃)alkyl, methoxycarbonyl, carboxamido, methanesulfonylamino, and amino(C₁-C₃)alkyl. In some of these embodiments, R¹ is optionally substituted phenyl and n is zero; in others R¹ is optionally substituted phenyl and n is one.

In some embodiments, R² is selected from the group consisting of hydrogen, (C₁-C₃)alkyl, halo(C₁-C₆)alkyl, (C₁-C₆)acyl, —C(═O)O(C₁-C₆)alkyl, —C(═O)NR²⁰R²¹, (C₁-C₆)oxaalkyl, —CH₂NR³⁰R³¹, and (C₁-C₆)hydroxyalkyl. In some of these embodiments R²⁰ is chosen from hydrogen and methyl, and and R²¹ is chosen from hydrogen, methyl, (C₁-C₆)oxaalkyl, dimethylamino(C₁-C₆)alkyl, and —(CH₂)ₘ-Het. In others, R²⁰ and R²¹ taken together with the nitrogen to which they are attached form a 4-7-membered aliphatic heterocycle. Exemplary aliphatic heterocycles include piperidine, piperazine, morpholine, pyrrolidine, azetidine, azepine and the like.

In some embodiments R² is chosen from hydrogen, methyl, ethyl, propyl, cyclopropyl, hydroxymethyl, and trifluoromethyl. In some embodiments, R² is gem-dimethyl. In some embodiments, R² is oxo (═O).

In some embodiments R² is a divalent three or four carbon residue that forms a fused ring by attachment back into the thiaza heterocyle. Examples include:

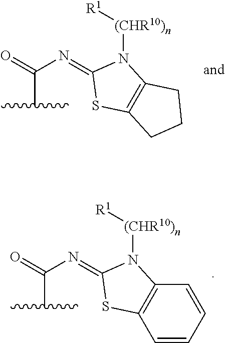

When R² is a divalent three or four carbon residue that forms a fused ring, R¹ may provide a point of attachment for the ring. An example is:

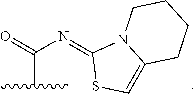 [SMILES: *C(=O)/N=c1\scc2n1CCCC2]

In some embodiments R⁴ is chosen from hydrogen, fluoro, chloro and methyl.

It is to be understood that in various embodiments, the pharmaceutical compositions of the present inventions comprise one or more pharmaceutically acceptable excipients, including, but not limited to, one or more binders, bulking agents, buffers, stabilizing agents, surfactants, wetting agents, lubricating agents, diluents, disintegrants, viscosity enhancing or reducing agents, emulsifiers, suspending agents, preservatives, antioxidants, opacifying agents, glidants, processing aids, colorants, sweeteners, taste-masking agents, perfuming agents, flavoring agents, diluents, polishing agents, polymer matrix systems, plasticizers and other known additives to provide an elegant presentation of the drug or aid in the manufacturing of a medicament or pharmaceutical product comprising a composition of the present inventions. Examples of carriers and excipients well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

In various embodiments, non-limiting examples of excipients include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), hydroxypropyl cellulose, titanium dioxide, talc, calcium carbonate (e.g., granules or powder), microcrystalline cellulose, powdered cellulose, dextrates, kaolin, silicic acid, sorbitol, starch, pre-gelatinized starch, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, a syloid silica gel (AEROSIL200, manufactured by W.R Grace Co. of Baltimore, MD), a coagulated aerosol of synthetic silica (marketed by Degussa Co. of Plano, TX), CAB-O-SIL (a pyrogenic silicon dioxide product sold by Cabot Co. of Boston, MA), colorants and mixtures thereof.

The terms “subject” or “subject in need thereof” are used interchangeably herein. These terms refer to a patient who has been diagnosed with the underlying disorder to be treated. Ordinarily, the patient will be a human. The subject may currently be experiencing symptoms associated with the disorder or may have experienced symptoms in the past. Additionally, a “subject in need thereof” may be a patient at risk of developing a particular disease, or to a patient reporting one or more of the physiological systems of a disease, even though a diagnosis of this disease may not have been made.

As used herein, the terms “treatment” or “treating” are used interchangeably. These terms refer to an approach for obtaining beneficial or desired results including, but not limited to, therapeutic benefit. Therapeutic benefit includes eradication or amelioration of the underlying disorder being treated; it also includes the eradication or amelioration of one or more of the symptoms associated with the underlying disorder such that an improvement is observed in the patient, notwithstanding that the patient may still be afflicted with the underlying disorder.

As used herein, the term “optionally substituted” may be used interchangeably with “unsubstituted or substituted”. The term “substituted” refers to the replacement of one or more hydrogen atoms in a specified group with a specified radical. For example, substituted aryl, heterocyclyl etc. refer to aryl or heterocyclyl wherein one or more H atoms in each residue are replaced with halogen, haloalkyl, alkyl, (C₁₋₈)hydrocarbyl, acyl, alkoxyalkyl, hydroxyloweralkyl, carbonyl, phenyl, heteroaryl, benzenesulfonyl, hydroxy, loweralkoxy, haloalkoxy, oxaalkyl, carboxy, alkoxycarbonyl [i.e. —C(═O)O-alkyl], carboxamido [i.e. —C(═O)NH₂], alkylaminocarbonyl [i.e. —C(═O)NH-alkyl], cyano, acetoxy, nitro, amino, alkylamino, dialkylamino, dialkylaminoalkyl, dialkylaminoalkoxy, heterocyclylalkoxy, arylalkyl, (cycloalkyl)alkyl, heterocyclyl, heterocyclylalkyl, alkylaminoalkyl, heterocyclylaminoalkyl, heterocyclylalkylaminoalkyl, cycloalkylaminoalkyl, cycloalkylalkylaminoalkyl, arylaminoalkyl, and arylalkylaminoalkyl, mercapto, alkylthio, alkylsulfinyl, benzyl, heterocyclyl, phenoxy, benzyloxy, heteroaryloxy, aminosulfonyl, amidino, guanidino, ureido, —SO₂alkyl, —SO₂NH₂, or —SO₂NHalkyl. Preferred substitutents are halogen, cyano, hydroxy, nitro, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, heteroaryl, benzenesulfonyl, (C₁-C₃)alkoxycarbonyl [i. e. —C(═O)O(C₁-C₃)alkyl], carboxamido [i.e. —C(═O)NH₂], (C₁-C₃)alkylaminocarbonyl [i.e. —C(═O)NH—(C₁-C₃)alkyl], (C₁-C₃)alkylamino, di(C₁-C₃)alkylamino, amino(C₁-C₃)alkyl, (C₁-C₃)alkylamino(C₁-C₃)alkyl (C₁-C₃)dialkylamino(C₁-C₃)alkyl, (C₁-C₃)alkylthio, (C₁-C₃)alkylsulfonylamino, (C₁-C₃)alkylsulfinyl, (C₁-C₃)alkylsulfonyl, phenoxy, and benzyloxy.

Unless otherwise specified, alkyl is a linear or branched hydrocarbyl. Unless otherwise specified, an unsubstituted alkyl has from 1 to 20 carbon atoms (e.g., 1 to 6 carbon atoms). Examples of alkyl groups include methyl, ethyl, propyl, isopropyl, butyl, s- and t-butyl and the like.

A hydrocarbon or hydrocarbyl (as a substituent) includes alkyl, cycloalkyl, polycycloalkyl, alkenyl, alkynyl, aryl and combinations thereof. Examples include cyclopropylmethyl, benzyl, phenethyl, cyclohexylmethyl, camphoryl and naphthylethyl. Hydrocarbon refers to any substituent comprised of hydrogen and carbon as the only elemental constituents. Cycloalkyl is a subset of hydrocarbyl and includes cyclic hydrocarbon groups of from 3 to 8 carbon atoms. Examples of cycloalkyl groups include c-propyl, c-butyl, c-pentyl, norbornyl and the like.

Unless otherwise specified, the term “carbocycle” is a ring system in which the ring atoms are all carbon but of any oxidation state. Thus (C₃-C₈) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Alkoxy or alkoxyl is a subset of oxaalkyl that refers to groups of from 1 to 8 carbon atoms of a straight or branched configuration attached to the parent structure through an oxygen. Examples include methoxy, ethoxy, propoxy, isopropoxy, cyclopropyloxy, cyclohexyloxy and the like. Lower-alkoxy refers to groups containing one to four carbons. For the purpose of this application, alkoxy and lower alkoxy include methylenedioxy and ethylenedioxy

Unless otherwise specified, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include formyl, acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself is called “oxo”.

Aryl and heteroaryl mean (i) a phenyl group (or benzene) or a monocyclic 5- or 6-membered heteroaromatic ring containing 1-4 heteroatoms selected from O, N, or S; (ii) a bicyclic 9- or 10-membered aromatic or heteroaromatic ring system containing 0-4 heteroatoms selected from O, N, or S; or (iii) a tricyclic 13- or 14-membered aromatic or heteroaromatic ring system containing 0-5 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

Arylalkyl refers to a substituent in which an aryl residue is attached to the parent structure through alkyl. Examples are benzyl, phenethyl and the like. Heteroarylalkyl refers to a substituent in which a heteroaryl residue is attached to the parent structure through alkyl. In one embodiment, the alkyl group of an arylalkyl or a heteroarylalkyl is an alkyl group of from 1 to 6 carbons. Examples include, e.g., pyridinylmethyl, pyrimidinylethyl and the like.

Heterocycle means a cycloalkyl or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic (i.e. aliphatic) or aromatic. Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Examples of heteroaromatic rings include: furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, benzothiazole, triazole, tetrazole, pyridine, quinoline, isoquinoline, pyrazine, quinoxaline, acridine, pyrimidine, quinazoline, pyridazine, cinnoline, phthalazine, and triazine. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

An oxygen heterocycle is a heterocycle containing at least one oxygen in the ring; it may contain additional oxygens, as well as other heteroatoms. A sulphur heterocycle is a heterocycle containing at least one sulphur in the ring; it may contain additional sulphurs, as well as other heteroatoms. Oxygen heteroaryl is a subset of oxygen heterocycle; examples include furan and oxazole. Sulphur heteroaryl is a subset of sulphur heterocycle; examples include thiophene and thiazine. A nitrogen heterocycle is a heterocycle containing at least one nitrogen in the ring; it may contain additional nitrogens, as well as other heteroatoms. Aliphatic nitrogenous heterocycles include piperidine, piperazine, morpholine, pyrrolidine, thiomorpholine, azetidine, azepine, and oxazepine. Nitrogen heteroaryl is a subset of nitrogen heterocycle; examples include pyridine, pyrrole and thiazole.

As used herein, and as would be understood by the person of skill in the art, the recitation of “a compound”—unless expressly further limited—is intended to include salts of that compound. Thus, for example, the recitation “a compound of formula I” as applied to Example 69, would include both the free base and its salt:

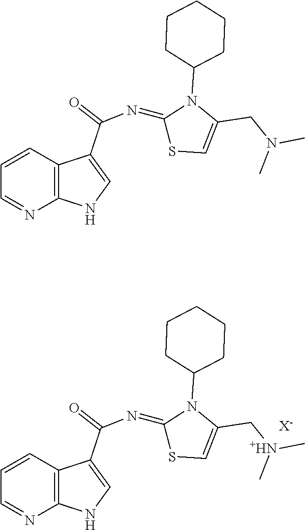

in which X⁻ is any counterion. In a particular embodiment, the term “compound of formula I” refers to the compound or a pharmaceutically acceptable salt thereof. The term “pharmaceutically acceptable salt” refers to salts prepared from pharmaceutically acceptable non-toxic acids or bases including inorganic acids and bases and organic acids and bases. When the compounds of the present invention are basic, as shown in the depiction above in this paragraph, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, for example when R¹ is COOH, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

### Methods of Treatment

The compositions described herein may be administered to a subject having or at risk of developing hearing loss (e.g., sensorineural hearing loss) and/or vestibular dysfunction by a variety of routes, such as local administration to the middle or inner ear (e.g., administration to or through the oval window, round window, or semicircular canal (e.g., the horizontal canal), or by transtympanic or intratympanic injection), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing hearing loss and/or vestibular dysfunction (e.g., subjects having or at risk of developing hearing loss, vestibular dysfunction, or both). The compositions and methods described herein can be used to treat subjects having or at risk of developing damage to cochlear hair cells (e.g., damage related to acoustic trauma, disease or infection, head trauma, ototoxic drugs, or aging), subjects having or at risk of developing damage to vestibular hair cells (e.g., damage related to disease or infection, head trauma, ototoxic drugs, or aging), subjects having or at risk of developing sensorineural hearing loss, deafness, or auditory neuropathy, subjects having or at risk of developing vestibular dysfunction (e.g., dizziness, vertigo, loss of balance, bilateral vestibulopathy, oscillopsia, or a balance disorder), subjects having tinnitus (e.g., tinnitus alone, or tinnitus that is associated with sensorineural hearing loss or vestibular dysfunction), subjects having a genetic mutation associated with hearing loss and/or vestibular dysfunction, or subjects with a family history of hereditary hearing loss, deafness, auditory neuropathy, tinnitus, or vestibular dysfunction. In some embodiments, the subject has or is at risk of developing hearing loss and/or vestibular dysfunction that is associated with or results from loss of hair cells (e.g., cochlear or vestibular hair cells). The methods described herein may include a step of screening a subject for one or more mutations in genes known to be associated with hearing loss and/or vestibular dysfunction prior to treatment with or administration of the compositions described herein. A subject can be screened for a genetic mutation using standard methods known to those of skill in the art (e.g., genetic testing). The methods described herein may also include a step of assessing hearing and/or vestibular function in a subject prior to treatment with or administration of the compositions described herein. Hearing can be assessed using standard tests, such as audiometry, auditory brainstem response (ABR), electrocochleography (ECOG), and otoacoustic emissions. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess hearing and/or vestibular function in a subject after treatment with or administration of the compositions described herein. The compositions and methods described herein may also be administered as a preventative treatment to patients at risk of developing hearing loss and/or vestibular dysfunction, e.g., patients who have a family history of hearing loss or vestibular dysfunction (e.g., inherited hearing loss or vestibular dysfunction), patients carrying a genetic mutation associated with hearing loss or vestibular dysfunction who do not yet exhibit hearing impairment or vestibular dysfunction, or patients exposed to risk factors for acquired hearing loss (e.g., acoustic trauma, disease or infection, head trauma, ototoxic drugs, or aging) or vestibular dysfunction (e.g., disease or infection, head trauma, ototoxic drugs, or aging).

The compositions and methods described herein can be used to induce or increase hair cell regeneration in a subject (e.g., cochlear and/or vestibular hair cell regeneration). Subjects that may benefit from compositions that induce or increase hair cell regeneration include subjects suffering from hearing loss or vestibular dysfunction as a result of loss of hair cells (e.g., loss of hair cells related to trauma (e.g., acoustic trauma or head trauma), disease or infection, ototoxic drugs, or aging), and subjects with abnormal hair cells (e.g., hair cells that do not function properly when compared to normal hair cells), damaged hair cells (e.g., hair cell damage related to trauma (e.g., acoustic trauma or head trauma), disease or infection, ototoxic drugs, or aging), or reduced hair cell numbers due to genetic mutations or congenital abnormalities.

The compositions and methods described herein can also be used to prevent or reduce hearing loss and/or vestibular dysfunction caused by ototoxic drug-induced hair cell damage or death (e.g., cochlear hair cell and/or vestibular hair cell damage or death) in subjects who have been treated with ototoxic drugs, or who are currently undergoing or soon to begin treatment with ototoxic drugs. Ototoxic drugs are toxic to the cells of the inner ear, and can cause sensorineural hearing loss, vestibular dysfunction (e.g., vertigo, dizziness, imbalance, bilateral vestibulopathy, oscillopsia, or a balance disorder), tinnitus, or a combination of these conditions. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods and compositions described herein can be used to treat bilateral vestibulopathy or oscillopsia. Bilateral vestibulopathy and oscillopsia can be induced by aminoglycosides (e.g., the methods and compositions described herein can be used to promote or increase hair cell regeneration in a subject having or at risk of developing aminoglycoside-induced bilateral vestibulopathy or oscillopsia).

Treatment may include administration of a composition containing a compound described herein in various unit doses. Each unit dose will ordinarily contain a predetermined-quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the inner ear (e.g., the cochlea and/or vestibular system).

The compositions described herein are administered in an amount sufficient to improve hearing, improve vestibular function (e.g., improve balance or reduce dizziness or vertigo), reduce tinnitus, treat bilateral vestibulopathy, treat oscillopsia, treat a balance disorder, increase or induce hair cell regeneration (e.g., cochlear and/or vestibular hair cell regeneration), increase hair cell numbers, activate YAP, and/or inhibit LATS. Hearing may be evaluated using standard hearing tests (e.g., audiometry, ABR, electrocochleography (ECOG), and otoacoustic emissions) and may be improved compared to hearing measurements obtained prior to treatment. Vestibular function may be evaluated using standard tests for balance and vertigo (e.g., eye movement testing (e.g., ENG or VNG), posturography, VOR testing (e.g., head impulse testing (Halmagyi-Curthoys testing, e.g., VHIT), or caloric reflex testing), rotary-chair testing, ECOG, VEMP, and specialized clinical balance tests) and may be improved compared to measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss and/or vestibular dysfunction (e.g., in subjects who carry a genetic mutation associated with hearing loss or vestibular dysfunction, who have a family history of hearing loss or vestibular dysfunction (e.g., hereditary hearing loss or vestibular dysfunction), or who have been exposed to risk factors associated with hearing loss or vestibular dysfunction (e.g., ototoxic drugs, head trauma, disease or infection, or acoustic trauma) but do not exhibit hearing impairment or vestibular dysfunction (e.g., vertigo, dizziness, or imbalance), or in subjects exhibiting mild to moderate hearing loss or vestibular dysfunction). These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Preparation of Compounds

The following abbreviations are used in the synthetic routes: THF (tetrahydrofuran), MeOH (methanol), DCM (dicholoromethane), DMF (N,N-dimethylformamide), ACN (acetonitrile), EtOH (ethanol), EtOAc (ethyl acetate), IPA (2-propanol), DMSO (dimethyl sulfoxide), MTBE (methyl tert-butyl ether), TEA (triethylamine), DIPEA (N,N-diisopropylethylamine), TMEDA (tetramethylethylenediamine), DMAP (N,N-dimethylpyridin-4-amine), EDCI (N-(3-Dimethylaminopropyl)-N′-ethylcarbodiimide hydrochloride), HOBt (1-Hydroxybenzotriazole hydrate), HBTU ((2-(1H-benzotriazol-1-yl)-1,1,3,3-tetramethyluronium hexafluorophosphate), T₃P (propanephosphonic acid anhydride), TBAI (tetrabutylammonium iodide), LAH (lithium aluminum hydride), TFA (trifluoroacetic acid).

Preparative HPLC purification refers to the use of a water/acetonitrile gradient with or without the use of additives such as HCl, formic acid, TFA, or NH₄HCO₃ using an appropriate hydrophobic stationary phase.

The N-benzyl thiazole compound

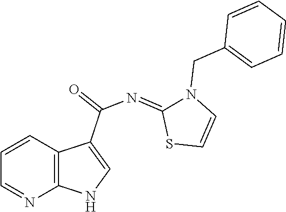 [SMILES: O=C(/N=c1\sccn1Cc1ccccc1)c1c[nH]c2ncccc12]

was tested in vitro to explore its mechanism of action and its utility in regenerating hair cells of the ear. The effects of the compounds were tested on utricles isolated from mice eight to twelve weeks of age. Internal ears were dissected from mice euthanized with fluothane and placed into ice-cold Hank's balanced salt solution, and cultured as previously described by Gnedeva, K. & Hudspeth [Proc. Natl. Acad Sci. 112, 14066-14071 (2015)]. For proliferation assays, utricles were cultured with 10 μM 5-ethynyl-2′-deoxyuridine (EdU) that was detected with click chemistry.

Immunohistochemical analysis demonstrated that the N-benzyl thiazole drove robust Yap nuclear translocation in supporting cells after 24 hr of treatment at a concentration of 10 μM (quantified as a ratio to the constitutively expressed protein Sall2; control=0.6; TO1-treated=1.0; p<0.0001 by an unpaired, two-tailed t-test, n=570 control nuclei and 680 treated nuclei), and it caused a striking reduction in the level of Yap phosphorylation as detected by western blot. After 5 days of treatment, the N-benzyl thiazole evoked robust re-entry into the cell cycle of adult utricular supporting cells, yielding hundreds of EdU+daughter supporting cells (control=20 EdU+ supporting cells; the N-benzyl thiazole-treated=250 EdU+supporting cells; p=0.021 by an unpaired, one-tailed 1-test, control n=2, TO1 n=3).

### General Schemes

The compounds of the present invention can be prepared as illustrated in the General Schemes I-IV and in greater detail in Schemes 1-65 below. Detailed description for the synthesis of the intermediates and exemplified compounds are also disclosed below.

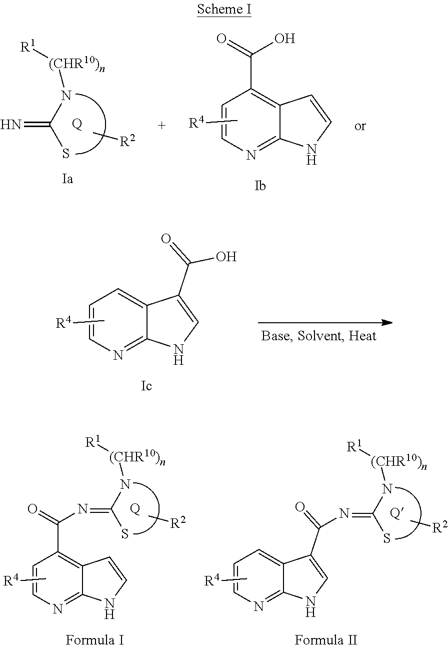

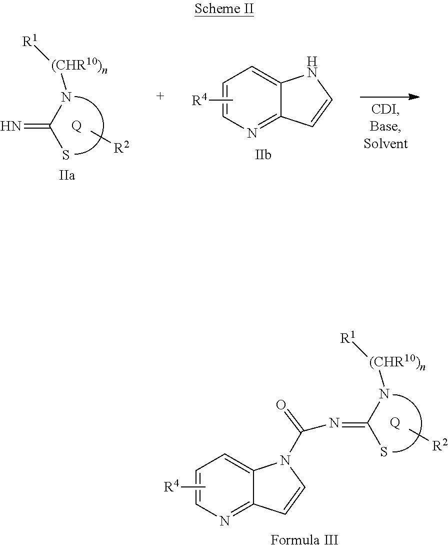

As shown in Scheme I-II, compounds of formula (Ia-Ia) containing an imine group when treated with compounds of formula (Ib-IIb) under coupling conditions known to one skilled in the art, will provide compounds of formula (I-III). Typical conditions for the reaction of carboxylic acid compounds of formula (Ib-Ic) with compounds of formula (Ia) include but are not limited to stirring an equimolar mixture of the compounds with a coupling reagent such as but not limited to N-(3-dimethylaminopropyl)-N′-ethylcarbodiimide hydrochloride (EDCI), 1-hydroxybenzotriazole hydrate (HOBt), ((2-(1H-benzotriazol-1-yl)-1,1,3,3-tetramethyluronium hexafluorophosphate (HBTU), propanephosphonic acid anhydride (T₃P), in the presence of a base such as but not limited to N,N-di-isopropylethylamine (DIPEA) in solvents such as but not limited to DMF, EtOAc and pyridne. Typical reactions can be carried out be carried out between 25-110° C. for 1-12 hours. Typical conditions for the reaction of azaindole compounds of formula (IIb) with compounds of formula (IIa) include but are not limited to stirring an equimolar mixture of the compounds with a coupling reagent such as but not limited to 1,1-carbonyldiimidazole (CDI), in the presence of a base such as but not limited to NaH in solvents such as but not limited to THF. Typical reactions can be carried out be carried out between 25° C. for 1-2 hours.

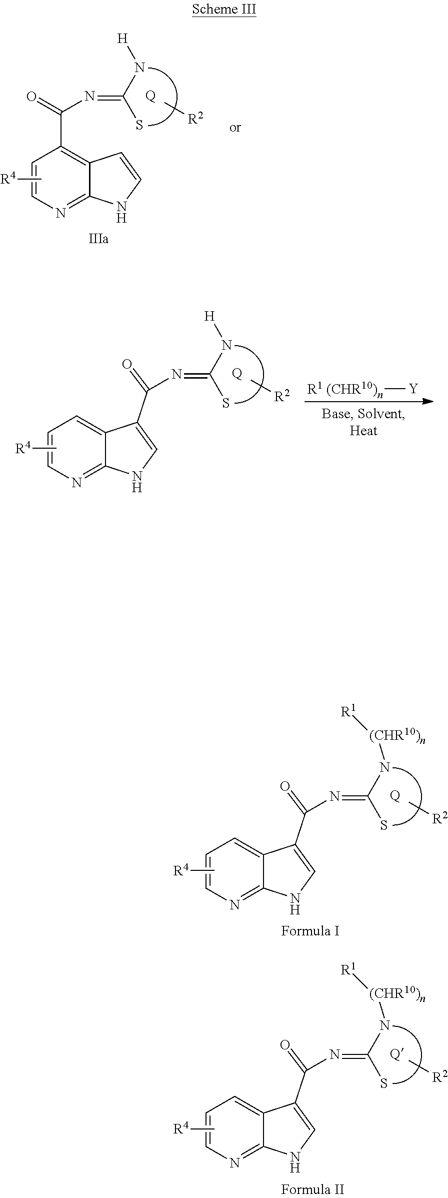

As shown in Scheme III, compounds of formula (IIIa) may be converted into compounds of formula (I-III) which are representative compounds of the present invention. Typical conditions include, but not limited to, the treatment of compounds of formula (MIIa) with potassium carbonate (K₂CO₃) in DMF at 25° C., followed by the addition of reagents such as R¹—(CHR¹⁰)ₙ— Y, where R₁ is defined in formula (I-II) and Y is chloro, bromo, iodo, mesyl or tosylate. Typical reactions can be carried out at 100° C. in a microwave reactor to facilitate the alkylation.

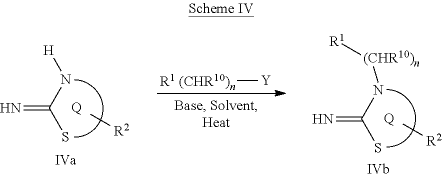

Compounds of formula (Ia, IIa) in Scheme I-II may be prepared according to the methods outlined in Scheme IV. Compounds of formula (IVa) when treated with potassium carbonate (K₂CO₃) in DMF at 25° C., followed by the addition of reagents such as R¹—(CHR¹⁰)ₙ— Y, where R₁ is defined in formula (I-III) and Y is chloro, bromo, iodo, mesyl or tosylate will provide compounds of formula (IVb). Typical reactions can be carried out at 100° C. in a microwave reactor to facilitate the alkylation.

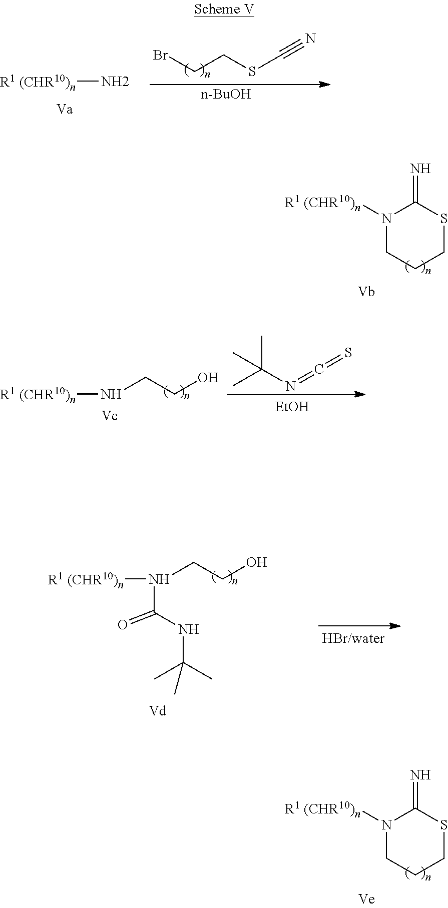

Scheme V describeds other alternative methods of preparation of compounds of formula (Ia-IIa) in Scheme I-II. Compounds of formula (Va) when treated with 1-bromo-2-thiocyanatoethane or 1-bromo-3-thiocyanatopropane will provide compound formula (Vb). Amino alcohol compounds of formula (Vc) when treated with 2-isothiocyanato-2-methylpropane in EtOH will provide compounds of formula (Vd). Compounds of formula (Vd) when treated with hydrogen bromide (HBr) in the water will provide compounds of formula (Ve).

The following abbreviations are used in the synthetic routes: THF (THF), MeOH (methanol), EtOH (ethanol), DCM (dicholoromethane), DCE (1,2-dichloroethane), DMF (N,N-dimethylformamide), ACN (acetonitrile), EtOH (ethanol), EtOAc (EtOAc), IPA (2-propanol), DMSO (dimethyl sulfoxide), MTBE (methyl tert-butyl ether), Et₃N (triethylamine), Py (pyridine), DIPEA (N,N-diisopropylethylamine), TMEDA (tetramethylethylenediamine), DMAP (N,N-dimethylpyridin-4-amine), TBD (triazabicyclodecene or 1,5,7-triazabicyclo[4.4.0]dec-5-ene), EDCI (N-(3-Dimethylaminopropyl)-N′-ethylcarbodiimide hydrochloride), HOBt (1-Hydroxybenzotriazole hydrate), HBTU ((2-(1H-benzotriazol-1-yl)-1,1,3,3-tetramethyluronium hexafluorophosphate), T₃P (propanephosphonic acid anhydride), CDI (1,1′-carbonyldiimidazole), TBAI (tetrabutylammonium iodide), TBAF (tetra-n-butylammonium fluoride), LAH (lithium aluminum hydride), DIBAL-H (diisobutylaluminium hydride), XPhos (2-dicyclohexylphosphino-2′,4′,6′-triisopropylbiphenyl), Dess Martin periodinane (1,1,1-tris(acetyloxy)-1,1-dihydro-1,2-benziodoxol-3-(1H)-one), TFA (trifluoroacetic acid), FA (formic acid), HBr (hydrobromic acid), SOCl₂ (thionyl chloride), BOP—Cl (bis(2-oxo-3-oxazolidinyl)phosphinic chloride), TosCl (p-toluenesulfonyl chloride), Tos-OH (p-toluenesulfonic acid), MsCl (methanesulfonyl chloride), Ms₂O (methanesulfonic anhydride), Boc₂O (di-tert-butyl dicarbonate), SEM-Cl (2-(trimethylsilyl)ethoxymethyl chloride), TMSBr (bromotrimethylsilane), BnBr (benzyl bromide).

Preparative HPLC purification refers to the use of a water/acetonitrile gradient with or without the use of additives such as HCl, formic acid, TFA, or NH₄HCO₃ using an appropriate hydrophobic stationary phase.

The carboxylic acids in Table A used for examples are commercially available. Azaindole A5 was prepared as depicted in Scheme 1.

TABLE A

| Azaindoles | Structure | Reference |
|---|---|---|
| A1 | 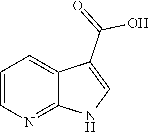 [SMILES: O=C(O)c1c[nH]c2ncccc12] | 156270-06-3 |
| A2 | 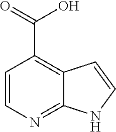 [SMILES: O=C(O)c1ccnc2[nH]ccc12] | 1000340-27-1 |
| A3 | 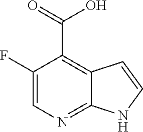 [SMILES: O=C(O)c1c(F)cnc2[nH]ccc12] | 1228666-41-8 |
| A4 | 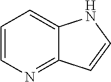 | 272-49-1 |

### Preparation of 1H-Pyrrolo[3,2-b] pyridine-2-carbonitrile (A5)

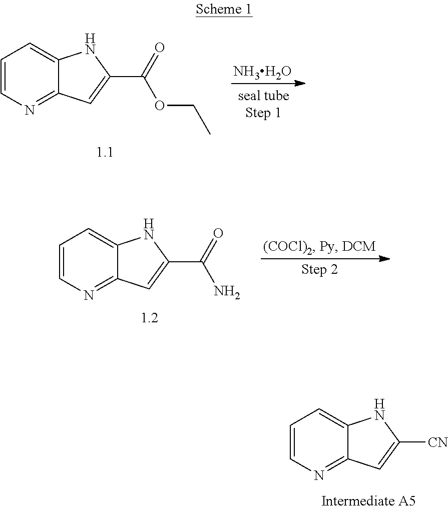

### Step 1. 1H-Pyrrolo[3,2-b]pyridine-2-carboxamide (1.2)

A solution of ethyl 1H-pyrrolo[3,2-b] pyridine-2-carboxylate (1.1) (2.00 g, 10.5 mmol) in ammonium hydroxide (20 mL, 37% in water) was stirred at 70° C. for 12 hours in a sealed tube. The resulting precipitate was collected by filtration, washed with water (50 mL) and dried under reduced pressure to obtain 1.50 g of the title compound (1.2) as a solid.

¹H NMR (400 MHz, DMSO-d₆) δ 11.6 (br. s, 1H), 8.38 (dd, J=1.6 Hz, 4.4 Hz, 1H), 8.12 (s, 1H), 7.77 (d, J=8.0 Hz, 1H), 7.54 (s, 1H), 7.27 (s, 1H), 7.18 (dd, J=4.4 Hz, 8.4 Hz, 1H).

### Step 2. 1H-Pyrrolo[3,2-b] pyridine-2-carbonitrile (A5)

To a solution of 1H-pyrrolo[3,2-b] pyridine-2-carboxamide (1.2) (1.00 g, 6.20 mmol) in DCM (30 mL) was added pyridine (1.47 g, 18.6 mmol) and oxalyl chloride (1.58 g, 12.4 mmol) at 0° C. The resulting mixture was warmed up to 20° C. and stirring continued for 4 hours before quenching with water (10 mL). The precipitate formed was collected by filtration and dried under reduced pressure. The residue was purified by preparative HPLC (Phenomenex Gemini, 25×150 mm, 10 mm; mobile phase A: water/0.05% NH₄OH, mobile phase B: MeCN; gradient: 1% B to 31% B over 10 min) to afford 30.0 mg of A5 as a solid.

LCMS (m/z [M+H]⁺): 144.1

### Preparation of 3-Benzylthiazolidin-2-imine (B1)

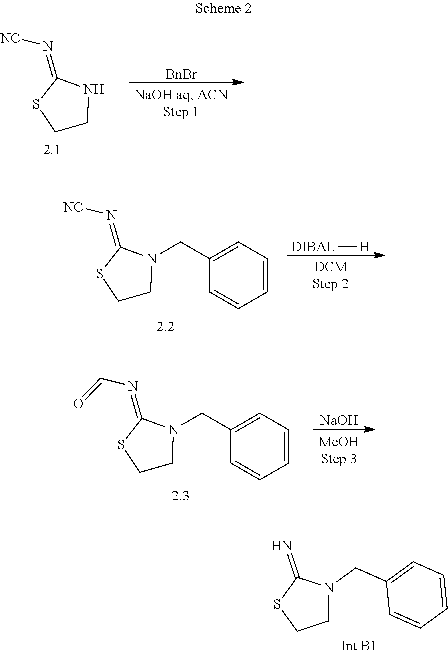

### Step 1. (Z)—N-(3-Benzylthiazolidin-2-ylidene) cyanamide (2.2)

To a solution of (Z)—N-(thiazolidin-2-ylidene) cyanamide (2.1) (0.318 g, 2.50 mmol) and sodium hydroxide (1.0 M, 14 mL) was added benzyl bromide (0.297 mL, 2.50 mmol) in acetonitrile (20 mL). The resulting mixture was stirred at 20° C. for 12 hours. The reaction was diluted with water (50 mL) and extracted with EtOAc (3×50 mL). The organic extracts were combined, washed with brine (3×100 mL), dried over anhydrous Na₂SO₄, filtered and evaporated. The residue was purified by silica gel column chromatography, eluting with 20% EtOAc in petroleum ether to afford 0.385 g of the title compound (2.2) as a solid.

¹H NMR (400 MHz, CDCl₃) δ 7.32-7.23 (m, 3H), 7.21-7.16 (m, 2H), 4.54 (s, 2H), 3.68 (t, J=7.6 Hz, 2H), 3.27 (t, J=7.6 Hz, 2H)

LCMS (m/z [M+H]⁺): 218.5

### Step 2. (Z)—N-(3-Benzylthiazolidin-2-ylidene) formamide (2.3)

To a solution of (Z)-(3-benzylthiazolidin-2-ylidene) cyanamide (2.2) (0.185 g, 0.851 mmol) in CH₂Cl₂ (2 mL) at −70° C. was added diisobutylaluminum hydride (1 M, 1.70 mL). The resulting mixture was stirred at −70° C. for 2 hours under a nitrogen atmosphere before quenching with an ice cold saturated aqueous ammonium chloride solution(40 mL) followed by dilution with EtOAc (40 mL), and filtration. The filtrate was adjusted to pH=9 with saturated aqueous sodium carbonate and then extracted with EtOAc (3×50 mL). The combined organic phases were washed with brine (4×40 mL), dried over anhydrous Na₂SO₄, filtered, and evaporated to afford 0.160 g of the crude compound (2.3) as an oil.

LCMS (m/z [M+H]⁺): 221.1

### Step 3. 3-Benzylthiazolidin-2-imine (B1)

To a solution of (Z)—N-(3-benzylthiazolidin-2-ylidene) formamide (2.3) (0.160 g, 0.726 mmol) in MeOH (1 mL) at 0° C. was added a solution of sodium hydroxide (0.300 g, 7.50 mmol) in water (1 mL) and the resulting mixture stirred at 0° C. for 1 hour, diluted with saturated aqueous sodium bicarbonate (50 mL) and extracted with EtOAc (3×50 mL). The combined organic phases were washed with brine (3×100 mL), dried over anhydrous Na₂SO₄, filtered, and evaporated to afford 0.100 g of B1 as an oil.

¹H NMR (400 MHz, CDCl₃) δ 7.38-7.28 (m, 5H), 4.58 (s, 2H), 3.50 (t, J=6.8 Hz, 2H), 3.14 (t, J=6.8 Hz, 2H)

LCMS (m/z [M+H]⁺): 193.1

### Preparation of 2-(2-Iminothiazolidin-3-yl)-N,N-dimethylacetamide (B2)

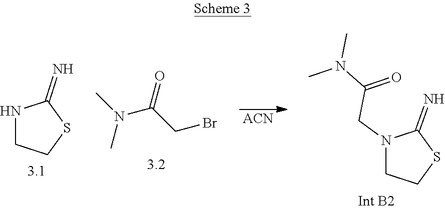

### 2-(2-Iminothiazolidin-3-yl)-N,N-dimethylacetamide (B2)

A solution of thiazolidin-2-imine (3.1) (0.500 g, 4.89 mmol) and 2-bromo-N, N-dimethyl-acetamide (3.2) (0.812 g, 4.89 mmol) in acetonitrile (10 mL) was stirred at 50° C. for 12 hours. The resulting precipitate was collected, washed with acetonitrile (10 mL) and dried under reduced pressure to afford 0.600 g of B2 as a solid.

¹H NMR (400 MHz, DMSO-d₆) δ 9.68 (br. s, 1H), 4.56 (s, 2H), 3.93 (t, J=7.6 Hz, 2H), 3.51 (t, J=7.6 Hz, 2H), 2.92 (s, 3H), 2.85 (s, 3H)

LCMS (m/z [M+H]⁺): 188.1

### Preparation of 2-Imino-3-phenyl-2,3-dihydro-4H-1,3-thiazin-4-one (B3)

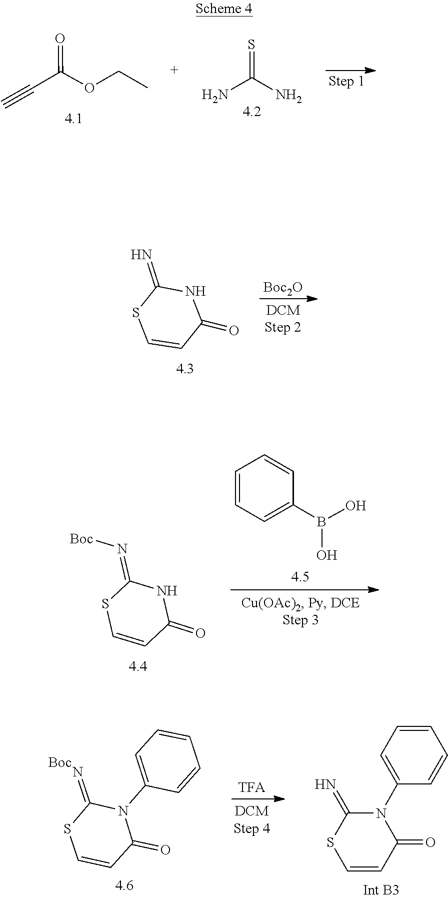

### Step 1. 2-Imino-2,3-dihydro-4H-1,3-thiazin-4-one (4.3)

A solution of thiourea (4.2) (10.0 g, 131 mmol) in MeOH (65 mL) was cooled to 0-5° C., ethyl propiolate (4.1) (19.3 g, 197 mmol, 19.3 mL) added and the resulting solution was kept in the refrigerator at 0-5° C. for 16 hrs. The resulting precipitate was filtered and triturated with water (60 mL). The product was dissolved in water and the undissolved solid impurity was filtered and discarded. The aqueous solution was concentrated under reduced pressure to afford 2.30 g of the title compound (4.3) as a white solid.

¹H NMR (400 MHz, DMSO-d₆) δ 8.21 (br. s, 2H), 7.67 (d, J=10.4 Hz, 1H), 6.32 (d, J=10.0 Hz, 1H)

LCMS (m/z [M+H]⁺): 129.4

### Step 2. tert-Butyl (Z)-(4-oxo-3,4-dihydro-2H-1,3-thiazin-2-ylidene)carbamate (4.4)

To a mixture of 2-imino-2,3-dihydro-4H-1,3-thiazin-4-one (4.3) (2.30 g, 17.9 mmol), Et₃N (5.45 g, 53.8 mmol) in THF (20 mL) and H₂O (2 mL) was added Boc₂O (4.70 g, 21.5 mmol) at 25° C. The resulting mixture was stirred for 24 hours at 25° C. and continued to stir at 70° C. for 12 hr. The reaction mixture was allowed to cool, poured into water (50 mL) and extracted with EtOAc (3×30 mL). The combined organic phases were washed with brine (3×20 mL), dried over Na₂SO₄, filtered, and evaporated. The residue was purified by flash silica chromatography using an elution gradient 10 to 50% EtOAc in petroleum ether to afford 0.700 g of the title compound (4.4) as a solid.

¹H NMR (400 MHz, DMSO-d₆) δ 11.72 (br. s, 1H), 7.93 (d, J=10.4 Hz, 1H), 6.55 (d, J=10.4 Hz, 1H), 1.45 (s, 9H)

### Step 3. tert-Butyl (Z)-(4-oxo-3-phenyl-3,4-dihydro-2H-1,3-thiazin-2-ylidene) carbamate (4.6)

To a mixture of tert-butyl (Z)-(4-oxo-3,4-dihydro-2H-1,3-thiazin-2-ylidene) carbamate (4.4) (0.700 g, 3.07 mmol), phenylboronic acid (4.5) (0.75 g, 6.13 mmol) and pyridine (0.97 g, 12.3 mmol) in dichloroethane (20 mL) was added copper acetate (1.11 g, 6.13 mmol) at 25° C. The resulting mixture was stirred for 12 hours at 50° C. under an oxygen atmosphere and then poured into ice-water (20 mL). The aqueous phase was extracted with EtOAc (3×15 mL). The combined organic phases were washed with brine (20 mL), dried with anhydrous Na₂SO₄, filtered, and evaporated. The residue was purified by flash silica chromatography using an elution gradient of 10 to 50% EtOAc in petroleum ether to afford 0.700 g of the title compound (4.6) as a solid.

¹H NMR (400 MHz, CDCl₃) δ 7.53 (d, J=10.0 Hz, 1H), 7.41-7.37 (m, 3H), 7.12 (d, J=8.8 Hz, 2H), 6.64 (d, J=10.0 Hz, 2H), 1.48 (s, 9H)

LCMS (m/z [M+H]⁺): 305.1

### Step 4. 2-Imino-3-phenyl-2,3-dihydro-4H-1,3-thiazin-4-one (B3)

To a mixture of tert-butyl (Z)-(4-oxo-3-phenyl-3,4-dihydro-2H-1,3-thiazin-2-ylidene) carbamate (4.6) (0.100 g, 0.330 mmol) in DCM (2 mL) was added TFA (3.08 g, 27.0 mmol) at 0° C. The reaction mixture was stirred for 0.5 hour at 25° C. and then poured into a sat. solution of sodium bicarbonate (20 mL). The aqueous phase was extracted with EtOAc (3×20 mL). The combined organic phases were washed with brine (20 mL), dried over anhydrous Na₂SO₄, filtered, and evaporated. The residue was purified by flash silica chromatography using an elution gradient of 5 to 50% EtOAc in petroleum ether to afford 0.06 g of B3 as a yellow oil.

¹H NMR (400 MHz, CDCl₃) δ 7.38 (t, J=8.0 Hz, 2H), 7.22-7.16 (m, 2H), 6.98 (d, J=7.6 Hz, 2H), 6.38 (d, J=10.4 Hz, 1H)

LCMS (m/z [M+H]⁺): 205.1

The preparation of intermediates C1-C37 in Table C is described in methods shown below in Schemes 5-34.

TABLE C

| Int. | Structure | LCMS | 1H NMR |
|---|---|---|---|
| C1 | 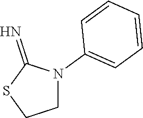 [SMILES: N=C1SCCN1c1ccccc1] | 179.2 | (CDCl₃, 400 MHz) δ 11.16 (br. s, 1H), 7.63 − 7.51 (m, 3H), 7.41 (d, J = 7.2 Hz, 2H), 4.37 (t, J = 7.6 Hz, 2H), 3.67 (t, J = 7.6 Hz, 2H). |
| C2 | 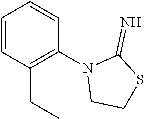 | 207.2 | (DMSO-d₆, 400 MHz) δ 9.90 (br. s, 1H), 8.78 (br. s, 1H), 7.55 − 7.47 (m, 3H), 7.45 − 7.39 (m, 1H), 4.36 − 4.28 (m, 1H), 4.23 − 4.19 (m, 1H), 3.79 − 3.64 (m, 2H), 2.58 − 2.52 (m, 2H), 1.20 (t, J = 7.6 Hz, 3H). |
| C3 | 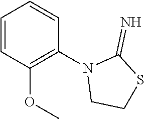 | 209.0 | NA |
| C4 | 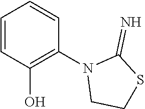 [SMILES: N=C1SCCN1c1ccccc1O] | 209.0 | (DMSO-d₆, 400 MHz) δ 9.22 (br. s, 1H), 7.24 (dd, J = 1.6, 8.0 Hz, 1H), 7.15 − 7.11 (m, 1H), 6.91 − 6.83 (m, 2H), 4.10 (t, J = 7.2 Hz, 2H), 3.44 (t, J = 6.8 Hz, 2H). |
| C5 | 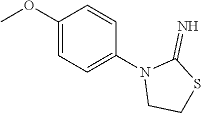 | NA | (DMSO-d₆, 400 MHz) δ 7.39 (d, J = 8.0 Hz, 2H), 6.91 (d, J = 8.8 Hz, 2H), 3.98 (t, J = 6.8 Hz, 2H), 3.73 (s, 3H), 3.25 (t, J = 6.8 Hz, 2H). |
| C6 | 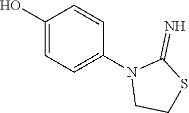 [SMILES: N=C1SCCN1c1ccc(O)cc1] | NA | (DMSO-d₆, 400 MHz) δ 9.98 (br. s, 1H), 8.70 (br. s, 1H), 7.31 − 7.27 (m, 2H), 6.93 − 6.89 (m, 2H), 4.26 (t, J = 7.6 Hz, 2H), 3.61 (t, J = 7.6 Hz, 2H). |
| C7 | 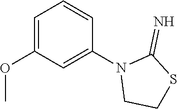 | 209.2 | NA |
| C8 | 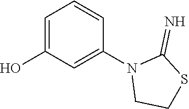 [SMILES: N=C1SCCN1c1cccc(O)c1] | 195.2 | NA |
| C9 | 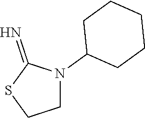 [SMILES: N=C1SCCN1C1CCCCC1] | 185.1 | (D₂O, 400 MHz) δ 3.76 − 3.66 (m, 1H), 3.56 − 3.53 (m, 2H), 3.46 − 3.43 (m, 2H), 1.86 − 1.72 (m, 5H), 1.68 − 1.51 (m, 4H), 1.33 − 1.21 (m, 1H). |
| C10 | 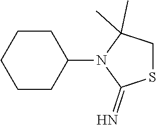 | 212.9 | (DMSO-d₆, 400 MHz) δ 9.60 (br. s, 1H), 8.39 (br. s, 1H), 3.57 − 3.44 (m, 1H), 3.36 (s, 2H), 2.12 − 1.96 (m, 2H), 1.81 − 1.72 (m, 2H), 1.61 − 1.29 (m, 12H) |
| C11 | 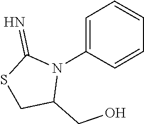 [SMILES: N=C1SCC(CO)N1c1ccccc1] | NA | (CD₃OD, 400 MHz) δ 7.67 − 7.56 (m, 3H), 7.54 − 7.47 (m, 2H), 4.74 − 4.68 (m, 1H), 3.95 − 3.87 (m, 1H), 3.80 − 3.70 (m, 1H), 3.65 − 3.59 (m, 2H). |
| C12 | 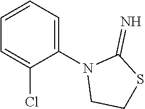 [SMILES: N=C1SCCN1c1ccccc1Cl] | 213.2 | (DMSO-d₆, 400 MHz) δ 10.1(br. s, 1H),9.13 (br. s, 1H), 7.75 (dd, J = 2.4, 6.4 Hz, 2H), 7.63 − 7.54 (m, 2H), 4.35 − 4.19 (m, 2H), 3.79 − 3.66 (m, 2H). |
| C13 | 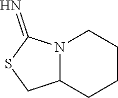 | 156.9 | (DMSO-d₆, 400 MHz) δ 8.60 (br. s, 1H), 3.96 − 3.91 (m, 1H), 3.63 − 3.59 (m, 2H), 3.46 − 3.42 (m, 2H), 1.91 − 1.78 (m, 2H), 1.56 − 1.30 (m, 4H). |
| C14 | 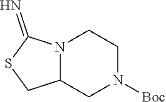 [SMILES: CC(C)(C)OC(=O)N1CCN2C(=N)SCC2C1] | 258.0 | (DMSO-d₆, 400 MHz) δ 9.53 (br. s, 1H), 4.22 − 4.19 (m, 1H), 4.07 − 3.92 (m, 3H), 3.68 (dd, J = 7.6, 11.2Hz, 1H), 3.25 − 3.09 (m, 2H), 2.91 − 2.85 (m, 2H), 1.42 (s, 9H). |
| C15 | 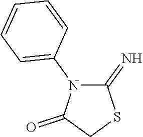 [SMILES: N=C1SCC(=O)N1c1ccccc1] | 193.1 | (CDCl₃, 400 MHz) δ 8.07 (br s, 1H), 7.50 (d, J = 7.6 Hz, 2H), 7.35 (t, J = 7.2 Hz, 2H), 7.18 (t, J = 8.0 Hz, 1H), 3.88 (s, 2H). |
| C16 | 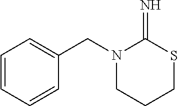 [SMILES: N=C1SCCCN1Cc1ccccc1] | 207.1 | (DMSO-d₆, 400 MHz) δ 9.38 (br. s, 2H), 7.44 − 7.40 (m, 2H), 7.37 − 7.33 (m, 1H), 7.31 − 7.28 (m, 2H), 4.89 (s, 2H), 3.49 (t, J = 5.2Hz, 2H), 3.21 (t, J = 6.0 Hz, 2H), 2.13 − 2.07 (m, 2H). |
| C17 | 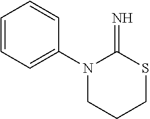 [SMILES: N=C1SCCCN1c1ccccc1] | 193.1 | (D₂O, 400 MHz) δ 7.57 − 7.50 (m, 3H), 7.41 − 7.35 (m, 2H), 3.81 (t, J = 6.0 Hz, 2H), 3.29 (t, J = 6.0 Hz, 2H), 2.47 − 2.28 (m, 2H). |
| C18 | 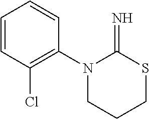 [SMILES: N=C1SCCCN1c1ccccc1Cl] | 226.9 | (DMSO-d₆ + D₂O, 400 MHz) δ 7.76 − 7.72 (m, 1H), 7.68 − 7.64 (m, 1H), 7.61 − 7.54 (m, 2H), 3.81 − 3.73 (m, 1H), 3.64 − 3.58 (m, 1H), 3.41 − 3.28 (m, 2H), 2.43 − 2.23 (m, 1H), 2.30 − 2.20 (m, 1H). |
| C19 | 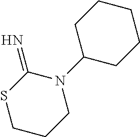 [SMILES: N=C1SCCCN1C1CCCCC1] | 198.9 | (CDCl₃, 400 MHz) δ 9.52 (br. s, 1H), 9.16 (br. s, 1H), 3.79 − 3.71 (m, 2H), 3.22 − 3.08 (m, 2H), 3.06 − 2.91 (m, 1H), 2.29 − 2.15 (m, 2H), 2.14 − 2.03 (m, 1H), 1.84 − 1.66(m, 6H), 1.57 − 1.39 (m, 2H), 1.28 − 1.23 (m, 1H). |
| C20 | 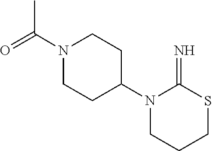 [SMILES: CC(=O)N1CCC(N2CCCSC2=N)CC1] | NA | (CD₃OD, 400 MHz) δ 4.74 − 4.65 (m, 1H), 4.35 − 4.24 (m, 1H), 4.13 − 4.04 (m, 1H), 3.62 − 3.53 (m, 2H), 3.29 − 2.23 (m, 1H), 3.22 − 3.18 (m, 2H), 2.73 − 2.68 (m, 1H), 2.25 − 2.21 (m, 2H), 2.17 (s, 3H), 1.97 − 1.88 (m, 2H), 1.87 − 1.83 (m, 2H). |
| C21 | 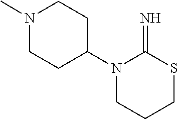 | 214.1 | (CD₃OD, 400 MHz) δ 3.73 − 3.66 (m, 5H), 3.24 (t, J = 7.6 Hz, 2H), 3.08 (t, J = 7.6 Hz, 1H), 2.93 (s, 3H), 2.47 − 2.43 (m, 2H), 2.15 − 2.14 (m, 2H), 2.02 − 1.98 (m, 2H), 1.96 − 1.89 (m, 1H). |
| C22 | 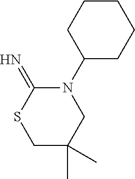 | 226.8 | (CD₃OD, 400 MHz) δ 7.71 (d, J = 8.0 Hz, 1H), 7.24 (d, J = 8.0 Hz, 1H), 3.57 − 3.47 (m, 1H), 3.21 (s, 2H), 3.04 (s, 2H), 1.98 − 1.91 (m, 2H), 1.84 − 1.78 (m, 2H), 1.68 − 1.64 (m, 1H), 1.43 − 1.28 (m, 5H), 1.17 (s, 6H). |
| C23 | 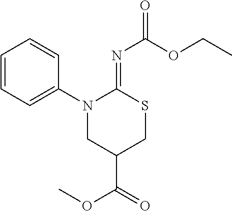 [SMILES: CCOC(=O)/N=C1\SCC(C(=O)OC)CN1c1ccccc1] | NA | (DMSO-d₆, 400 MHz) δ 7.48 − 7.38 (m, 2H), 7.34 − 7.28 (m, 1H), 7.25 (d, J = 7.6 Hz, 2H), 3.96 − 3.80 (m, 4H), 3.69 (s, 3H), 3.59 − 3.46 (m, 1H), 3.42 − 3.35 (m, 1H), 3.30 − 3.24 (m, 1H), 1.06 (t, J = 6.8 Hz, 3H). |
| C24 | 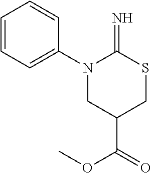 [SMILES: COC(=O)C1CSC(=N)N(c2ccccc2)C1] | NA | NA |
| C25 | 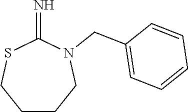 [SMILES: N=C1SCCCCN1Cc1ccccc1] | 221.0 | (DMSO-d₆, 400 MHz) δ 7.33 − 7.31 (m, 3H), 7.28 − 7.19 (m, 2H), 4.58 (s, 2H), 3.39 − 3.36 (m, 2H), 2.88 − 2.85 (m, 2H), 1.84 − 1.75 (m, 2H), 1.53 − 1.47 (m, 2H). |
| C26 | 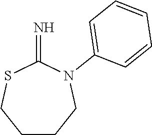 [SMILES: N=C1SCCCCN1c1ccccc1] | 207.2 | (DMSO-d₆, 400 MHz) δ 9.89 (br. s, 1 H), 8.45 (br. s, 1 H), 7.58 − 7.50 (m, 5 H), 4.07 − 4.04 (m, 2 H), 3.41 − 3.36 (m, 2 H), 2.00 − 1.99 (m, 2 H), 1.72 − 1.71 (m, 2 H). |
| C27 | 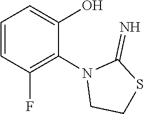 [SMILES: N=C1SCCN1c1c(O)cccc1F] | NA | (CD₃OD, 400 MHz) δ 6.93 (q, J = 8.4 Hz, 1H), 6.48 (d, J = 8.4 Hz, 1H), 6.18 − 6.13 (m, 1H), 4.09 (d, J = 2.0 Hz, 1H), 3.82 − 3.78 (m, 1H), 3.56 − 3.48 (m, 1H), 3.27 − 3.24 (m, 1H). |
| C28 | 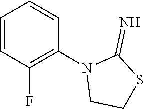 [SMILES: N=C1SCCN1c1ccccc1F] | 197.1 | (DMSO-d₆, 400 MHz) δ 10.16 (br s, 1H), 9.25 (br s, 1H), 7.71 − 7.67 (m, 1H), 7.62 − 7.58 (m, 1H), 7.53 − 7.48 (m, 1H), 7.71 − 7.67 (m, 1H), 7.43 − 7.39 (m, 1H), 4.33 (s, 2H), 3.70 (t, J = 7.6 Hz, 2H). |
| C29 | 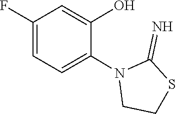 [SMILES: N=C1SCCN1c1ccc(F)cc1O] | 213.1 | (DMSO-d₆, 400 MHz) δ 7.45 − 7.41 (m, 1H), 6.82 − 6.78 (m, 2H), 4.17 (t, J = 7.6 Hz, 2H), 3.61 (t, J = 7.6 Hz, 2H). |
| C30 | 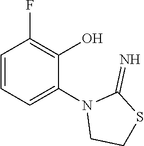 [SMILES: N=C1SCCN1c1cccc(F)c1O] | NA | (DMSO-d₆, 400 MHz) δ 10.73 (br. s, 1H), 9.93 (d, J = 6.4 Hz, 1H), 8.91 (s, 1H), 7.38 − 7.24 (m, 2H), 7.00 − 6.94 (m, 1H), 4.25 − 4.21 (m, 2H), 3.67 − 3.65 (m, 2H). |
| C31 | 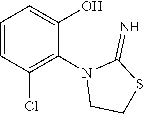 [SMILES: N=C1SCCN1c1c(O)cccc1Cl] | NA | (DMSO-d₆, 400 MHz) δ 10.97 (br.s, 1 H), 10.02 (s, 1 H), 7.40 − 7.36 (m, 1 H), 7.11 (d, J = 7.2 Hz, 1H), 7.03 (d, J = 8.4 Hz, 1 H), 4.21 − 4.10 (m, 2 H), 3.72 − 3.69 (m, 2 H). |
| C32 | 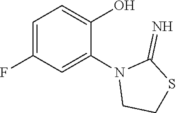 [SMILES: N=C1SCCN1c1cc(F)ccc1O] | NA | NA |
| C33 | 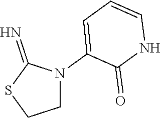 [SMILES: N=C1SCCN1c1ccc[nH]c1=O] | 196.2 | (DMSO-d₆, 400 MHz) δ 11.92 (br.s, 1H), 7.50 (dd, J = 2.0, 2.0 Hz, 1H), 7.39 (d, J = 5.2 Hz, 1H), 6.23 (t, J = 6.8 Hz, 1H), 3.95 (t, J = 3.2 Hz, 2H), 3.42 (t, J = 7.2 Hz, 2H). |
| C34 | 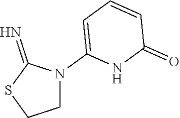 [SMILES: N=C1SCCN1c1cccc(=O)[nH]1] | NA | NA |
| C35 | 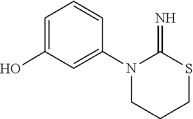 [SMILES: N=C1SCCCN1c1cccc(O)c1] | NA | NA |
| C36 | 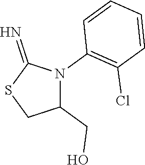 [SMILES: N=C1SCC(CO)N1c1ccccc1Cl] | NA | (DMSO-d₆, 400 MHz) δ 7.60 (t, J = 5.2 Hz, 1H), 7.64 − 7.41 (m, 3H), 5.03 (t, J = 4.8 Hz, 1H), 4.21 − 4.20 (m, 1H), 3.62 − 3.58 (m, 1H), 3.41 − 3.37 (m, 4H). |
| C37 | 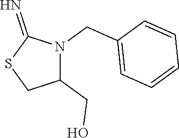 [SMILES: N=C1SCC(CO)N1Cc1ccccc1] | 223.3 | (DMSO-d₆, 400 MHz) δ 9.86 (s, 1H), 9.41 (s, 1H), 7.45 − 7.30 (m, 5H), 5.02 (s, 1H), 5.00 (d, J = 16.4 Hz, 1H), 4.66 (d, J = 16.4 Hz, 1H), 4.19 − 4.16 (m, 1H), 3.71 − 3.66 (m, 2H), 3.42- 3.39 (m, 1H), 3.38 − 3.36 (m, 1H). |

### Preparation of 3-phenylthiazolidin-2-imine (C1)

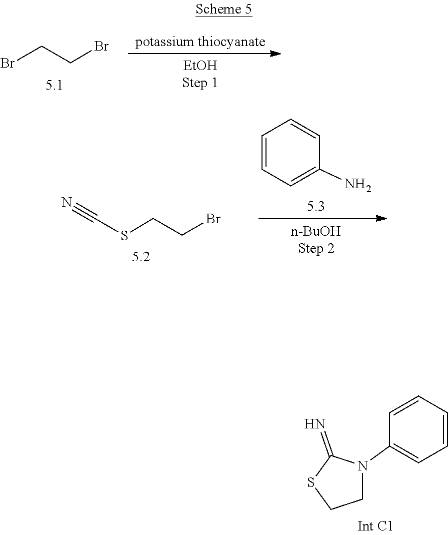

### Step 1. 1-Bromo-2-thiocyanatoethane (5.2)

To a solution of potassium thiocyanate (5.50 g, 56.6 mmol) in EtOH (30 mL) was added 1,2-dibromoethane (5.1) (31.9 g, 169 mmol). The resulting mixture was stirred at 80° C. for 16 hours, allowed to cool to 20° C., and filtered. The filtrate was concentrated under reduced pressure. The residue was purified by flash silica chromatography using an elution gradient of 20-25% EtOAc in petroleum ether to afford 3.60 g of the title compound (5.2) as an oil.

¹H NMR (400 MHz, CDCl₃) δ 3.69 (t, J=7.6 Hz, 2H), 3.362 (t, J=7.6 Hz, 2H)

### Step 2. 3-Phenylthiazolidin-2-imine (C1)

To a solution of 1-bromo-2-thiocyanatoethane (5.2) (1.00 g, 6.02 mmol) in n-butanol (15 mL) was added aniline (5.3) (0.560 g, 6.02 mmol) and the resulting mixture stirred at 120° C. for 3 hours. The reaction mixture was allowed to cool to 20° C., water (5 mL) was added and the mixture concentrated under reduced pressure. The residue was purified by reversed phase flash column chromatography (0.1% NH₃·H₂O/MeCN/water) to afford 0.50 g of the title C1 as a solid.

### Preparation of 3-(2-ethylphenyl) thiazolidin-2-imine (C2)

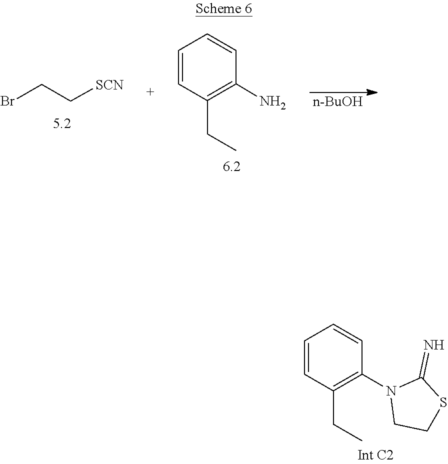

### 3-(2-Ethylphenyl) thiazolidin-2-imine (C2)

To a solution of 2-ethylaniline (6.2) (0.730 g, 6.02 mmol) in n-butanol (10 mL) was added 1-bromo-2-thiocyanatoethane (5.2) (1.00 g, 6.02 mmol) at 20° C. The resulting mixture was stirred at 130° C. for 1 hour. The reaction mixture was allowed to cool and was filtered. The filter cake was washed with ACN (10 mL) and then purified by reversed-phase flash (0.1% FA/ACN/water) to afford 0.50 g of the title compound C2 as the hydrobromic salt as a light-yellow solid.

### Preparation of 3-(2-methoxyphenyl)thiazolidin-2-imine (C3) and 2-(2-iminothiazolidin-3-yl)phenol (C4)

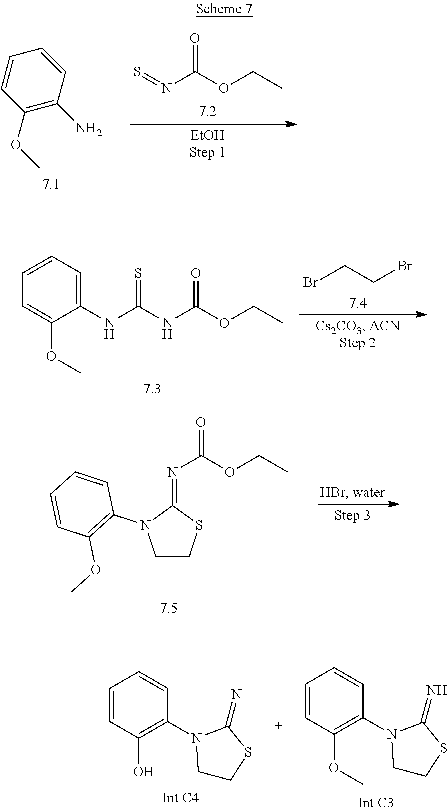

### Step 1. Ethyl N-[(2-methoxyphenyl)carbamothioyl]carbamate (7.3)

A mixture of 2-methoxyaniline (7.1) (1.00 g, 8.12 mmol) and O-ethyl carbonisothiocyanatidate (7.2) (1.06 g, 8.12 mmol) in EtOH (15 mL) was stirred at 20° C. for 1 hour. The mixture was filtered, the solid washed with EtOH (20 mL), and dried to afford 1.2 g of the title compound (7.3) as a solid.

LCMS (m/z [M+H]⁺): 255.0

### Step 2. Ethyl (Z)-(3-(2-methoxyphenyl)thiazolidin-2-ylidene)carbamate (7.5)

To a solution of ethyl ethyl N-[(2-methoxyphenyl)carbamothioyl]carbamate (7.3) (1.20 g, 4.72 mmol) and 1,2-dibromoethane (7.4) (0.886 g, 4.72 mmol) in ACN (15 mL) was added cesium carbonate (3.07 g, 9.44 mmol) and the mixture stirred at 50° C. for 12 hours. The mixture was filtered, and the filtrate was concentrated under reduced pressure. The residue was purified by silica gel column chromatography, eluting with 0 to 50% EtOAc in petroleum ether to afford 1.30 g of the title compound (7.5) as a solid.

¹H NMR (400 MHz, DMSO-d₆) δ 7.39-7.35 (m, 1H), 7.24 (dd, J=1.6, 8.0 Hz, 1H), 7.15 (dd, J=1.2, 8.4 Hz, 1H), 7.03-6.99 (m, 1H), 3.95-3.87 (m, 4H), 3.79 (s, 3H), 1.10 (t, J=6.8 Hz, 3H)

### Step 3. 3-(2-Methoxyphenyl)thiazolidin-2-imine (C3) and 2-(2-iminothiazolidin-3-yl)phenol (C4)

A solution of ethyl (Z)-(3-(2-methoxyphenyl)thiazolidin-2-ylidene)carbamate (7.5) (1.30 g, 4.64 mmol) in hydrobromic acid (5 mL, 40% in water) was stirred at 100° C. for 24 hour. The mixture was adjusted pH to 8 with solid NaOH, then concentrated under reduced pressure. The residue was purified by reversed-phase flash chromatyography (0.1% NH₃·H₂O/ACN condition) to afford 0.50 g of 2-(2-iminothiazolidin-3-yl)phenol (C4) as a solid and 0.080 g of 3-(2-methoxyphenyl)thiazolidin-2-imine (C3) as a solid.

¹H NMR for C4: (400 MHz, DMSO-d₆) δ 9.22 (br. s, 1H), 7.24 (dd, J=1.6, 8.0 Hz, 1H), 7.15-7.11 (m, 1H), 6.91-6.83 (m, 2H), 4.10 (t, J=7.2 Hz, 2H), 3.44 (t, J=6.8 Hz, 2H)

LCMS (m/z [M+H]⁺) for (C3): 209.0

### Preparation of 3-(4-Methoxyphenyl)thiazolidin-2-imine (C5)

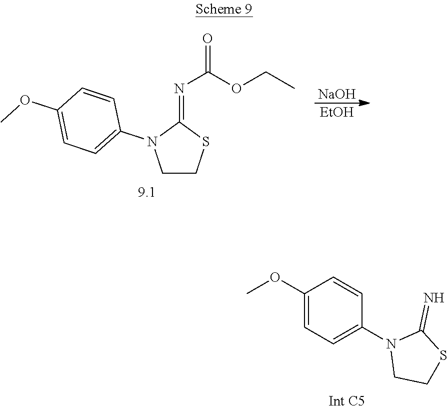

Ethyl (Z)-(3-(4-methoxyphenyl)thiazolidin-2-ylidene)carbamate (9.1) was prepared following the procedure described in Scheme 7.

¹H NMR (400 MHz, DMSO-d₆) δ 7.33-7.31 (m, 2H), 6.99-6.97 (m, 2H), 4.05 (t, J=7.6 Hz, 2H), 3.96 (q, J=6.8 Hz, 2H), 3.77 (s, 3H), 3.29 (t, J=7.6 Hz, 2H), 1.13 (t, J=7.2 Hz, 3H)

### 3-(4-Methoxyphenyl)thiazolidin-2-imine (C5)

To a solution of ethyl (Z)-(3-(4-methoxyphenyl)thiazolidin-2-ylidene)carbamate (9.1) (0.500 g, 1.78 mmol) in EtOH (10 mL) was added sodium hydroxide (1.43 g, 35.6 mmol). The mixture was stirred at 60° C. for 1 hour, then concentrated under reduced pressure. The residue was purified by reversed phase flash column chromatography (0.1% NH₃·H₂O condition) to afford 0.35 g of 3-(4-methoxyphenyl)thiazolidin-2-imine (C5) as a solid.

¹H NMR (400 MHz, DMSO-d₆) δ 7.39 (d, J=8.0 Hz, 2H), 6.91 (d, J=8.8 Hz, 2H), 3.98 (t, J=6.8 Hz, 2H), 3.73 (s, 3H), 3.25 (t, J=6.8 Hz, 2H)

### Preparation of 4-(2-iminothiazolidin-3-yl)phenol (C6)

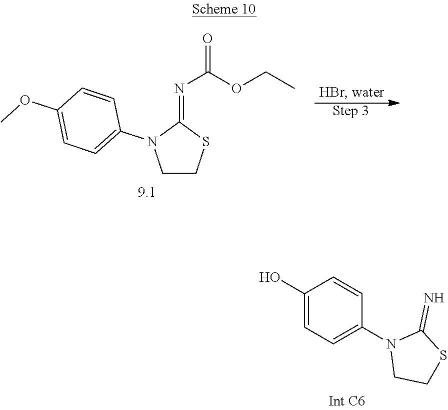

A solution of ethyl (Z)-(3-(4-methoxyphenyl)thiazolidin-2-ylidene)carbamate (9.1) (0.500 g, 1.78 mmol) in hydrobromic acid (10 mL, 40% in water) was stirred at 100° C. for 12 hours, then cooled to room temperature and concentrated under reduced pressure. The residue was purified by reversed phase flash column chramotography (0.1% NH₃·H₂O/ACN condition) to afford 0.28 g of 4-(2-iminothiazolidin-3-yl)phenol (C6) as a solid.

¹H NMR (400 MHz, DMSO-d₆) δ 9.98 (br. s, 1H), 8.70 (br. s, 1H), 7.31-7.27 (m, 2H), 6.93-6.89 (m, 2H), 4.26 (t, J=7.6 Hz, 2H), 3.61 (t, J=7.6 Hz, 2H)

### Preparation of 3-(3-methoxyphenyl)thiazolidin-2-imine (C7)

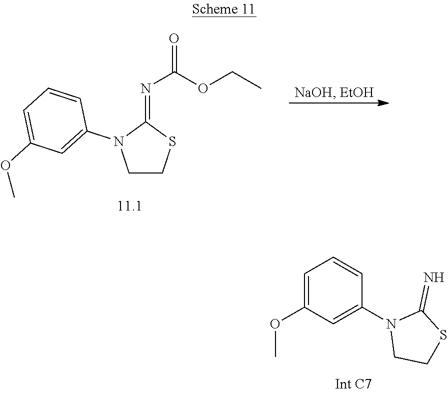

3-(3-Methoxyphenyl)thiazolidin-2-imine (11.1) was prepared following the procedure described in Scheme 7.

¹H NMR (400 MHz, DMSO-d₆) δ 7.33 (d, J=8.0 Hz, 1H), 7.03 (t, J=2.0 Hz, 1H)·7.00-6.98 (m, 1H), 6.89-6.76 (m, 1H), 4.10 (t, J=7.6 Hz, 2H), 4.00-3.95 (m, 2H), 3.78 (s, 3H), 3.29 (t, J=7.6 Hz, 2H), 1.13 (t, J=6.8 Hz, 3H)

### 3-(3-Methoxyphenyl)thiazolidin-2-imine (C7)

To a solution of ethyl (Z)-(3-(3-methoxyphenyl)thiazolidin-2-ylidene)carbamate (11.1) (0.400 g, 1.43 mmol) in EtOH (10 mL) was added sodium hydroxide (1.14 g, 28.5 mmol) and the mixture stirred at 60° C. for 2 hours. Water (30 mL) was added and the mixture extracted with EtOAc (2×20 mL). The combined organic layers were washed with brine (2×30 mL), dried over anhydrous Na₂SO₄, filtered, and concentrated. The residue was purified by reversed-phase flash chromatography (0.1% NH₃·H₂O/ACN condition) to afford 0.16 g of 3-(3-methoxyphenyl)thiazolidin-2-imine (C7) as an oil.

LCMS (m/z [M+H]⁺): 209.2

### Preparation of 3-(2-iminothiazolidin-3-yl)phenol (C8)

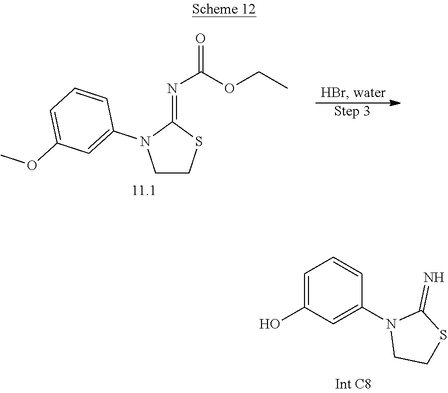

A solution of ethyl (Z)-(3-(3-methoxyphenyl)thiazolidin-2-ylidene)carbamate (11.1) (1.00 g, 3.57 mmol) in hydrobromic acid (9.69 mL, 71.3 mmol, 40% in water) was stirred at 100° C. for 12 hours, then cooled to room temperature and concentrated under reduced pressure. The residue was purified by reversed-phase flash chromatography (0.1% FA/MeCN condition) to afford 0.25 g of 3-(2-iminothiazolidin-3-yl)phenol (C8) as a solid.

LCMS (m/z [M+H]⁺): 195.2

### Preparation of 3-Cyclohexylthiazolidin-2-imine (C9)

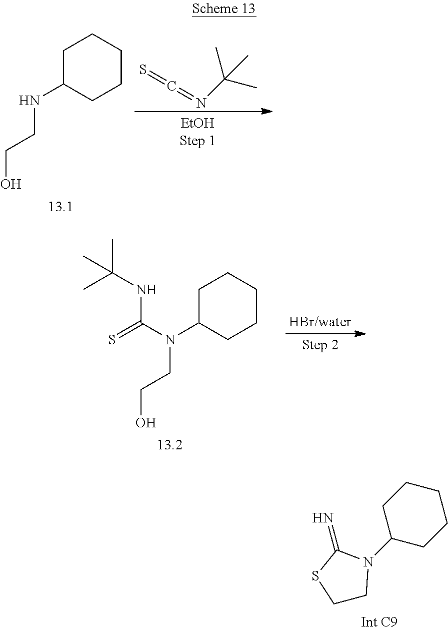

### Step 1. 3-(tert-Butyl)-1-cyclohexyl-1-(2-hydroxyethyl)thiourea (13.2)

A mixture of 2-(cyclohexylamino)ethan-1-ol (13.1) (0.300 g, 2.09 mmol) and 2-isothiocyanato-2-methylpropane (0.265 mL, 2.09 mmol) in EtOH (10 mL) was stirred at 20° C. for 12 hours. The reaction mixture was concentrated under reduced pressure to afford 0.550 g of 3-(tert-Butyl)-1-cyclohexyl-1-(2-hydroxyethyl)thiourea (13.2) as an oil.

¹H NMR (400 MHz, CDCl₃) δ 4.97-4.84 (m, 1H), 3.81 (s, 2H), 3.57-3.53 (m, 2H), 2.58-2.47 (m, 1H), 1.99-1.86 (m, 4H), 1.83-1.74 (m, 4H), 1.69-1.61 (m, 2H), 1.55 (s, 9H)

LCMS (m/z [M+H]⁺): 259.1

### Step 2. 3-Cyclohexylthiazolidin-2-imine (C9)

A mixture of 3-(tert-butyl)-1-cyclohexyl-1-(2-hydroxyethyl)thiourea (13.2) (0.300 g, 1.16 mmol) in hydrobromic acid (6 mL, 44.20 mmol, 40% in water) was stirred at 100° C. for 2 hours, then cooled to room temperature and concentrated under reduced pressure to afford 0.20 g of 3-cyclohexylthiazolidin-2-imine (C9) hydrobromic salt as an oil.

### Preparation of 3-cyclohexyl-4,4-dimethylthiazolidin-2-imine (C10)

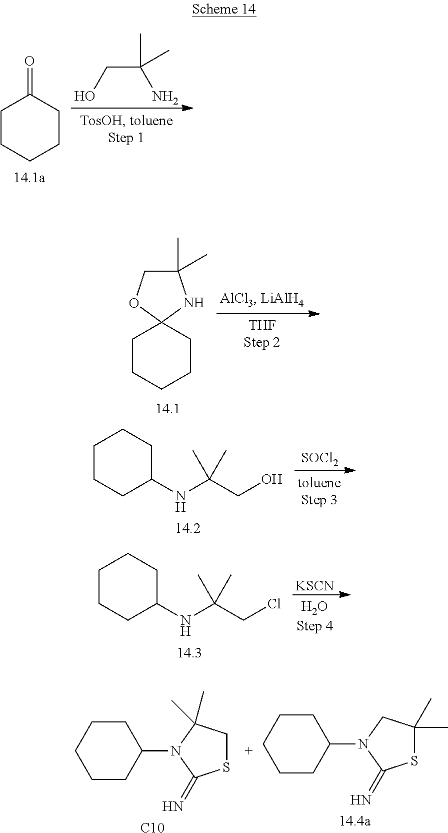

### Step 1.3, 3-Dimethyl-1-oxa-4-azaspiro[4.5]decane (14.1)

To a mixture of 2-amino-2-methyl-propan-1-ol (3.00 g, 33.6 mmol) and cyclohexanone (14.1a) (4.53 mL, 43.7 mmol) in toluene (9 mL) was added 4-methylbenzenesulfonic acid (0.579 g, 3.37 mmol). The resulting mixture was stirred at 110° C. for 12 hours, then cooled to room temperature and concentrated under reduced pressure. The residue was purified by silica column chromatography, using an elution gradient of 10 to 50% EtOAc in petroleum ether to afford 3.90 g of the title compound (14.1) as an oil.

¹H-NMR (400 MHz, CDCl₃) δ 3.57 (s, 2H), 1.73-1.65 (m, 2H), 1.61-1.48 (m, 6H), 1.48-1.40 (m, 1H), 1.36-1.27 (m, 1H), 1.24 (s, 6H)

### Step 2. 2-(Cyclohexylamino)-2-methylpropan-1-ol (14.2)

To a mixture of aluminum chloride (3.15 g, 23.6 mmol) and lithium aluminium hydride (0.897 g, 23.6 mmol) in THF (20 mL) was added a solution of 3-dimethyl-1-oxa-4-azaspiro[4.5]decane (14.1) (2.00 g, 11.8 mmol) in THF (8 mL) at 0° C. The resulting mixture was stirred at 20° C. under nitrogen for 1 hour. Water was added (0.5 mL), followed by NaOH (15%, 0.5 mL), water (1.5 mL) and more NaOH (1 M, 150 mL). The resulting mixture was filtered, and the filtrate extracted with EtOAc (2×50 mL). The combined organic layers were dried over anhydrous Na₂SO₄, filtered, and evaporated to afford 1.60 g of the title compound (14.2) as a solid.

¹H NMR (400 MHz, CDCl₃) δ 3.21 (s, 2H), 2.54-2.42 (m, 1H), 1.84-1.75 (m, 2H), 1.74-1.66 (m, 2H), 1.62-1.53 (m, 1H), 1.32-1.21 (m, 3H), 1.15-1.09 (m, 2H), 1.06 (s, 6H) Step 3. N-(1-Chloro-2-methylpropan-2-yl)cyclohexanamine (14.3)

A mixture of 2-(cyclohexylamino)-2-methyl-propan-1-ol (14.2) (1.40 g, 8.17 mmol) and thionyl chloride (1.78 mL, 24.5 mmol) in toluene (20 mL) was stirred at 80° C. for 12 hours. The solvent was evaporated to afford 1.80 g of crude title compound (14.3) hydrochloric salt as a solid.

¹H-NMR (400 MHz, DMSO-d₆) δ 9.07-8.82 (m, 2H), 3.99 (s, 2H), 3.33-3.24 (m, 1H), 2.09-1.99 (m, 2H), 1.79-1.70 (m, 2H), 1.59-1.46 (m, 3H), 1.43 (s, 6H), 1.37-1.25 (m, 2H), 1.14-0.99 (m, 1H)

### Step 4. 3-Cyclohexyl-4,4-dimethylthiazolidin-2-imine (C10)

A mixture of N-(2-chloro-1,1-dimethyl-ethyl)cyclohexanamine (14.3) (0.500 g, 2.21 mmol) and potassium thiocyanate (0.214 g, 2.21 mmol) in water (6 ML) was stirred at 80° C. for 12 hours then cooled to room temperature. A saturated aqueous solution of sodium carbonate (50 mL) was added to the cooled reaction mixture which was then extracted with EtOAc (3×30 mL). The organic phases were combined, dried over anhydrous Na₂SO₄, filtered, and evaporated. The residue was purified by preparative HPLC (Phenomenex Luna Cis 30×75 mm, 3 mm; mobile phase A: water/0.1% TFA, mobile phase B: ACN/0.1% TFA; gradient: 16% B to 36% B over 7 min) to afford 0.20 g of 3-cyclohexyl-4,4-dimethylthiazolidin-2-imin (C10) trifluoroacetic salt as an oil.

### Preparation of (2-Imino-3-phenylthiazolidin-4-yl)methanol (C11)

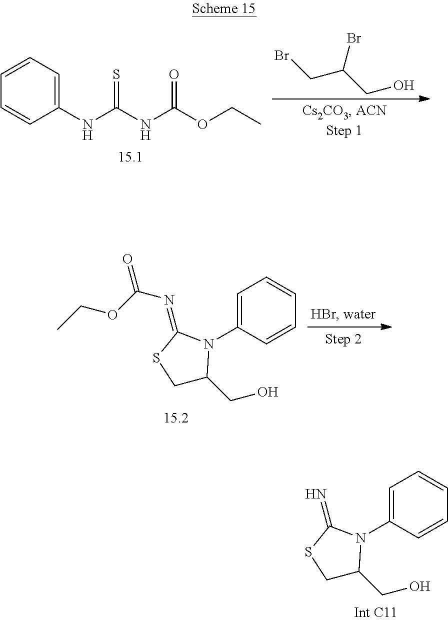 [SMILES: CCOC(=O)NC(=S)Nc1ccccc1 ; OCC(Br)CBr ; CCOC(=O)/N=C1\SCC(CO)N1c1ccccc1 ; N=C1SCC(CO)N1c1ccccc1]

### Step 1. Ethyl (Z)-(4-(hydroxymethyl)-3-phenylthiazolidin-2-ylidene)carbamate (15.2)

To a solution of ethyl N-(phenylcarbamothioyl)carbamate (15.1) (2.06 g, 9.18 mmol) and cesium carbonate (4.00 g, 13.8 mmol) in acetonitrile (20 mL) was added 2,3-dibromopropan-1-ol (0.690 mL, 18.4 mmol) at 25° C. and the reaction mixture was stirred for 12 hours. The resulting precipitate was filtered and was rinsed with EtOAc (80 mL). The combined filtrate was concentrated under reduced pressure. The residue was triturated with EtOAc (8 mL) to afford 2.20 g of the title compound (15.2) as a solid.

¹H-NMR (400 MHz, DMSO-d₆) δ 7.49-7.41 (m, 2H), 7.40-7.31 (m, 3H), 5.05 (t, J=5.6 Hz, 1H), 4.41-4.32 (m, 1H), 3.93 (q, J=7.2 Hz, 2H), 3.55-3.47 (m, 1H), 3.46-3.35 (m, 2H), 3.30-3.27 (m, 1H), 1.10 (t, J=7.2 Hz, 3H)

### Step 2. (2-Imino-3-phenylthiazolidin-4-yl)methanol (C11)

A solution of (Z)-ethyl (4-(hydroxymethyl)-3-phenylthiazolidin-2-ylidene)carbamate (15.2) (1.20 g, 4.28 mmol) in hydrobromic acid (12 mL, 40% in water) was stirred at 100° C. for 12 hours. The cooled mixture was concentrated under reduced pressure and the residue was lyophilized to afford 1.50 g of crude (2-Imino-3-phenylthiazolidin-4-yl)methanol (C11) as an oil which was used without further purification.

### Preparation of 3-(2-chlorophenyl)thiazolidin-2-imine (C12)

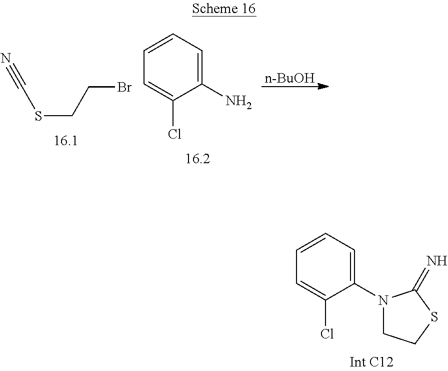

A mixture of 1-bromo-2-thiocyanatoethane (16.1) (1.00 g, 6.02 mmol) and 2-chloroaniline (16.2) (0.768 g, 6.02 mmol) in n-butanol (10 mL) was stirred at 120° C. for 3 hours. The resulting precipitate was collected, washed with acetonitrile (10 mL) and dried under reduced pressure to afford 0.50 g of 3-(2-chlorophenyl)thiazolidin-2-imine (C12) hydrobromic salt as a solid.

### Preparation of hexahydro-3H-thiazolo[3,4-a]pyridin-3-imine (C13)

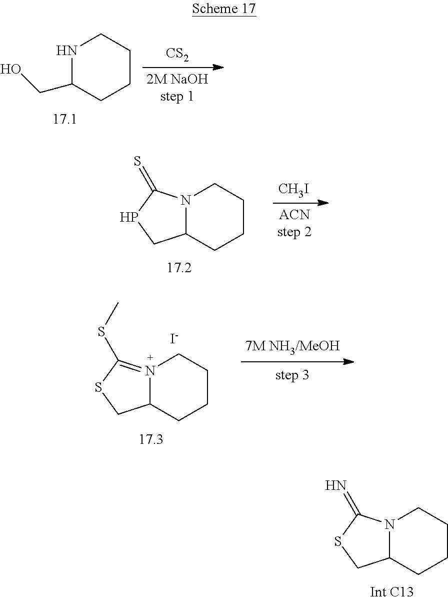

### Step 1. Hexahydro-[1,3]azaphospholo[1,5-a]pyridine-3(2H)-thione (17.2)

To a solution of piperidin-2-ylmethanol (17.1) (0.400 g, 3.47 mmol) in NaOH (20.0 mL, 2 M) was added carbon disulfide (2.10 mL, 34.7 mmol) at 25° C. and the reaction mixture stirred at 25° C. for 3 hours. Water (80 mL) was added to the reaction mixture which was then extracted with DCM (3×30 mL). The combined organic layers were washed with brine (50 mL), dried over anhydrous Na₂SO₄, filtered, and evaporated. The residue was purified by silica column chromatography, eluting with 2 to 20% EtOAc in petroleum ether to afford 0.70 g of the title compound (17.2) as a solid.

¹H NMR (400 MHz, DMSO-d₆) δ 4.56-4.51 (m, 1H), 4.16-4.09 (m, 1H), 3.53-3.48 (m, 1H), 3.03-2.98 (m, 1H), 2.92-2.85 (m, 1H), 1.97-1.93 (m, 1H), 1.80-1.73 (m, 2H), 1.50-1.42 (m, 2H), 1.37-1.30 (m, 1H)

LCMS (m/z [M+H]⁺): 173.8

### Step 2. 3-(Methylthio)-1,5,6,7,8,8a-hexahydrothiazolo[3,4-a]pyridin-4-ium iodide (17.3)

To a solution of tetrahydro-1H-thiazolo[3,4-a]pyridine-3(5H)-thione (17.2) (0.250 g, 1.40 mmol) in acetonitrile (3.0 mL) was added iodomethane (0.348 mL, 5.59 mmol) at 0° C. The resulting mixture was stirred at 40° C. for 12 h and the solvent evaporated to afford 0.23 g of the title compound (17.3) as a solid.

¹H NMR (400 MHz, DMSO-d₆) δ 4.60-4.52 (m, 1H), 4.05-3.94 (m, 2H), 3.48-3.40 (m, 2H), 2.90 (s, 3H), 2.14-2.08 (m, 1H), 1.91-1.82 (m, 2H), 1.72-1.61 (m, 1H), 1.59-1.46 (m, 2H).

LCMS (m/z [M+H]⁺): 187.8

### Step 3. Hexahydro-3H-thiazolo[3,4-a]pyridin-3-imine (C13)

A solution of 3-(methylthio)-1,5,6,7,8,8a-hexahydrothiazolo[3,4-a]pyridin-4-ium iodide (17.3) (0.230 g, 0.730 mmol) in ammonia (2.56 mL, 7M in MeOH) was stirred at 25° C. for 3 hours. The solvent was removed and the residue purified by reversed-phase flash chromatography (0.1% NH₃·H₂O in water, MeCN) to afford 0.030 g of hexahydro-3H-thiazolo[3,4-a]pyridin-3-imine (C13) as a solid.

### Preparation of tert-butyl 3-iminotetrahydro-3H-thiazolo[3,4-a]pyrazine-7(1H)-carboxylate (C14)

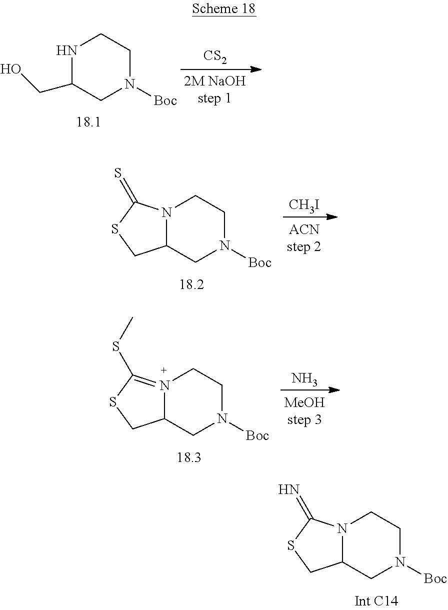

### Step 1. tert-Butyl 3-thioxotetrahydro-3H-thiazolo[3,4-a]pyrazine-7(1H)-carboxylate (18.2)

To a solution of tert-butyl 3-(hydroxymethyl)piperazine-1-carboxylate (18.1) (1.50 g, 6.94 mmol) and sodium hydroxide (4.80 g, 120 mmol) in water (60 mL) was added carbon disulfide (5.28 g, 69.3 mmol) at 25° C. The resulting mixture was stirred at 25° C. for 2 hours and then extracted with DCM (3×40 mL). The combined organic layers were washed with brine (2×50 mL), dried over anhydrous Na₂SO₄, filtered, and evaporated. The residue was purified by silica gel column chromatography, eluting with 30 to 50% EtOAc in petroleum ether to afford 1.50 g of the title compound (18.2) as a solid.

¹H NMR (400 MHz, DMSO-d₆) δ 4.46-4.44 (m, 1H), 4.32-4.25 (m, 2H), 4.09-3.98 (m, 1H), 3.53 (dd, J=8.0 Hz, 11.2 Hz, 1H), 3.09 (dd, J=9.6 Hz, 11.2 Hz, 1H), 3.02-2.96 (m, 1H), 2.94-2.82 (m, 2H), 1.42 (s, 9H)

LCMS (m/z [M+H]⁺): 219.1

### Step 2. 7-(tert-Butoxycarbonyl)-3-(methylthio)-1,5,6,7,8,8a-hexahydrothiazolo[3,4-a]pyrazin-4-ium (18.3)

To a solution of tert-butyl 3-thioxotetrahydro-1H-thiazolo[3,4-a]pyrazine-7(3H)-carboxylate (18.2) (1.45 g, 5.28 mmol) in acetonitrile (20 mL) was added iodomethane (3.00 g, 21.1 mmol) at 0° C. The reaction mixture was stirred at 40° C. for 12 hours then concentrated under reduced pressure to afford 2.00 g of the title compound (18.3) as a solid.

¹H NMR (400 MHz, DMSO-d₆) δ 4.57-4.53 (m, 1H), 4.28-4.25 (m, 1H), 4.04-4.01 (m, 1H), 4.00-3.95 (m, 2H), 3.46-3.32 (m, 2H), 3.05-2.94 (m, 2H), 2.85 (s, 3H), 1.36 (s, 9H)

### Step 3. tert-Butyl 3-iminotetrahydro-3H-thiazolo[3,4-a]pyrazine-7(1H)-carboxylate (C14)

Ammonia gas (15 psi) was bubbled into MeOH (10 mL) for 5 minutes at 0° C. To the solution was added 7-(tert-butoxycarbonyl)-3-(methylthio)-1, 5, 6, 7, 8, 8a-hexahydrothiazolo [3, 4-a]pyrazin-4-ium (18.3) (0.670 g, 1.66 mmol) at 25° C. The resulting mixture was stirred at 25° C. for 3 hours, and then the solvent was removed. The residue was purified by reversed-phase flash chromatography (0.1% NH₃·H₂O/MeCN condition) to afford 0.38 g of tert-butyl 3-iminotetrahydro-3H-thiazolo[3,4-a]pyrazine-7(1H)-carboxylate (C14) as an oil.

### Preparation of 2-Imino-3-phenylthiazolidin-4-one (C15)

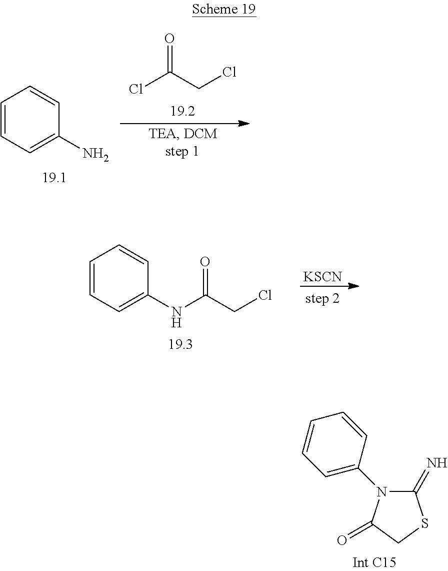

### Step 1. 2-Chloro-N-phenylacetamide (19.3)

To a solution of aniline (19.1) (1.00 g, 10.7 mmol), Et₃N (1.20 g, 11.8 mmol) and dimethylaminopyridine (0.131 g, 1.07 mmol) in DCM (10 mL) was added 2-chloroacetyl chloride (19.2) (1.33 g, 11.8 mmol) at 0° C. The resulting mixture was stirred at 25° C. for 4 hours, and then washed with 1M hydrochloric acid (20 mL) followed by brine (20 mL) then dried over anhydrous Na₂SO₄, filtered, and evaporated to afford 1.50 g of 2-chloro-N-phenylacetamide (19.3) as a solid.

¹H NMR (400 MHz, CDCl₃) δ 8.24 (br. s, 1H), 7.56 (d, J=8.0 Hz, 2H), 7.37 (t, J=8.0 Hz, 2H), 7.19 (t, J=7.2 Hz, 1H), 4.20 (s, 2H)

LCMS (m/z [M+H]⁺): 170.1

### Step 2. 2-Imino-3-phenylthiazolidin-4-one (C15)

A mixture of 2-chloro-N-phenylacetamide (19.3) (0.500 g, 2.95 mmol) and potassium thiocyanate (0.573 g, 5.90 mmol) in acetone (10 mL) was stirred at 65° C. for 3 hours then cooled to room temperature. Saturated aqueous sodium bicarbonate solution (100 mL) was added to the cooled mixture which was extracted with EtOAc (3×50 mL). The combined organic layers were dried over anhydrous Na₂SO₄, filtered, and evaporated. The residue was purified by silica gel column chromatography, eluting with 10 to 20% EtOAc in petroleum ether to afford 0.50 g of 2-imino-3-phenylthiazolidin-4-one (C15) as a solid.

### Preparation of 3-Benzyl-1,3-thiazinan-2-imine (C16)

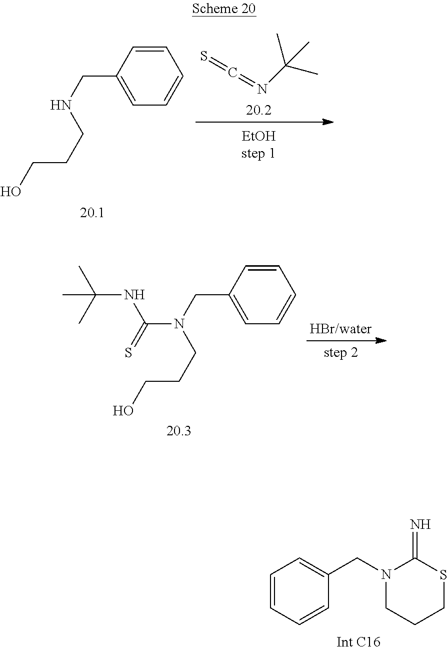

### Step 1. 1-Benzyl-3-(tert-butyl)-1-(3-hydroxypropyl)thiourea (20.3)

A mixture of 3-(benzylamino)propan-1-ol (20.1) (0.600 g, 3.63 mmol) and 2-isothiocyanato-2-methylpropane (20.2) (0.418 g, 3.63 mmol) in EtOH (10 mL) was stirred at 25° C. for 2 hours, then the solvent was evaporated to afford 1.02 g of the title compound (20.3) as an oil.

¹H NMR (400 MHz, CDCl₃) δ 7.40-7.36 (m, 2H), 7.34-7.30 (m, 1H), 7.25-7.23 (m, 2H), 5.65 (br. s, 1H), 4.73 (s, 2H), 4.08 (t, J=6.0 Hz, 2H), 3.74-3.69 (m, 1H), 3.51-3.48 (m, 1H), 1.84-1.78 (m, 2H), 1.39 (s, 9H)

LCMS (m/z [M+H]⁺): 281.5

### Step 2. 3-Benzyl-1,3-thiazinan-2-imine (C16)

A solution of 1-benzyl-3-(tert-butyl)-1-(3-hydroxypropyl)thiourea (20.3) (0.300 g, 1.07 mmol) in hydrobromic acid (16.1 mmol, 2.18 mL, 40% in water) was stirred at 100° C. for 1 hour, then the solvent was evaporated. The residue was purified by reversed-phase flash chromatography (0.1% HCl/MeCN condition) to afford 0.258 g of 3-benzyl-1,3-thiazinan-2-imine (C16) as the hydrochloric salt as a solid.

### Preparation of 3-Phenyl-1,3-thiazinan-2-imine (C17)

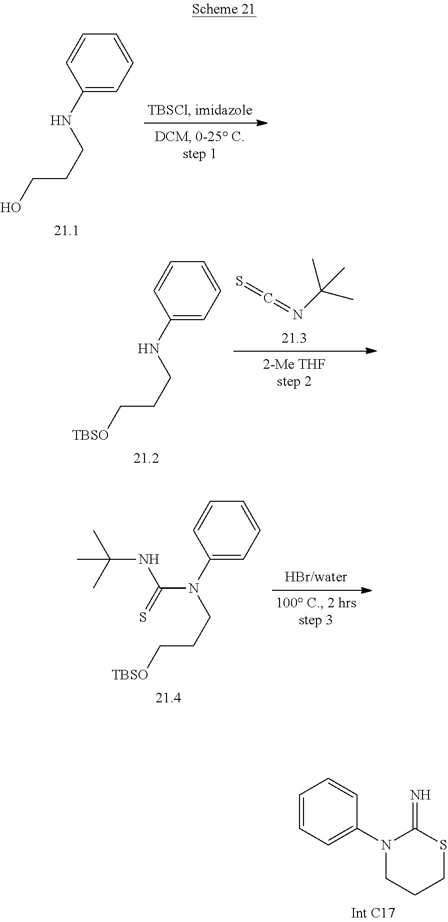

### Step 1. N-(3-((tert-Butyldimethylsilyl)oxy)propyl)aniline (21.2)

To a solution of 3-(phenylamino)propan-1-ol (21.1) (3.00 g, 19.8 mmol) and imidazole (2.03 g, 29.7 mmol) in DCM (20 mL) was added tert-butylchlorodimethylsilane (4.49 g, 29.7 mmol) at 0° C. The resulting mixture was stirred at 25° C. for 2 hours, water (40 mL) was added and the mixture extracted with DCM (3×50 mL). The combined organic phases were washed with brine (3×30 mL), dried over anhydrous Na₂SO₄, filtered, and concentrated. The residue was purified by silica gel column chromatography, eluting with 5 to 20% EtOAc in petroleum ether to afford 5.15 g of the title compound (21.2) as an oil.

¹H NMR (400 MHz, CDCl₃) δ 7.18 (t, J=8.4 Hz, 2H), 6.69 (t, J=7.2 Hz, 1H), 6.62 (d, J=8.0 Hz, 2H), 3.78 (t, J=5.6 Hz, 2H), 3.25 (t, J=6.4 Hz, 2H), 1.89-1.82 (m, 2H), 0.94 (s, 9H), 0.08 (s, 6H)

LCMS (m/z [M+H]⁺): 266.1

### Step 2. 3-(tert-Butyl)-1-(3-((tert-butyldimethylsilyl)oxy)propyl)-1-phenylthiourea (21.4)

To a solution of N-(3-((tert-butyldimethylsilyl)oxy)propyl)aniline (21.2) (4.00 g, 15.0 mmol) in 2-methyl THF (30 mL) was added potassium tert-butoxide (1.69 g, 15.1 mmol) at 50° C. under nitrogen, followed by a solution of 2-isothiocyanato-2-methylpropane (21.3) (2.08 g, 18.1 mmol) in 2-methyl THF (10 mL). The resulting mixture was stirred at 50° C. for 2 hours, and then concentrated. The residue was purified by reversed-phase flash chromatography (0.1% FA condition) to afford 0.52 g of the title compound (21.4) as an oil.

¹H NMR (400 MHz, CDCl₃) δ 7.52-7.43 (m, 2H), 7.42-7.33 (m, 1H), 7.23-7.09 (m, 2H), 4.32-4.12 (m, 2H), 3.65 (t, J=6.0 Hz, 2H), 1.91-1.85 (m, 2H), 1.39 (s, 9H), 0.84 (s, 9H), 0.12 (s, 6H)

LCMS (m/z [M+H]⁺): 381.9

### Step 3. 3-Phenyl-1,3-thiazinan-2-imine (C17)

A solution of 3-(tert-butyl)-1-(3-((tert-butyldimethylsilyl)oxy)propyl)-1-phenylthiourea (21.4) (0.200 g, 0.525 mmol) in hydrobromic acid (4.47 g, 40% in water) was stirred at 100° C. for 2 hours and then the cooled mixture was concentrated under reduced pressure. The residue was purified by preparative HPLC (Welch Xtimate C₁₈, 25×150 mm, 5 mm; mobile phase A: water/0.05% HCl, mobile phase B: MeCN; gradient: 0% B to 13% B over 10 min) to afford 0.12 g of 3-phenyl-1,3-thiazinan-2-imine (C17) hydrochloride salt as an oil.

### Preparation of 3-(2-Chlorophenyl)-1,3-thiazinan-2-imine (C18)

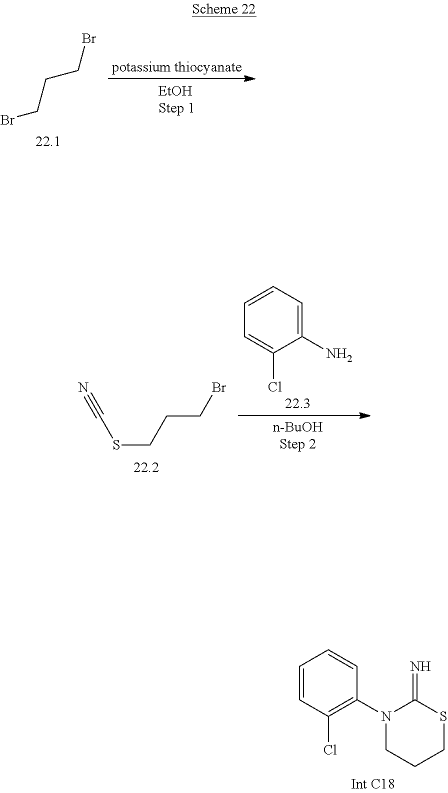 [SMILES: BrCCCBr ; Nc1ccccc1Cl ; N#CSCCCBr ; N=C1SCCCN1c1ccccc1Cl]

### Step 1. 1-Bromo-3-thiocyanatopropane (22.2)

A mixture of 1,3-dibromopropane (22.1) (18.7 g, 92.6 mmol) and thiocyanate potassium (3.00 g, 30.9 mmol) in EtOH (30 mL) was stirred at 80° C. for 16 hours, and then concentrated under reduced pressure. The residue was purified by silica gel column chromatography, eluting with 2 to 10% EtOAc in petroleum ether to afford 2.00 g of 1-bromo-3-thiocyanatopropane (22.2) as an oil.

¹H NMR (400 MHz, CDCl₃) δ 3.57 (t, J=6.0 Hz, 2H), 3.14 (t, J=6.8 Hz, 2H), 2.42-2.33 (m, 2H)

### Step 2. 3-(2-Chlorophenyl)-1,3-thiazinan-2-imine (C18)

A mixture of 1-bromo-3-thiocyanatopropane (22.2) (0.500 g, 2.78 mmol) and 2-chloroaniline (22.3) (0.354 g, 2.78 mmol) in n-butanol (10 mL) was stirred at 120° C. for 2 hours. The resulting precipitate was collected, washed with acetonitrile (10 mL) and dried under reduced pressure to afford 0.20 g of 3-(2-chlorophenyl)-1,3-thiazinan-2-imine (C18) hydrobromide salt as a solid.

### Preparation of 3-Cyclohexyl-1,3-thiazinan-2-imine (C19)

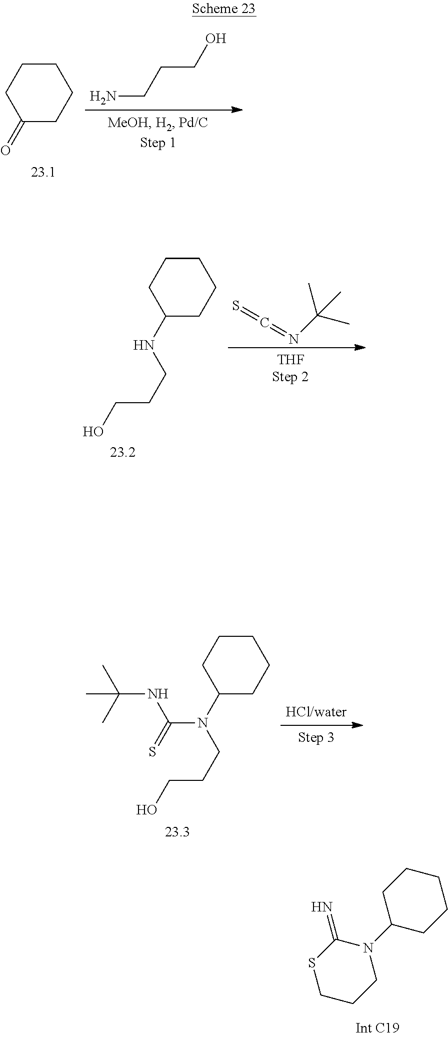

### Step 1. 3-(Cyclohexylamino)propan-1-ol (23.2)

A mixture of cyclohexanone (23.1) (1.23 g, 12.5 mmol) and 3-aminopropan-1-ol (1.00 g, 13.3 mmol) in MeOH (10 mL) was stirred at 25° C. for 2 hours. Pd/C (0.100 g, 10% on charcoal, wet) was added. The mixture was degassed and purged with hydrogen three times. The reaction mixture was stirred at 25° C. for 12 hours under hydrogen atmosphere (15 psi). The mixture was filtered through Celite, and the filtrate was concentrated under reduced pressure to afford 2.00 g of 3-(cyclohexylamino)propan-1-ol (23.2) as a solid.

¹H NMR (400 MHz, CDCl₃) δ 3.81 (t, J=5.2 Hz, 2H), 2.92 (J=5.6 Hz, 2H), 2.90-2.65 (m, 3H), 2.48-2.41 (m, 1H), 1.97-1.86 (m, 2H), 1.78-1.66 (m, 4H), 1.63-1.58 (m, 1H), 1.33-1.01 (m, 5H)

LCMS (m/z [M+H]⁺): 157.9

### Step 2. 3-(tert-Butyl)-1-cyclohexyl-1-(3-hydroxypropyl)thiourea (23.3)

To a solution of 3-(cyclohexylamino)propan-1-ol (23.2) (0.500 g, 3.18 mmol) in THF (15 mL) was added 2-isothiocyanato-2-methylpropane (0.381 g, 3.31 mmol). The reaction mixture was stirred at 25° C. for 12 hours and then concentrated under reduced pressure to afford 0.85 g of the title compound (23.3) as a gum.

¹H NMR (CDCl₃, 400 MHz) δ 5.95 (br. s, 1H), 4.89 (br. s, 1H), 3.68 (t, J=5.2 Hz, 2H), 3.58 (t, J=7.2 Hz, 2H), 2.65-2.36 (m, 1H), 1.89-1.78 (m, 8H), 1.59-1.55 (m, 9H), 1.41-1.38 (m, 2H)

### Step 3. 3-Cyclohexyl-1,3-thiazinan-2-imine (C19)

A mixture of 3-(tert-butyl)-1-cyclohexyl-1-(3-hydroxypropyl)thiourea (23.3) (0.200 g, 0.734 mmol) in hydrochloric acid (12 M, 2 mL) and water (2.8 mL) was stirred at 100° C. for 2 hours. The mixture was concentrated under reduced pressure to afford 0.12 g of 3-cyclohexyl-1,3-thiazinan-2-imine (C19) as the hydrochloride as a gum.

### Preparation of 1-(4-(2-Imino-1,3-thiazinan-3-yl)piperidin-1-yl)ethan-1-one (C20)

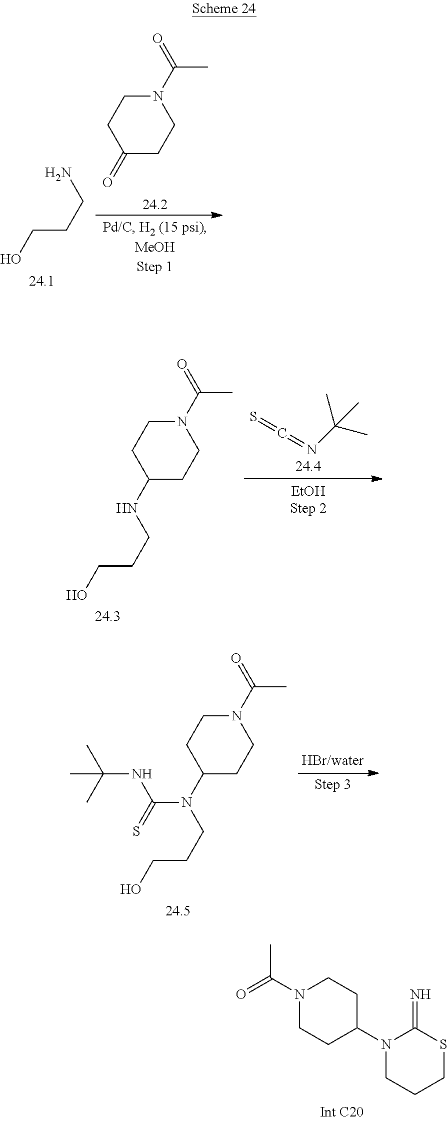

### Step 1. 1-(4-((3-Hydroxypropyl)amino)piperidin-1-yl)ethan-1-one (24.3)

A solution of 3-aminopropan-1-ol (24.1) (1.00 g, 13.3 mmol) and 1-acetylpiperidin-4-one (24.2) (1.88 g, 13.3 mmol) in MeOH (30 mL) was stirred at 25° C. for 2 hours. Pd/C (0.200 g, 10% on active charcoal, wet) was added at 25° C. under a nitrogen atmosphere. The mixture was degassed, purged with hydrogen three times, and stirred at 25° C. for 12 hours under a hydrogen atmosphere (15 psi). The mixture was filtered through Celite and the filtrate was concentrated under reduced pressure to afford 2.96 g of the title compound (24.3) as an oil.

¹H NMR (400 MHz, CDCl₃) δ 3.92-3.72 (m, 3H), 3.48-3.37 (m, 2H), 3.03-2.85 (m, 2H), 2.09 (s, 3H), 2.05-1.83 (m, 4H), 1.79-1.52 (m, 4H)

LCMS (m/z [M+H]⁺): 201.1

### Step 2. 1-(1-Acetylpiperidin-4-yl)-3-(tert-butyl)-1-(3-hydroxypropyl)thiourea (24.5)

A mixture of 1-(4-((3-hydroxypropyl)amino)piperidin-1-yl)ethanone (24.3) (0.500 g, 2.50 mmol) and 2-isothiocyanato-2-methylpropane (24.4) (0.288 g, 2.50 mmol) in EtOH (10 mL) was stirred at 25° C. for 12 hours and then concentrated under reduced pressure to afford 0.70 g of crude title compound (24.5) as an oil which was used directly without further purification.

LCMS (m/z [M+H]⁺): 316.2

### Step 3. 1-(4-(2-Imino-1,3-thiazinan-3-yl)piperidin-1-yl)ethan-1-one (C20)

A solution of 1-(1-acetylpiperidin-4-yl)-3-(tert-butyl)-1-(3-hydroxypropyl)thiourea (24.5) (0.650 g, 2.06 mmol) in hydrobromic acid (8.34 g, 41.2 mmol, 40% purity in water) was stirred at 100° C. for 0.5 hour and then the cooled mixture was adjusted to pH=7 with potassium carbonate powder. The residue was purified by reversed phase flash chromatography (0.1% HCl/MeCN condition) to afford 0.45 g of 1-(4-(2-imino-1,3-thiazinan-3-yl)piperidin-1-yl)ethan-1-one (C20) hydrochloride as a solid.

### Preparation of 3-(1-Methylpiperidin-4-yl)-1,3-thiazinan-2-imine (C21)

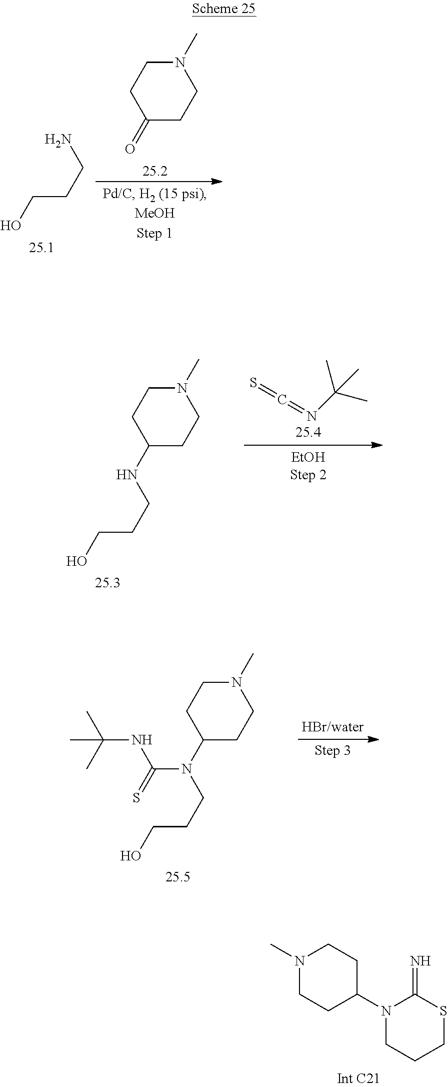

### Step 1. 3-((1-Methylpiperidin-4-yl)amino)propan-1-ol (25.3)

A mixture of 3-aminopropan-1-ol (25.1) (1.00 g, 13.3 mmol) and 1-methylpiperidin-4-one (25.2) (1.51 g, 13.3 mmol) in MeOH (20 mL) was stirred at 20° C. for 2 hours under a nitrogen atmosphere. Pd/C (0.100 g, 13.3 mmol, 10% on charcoal) was added to the mixture under a nitrogen atmosphere. The mixture was degassed and purged with hydrogen three times. The mixture was then stirred at 20° C. for 12 hours under a hydrogen atmosphere (15 psi), filtered through Celite, and the filtrate was evaporated to afford 1.80 g of crude title compound (25.3) as an oil.

¹H NMR (400 MHz, CDCl₃) δ 3.88 (t, J=5.6 Hz, 2H), 2.99 (t, J=9.6 Hz, 2H), 2.82-2.74 (m, 1H), 2.51-2.44 (m, 2H), 2.40-2.33 (m, 2H), 2.28 (s, 3H), 1.94-1.89 (m, 2H), 1.84-1.74 (m, 2H), 1.70-1.65 (m, 1H), 1.41-1.33 (m, 1H)

### Step 2. 3-(tert-Butyl)-1-(3-hydroxypropyl)-1-(1-methylpiperidin-4-yl)thiourea (25.5)

To a solution of 3-[(1-methyl-4-piperidyl)amino]propan-1-ol (25.3) (0.500 g, 2.90 mmol) in EtOH (5 mL) was added 2-isothiocyanato-2-methyl-propane (25.4) (0.330 g, 2.90 mmol) at 25° C. The mixture was stirred at 25° C. for 2 hours and then concentrated under reduced pressure. The residue was purified by reversed-phase flash chromatography (0.1% FA/MeCN condition) to afford 0.60 g of the title compound (25.5) as an oil.

¹H NMR (400 MHz, CD₃OD) δ 5.74-5.62 (m, 1H), 3.58 (t, J=5.6 Hz, 2H), 3.50-3.47 (m, 2H), 3.16-3.09 (m, 2H), 3.04-2.98 (m, 2H), 2.53 (s, 3H), 1.90-1.84 (m, 2H), 1.82-1.69 (m, 4H), 1.55 (s, 9H)

LCMS (m/z [M+H]⁺): 288.0

### Step 3. 3-(1-Methylpiperidin-4-yl)-1,3-thiazinan-2-imine (C21)

A mixture of 3-(tert-butyl)-1-(3-hydroxypropyl)-1-(1-methylpiperidin-4-yl)thiourea (25.5) (0.600 g, 2.09 mmol) in hydrobromic acid (18.6 g, 92.0 mmol, 40% in water) was stirred at 100° C. for 2 hours and then the cooled mixture was concentrated under reduced pressure. The residue was purified by reversed phase flash chromatography (0.1% NH₃·H₂O/MeCN condition) to afford 0.26 g of 3-(1-methylpiperidin-4-yl)-1,3-thiazinan-2-imine (C21) as a solid.

### Preparation of 3-Cyclohexyl-5,5-dimethyl-1,3-thiazinan-2-imin (C22)

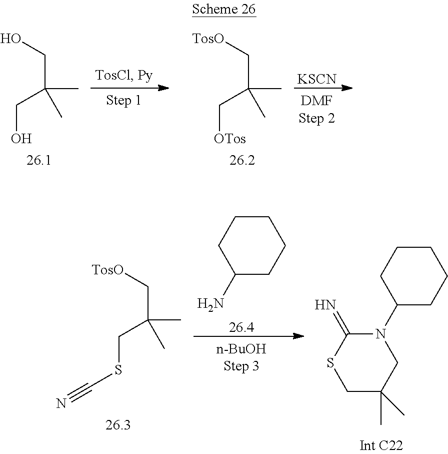

### Step 1. 2,2-Dimethylpropane-1,3-diyl bis(4-methylbenzenesulfonate) (26.2)

To a solution of 4-methylbenzenesulfonyl chloride (16.5 g, 86.5 mmol) in pyridine (20 mL) at 0° C. was added 2,2-dimethylpropane-1,3-diol (26.1) (3.00 g, 28.8 mmol) in pyridine (20 mL). The mixture was stirred at 20° C. for 16 hours. Water (200 mL) was added to the reaction mixture which was then extracted with EtOAc (3×100 mL). The combined organic layers were washed with hydrochloric acid (1M, 3×200 mL) followed by brine (3×300 mL) then dried over anhydrous Na₂SO₄, filtered, and concentrated to dryness to afford 11.9 g of the title compound (26.2) as a solid.

¹H NMR (400 MHz, DMSO-d₆) δ 7.74 (d, J=8.4 Hz, 4H), 7.47 (d, J=8.4 Hz, 4H), 3.72 (s, 4H), 2.43 (s, 6H), 0.78 (s, 6H)

LCMS (m/z [M+H]⁺): 413.1

### Step 2. 2,2-Dimethyl-3-thiocyanatopropyl 4-methylbenzenesulfonate (26.3)

A mixture of 2,2-dimethylpropane-1,3-diyl bis(4-methylbenzenesulfonate) (26.2) (10.0 g, 24.2 mmol) in DMF (10 mL) and potassium thiocyanate (0.800 g, 8.23 mmol) was stirred at 100° C. for 24 hours. The resulting precipitate was collected by filtration. The filter cake was purified by reversed-phase flash chromatography (0.1% TFA/MeCN condition) to afford 0.73 g of the title compound (26.3) as an oil.

¹H NMR (400 MHz, CDCl₃) δ 7.80 (d, J=8.0 Hz, 2H), 7.39 (d, J=8.0 Hz, 2H), 3.82 (s, 2H), 2.99 (s, 2H), 2.48 (s, 3H), 1.08 (s, 6H)

LCMS (m/z [M+H]⁺): 317.1

### Step 3. 3-Cyclohexyl-5,5-dimethyl-1,3-thiazinan-2-imine (C22)

A mixture of 2,2-dimethyl-3-thiocyanatopropyl 4-methylbenzenesulfonate (26.3) (0.330 g, 1.10 mmol) in butanol (3 mL) and cyclohexanamine (26.4) (0.165 g, 1.66 mmol) was stirred at 149° C. for 1 hour under microwave. The solvent was evaporated, and the residue was purified by reversed-phase HPLC (0.1% TFA/MeCN condition) to afford 0.105 g of 3-cyclohexyl-5,5-dimethyl-1,3-thiazinan-2-imine (C22) as the trifluoroacetic salt as an oil.

### Preparation of Methyl (Z)-2-((ethoxycarbonyl)imino)-3-phenyl-1,3-thiazinane-5-carboxylate (C23) and methyl 2-imino-3-phenyl-1,3-thiazinane-5-carboxylate (C24)

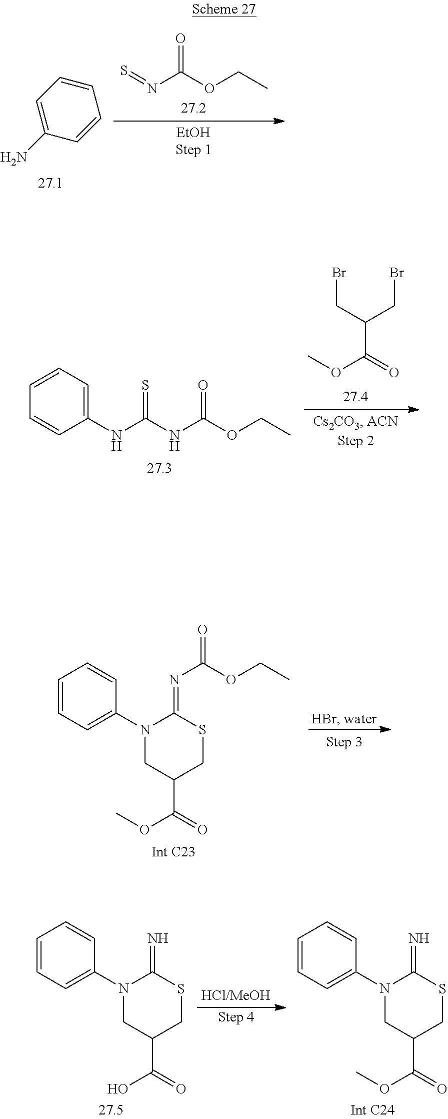

### Step 1. N-(Phenylcarbamothioyl)carbamate (27.3)

A mixture of aniline (27.1) (9.80 mL, 107 mmol) and O-ethyl carbonisothiocyanatidate (27.2) (12.7 mL, 107 mmol) in EtOH (100 mL) was stirred at 20° C. for 2 hours. The resulting precipitate was collected to afford 20.0 g of the title compound (27.3) as a solid.

¹H NMR (400 MHz, DMSO-d₆) δ 11.55 (br. s, 1H), 11.25 (br. s, 1H), 7.60 (d, J=7.6 Hz, 2H), 7.43-7.35 (m, 2H), 7.27-7.22 (m, 1H), 4.21 (q, J=7.0 Hz, 2H), 1.26 (t, J=7.2 Hz, 3H)

### Step 2. Methyl (Z)-2-((ethoxycarbonyl)imino)-3-phenyl-1,3-thiazinane-5-carboxylate (C23)

To a solution of ethyl N-(phenylcarbamothioyl)carbamate (27.3) (2.00 g, 8.92 mmol) in acetonitrile (20 mL) was added cesium carbonate (5.81 g, 17.8 mmol) followed by methyl 3-bromo-2-(bromomethyl)propanoate (27.4) (2.32 g, 8.92 mmol) at 20° C. The resulting mixture was stirred at 50° C. for 6 hours. The resulting precipitate was removed, and the filtrate was concentrated under reduced pressure. The residue was purified by silica gel chromatography, eluting with 10 to 50% EtOAc in petroleum ether to afford 1.60 g of methyl (Z)-2-((ethoxycarbonyl)imino)-3-phenyl-1,3-thiazinane-5-carboxylate (C23) as a solid.

### Step 3. 2-Imino-3-phenyl-1,3-thiazinane-5-carboxylic acid (27.5)

A mixture of methyl (Z)-2-((ethoxycarbonyl)imino)-3-phenyl-1,3-thiazinane-5-carboxylate (C23) (1.60 g, 4.96 mmol) in hydrobromic acid (15 mL) was stirred at 100° C. for 12 hours. The cooled reaction mixture was concentrated under reduced pressure to afford 1.17 g of crude 2-imino-3-phenyl-1, 3-thiazinane-5-carboxylic acid (27.5) hydrobromide as an oil, which was used without purification.

### Step 4. Methyl 2-imino-3-phenyl-1,3-thiazinane-5-carboxylate (C24)

A mixture of 2-imino-3-phenyl-1,3-thiazinane-5-carboxylic acid (27.5) hydrobromide (1.17 g, 3.69 mmol) in 4M HCl solution in MeOH (10 mL, 40.0 mmol) was stirred at 60° C. for 1.5 hours. The mixture was concentrated to dryness to afford 1.24 g of crude methyl 2-imino-3-phenyl-1,3-thiazinane-5-carboxylate (C24) hydrochloride as a solid, which was used without purification.

### Preparation of 3-Benzyl-1,3-thiazepan-2-imine (C25)

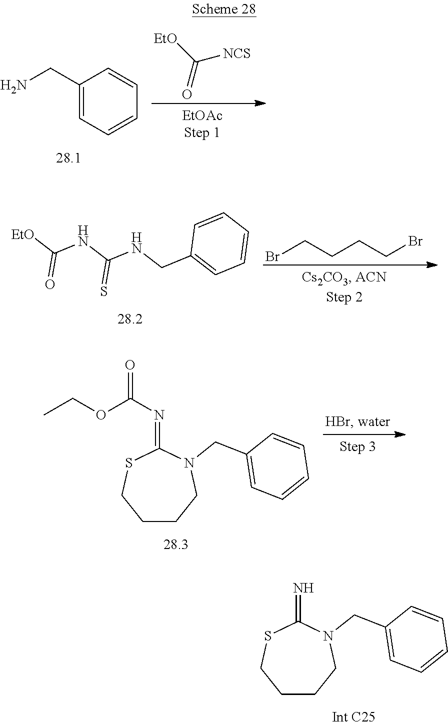

### Step 1. Ethyl N-(benzylcarbamothioyl)carbamate (28.2)

A mixture of phenylmethanamine (28.1) (2.03 mL, 18.7 mmol) in EtOAc (20 mL) and O-ethyl carbonisothiocyanatidate (2.21 mL, 18.7 mmol) was stirred at 20° C. for 1 hour. The reaction mixture was filtered, and the filter cake was collected and triturated with MeOH (10 mL) to afford 4.50 g of the crude title compound (28.2) as a solid which was used without further purification.

¹H NMR (400 MHz, DMSO-d₆) δ 11.06 (br. s, 1H), 10.23 (br. t, J=5.6 Hz, 1H), 7.35-7.27 (m, 5H), 4.81 (d, J=5.6 Hz, 2H), 4.14 (q, J=7.2 Hz, 2H), 1.21 (t, J=7.2 Hz, 3H)

LCMS (m/z [M+H]⁺): 239.0

### Step 2. Ethyl (Z)-(3-benzyl-1,3-thiazepan-2-ylidene)carbamate (28.3)

To a solution of ethyl N-(benzylcarbamothioyl)carbamate (28.2) (1.00 g, 4.20 mmol) in acetonitrile (100 mL) was added cesium carbonate (2.73 g, 8.39 mmol) and a solution of 1,4-dibromobutane (0.506 mL, 4.20 mmol) in acetonitrile (50 mL) at −5° C. The resulting mixture was stirred at 25° C. for 12 hours. The reaction was quenched by ice-water (50 mL) and extracted with EtOAc (3×100 mL). The combined organic layers were washed with brine (2×100 mL), dried over anhydrous Na₂SO₄, filtered, and concentrated. The residue was purified by reversed phase flash chromatography (0.1% TFA/MeCN condition) to afford 0.50 g of the title compound (28.3) as an oil.

¹H NMR (400 MHz, DMSO-d₆) δ 7.37-7.28 (m, 5H), 4.66 (s, 2H), 4.12 (q, J=6.8 Hz, 2H), 3.53-3.50 (m, 2H), 2.92-2.87 (m, 2H), 1.85-1.84 (m, 2H), 1.55-1.54 (m, 2H), 1.18 (t, J=6.8 Hz, 3H)

LCMS (m/z [M+H]⁺): 293.1

### Step 3. 3-Benzyl-1,3-thiazepan-2-imine (C25)

A mixture of hydrobromic acid (4.00 mL, 40% in water) and ethyl (Z)-(3-benzyl-1,3-thiazepan-2-ylidene)carbamate (28.3) (0.400 g, 1.37 mmol) was stirred at 100° C. for 12 hours. The reaction mixture was neutralized by a saturated solution of sodium carbonate and extracted with EtOAc (3×40 mL). The combined organic layers were washed with brine (2×50 mL), dried over anhydrous Na₂SO₄, filtered, and concentrated. The residue was purified by reversed phase flash chromatography (0.1% NH₃·H₂O/MeCN condition) to afford 0.15 g of 3-benzyl-1,3-thiazepan-2-imine (C25) as an oil.

### Preparation of 3-Phenyl-1,3-thiazepan-2-imine (C26)

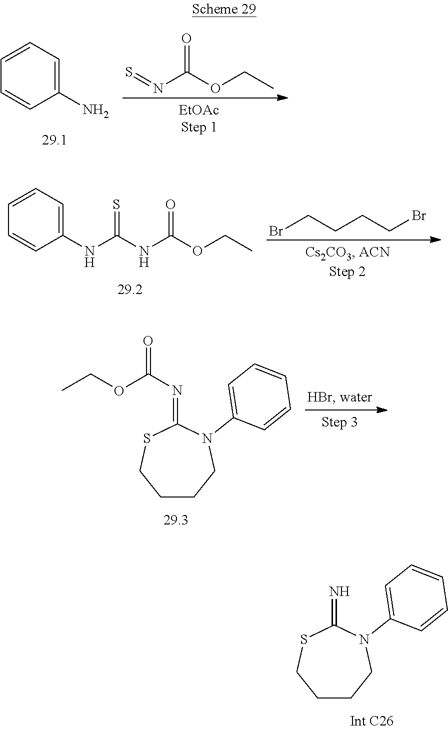

### Step 1. Ethyl N-(phenylcarbamothioyl)carbamate (29.2)

To a solution of aniline (29.1) (2.20 g, 23.6 mmol) in EtOAc (20 mL) was added ethyl N-(thioxomethylene)carbamate (3.10 g, 23.6 mmol) drop-wise at 0° C. The resulting mixture was stirred at 20° C. for 1 hour. The formed precipitate was collected by filtration and dried under reduced pressure to afford 3.70 g of the title compound (29.2) as a solid.

¹H NMR (400 MHz, CDCl₃) δ 11.46 (br. s, 1H), 8.29 (br. s, 1H), 7.64-7.42 (m, 2H), 7.40-7.29 (m, 2H), 7.24-7.16 (m, 1H), 4.28 (q, J=7.2 Hz, 2H), 1.35 (t, J=7.2 Hz, 3H)

LCMS (m/z [M+H]⁺): 225.1

### Step 2. Ethyl (Z)-(3-phenyl-1,3-thiazepan-2-ylidene)carbamate (29.3)

To a solution of ethyl N-(phenylcarbamothioyl)carbamate (29.2) (3.70 g, 16.5 mmol) in acetonitrile (500 mL) at −5° C. was added cesium carbonate (10.9 g, 33.0 mmol), followed by 1,4-dibromobutane (3.56 g, 16.5 mmol). The reaction mixture was stirred at −5° C. for 1 hour and then at 20° C. for 12 hours. Water (50 mL) was added into the reaction mixture which was then extracted with EtOAc (2×50 mL). The combined organic layers were dried over anhydrous Na₂SO₄, filtered, and concentrated. The residue was purified by silica gel column chromatography, eluting with 10 to 20% EtOAc in petroleum ether to afford 0.80 g of the tile compound (29.3) as a solid.

¹H NMR (400 MHz, CDCl₃) δ 7.34-7.29 (m, 2H), 7.18-7.15 (m, 3H), 4.07-3.96 (m, 4H), 3.02-2.97 (m, 2H), 2.05-1.98 (m, 2H), 1.76-1.63 (m, 2H), 1.19-1.15 (m, 3H)

LCMS (m/z [M+H]⁺): 279.1

### Step 3. 3-Phenyl-1,3-thiazepan-2-imine (C26)

A solution of (Z)-ethyl (3-phenyl-1,3-thiazepan-2-ylidene)carbamate (29.3) (0.800 g, 2.87 mmol) in hydrobromic acid (8.00 mL, 58.9 mmol, 40% in water) was stirred at 100° C. for 12 hours and then the cooled mixture was concentrated under reduced pressure. The residue was purified by reversed phase flash chromatography (0.1% HCl/MeCN condition) to afford 0.30 g of 3-phenyl-1,3-thiazepan-2-imine (C26) hydrobromide as a solid.

### Preparation of 3-fluoro-2-(2-iminothiazolidin-3-yl)phenol (C27)

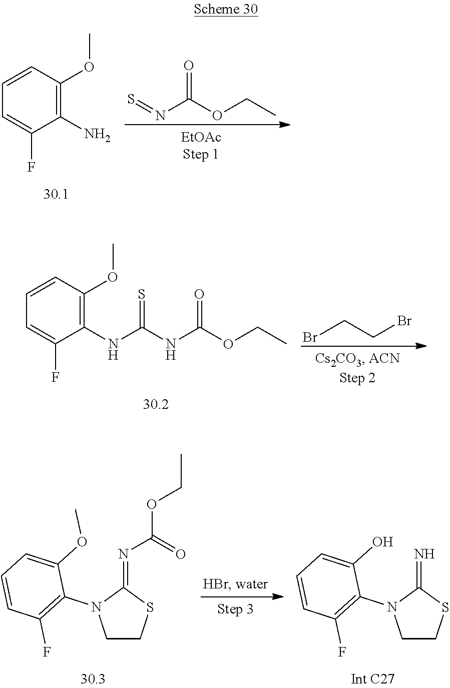

### Step 1. Ethyl N-[(2-fluoro-6-methoxy-phenyl)carbamothioyl]carbamate (30.2)

To a solution of 2-fluoro-6-methoxyaniline (30.1) (0.500 g, 3.54 mmol) in ethanol (15 mL) was added O-ethyl carbonisothiocyanatidate (0.464 g, 3.54 mmol). The resulting mixture was stirred at 25° C. for 1 hour. The reaction mixture was filtered, and the filter cake was collected to afford 0.80 g of ethyl N-[(2-fluoro-6-methoxy-phenyl)carbamothioyl]carbamate (30.2) as an off-white solid.

¹H NMR (400 MHz, DMSO-d₆) δ 11.40 (s, 1H), 10.72 (s, 1H), 7.36-7.30 (m, 1H), 6.93 (d, J=8.4 Hz, 1H), 6.89-6.84 (m, 1H), 4.21 (q, J=7.2 Hz, 2H), 3.80 (s, 3H), 1.26 (t, J=6.8 Hz, 3H).

### Step 2. (Z)-Ethyl (3-(2-fluoro-6-methoxyphenyl)thiazolidin-2-ylidene)carbamate (30.3)

To a solution of ethyl N-[(2-fluoro-6-methoxy-phenyl)carbamothioyl]carbamate (30.2) (0.800 g, 2.94 mmol) in acetonitrile (20 mL) was added cesium carbonate (1.910 g, 5.88 mmol) and 1,2-dibromoethane (0.550 g, 2.94 mmol). The resulting mixture was stirred at 50° C. for 12 hours. The reaction mixture was filtered and the filtrate was concentrated under reduced pressure to afford 0.750 g of (Z)-ethyl (3-(2-fluoro-6-methoxyphenyl)thiazolidin-2-ylidene)carbamate (30.3) as a yellow oil.

¹H NMR (400 MHz, DMSO-d₆) δ 7.45-7.39 (m, 1H), 7.01 (d, J=8.4 Hz, 1H), 6.94 (t, J=8.8 Hz, 1H), 3.98-3.91 (m, 3H), 3.83 (s, 3H), 3.80-3.73 (m, 1H), 3.37 (t, J=7.6 Hz, 2H), 1.11 (t, J=6.8 Hz, 3H).

### Step 3. 3-Fluoro-2-(2-iminothiazolidin-3-yl)phenol (C27)

To a solution of ethyl (Z)-ethyl (3-(2-fluoro-6-methoxyphenyl)thiazolidin-2-ylidene)carb-amate (30.3) (0.750 g, 2.51 mmol) was added hydrobromic acid (147 mmol, 20 mL, 40% purity in water). The resulting mixture was stirred at 100° C. for 12 hours. After cooled to rt, the reaction mixture was concentrated under reduced pressure. After adjusted to pH 7 with saturated sodium bicarbonate aqueous solution. The residue was purified by reversed-phase flash chromatography (0.1% NH₃·H₂O/MeCN condition) to afford 0.310 g of 3-fluoro-2-(2-iminothiazolidin-3-yl)phenol (C27) as a white solid.

### Preparation of 4-fluoro-2-(2-iminothiazolidin-3-yl)phenol (C32)

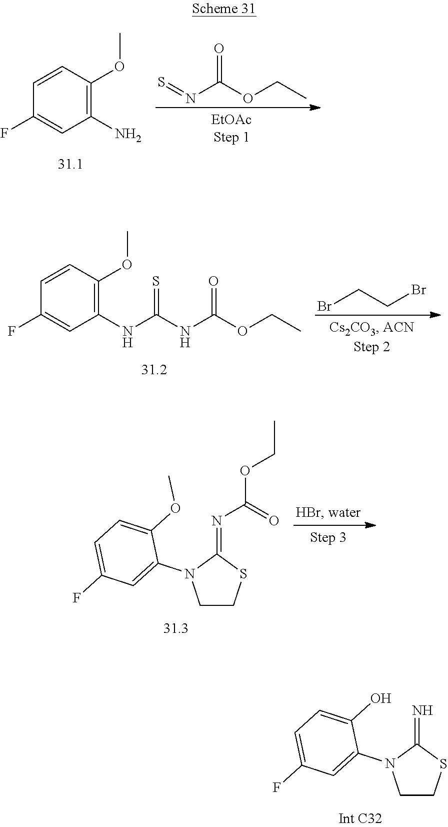

### Step 1. Ethyl N-[(5-fluoro-2-methoxy-phenyl)carbamothioyl]carbamate (31.2)

To a solution of 5-fluoro-2-methoxyaniline (31.1) (1.88 g, 13.32 mmol) and O-ethyl carbonisothiocyanatidate (1.75 g, 13.32 mmol) in ethanol (10 mL) was stirred at 20° C. for 1 hour. Solid precipitates was collected by filtration to afford 2.10 g of ethyl N-[(5-fluoro-2-methoxy-phenyl)carbamothioyl]carbamate (31.2) as a yellow solid. LCMS (m/z [M+H]⁺): 273.0

### Step 2. (Z)-ethyl (3-(5-fluoro-2-methoxyphenyl)thiazolidin-2-ylidene)carbamate (31.3)

To a solution of ethyl N-[(5-fluoro-2-methoxy-phenyl)carbamothioyl]carbamate (31.2) (2.10 g, 7.71 mmol) in acetonitrile (20 mL) was added 1,2-dibromoethane (1.45 g, 7.71 mmol) and cesium carbonate (7.54 g, 23.14 mmol)). The resulting mixture was stirred at 50° C. for 2 hours. The reaction mixture was poured into water (50 mL) and extracted with ethyl acetate (3×30 mL). The combined organic phases were dried over anhydrous sodium sulfate, filtered and the filtrate concentrated under reduced pressure to afford 2.10 g of (Z)-ethyl (3-(5-fluoro-2-methoxyphenyl)thiazolidin-2-ylidene)carbamate (31.3) as a yellow solid. LCMS (m/z [M+H]⁺): 299.3

### Step 3. 4-fluoro-2-(2-iminothiazolidin-3-yl)phenol (C32)

To a solution of ethyl (Z)-ethyl (3-(5-fluoro-2-methoxyphenyl)thiazolidin-2-ylidene)carbamate (31.3) (2.10 g, 7.04 mmol) in hydrobromic acid (184.15 mmol, 25.00 mL, 40% purity in water). The resulting mixture was stirred at 100° C. for 24 hours. The reaction mixture was concentrated under reduced pressure. The residue was purified by preparative HPLC (column: Phenomenex C18 50×250 mm, 10 μm; mobile phase A: water/10 mM NH₄HCO₃, mobile phase B: ACN; gradient: 0% B to 20% B over min to afford 0.080 g of 4-fluoro-2-(2-iminothiazolidin-3-yl)phenol (C32) as a white solid.

### Preparation of 6-(2-iminothiazolidin-3-yl)pyridin-2-ol (C34)

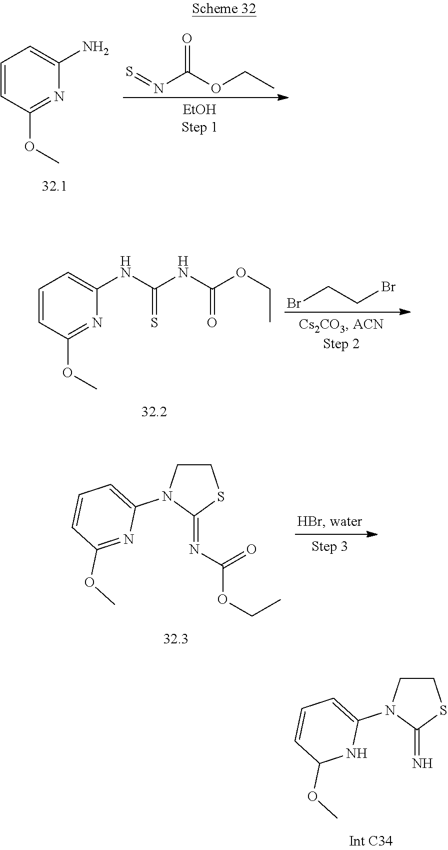

### Step 1. ethyl N-[(6-methoxy-2-pyridyl)carbamothioyl]carbamate (32.2)

To a solution of 6-methoxypyridin-2-amine (32.1) (7.00 g, 56.4 mmol) in ethanol (25 mL) was added ethyl N-(thioxomethylene)carbamate (7.44 g, 56.7 mmol) at 0° C. Te resulting mixture was stirred at 25° C. for 12 hours. The reaction solution was concentrated under reduced pressure to afford 14.08 g of ethyl N-[(6-methoxy-2-pyridyl)carbamothioyl]carbamate (32.2) as a yellow solid.

¹H NMR (400 MHz, DMSO-d₆) δ 12.04 (br s, 1H), 11.56 (br s, 1H), 7.77 (t, J=8.4 Hz, 1H), 6.66 (t, J=8.0 Hz, 1H), 5.87-5.83 (m, 1H), 4.21 (q, J=7.2 Hz, 2H), 3.84 (s, 3H), 1.25 (t, J=7.2 Hz, 3H).

### Step 2. ethyl (NZ)—N-[3-(6-methoxy-2-pyridyl)thiazolidin-2-ylidene]carbamate (32.3)

To a solution of ethyl N-[(6-methoxy-2-pyridyl)carbamothioyl]carbamate (32.2) (14.8 g, 56.1 mmol) and 1,2-dibromoethane (10.5 g, 56.1 mmol) in acetonitrile (80 mL) was added cesium carbonate (36.5 g, 112 mmol) at 25° C. The resulting mixture was stirred at 40° C. for 4 hours. The reaction mixture was filtered and the filtrate was concentrated under reduced pressure. The residue was purified by chromatography (Silica gel, petroleum ether: ethyl acetate=1:0 to 3:1) to afford 10.8 g of ethyl (NZ)—N-[3-(6-methoxy-2-pyridyl)thiazolidin-2-ylidene]carbamate (32.3) as a yellow solid. LCMS (m/z [M+H]⁺): 282.0

¹H NMR (400 MHz, DMSO-d₆) δ 7.48-7.29 (m, 4H), 4.02-3.93 (m, 4H), 3.37 (t, J=7.6 Hz, 2H), 1.25 (t, J=7.2 Hz, 3H).

### Step 3. 6-(2-iminothiazolidin-3-yl)pyridin-2-ol (C34)

Ethyl (NZ)—N-[3-(6-methoxy-2-pyridyl)thiazolidin-2-ylidene]carbamate (32.3) (1.76 g, 5.46 mmol) and hydrobromic acid (5 mL, 48% purity in water) were mixed at 25° C. The resulting mixture was stirred at 100° C. for 12 hours under nitrogen atmosphere. The reaction solution was concentrated under reduced pressure. Methanol (15 mL) was added and the resulting solution was stirred for 15 minutes. White solid precipitates was collected by filteration to afford 0.483 g of 6-(2-iminothiazolidin-3-yl)pyridin-2-ol as a hydrobromate (C34) as a white solid.

### Preparation of 3-(2-imino-1,3-thiazinan-3-yl)phenol (C35)

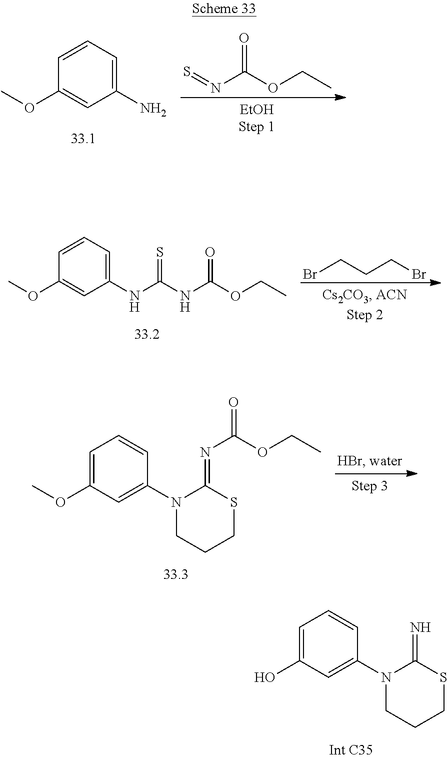

### Step 1. ethyl N-[(3-methoxyphenyl)carbamothioyl]carbamate (33.2)

To a solution of 3-methoxyaniline (33.1) (1.00 g, 8.12 mmol) in ethanol (15 mL) was added ethyl N-(thioxomethylene)carbamate (1.06 g, 8.12 mmol) at 0° C. The resulting mixture was stirred at 25° C. for 1 hour. The reaction mixture was filtered to afford 1.40 g of ethyl N-[(3-methoxyphenyl)carbamothioyl]carbamate (33.2) as a white solid.

¹H NMR (400 MHz, DMSO-d₆) δ 11.55 (br. s, 1H), 11.24 (br. s, 1H), 7.35 (t, J=2.0 Hz, 1H), 7.29 (t, J=8.0 Hz, 1H), 7.14-7.12 (m, 1H), 6.84-6.81 (m, 1H), 4.21 (q, J=7.2 Hz, 2H), 3.75 (s, 3H), 1.26 (d, J=6.8 Hz, 3H). LCMS (m/z [M+H]⁺): 255.1

### Step 2. Ethyl (NZ)—N-[3-(3-methoxyphenyl)-1,3-thiazinan-2-ylidene]carbamate (33.3)

To a solution of ethyl N-[(3-methoxyphenyl)carbamothioyl]carbamate (3) (0.700 g, 2.75 mmol) in acetonitrile (15 mL) was added 1,3-dibromopropane (33.2) (0.560 g, 2.75 mmol) and cesium carbonate (1.79 g, 5.51 mmol) at 25° C. The resulting mixture was stirred at 50° C. for 4 hours. The reaction mixture was filtered and the filtrate was concentrated under reduced pressure to afford 0.800 g of ethyl (NZ)—N-[3-(3-methoxyphenyl)-1,3-thiazinan-2-ylidene]carbamate (33.3) as a white solid.

¹H NMR (400 MHz, DMSO-d₆) δ 7.31 (t, J=8.0 Hz, 1H), 6.88-6.80 (m, 3H), 3.84 (q, J=6.8 Hz, 2H), 3.76 (s, 3H), 3.68 (t, J=6.0 Hz, 2H), 3.09 (t, J=6.0 Hz, 2H), 2.23-2.20 (m, 2H), 1.06 (t, J=7.2 Hz, 3H). LCMS (m/z [M+H]⁺): 295.0

### Step 3. 3-(2-Imino-1,3-thiazinan-3-yl)phenol (C35)

A solution of ethyl (NZ)—N-[3-(3-methoxyphenyl)-1,3-thiazinan-2-ylidene]carbamate (33.3) (0.800 g, 2.72 mmol) in hydrobromic acid (10 mL, 40% purity in water) was stirred at 100° C. for 12 hours. The reaction mixture was concentrated under reduced pressure. The crude product was triturated with acetonitrile (20 mL) to afford 0.550 g of 3-(2-imino-1,3-thiazinan-3-yl)phenol as hydrobromide (C35) as a light yellow solid.

### Preparation of (3-(2-chlorophenyl)-2-iminothiazolidin-4-yl)methanol (C36)

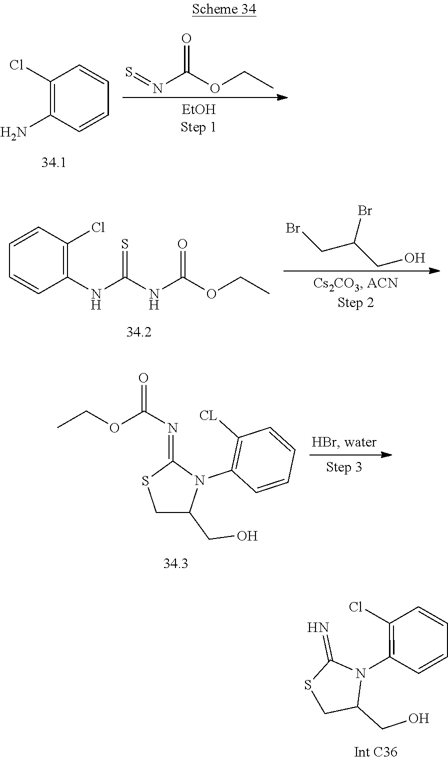

### Step 1. Ethyl N-[(2-chlorophenyl)carbamothioyl]carbamate (34.2)

To a solution of 2-chloroaniline (34.1) (1.00 g, 7.84 mmol) in ethanol (12 mL) was added ethyl O-ethyl carbonisothiocyanatidate (1.03 g, 7.84 mmol) at 0° C. and the solution was stirred at 25° C. for 1 hour. The reaction mixture was filtered and the filter cake was collected to afford 1.23 g of ethyl N-[(2-chlorophenyl)carbamothioyl]carbamate (34.2) as a white solid.

¹H NMR (400 MHz, DMSO-d₆) δ 11.57-11.44 (m, 2H), 7.54 (dd, J=1.6 Hz, 8.0 Hz, 1H), 7.21-7.07 (m, 1H), 7.35-7.29 (m, 2H), 4.24 (q, J=6.8 Hz, 2H), 1.24 (t, J=7.2 Hz, 3H). LCMS (m/z [M+H]⁺): 259.1

### Step 2. (Z)-ethyl (3-(2-chlorophenyl)-4-(hydroxymethyl)thiazolidin-2-ylidene)carbamate (34.3)

To a solution of ethyl N-[(2-chlorophenyl)carbamothioyl]carbamate (34.2) (0.620 g, 2.40 mmol) in acetonitrile (8 mL) was added cesium carbonate (1.56 g, 4.79 mmol) and 2,3-dibromopropan-1-ol (0.522 g, 2.40 mmol), and then the solution was stirred at 25° C. for 1 hour. The mixture was filtered and filtrate was concentrated under reduced pressure. The residue was purified by column chromatography (Silica gel, petroleum ether/ethyl acetate=99:1 to 2:3) to afford 0.305 g of (Z)-ethyl (3-(2-chlorophenyl)-4-(hydroxymethyl)thiazolidin-2-ylidene)carbamate (34.3) as a yellow oil.

¹H NMR (400 MHz, CDCl₃) δ 7.51-7.49 (m, 1H), 7.30-7.27 (m, 3H), 4.37 (s, 1H), 4.15 (q, J=6.4 Hz, 2H), 3.77-3.75 (m, 1H), 3.67-3.65 (m, 1H), 3.56-3.55 (m, 1H), 3.43-3.41 (m, 1H), 1.27 (t, J=7.2 Hz, 3H).

### Step 3. (3-(2-chlorophenyl)-2-iminothiazolidin-4-yl)methanol (C36)

A solution of (Z)-ethyl (3-(2-chlorophenyl)-4-(hydroxymethyl)thiazolidin-2-ylidene)carbamate (34.3) (0.385 g, 1.22 mmol) in hydrobromic acid (0.5 mL, 40% purity in water) was stirred at 100° C. for 1 hour. The reaction mixture was concentrated under reduced pressure. The crude product was purified by reversed-phase flash chromatography (0.1% NH₃·H₂O/MeCN condition) to afford 0.250 g of (3-(2-chlorophenyl)-2-iminothiazolidin-4-yl)methanol (C36) as a white solid.

¹H NMR (400 MHz, DMSO-d₆) δ 7.60 (t, J=5.2 Hz, 1H), 7.64-7.41 (m, 3H), 5.03 (t, J=4.8 Hz, 1H), 4.21-4.20 (m, 1H), 3.62-3.58 (m, 1H), 3.41-3.37 (m, 4H).

Intermediates D1-D11 in Table D were prepared with methods depicted below in Schemes 35-36.

TABLE D

| | | Prep. | | |
|---|---|---|---|---|
| Int. | Structure | Method | LCMS | 1H NMR |
| D1 | 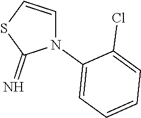 [SMILES: N=c1sccn1-c1ccccc1Cl] | Scheme 30 | 211.1 | (DMSO-d₆, 400 MHz) δ 7.82 (br. s, 1H), 7.65 − 7.61 (m, 1H), 7.51 − 7.43 (m, 3H), 6.76 (d, J = 4.8 Hz, 1H), 6.17 (d, J = 5.2 Hz, 1H). |
| D2 | 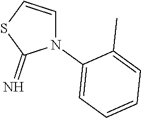 | Scheme 30 | 191.1 | (CDCl₃, 400 MHz) δ 7.40 − 7.27 (m, 4H), 6.42 (d, J = 5.2 Hz, 1H), 5.94 (d, J = 5.2 Hz, 1H), 4.73 (br. d, J = 2.4 Hz, 1H), 2.27 (s, 3H). |
| D3 | 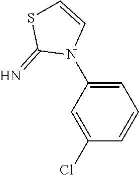 [SMILES: N=c1sccn1-c1cccc(Cl)c1] | Scheme 30 | 211.1 | (DMSO-d₆, 400 MHz) δ 8.30 (br. s, 1H), 7.80 (s, 1H), 7.56 − 7.54 (m, 1H), 7.45 (t, J = 8.0 Hz, 1H), 7.32 − 7.30 (m, 1H), 7.08 (d, J = 5.2 Hz, 1H), 6.21 (d, J = 5.2 Hz, 1H). |
| D4 | 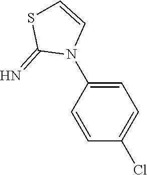 [SMILES: N=c1sccn1-c1ccc(Cl)cc1] | Scheme 30 | 211.1 | (DMSO-d₆, 400 MHz) δ 8.20 (br. s, 1H), 7.64 (d, J = 8.8 Hz, 2H), 7.48 (d, J = 8.8 Hz, 2H), 7.03 (d, J = 5.2 Hz, 1H), 6.20 (d, J = 5.2 Hz, 1H). |
| D5 | 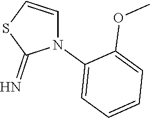 | Scheme 30 | 207.2 | (DMSO-d₆, 400 MHz) δ 7.62 (br. s, 1H), 7.39 − 7.35 (m, 1H), 7.33 − 7.30 (m, 1H), 7.17 (d, J = 8.4 Hz, 1H), 7.01 (t, J = 7.2Hz, 1H), 6.66 (d, J = 5.2 Hz, 1H), 6.05 (d, J = 5.2 Hz, 1H), 3.79 (s, 3H). |
| D6 | 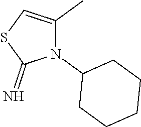 | Scheme 31 | 197.0 | (METHANOL-d₄, 400 MHz) δ 5.80 (s, 1H), 2.22 (s, 3H), 2.20 − 2.09 (m, 1H), 1.94 − 1.64 (m, 6H), 1.52 − 1.39 (m, 2H), 1.38 − 1.23 (m, 2H). |
| D7 | 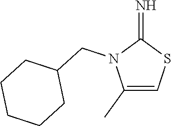 | Scheme 31 | 210.9 | (DMSO-d₆, 400 MHz) δ 7.40 (br. s, 1H), 5.60 (d, J = 1.2 Hz, 1H), 3.42 (d, J = 7.2 Hz, 2H), 2.01 (s, 3H), 1.88 − 1.77 (m, 1H), 1.68 − 1.59 (m, 3H), 1.57-1.54 (m, 2H), 1.18 − 1.09 (m, 3H), 0.98 − 0.92 (m, 2H). |
| D8 | 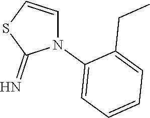 | Scheme 30 | 205.3 | (CDCl₃, 400 MHz) δ 7.39 (d, J = 4.0 Hz, 2H), 7.34 − 7.28 (m, 1H), 7.27 − 7.23 (m, 1H), 6.40 (d, J = 4.8 Hz, 1H), 5.91 (d, J = 5.2 Hz, 1H), 2.62 (q, J = 7.6 Hz, 2H), 1.21 (t, J = 7.2 Hz, 3H). |
| D9 | 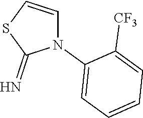 [SMILES: N=c1sccn1-c1ccccc1C(F)(F)F] | Scheme 30 | 245.0 | (CDCl₃, 400 MHz) δ 7.83 (d, J = 7.6 Hz, 1H), 7.73 − 6.69 (m, 1H), 7.58 (t, J = 7.6 Hz, 1H), 7.51 (d, J = 8.0 Hz, 1H), 6.42 (d, J = 4.8 Hz, 1H), 5.94 (d, J = 4.8 Hz, 1H). |
| D10 | 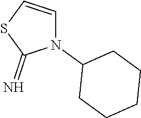 [SMILES: N=c1sccn1C1CCCCC1] | Scheme 30 | 183.0 | (CDCl₃, 400 MHz) δ 6.52 (d, J = 4.8 Hz, 1H), 5.77 (d, J = 4.8 Hz, 1H), 4.29- 4.19 (m, 1H), 2.05 − 2.00 (m, 2H), 1.88 − 1.82 (m, 2H), 1.50 − 1.39 (m, 4H), 1.20 − 1.15 (m, 2H). |
| D11 | 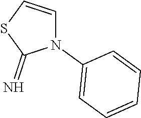 [SMILES: N=c1sccn1-c1ccccc1] | Scheme 30 | 176.2 | NA |

### Preparation of 3-(2-Ethylphenyl)thiazol-2(3H)-imine (D8)

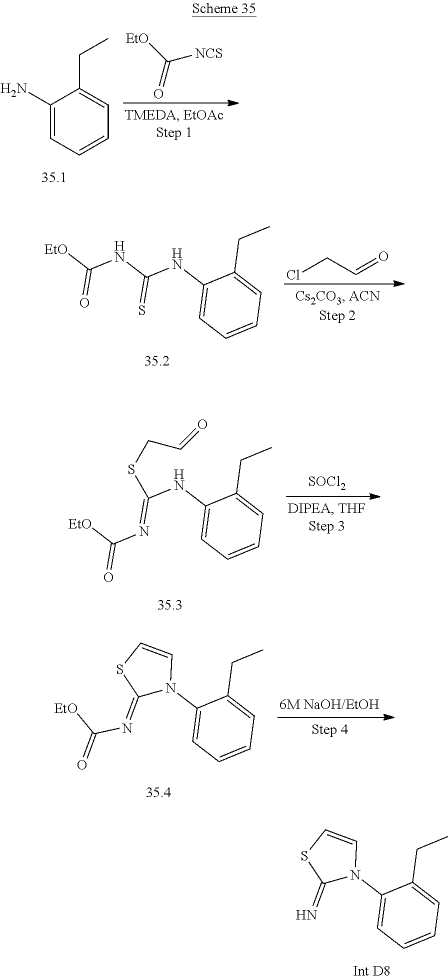

### Step 1. Ethyl N-[(2-ethylphenyl)carbamothioyl]carbamate (35.2)

To a mixture of 2-ethylaniline (35.1) (1.00 g, 8.25 mmol) and O-ethyl carbonisothiocyanatidate (1.08 g, 8.25 mmol) in EtOAc (20 mL) was added tetramethylethylenediamine (0.096 g, 0.825 mmol) at 25° C. The reaction mixture was stirred at 25° C. for 5 hours, and then concentrated under reduced pressure. The residue was triturated with petroleum ether (50 mL) to afford 1.85 g of the title compound (35.2) as a solid.

¹H NMR (400 MHz, CD₃OD) δ 7.50 (d, J=2.0 Hz, 1H), 7.28 (d, J=1.6 Hz, 1H), 7.25-7.21 (m, 2H), 4.28 (q, J=7.2 Hz, 2H), 2.60 (q, J=7.6 Hz, 2H), 1.35 (t, J=7.2 Hz, 3H), 1.19 (t, J=7.6 Hz, 3H) LCMS (m/z [M+H]⁺): 253.2

### Step 2. Ethyl (Z)-(((2-ethylphenyl)amino)((2-oxoethyl)thio)methylene)carbamate (35.3)

To a mixture of ethyl N-[(2-ethylphenyl)carbamothioyl]carbamate (35.2) (1.80 g, 7.13 mmol) and cesium carbonate (4.65 g, 14.3 mmol) in acetonitrile (20 mL) was added 2-chloroacetaldehyde (14.0 g, 71.3 mmol) at 0° C. The reaction mixture was stirred at 25° C. for 3 hours then quenched with water (40 mL) followed by extraction with EtOAc (3×50 mL). The combined organic layers were washed with brine (2×30 mL), dried over anhydrous Na₂SO₄, filtered, and concentrated to afford 1.70 g of the crude title compound (35.3) as an oil.

¹H NMR (400 MHz, CDCl₃) δ 7.44-7.28 (m, 4H), 3.70-3.61 (m, 2H), 3.38-3.22 (m, 2H), 2.57-2.41 (m, 2H), 1.25-1.18 (m, 6H) LCMS (m/z [M+H]⁺): 295.1

### Step 3. Ethyl (Z)-(3-(2-ethylphenyl)thiazol-2(3H)-ylidene)carbamate (35.4)

To a solution of (Z)-ethyl (((2-ethylphenyl)amino)((2-oxoethyl)thio)methylene)carbamate (35.3) (1.70 g, 5.78 mmol) in THF (20 mL) was added DIEA (2.24 g, 17.3 mmol) and thionyl chloride (0.760 g, 6.35 mmol) at 0° C. The mixture was stirred at 25° C. for 3 hours then quenched with water (40 mL) and extracted with DCM (3×50 mL). The combined organic layers were washed with brine (2×40 mL), dried over anhydrous Na₂SO₄, filtered, and concentrated. The residue was purified by silica gel column chromatography, eluting with 10 to 30% EtOAc in petroleum ether to afford 0.47 g of the title compound (35.4) as an oil.

¹H NMR (400 MHz, CDCl₃) δ 7.42-7.38 (m, 2H), 7.31-7.29 (m, 1H), 7.21 (d, J=8.0 Hz, 1H), 6.86 (d, J=4.8 Hz, 1H), 6.69 (d, J=4.8 Hz, 1H), 4.20 (q, J=7.2 Hz, 2H), 2.52-2.40 (m, 2H), 1.28 (t, J=7.2 Hz, 3H), 1.14 (t, J=7.6 Hz, 3H) LCMS (m/z [M+H]⁺): 277.2

### Step 4. 3-(2-Ethylphenyl)thiazol-2(3H)-imine (D8)

To a solution of (Z)-ethyl (3-(2-ethylphenyl)thiazol-2(3H)-ylidene)carbamate (35.4) (0.470 g, 1.58 mmol) in EtOH (10 mL) was added sodium hydroxide (1.36 g, 34.0 mmol) at 25° C. The mixture was stirred at 60° C. for 1 hour, and then quenched with water (30 mL), extracted with DCM (3×50 mL). The combined organic layers were washed with brine (2×40 mL), dried over anhydrous Na₂SO₄, filtered, and concentrated. The residue was purified by reversed phase flash chromatography (0.1% NH₃·H₂O/MeCN condition) to afford 0.14 g of 3-(2-ethylphenyl)thiazol-2(3H)-imine (D8) as an oil.

### Preparation of 3-cyclohexyl-4-methylthiazol-2(3H)-imine (D6)

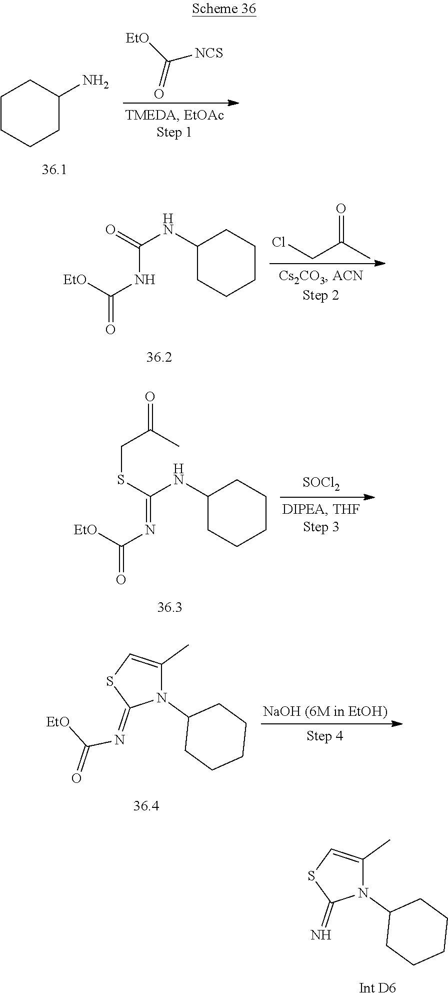

### Step 1. Ethyl N-(cyclohexylcarbamothioyl)carbamate (36.2)

To a mixture of cyclohexylamine (36.1) (0.800 g, 8.07 mmol) and O-ethyl carbonisothiocyanatidate (1.11 g, 8.47 mmol) in EtOAc (10 mL) was added TMEDA (93 mg, 0.806 mmol) in at 25° C. The mixture was stirred for 6 hours and then concentrated under reduced pressure. The residue was purified by column chromatography on silica gel (column height: 250 mm, diameter: 100 mm, 100-200 mesh silica gel, petroleum ether/EtOAc=100/1 to 3/1) to afford 1.80 g of the title compound (36.2) as a gum.

¹H NMR (400 MHz, CD₃Cl) δ 9.64 (br. s, 1H), 7.96 (br. s, 1H), 4.29-4.22 (m, 2H), 2.09-2.01 (m, 2H), 1.77-1.68 (m, 2H), 1.62-1.63 (m, 1H), 1.49-1.33 (m, 4H), 1.33-1.19 (m, 5H) LCMS (m/z [M+H]⁺): 231.1

### Step 2. (Z)-Ethyl ((cyclohexylamino)((2-oxopropyl)thio)methylene)carbamate (36.3)

To a mixture of ethyl N-(cyclohexylcarbamothioyl)carbamate (36.2) (0.800 g, 3.47 mmol) and cesium carbonate (1.92 g, 5.90 mmol) in acetonitrile (10 mL) was added 1-chloropropan-2-one (0.353 g, 3.82 mmol) at 25° C. The resulting mixture was stirred for 4 hours and then poured into ice-water (10 mL) and extracted with MTBE (100 mL×2). The combined organic phase was washed with brine (20 mL×2), dried over anhydrous sodium sulfate, filtered, and concentrated under reduced pressure to afford 900 mg of the crude title compound (36.3) as a solid, which was used without purification.

LCMS (m/z [M+H]⁺): 287.1

### Step 3. (Z)-Ethyl (3-cyclohexyl-4-methylthiazol-2(3H)-ylidene)carbamate (36.4)

To a mixture of (Z)-ethyl ((cyclohexylaminox(2-oxopropyl)thio)methylene)carbamate (36.3) (0.900 g, 3.14 mmol) and DIPEA (0.812 g, 6.29 mmol) in THF (20 mL) as added thionyl chloride (0.224 g, 1.89 mmol) at 0° C. The mixture was stirred at 25° C. for 2 hours then poured into ice-water (20 mL) and extracted with EtOAc (100 mL×3). The combined organic phase was washed with brine (20 mL), dried over anhydrous sodium sulfate, filtered, and concentrated under reduced pressure. The residue was purified by column chromatography on silica gel (petroleum ether/EtOAc=100/1 to 4/1) to afford 0.50 g of the title compound (36.4) as a solid.

¹H NMR (400 MHz, DMSO-d₆) δ 6.54 (br. s, 1H), 4.09-4.03 (m, 2H), 2.29-2.25 (m, 3H), 1.82-1.62 (m, 6H), 1.36-1.23 (m, 3H), 1.21-1.19 (m, 5H)

LCMS (m/z [M+H]⁺): 268.9.

### Step 4. 3-Cyclohexyl-4-methylthiazol-2(3H)-imine (D6)

To a mixture of (Z)-ethyl (3-cyclohexyl-4-methylthiazol-2(3H)-ylidene)carbamate (36.4) (0.200 g, 0.745 mmol) in EtOH (4 mL) was added NaOH (0.596 g, 14.9 mmol) at 25° C. The stirred mixture was heated to 50° C. for 1 hour then poured into ice-water (50 mL) and extracted with EtOAc (5×50 mL). The combined organic phase was washed with brine (10×2 mL), dried over anhydrous sodium sulfate, filtered, and concentrated under reduced pressure to afford 0.120 g of 3-cyclohexyl-4-methylthiazol-2(3H)-imine (D6) as a solid.

Intermediates E3-E17 in Table E were prepared with methods shown below in Schemes 37-50.

TABLE E

| Int. | Structure | LCMS | 1H NMR |
|---|---|---|---|
| E3 | 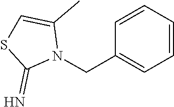 | NA | (DMSO-d₆, 400 MHz) δ 10.08 (br. s, 2H), 7.40 − 7.44 (m, 2H), 7.35 − 7.37 (m, 1H), 7.15 (d, J = 7.2 Hz, 2H), 6.79 (s, 1H), 5.42 (s, 2H), 2.13 (s, 3H). |
| E4 | 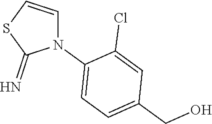 [SMILES: N=c1sccn1-c1ccc(CO)cc1Cl] | 241.0 | (CDCl₃, 400 MHz) δ 7.49 (d, J = 8.4 Hz, 1H), 7.45 (d, J = 2.0 Hz, 1H), 7.32 − 7.30 (m, 1H), 6.45 (d, J = 4.8 Hz, 1H), 5.98 (d, J = 4.8 Hz, 1H), 4.75 (br. s, 1H), 4.57 (s, 2H). |
| E5 | 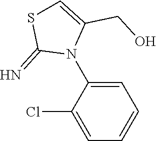 [SMILES: N=c1scc(CO)n1-c1ccccc1Cl] | 241.0 | (DMSO-d₆, 400 MHz) δ 7.69-7.67 (m, 1H), 7.54 − 7.50 (m, 4H), 6.32 (br. s, 1H), 5.21 (br. s, 1H), 3.85 − 3.83 (m, 2H). |
| E6 | 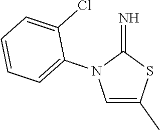 | 224.8 | (DMSO-d₆, 400 MHz) δ 7.71 (br. s, 1H), 7.64 − 7.58 (m, 1H), 7.49 − 7.40 (m, 3H), 6.51(q, J = 1.2 Hz, 1H), 2.03 (s, 3H). |
| E7 | 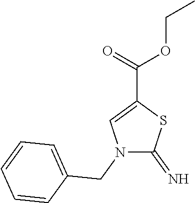 [SMILES: CCOC(=O)c1cn(Cc2ccccc2)c(=N)s1] | 263.0 | (DMSO-d₆, 400 MHz) δ 10.36 (br. s, 2H), 8.45 (s, 1H), 7.47 − 7.34 (m, 5H), 5.36 (s, 2H), 4.30 (q, 7.2 Hz, 2H), 1.27 (t, J = 7.2 Hz, 3H). |
| E8 | 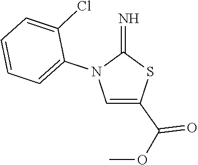 [SMILES: COC(=O)c1cn(-c2ccccc2Cl)c(=N)s1] | NA | (CD₃OD, 400 MHz) δ 8.26 (s, 1H), 7.79 (dt, J = 1.2, 8.4 Hz, 2H), 7.73 (dt, J = 1.6, 7.6 Hz, 1H), 7.65 (dt, J = 1.6, 8.0 Hz, 1H), 3.93 (s, 3H). |
| E9 | 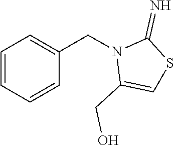 [SMILES: N=c1scc(CO)n1Cc1ccccc1] | NA | (DMSO-d₆, 400 MHz) δ 7.62 (s, 1H), 7.33 − 7.29 (m, 2H), 7.24 − 7.19 (m, 3H), 5.90 (s, 1H), 4.96 (s, 2H), 4.04 (s, 2H). |
| E10 | 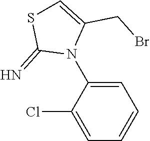 [SMILES: N=c1scc(CBr)n1-c1ccccc1Cl] | 305.0 | NA |
| E11 | 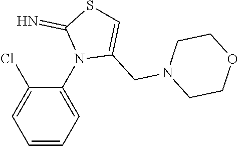 [SMILES: N=c1scc(CN2CCOCC2)n1-c1ccccc1Cl] | 310.1 | NA |
| E12 | 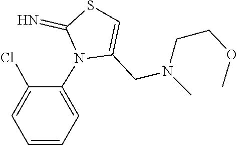 [SMILES: COCCN(C)Cc1csc(=N)n1-c1ccccc1Cl] | 312.0 | (DMSO-d₆, 400 MHz) δ 8.22 (s, 1H), 7.66 − 7.61 (m, 1H), 7.52 − 7.44 (m, 3H), 6.23(s, 1H), 3.09 (s, 3H), 3.04 − 2.98 (m, 2H), 2.96 − 2.88 (m, 2H), 2.29 − 2.20 (m, 2H), 2.01 (s, 3H). |
| E13 | 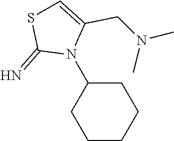 [SMILES: CN(C)Cc1csc(=N)n1C1CCCCC1] | 240.3 | NA |
| E14 | 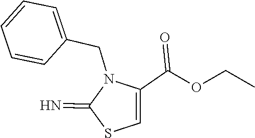 [SMILES: CCOC(=O)c1csc(=N)n1Cc1ccccc1] | 263.2 | (DMSO-d₆, 400 MHz) δ 9.97 (br. s, 1H), 7.98 (s, 1H), 7.40 − 7.37 (m, 2H), 7.34 − 7.29 (m, 1H), 7.13 (d, J = 7.2 Hz, 2H), 5.59 (s, 2H), 4.22 (q, J = 7.2 Hz, 2H), 1.19 (t, J = 7.2 Hz, 3H). |
| E15 | 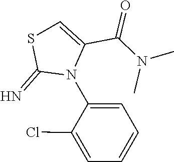 [SMILES: CN(C)C(=O)c1csc(=N)n1-c1ccccc1Cl] | 304.0 | (DMSO-d₆, 400 MHz) δ 8.04 (s, 1H), 7.53 − 7.51 (m, 1H), 7.38 − 7.36 (m, 3H), 6.52 (s, 1H), 3.11 (s, 3H), 2.71 (s, 3H). |
| E16 | 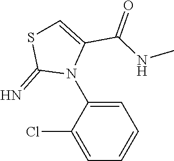 [SMILES: CNC(=O)c1csc(=N)n1-c1ccccc1Cl] | 268.0 | (DMSO-d₆, 400 MHz) δ 8.36 (br. s, 1H), 7.54 − 7.51 (m, 1H), 7.50 − 7.46 (m, 1H), 7.40 − 7.35 (m, 3H), 6.83 (br. s, 1H), 2.53 (s, 3H). |
| E17 | 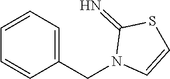 [SMILES: N=c1sccn1Cc1ccccc1] | NA | (CDCl₃, 400 MHz) δ 7.48 − 7.27 (m, 5H), 6.67 − 6.54 (m, 1H), 6.34 − 6.32 (m, 1H), 5.75 − 5.74 (m, 1H), 4.89 (s, 2H). |

### Preparation of 3-benzyl-4-methylthiazol-2(3H)-imine (E3)

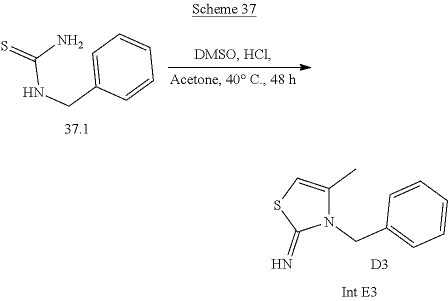

To a solution of benzylthiourea (37.1) (1.00 g, 6.02 mmol) in acetone (15 mL) was added HCl (1.20 g, 12.0 mmol, 1.18 mL, 36% purity) and DMSO (939 mg, 12.0 mmol, 939 μL) at 40° C. The reaction mixture was stirred for 48 hours then concentrated under reduced pressure. The residue was purified by reverse phase MPLC to afford 0.80 g of the 3-benzyl-4-methylthiazol-2(3H)-imine E3 as a solid.

### Preparation of (3-Chloro-4-(2-iminothiazol-3(2H)-yl)phenyl)methanol (E4)

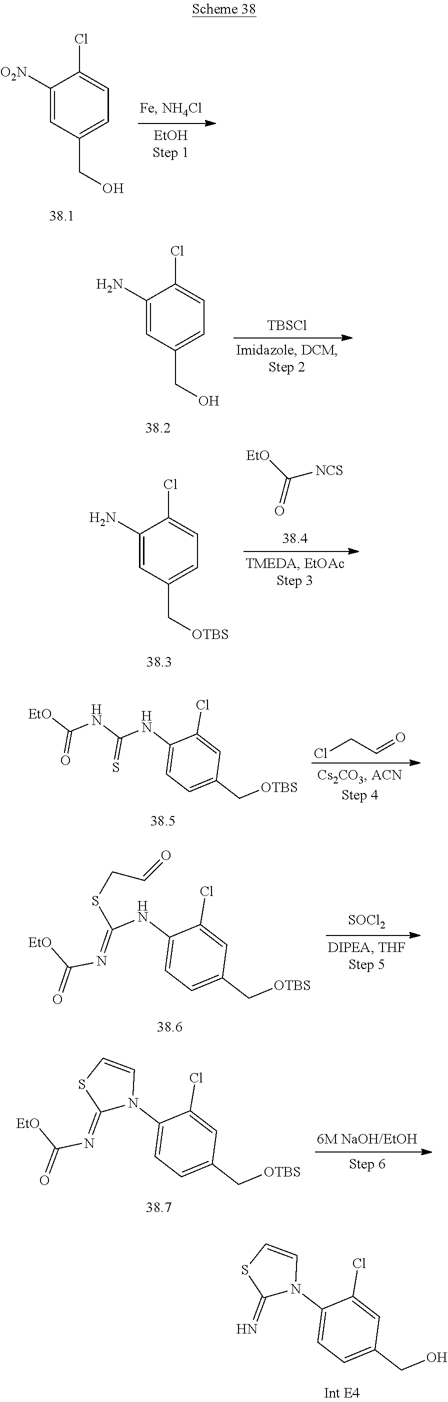

### Step 1. (3-Amino-4-chlorophenyl)methanol (38.2)

To a mixture of (4-chloro-3-nitrophenyl)methanol (38.1) (2.00 g, 10.6 mmol) and ammonium chloride (5.70 g, 106 mmol) in EtOH (30 mL) and water (10 mL) was added iron (2.98 g, 53.3 mmol) at 20° C. The resulting mixture was stirred at 70° C. for 2 hours, cooled, filtered through celite and the filter cake washed with EtOH (100 mL). The filtrate was concentrated to afford 1.50 g of (3-amino-4-chlorophenyl)methanol (38.2) as a solid.

¹H NMR (400 MHz, DMSO-d₆) δ 7.09 (d, J=8.0 Hz, 1H), 6.76 (s, 1H), 6.47 (dd, J=2.0 Hz, 8.0 Hz, 1H), 5.26 (br. s, 2H), 5.09 (t, J=6.0 Hz, 1H), 4.35 (d, J=5.6 Hz, 2H)

### Step 2. 5-(((tert-Butyldimethylsilyl)oxy)methyl)-2-chloroaniline (38.3)

A mixture of (3-amino-4-chlorophenyl)methanol (38.2) (1.50 g, 9.52 mmol) and imidazole (1.30 g, 19.0 mmol) in DCM (30 mL) was stirred at 0° C. for 1 hour and tert-butyldimethylsilyl chloride (1.72 g, 11.4 mmol) was added. The resulting mixture was stirred at 20° C. for 12 hours, poured into water (50 mL) and extracted with DCM (3×50 mL). The combined organic layers were washed with brine (50 mL), dried over Na₂SO₄, filtered, and concentrated. The residue was purified by silica gel column chromatography, eluting with 2 to 10% EtOAc in petroleum ether to afford 2.30 g of the title compound (38.3) as an oil.

¹H NMR (400 MHz, DMSO-d₆) δ 7.11 (d, J=8.0 Hz, 1H), 6.74 (d, J=1.2 Hz, 1H), 6.46 (dd, J=1.6 Hz, 8.0 Hz, 1H), 5.30 (br. s, 2H), 4.55 (s, 2H), 0.89 (s, 9H), 0.06 (s, 6H)

LCMS (m/z [M+H]⁺): 272.3

### Step 3. Ethyl N-[[4-[[tert-butyl(dimethyl)silyl]oxymethyl]-2-chloro-phenyl]carbamothioyl]carbamate (38.5)

To a mixture of 5-(((tert-butyldimethylsilyl)oxy)methyl)-2-chloroaniline (38.3) (1.20 g, 4.41 mmol) and O-ethyl carbonisothiocyanatidate (38.4) (0.578 g, 4.41 mmol) in EtOAc (20 mL) was added TMEDA (0.051 g, 0.441 mmol) at 20° C. The resulting mixture was stirred at 25° C. for 5 hours then the solvent was evaporated. The residue was triturated with 10% EtOAc in petroleum ether (20 mL) to afford 1.30 g of the title compound (38.5) as a solid.

¹H NMR (400 MHz, CDCl₃) δ 11.65 (br. s, 1H), 8.34 (s, 1H), 8.12 (br. s, 1H), 7.41 (d, J=8.4 Hz, 1H), 7.19 (d, J=8.0 Hz, 1H), 4.75 (s, 2H), 4.32 (q, J=7.2 Hz, 2H), 1.37 (t, J=7.2 Hz, 3H), 0.95 (s, 9H), 0.12 (s, 6H)

LCMS (m/z [M+H]⁺): 403.3

### Step 4. Ethyl (Z)-(((4-(((tert-butyldimethylsilyl)oxy)methyl)-2-chlorophenyl)amino)((2-oxoethyl)thio)methylene)carbamate (38.6)

To a mixture of ethyl N-[[4-[[tert-butyl(dimethyl)silyl]oxymethyl]-2-chloro-phenyl] carbamothioyl]carbamate (38.5) (1.30 g, 3.23 mmol) and cesium carbonate (2.10 g, 6.45 mmol) in acetonitrile (20 mL) was added 2-chloroacetaldehyde (6.33 g, 32.3 mmol, 40% in water) at 0° C. The resulting mixture was stirred at 25° C. for 3 hours then diluted with water (100 mL) and extracted with EtOAc (3×100 mL). The combined organic layers were washed with brine (100 mL), dried over anhydrous Na₂SO₄, filtered, and concentrated. The residue was purified by silica gel column chromatography, eluting with 5 to 30% EtOAc in petroleum ether to afford 1.30 g of the title compound (38.6) as a solid.

LCMS (m/z [M+H]⁺): 445.5

### Step 5. Ethyl (Z)-(3-(4-(((tert-butyldimethylsilyl)oxy)methyl)-2-chlorophenyl)thiazol-2(3H)-ylidene)carbamate (38.7)

To a solution of (Z)-(((4-(((tert-butyldimethylsilyl)oxy)methyl)-2-chlorophenyl)amino) ((2-oxoethyl)thio)methylene)carbamate (38.6) (1.00 g, 2.25 mmol) and DIPEA (0.871 g, 6.74 mmol) in THF (10 mL) was added thionyl chloride (0.294 g, 2.47 mmol) at 0° C. The resulting mixture was stirred at 25° C. for 2 hours. The reaction mixture was quenched with a saturated solution of sodium bicarbonate (100 mL) and extracted with EtOAc (3×100 mL). The organic layers were dried over anhydrous Na₂SO₄, filtered, and the filtrate concentrated. The residue was purified by silica gel column chromatography, eluting with 10 to 12% EtOAc in petroleum ether to afford 0.60 g of the title compound (38.7) as a solid.

¹H NMR (400 MHz, CDCl₃) δ 7.41 (d, J=8.0 Hz, 1H), 7.29 (s, 1H), 7.48 (s, 1H), 6.77 (d, J=4.8 Hz, 1H), 6.60 (d, J=4.8 Hz, 1H), 4.66 (br. s, 2H), 4.14-4.11 (m, 2H), 1.22-1.19 (m, 3H), 0.85 (s, 9H), 0.03 (s, 6H)

LCMS (m/z [M+H]⁺): 428.1

### Step 6. (3-Chloro-4-(2-iminothiazol-3(2H)-yl)phenyl)methanol (E4)

A mixture of ethyl (Z)-(3-(4-(((tert-butyldimethylsilyl)oxy)methyl)-2-chlorophenyl)thiazol-2(3H)-ylidene)carbamate (38.7) (0.600 g, 1.41 mmol) and NaOH (1.12 g, 28.1 mmol) in EtOH (6 mL) was stirred at 60° C. for 1 hour. The reaction mixture was quenched with a saturated aqueous solution of sodium bicarbonate (20 mL) and extracted with EtOAc (3×20 mL). The combined organic layers were dried over anhydrous Na₂SO₄, filtered, and the filtrate was concentrated. The residue was purified by reversed-phase flash chromatography (0.1% FA/MeCN condition) to afford 0.20 g of (3-chloro-4-(2-iminothiazol-3(2H)-yl)phenyl)methanol (E4) as a solid.

### Preparation of (3-(2-Chlorophenyl)-2-imino-23-dihydrothiazol-4-yl)methanol (E5)

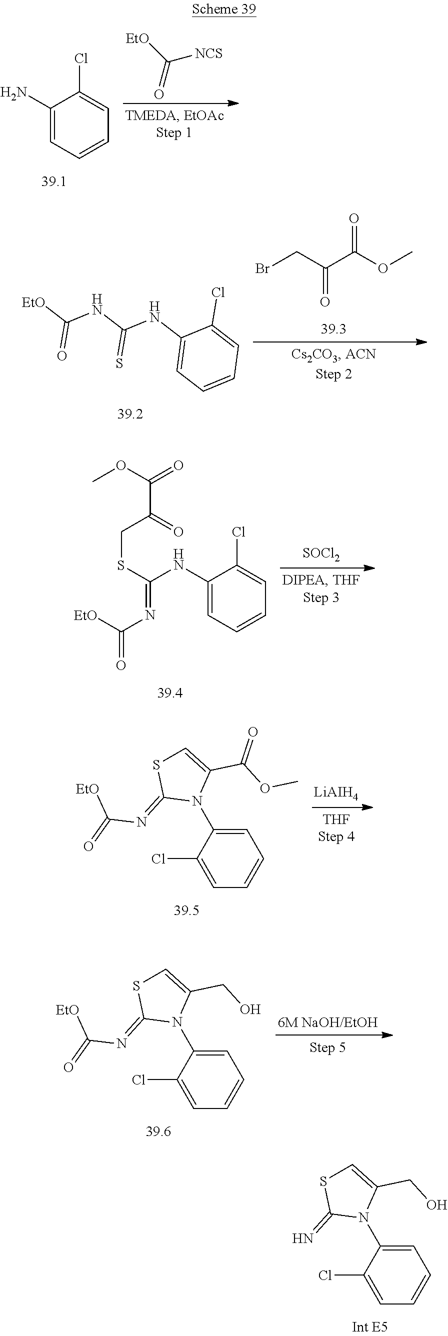

### Step 1. 3-(Cyclohexylmethyl)-4-methylthiazol-2(3H)-imine (39.2)

To a solution of 2-chloroaniline (39.1) (8.26 mL, 78.4 mmol) in EtOAc (100 mL) at 0° C. was added O-ethyl carbonisothiocyanatidate (10.3 g, 78.4 mmol) followed by TMEDA (1.18 mL, 7.84 mmol). The resulting mixture was stirred at 50° C. for 5 hours. The cooled reaction mixture was quenched by dropwise addition of MeOH (100 mL). The resulting precipitate was collected, triturated with MeOH (100 mL) at 25° C. for 10 minutes, then filtered to afford 16.5 g of the title compound (39.2) as a solid.

¹H NMR (400 MHz, DMSO-d₆) δ 11.60 (br. s, 1H), 11.47 (br. s, 1H), 7.94 (d, J=8.0 Hz, 1H), 7.57-7.55 (m, 1H), 7.41-7.36 (m, 1H), 7.33-7.29 (m, 1H), 4.23 (q, J=7.2 Hz, 2H), 1.27 (t, J=7.2 Hz, 3H)

LCMS (m/z [M+H]⁺): 258.9

### Step 2. Methyl (Z)-3-((N-(2-chlorophenyl)-N′-(ethoxycarbonyl)carbamimidoyl)thio)-2-oxopropanoate (39.4)

To a mixture of ethyl N-[(2-chlorophenyl)carbamothioyl]carbamate (39.2) (5.00 g, 19.3 mmol) and cesium carbonate (7.56 g, 23.2 mmol) in acetonitrile (100 mL) was added methyl 3-bromo-2-oxopropanoate (39.3) (2.67 mL, 25.1 mmol) at 25° C. The resulting mixture was stirred at 25° C. for 2 hours. The reaction mixture was quenched with water (100 mL) and extracted with EtOAc (3×200 mL). The combined organic layers were washed with brine (2×200 mL), dried over anhydrous Na₂SO₄, filtered, and the filtrate was concentrated under reduced pressure. The residue was purified by silica gel column chromatography, eluting with 10 to 50% EtOAc in petroleum ether to afford 2.80 g of the title compound (39.4) as an oil.

¹H NMR (400 MHz, CDCl₃) δ 7.53-7.49 (m, 1H), 7.47-7.44 (m, 1H), 7.35-7.31 (m, 2H), 4.90 (br. s, 1H), 4.16 (q, J=7.2 Hz, 2H), 4.00 (d, J=11.6 Hz, 1H), 3.75 (s, 3H), 3.47-3.42 (m, 1H), 1.26 (t, J=7.2 Hz, 3H)

LCMS (m/z [M+H]⁺): 358.9

### Step 3. Methyl (Z)-3-(2-chlorophenyl)-2-((ethoxycarbonyl)imino)-2,3-dihydrothiazole-4-carboxylate (39.5)

To a mixture of (Z)-methyl 3-((N-(2-chlorophenyl)-N′-(ethoxycarbonyl)carbamimidoyl) thio)-2-oxopropanoate (39.4) (2.80 g, 7.80 mmol) and DIPEA (2.04 mL, 11.7 mmol) in THF (15 mL) was added sulfonyl dichloride (0.849 mL, 11.7 mmol) at 0° C. The resulting mixture was stirred at 25° C. for 2 hours under nitrogen atmosphere, quenched with water (100 mL) and extracted with EtOAc (3×200 mL). The combined organic layers were washed with brine (2×200 mL), dried over anhydrous Na₂SO₄, filtered, and the filtrate was concentrated to afford 2.20 g of the title compound (39.5) as an oil, which was used without further purification.

¹H NMR (400 MHz, CDCl₃) δ 7.64 (s, 1H), 7.54-7.51 (m, 1H), 7.46-7.41 (m, 2H), 7.40-7.37 (m, 1H), 4.20 (q, J=7.2 Hz, 2H), 3.74 (s, 3H), 1.29 (t, J=7.2 Hz, 3H).

LCMS (m/z [M+H]⁺): 340.9.

### Step 4. Ethyl (Z)-(3-(2-chlorophenyl)-4-(hydroxymethyl)thiazol-2(3H)-ylidene)carbamate (39.6)

To a mixture of (Z)-methyl 3-(2-chlorophenyl)-2-((ethoxycarbonyl)imino)-2, 3-dihydro thiazole-4-carboxylate (39.5) (1.50 g, 4.40 mmol) in THF (20 mL) was added lithium aluminium hydride (0.250 g, 6.60 mmol) at 0° C. The mixture was stirred at 0° C. for 15 minutes. The reaction mixture was quenched with water (0.25 mL), NaOH (0.25 mL, 15% solution), 0.75 ml of water and iN HCl (50 ml), and allowed to stir for 5 minutes. The aqueous phase was extracted with EtOAc (4×150 mL). The combined organic phases were washed with brine (3×20 mL), dried over anhydrous Na₂SO₄, filtered, and the filtrate was concentrated to afford 1.10 g of the title compound (39.6) as a solid.

¹H NMR (400 MHz, CDCl₃) δ 7.58-7.55 (m, 1H), 7.46-7.41 (m, 3H), 6.66 (s, 1H), 4.30-4.27 (m, 1H), 4.19-4.12 (m, 3H), 1.27 (t, J=7.2 Hz, 3H).

LCMS (m/z [M+H]⁺): 313.0.

### Step 5. (3-(2-Chlorophenyl)-2-imino-2,3-dihydrothiazol-4-yl)methanol (E5)

To a mixture of (Z)-ethyl (3-(2-chlorophenyl)-4-(hydroxymethyl)thiazol-2(3H)-ylidene) carbamate (39.6) (1.60 g, 5.12 mmol) in EtOH (20 mL) was added NaOH (5.33 g, 133 mmol) at 25° C. The resulting mixture was stirred at 50° C. for 1 hour. The solvent was evaporated and the residue was purified by silica gel column chromatography, eluting with 1 to 10% MeOH in DCM, followed by reversed-phase HPLC (0.1% NH₃·H₂O/MeCN condition) to afford 0.60 g of (3-(2-chlorophenyl)-2-imino-2,3-dihydrothiazol-4-yl)methanol (E5) as a yellow solid.

### Preparation of 3-(2-Chlorophenyl)-5-methylthiazol-2(3H)-imine (E6)

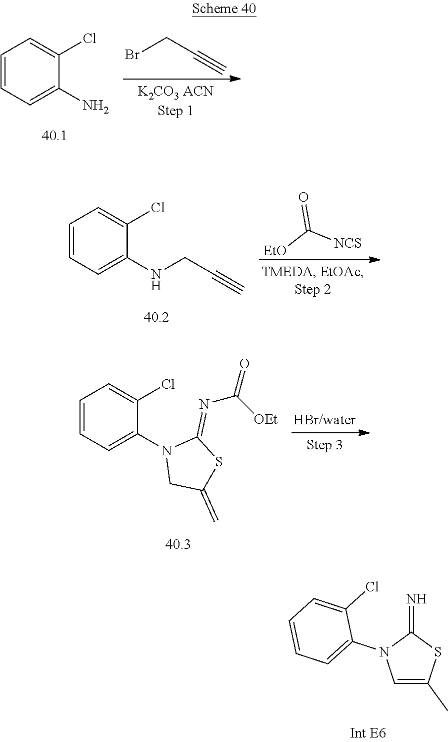

### Step 1. 2-Chloro-N-(prop-2-yn-1-yl)aniline (40.2)

To a solution of 2-chloroaniline (35.1) (10.0 g, 78.4 mmol) in acetonitrile (100 mL) was added potassium carbonate (13.0 g, 94.1 mmol) and 3-bromoprop-1-yne (14.0 g, 94.1 mmol, 80% in toluene). The resulting mixture was stirred for 2 hours at 20° C. and then at 60° C. for 12 hours. The reaction mixture was filtered and the filtrate was concentrated. The residue was purified by silica gel column chromatography, eluting with petroleum ether to afford 8.0 g of 2-chloro-N-(prop-2-yn-1-yl)aniline (40.2) as an oil.

¹H NMR (400 MHz, CDCl₃) δ 7.32-7.28 (m, 1H), 7.24-7.17 (m, 1H), 6.84-6.78 (m, 1H), 6.76-6.70 (m, 1H), 4.71-4.37 (m, 1H), 4.01 (s, 2H), 2.27 (t, J=2.4 Hz, 1H)

LCMS (m/z [M+H]⁺): 165.8

### Step 2. Ethyl (Z)-(3-(2-chlorophenyl)-5-methylenethiazolidin-2-ylidene)carbamate (40.3)

To a solution of 2-chloro-N-(prop-2-yn-1-yl)aniline (40.2) (4.00 g, 24.2 mmol) and O-ethyl carbonisothiocyanatidate (3.17 g, 24.2 mol) in EtOAc (40 mL) was added TMEDA (0.281 g, 2.42 mmol) at 25° C. The resulting mixture was stirred at 25° C. for 2 hours and then at 45° C. for 12 hours. The reaction mixture was concentrated, and the residue was purified by silica gel column chromatography, eluting with 5 to 20% EtOAc in petroleum ether to afford 4.90 g of crude the title compound (40.3) as a gum.

LCMS (m/z [M+H]⁺): 296.8

### Step 3. 3-(2-Chlorophenyl)-5-methylthiazol-2(3H)-imine (E6)

A mixture of (Z)-ethyl (3-(2-chlorophenyl)-5-methylenethiazolidin-2-ylidene)carbamate (40.3) (0.300 g, 1.01 mmol) in hydrobromic acid (5.96 g, 29.5 mmol, 40% purity in water) was stirred for 12 hours at 100° C. and then for an additional 14 hours at 105° C. The cooled reaction mixture was poured into a saturated aqueous solution of sodium bicarbonate (50 mL) and extracted with EtOAc (2×50 mL). The combined organic phases were washed with brine (50 mL), dried over anhydrous Na₂SO₄, filtered, and the filtrate was concentrated. The residue was purified by reversed phase flash chromatography (0.1% TFA/MeCN condition). The product fractions were poured into a saturated aqueous solution of sodium bicarbonate (50 mL) and extracted with EtOAc (2×50 mL). The combined organic phases were washed with brine (50 mL), dried over anhydrous Na₂SO₄, filtered, and the filtrate was concentrated to afford 0.08 g of 3-(2-chlorophenyl)-5-methylthiazol-2(3H)-imine (E6) as a gum.

### Preparation of ethyl 3-benzyl-2-imino-2,3-dihydrothiazole-5-carboxylate (E7)

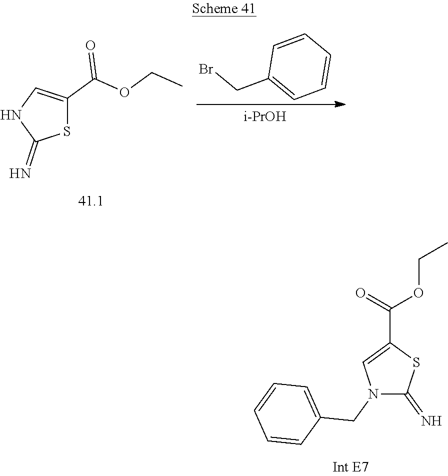

To a solution of ethyl 2-imino-2,3-dihydrothiazole-5-carboxylate (41.1) (1.00 g, 5.81 mmol) in isopropyl ether (10 mL) was added benzyl bromide (0.690 mL, 5.81 mmol) at 25° C. The resulting mixture was stirred at 80° C. for 12 hours and then cooled to 0° C. and stirred for 1 hour, The resulting precipitate was collected by filtration and dried under reduced pressure to afford 0.70 g of ethyl 3-benzyl-2-imino-2,3-dihydrothiazole-5-carboxylate (E7) as a hydrobromic salt as a solid.

### Preparation of methyl 3-(2-chlorophenyl)-2-imino-2,3-dihydrothiazole-5-carboxylate (E8)

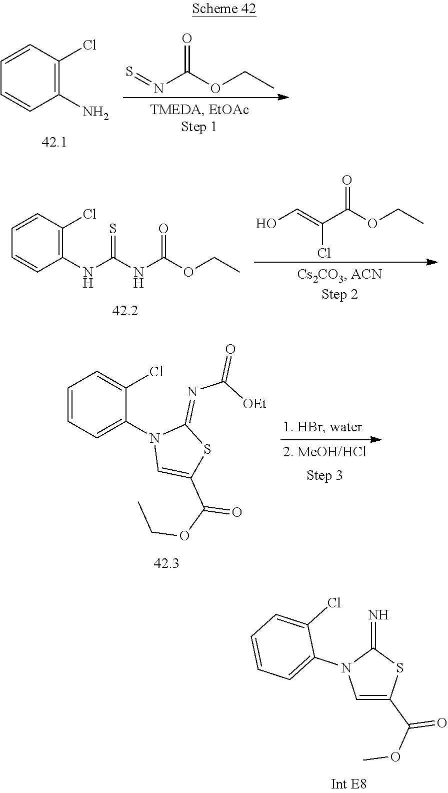

### Step 1. Ethyl N-[(2-chlorophenyl)carbamothioyl]carbamate (42.2)

To a mixture of 2-chloroaniline (42.1) (1.00 g, 7.84 mmol) and O-ethyl carbonisothiocyanatidate (1.03 g, 7.84 mmol) in EtOAc (10 mL) was added TMEDA (0.091 g, 0.784 mmol) at 25° C. The reaction mixture was stirred at 25° C. for 2 hours. The solvent was evaporated to afford 2.00 g of the title compound (42.2) as a solid.

¹H NMR (400 MHz, DMSO-d₆) δ 11.63 (br. s, 1H), 11.45 (br. s, 1H), 7.94 (d, J=8.0 Hz, 1H), 7.55 (dd, J=0.8, 8.0 Hz, 1H), 7.38 (td, J=1.6, 7.6 Hz, 1H), 7.31 (td, J=1.6, 8.0 Hz, 1H), 4.23 (q, J=7.2 Hz, 2H), 1.27 (t, J=7.2 Hz, 3H)

### Step 2. Ethyl (Z)-3-(2-chlorophenyl)-2-((ethoxycarbonyl)imino)-2,3-dihydrothiazole-5-carboxylate (42.3)

To a solution of ethyl N-[(2-chlorophenyl)carbamothioyl]carbamate (42.2) (1.55 g, 5.98 mmol) and cesium carbonate (3.90 g, 12.0 mmol) in acetonitrile (30 mL) was added (Z)-ethyl 2-chloro-3-hydroxyacrylate (0.900 g, 5.98 mmol) at 0° C. The resulting mixture was stirred at 25° C. for 3 hours and then at 60° C. for 12 hours. The reaction mixture was diluted with water (150 mL) and extracted with EtOAc (3×100 mL). The combined organic phases were washed with brine (80 mL), dried with anhydrous Na₂SO₄, filtered, and the filtrate was concentrated. The residue was triturated with MeOH (6 mL) and purified by silica gel column chromatography, eluting with 0 to 50% EtOAc in petroleum ether to afford 0.22 g of the title compound (42.3) as a solid.

¹H NMR (400 MHz, DMSO-d₆) δ 8.45 (s, 1H), 7.74 (dd, J=1.6 Hz, 8.0 Hz, 1H), 7.68 (dd, J=2.0 Hz, 8.0 Hz, 1H), 7.60 (td, J=2.0 Hz, 7.6 Hz, 1H), 7.55 (td, J=1.6 Hz, 7.6 Hz, 1H), 4.29 (q, J=7.2 Hz, 2H), 4.05 (qd, J=2.0 Hz, 7.2 Hz, 2H), 1.29 (t, J=7.2 Hz, 3H), 1.15 (t, J=7.2 Hz, 3H)

LCMS (m/z [M+H]⁺): 355.0

### Step 3. Methyl 3-(2-chlorophenyl)-2-imino-2,3-dihydrothiazole-5-carboxylate (E8)

A mixture of (Z)-ethyl 3-(2-chlorophenyl)-2-((ethoxycarbonyl)imino)-2,3-dihydrothiazole-5-carboxylate (42.3) (0.220 g, 0.570 mmol) in hydrobromic acid (5.96 g, 29.5 mmol, 40% in water) was stirred at 100° C. for 12 hours. The cooled reaction mixture was lyophilized. The residue was added into a 4M HCl solution in MeOH (4 mL, 16.0 mmol) and stirred at 60° C. for 12 hours. The mixture was concentrated under reduced pressure to afford 0.14 g of methyl 3-(2-chlorophenyl)-2-imino-2,3-dihydrothiazole-5-carboxylate (E8) as an oil.

### Preparation of (3-benzyl-2-imino-2,3-dihydrothiazol-4-yl)methanol (E9)

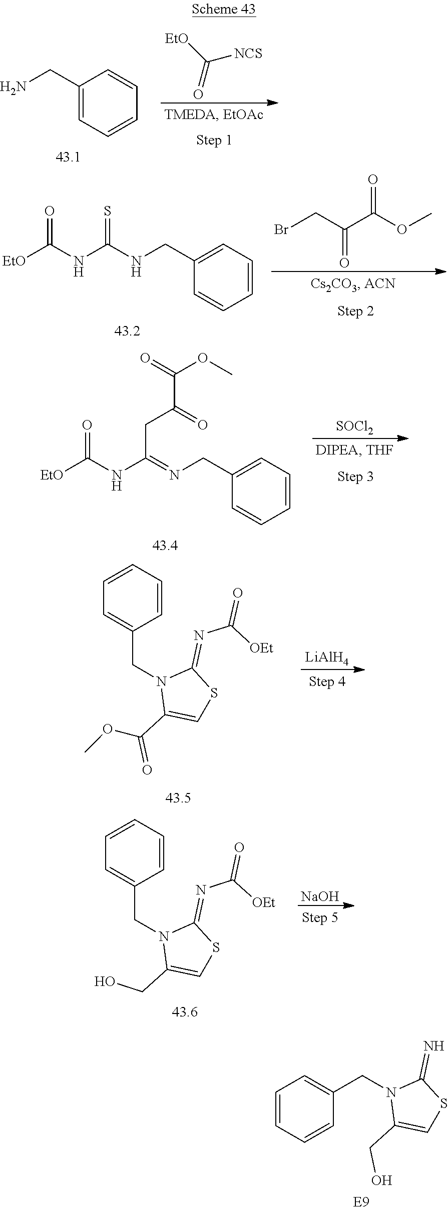

### Step 1. Ethyl N-(benzylcarbamothioyl)carbamate (43.2)

To a mixture of benyzlamine (43.1) (4.00 g, 37.3 mmol) and O-ethyl carbonisothiocyanatidate (4.90 g, 37.3 mmol) in EtOAc (50 mL) was added TMEDA (0.430 g, 3.73 mmol) at 25° C. The mixture was stirred at 25° C. for 1 hour and then concentrated under reduced pressure. The residue was triturated with 2% EtOAc in petroleum ether (50 mL) to afford 7.60 g of ethyl N-(benzylcarbamothioyl) carbamate (43.2) as a solid.

¹H NMR (400 MHz, CDCl₃) δ 9.97 (br. s, 1H), 7.40-7.28 (m, 5H), 4.87 (d, J=5.2 Hz, 2H), 4.24-4.19 (m, 2H), 1.31 (t, J=6.8 Hz, 3H)

LCMS (m/z [M+H]⁺): 239.1

### Step 2. Methyl (E)-4-(benzylimino)-4-((ethoxycarbonyl)amino)-2-oxobutanoate (43.4)

To a mixture of N-(benzylcarbamothioyl)carbamate (43.2) (7.60 g, 31.8 mmol) and cesium carbonate (20.7 g, 63.7 mmol) in acetonitrile (100 mL) was added methyl 3-bromo-2-oxo-propanoate (43.3) (7.50 g, 41.4 mmol) at 25° C. The reaction mixture was stirred at 25° C. for 2 hours then quenched with water (100 mL) and extracted with EtOAc (4×100 mL). The combined organic phases were washed with brine (3×50 mL), dried over anhydrous Na₂SO₄, filtered, and the filtrate was concentrated. The residue was purified by silica gel column chromatography, eluting with 10 to 30% EtOAc in petroleum ether to afford 5.50 g of the title compound (43.4) as a solid.

¹H NMR (400 MHz, CDCl₃) δ 7.30-7.21 (m, 5H), 5.28 (d, J=15.6 Hz, 1H), 4.73 (br s, 1H), 4.33 (d, J=15.6 Hz, 1H), 4.29-4.20 (m, 2H), 3.58 (d, J=12.4 Hz, 1H), 3.29-3.23 (m, 4H), 1.34 (t, J=7.2 Hz, 3H)

LCMS (m/z [M+H]⁺): 339.1

### Step 3. Methyl (Z)-3-benzyl-2-((ethoxycarbonyl)imino)-2,3-dihydrothiazole-4-carboxylate (43.5)

To a solution of (Z)-methyl 3-((N-benzyl-N′-(ethoxycarbonyl)carbamimidoyl)thio)-2-oxopropanoate (43.4) (5.50 g, 16.2 mmol) in THF (80 mL) was added DIPEA (4.20 g, 32.5 mmol) and thionyl chloride (2.10 g, 17.8 mmol) at 0° C. The mixture was stirred at 25° C. for 1 hour then quenched with water (100 mL) and extracted with EtOAc (4×100 mL). The combined organic layers were washed with brine (3×50 mL), dried over anhydrous Na₂SO₄, filtered, and the filtrate was concentrated. The residue was purified by silica gel column chromatography, eluting with 10 to 30% EtOAc in petroleum ether to afford 4.00 g of the title compound (43.5) as an oil.

¹H NMR (400 MHz, CDCl₃) δ 7.55 (s, 1H), 7.33-7.16 (m, 5H), 5.86 (s, 2H), 4.30-4.25 (m, 2H), 3.81 (s, 3H), 1.36 (t, J=7.2 Hz, 3H)

LCMS (m/z [M+H]⁺): 321.5

### Step 4. Ethyl (Z)-(3-benzyl-4-(hydroxymethyl)thiazol-2(3H)-ylidene)carbamate (43.6)

To a solution of methyl (Z)-3-benzyl-2-((ethoxycarbonyl)imino)-2,3-dihydrothiazole-4-carboxylate (43.5) (3.90 g, 12.2 mmol) in THF (40 mL) was added lithium aluminum hydride (0.560 g, 14.6 mmol) at 0° C. and the reaction mixture was continued to stir at 0° C. for 15 minutes. The reaction was diluted with THF (150 mL), quenched with water (4 mL), and followed by the addition of a 15% solution of NaOH (4 mL) and water (12 mL) at 0° C. The mixture was filtered, and the filter cake was washed with EtOAc (50 mL). The filtrate was dried with anhydrous Na₂SO₄, filtered, and concentrated. The residue was triturated with EtOAc (80 mL) to afford 2.50 g of the title compound (43.6) as a solid.

¹H NMR (400 MHz, DMSO-d₆) δ 7.38-7.25 (m, 3H), 7.12 (d, J=7.2 Hz, 2H), 6.85 (s, 1H), 5.77 (br. s, 1H), 5.40 (s, 2H), 4.30 (s, 2H), 4.07-4.02 (m, 2H), 1.18 (t, J=7.2 Hz, 3H)

LCMS (m/z [M+H]⁺): 292.9

### Step 5. (3-Benzyl-2-imino-2,3-dihydrothiazol-4-yl)methanol (E9)

To a solution of (Z)-ethyl (3-benzyl-4-(hydroxymethyl)thiazol-2(3H)-ylidene)carbamate (43.6) (1.00 g, 3.42 mmol) in EtOH (15 mL) was added NaOH (2.74 g, 68.4 mmol). The mixture was stirred at 30° C. for 2 hours, and then concentrated under reduced pressure. The residue was purified by reversed-phase flash chromatography (0.1% NH₃·H₂O/MeCN condition) to afford 0.35 g of (3-benzyl-2-imino-2,3-dihydrothiazol-4-yl)methanol (E9) as a solid.

### Preparation of 4-(Bromomethyl)-3-(2-chlorophenyl)thiazol-2(3H)-imine (E10) and 3-(2-Chlorophenyl)-4-(morpholinomethyl)thiazol-2(3H)-imine (E11)

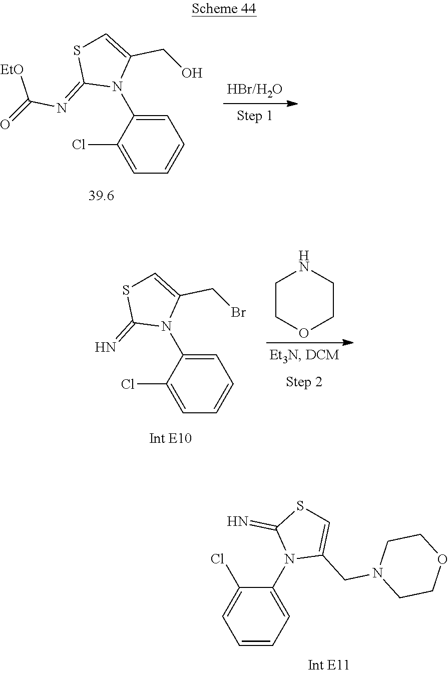

### Step 1. 4-(Bromomethyl)-3-(2-chlorophenyl)thiazol-2(3H)-imine (E10)

A solution of ethyl (Z)-ethyl (3-(2-chlorophenyl)-4-(hydroxymethyl)thiazol-2(3H)-ylidene) carbamate (39.6) (3.50 g, 11.2 mmol) in hydrobromic acid (1.80 g, 11.2 mmol, 50% in water) was stirred at 100° C. for 5 hours. The cooled reaction mixture was diluted with water (100 mL) and extracted with EtOAc (3×100 mL) to remove impurities. The aqueous layer was concentrated under reduced pressure to afford 2.00 g of 4-(bromomethyl)-3-(2-chlorophenyl)thiazol-2(3H)-imine as the hydrobromic salt (E10) as a black solid.

LCMS (m/z [M+H]⁺): 305.0

### Step 2 3-(2-Chlorophenyl)-4-(morpholinomethyl)thiazol-2(3H)-imine (E11)

A mixture of 4-(bromomethyl)-3-(2-chlorophenyl)thiazol-2(3H)-imine hydrobromide (E10) (1.00 g 3.30 mmol), morpholine (0.300 g 3.30 mmol) and Et₃N (0.700 g, 6.60 mmol) in DCM (10 mL) was stirred at 25° C. for 12 hours. The reaction mixture was concentrated under reduced pressure and the residue was purified by reversed-phase flash chromatography (0.1% FA/MeCN condition) to afford 0.20 g of 3-(2-chlorophenyl)-4-(morpholinomethyl)thiazol-2(3H)-imine (E11) as an oil.

### N-methylethan-1-amine (E12)

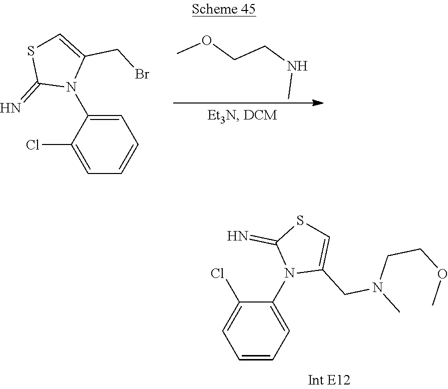

To a mixture of 4-(bromomethyl)-3-(2-chlorophenyl)thiazol-2(3H)-imine (E10) (0.500 g, 1.65 mmol) and 2-methoxy-N-methyl-ethylamine (0.180 g, 1.65 mmol) in DCM (5 mL) was added Et₃N (0.330 g, 3.29 mmol). The mixture was stirred at 25° C. for 12 hours, and then concentrated under reduced pressure. The residue was purified by reversed phase flash chromatography (0.1% FA/MeCN condition) to afford 0.20 g of N-((3-(2-chlorophenyl)-2-imino-2,3-dihydrothiazol-4-yl)methyl)-2-methoxy-N-methylethan-1-amine (E12) as an oil.

### Preparation of 1-(3-cyclohexyl-2-imino-2,3-dihydrothiazol-4-yl)-N,N-dimethylmethanamine (E13)

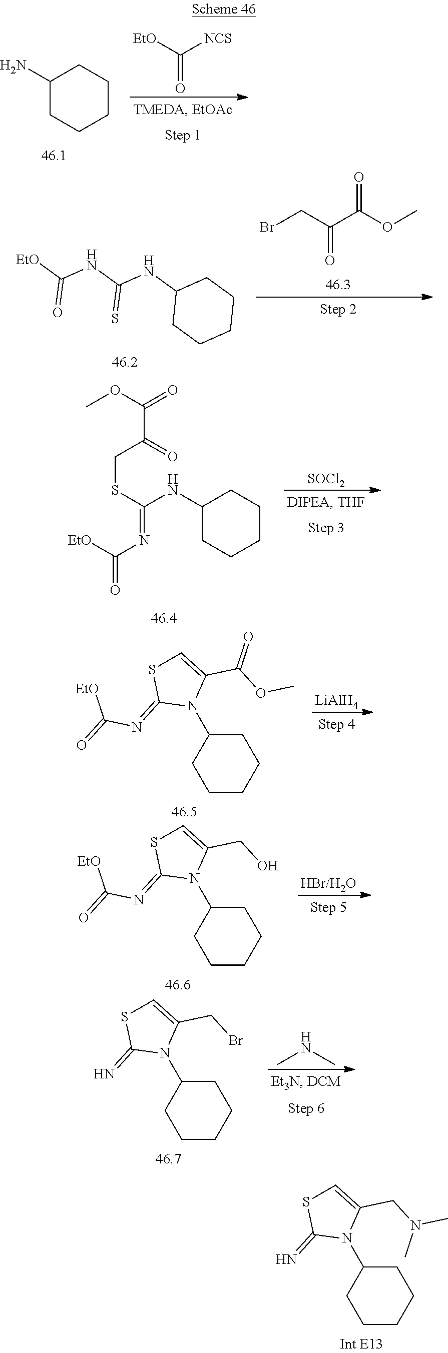

### Step 1. Ethyl N-(cyclohexylcarbamothioyl)carbamate (46.2)

A mixture of cyclohexylamine (46.1) (3.00 g, 30.2 mmol), O-ethyl carbonisothiocyanatidate (3.97 g, 30.2 mmol) and TMEDA (0.351 g, 3.02 mmol) in EtOAc (60 mL) was stirred at 20° C. for 2 hours. The solvent was evaporated and the residue purified by silica gel column chromatography, eluting with 5 to 20% EtOAc in petroleum ether to afford 6.50 g of ethyl N-(cyclohexylcarbamothioyl)carbamate (46.2) as a solid.

¹H NMR (400 MHz, CDCl₃) δ 9.65 (br. s, 1H), 7.96 (br. s, 1H), 4.24-4.18 (m, 2H), 2.09-2.02 (m, 2H), 1.76-1.68 (m, 2H), 1.65-1.58 (m, 1H), 1.49-1.33 (m, 4H), 1.33-1.20 (m, 5H). LCMS (m/z [M+H]⁺): 231.4

### Step 2. Methyl (Z)-3-((N-cyclohexyl-N′-(ethoxycarbonyl)carbamimidoyl)thio)-2-oxopropanoate (46.4)

To a mixture of ethyl N-(cyclohexylcarbamothioyl)carbamate (46.2) (6.00 g, 26.0 mmol), potassium carbonate (10.8 g, 78.1 mmol) in acetonitrile (100 mL) was added methyl 3-bromo-2-oxopropanoate (46.3) (7.07 g, 39.0 mmol) at 25° C. The reaction mixture was stirred at 25° C. for 2 hours, filtered through Celite, and rinsed with EtOAc (100 mL). The combined filtrates were concentrated and the residue purified by silica gel column chromatography, eluting with 10 to 30% EtOAc in petroleum ether to afford 4.00 g of the title compound (46.4) as an oil.

¹H NMR (400 MHz, CDCl₃) δ 4.58 (br. s, 1H), 4.21 (q, J=7.2 Hz, 2H), 3.90 (s, 3H), 3.70-3.57 (m, 1H), 3.45 (d, J=12.4 Hz, 1H), 3.21 (d, J=12.4 Hz, 1H), 2.27-2.07 (m, 2H), 1.81-1.72 (m, 2H), 1.72-1.63 (m, 2H), 1.33 (t, J=7.2 Hz, 3H), 1.25-1.09 (m, 4H).

LCMS (m/z [M+H]⁺): 331.4

### Step 3. Methyl (Z)-3-cyclohexyl-2-((ethoxycarbonyl)imino)-2,3-dihydrothiazole-4-carboxylate (46.5)

To a solution of (Z)-methyl 3-((N-cyclohexyl-N′-(ethoxycarbonyl)carbamimidoyl)thio)-2-oxopropanoate (46.4) (3.90 g, 11.8 mmol), DIPEA (3.81 g, 29.5 mmol) in THF (50 mL) was added thionyl chloride (2.11 g, 17.7 mmol) at 0° C. The resulting mixture was stirred at 25° C. for 1 hour. The solvent was evaporated and the residue purified by silica gel column chromatography, eluting with 5 to 20% EtOAc in petroleum ether to afford 2.10 g of the title compound (46.5) as a solid.

¹H NMR (400 MHz, CDCl₃) δ 7.45 (s, 1H), 5.15-5.00 (m, 1H), 4.27 (q, J=7.2 Hz, 2H), 3.89 (s, 3H), 2.70-2.54 (m, 2H), 1.91-1.82 (m, 2H), 1.81-1.72 (m, 2H), 1.70-1.62 (m, 1H), 1.44-1.40 (m, 1H), 1.37 (t, J=7.2 Hz, 3H), 1.35-1.17 (m, 2H)

LCMS (m/z [M+H]⁺): 313.3

### Step 4. Ethyl (Z)-(3-cyclohexyl-4-(hydroxymethyl)thiazol-2(3H)-ylidene)carbamate (46.6)

To a solution of (Z)-methyl 3-cyclohexyl-2-((ethoxycarbonyl)imino)-2,3-dihydrothiazole-4-carboxylate (46.5) (2.00 g, 6.40 mmol) in THF (30 mL) was added lithium aluminum hydride (0.291 g, 7.68 mmol) at 0° C. The mixture was stirred at 0° C. for 15 minutes, diluted with THF (150 mL), then quenched with ice water (100 mL) and extracted with EtOAc (3×150 mL). The combined organic layers were dried over anhydrous Na₂SO₄, filtered, and concentrated. The residue was triturated with EtOAc (20 mL) to afford 1.50 g of the title compound (46.6) as a solid.

¹H NMR (400 MHz, CDCl₃) δ 6.41 (br. s, 1H), 4.56 (br. s, 2H), 4.25 (q, J=7.2 Hz, 2H), 3.00-2.70 (m, 2H), 1.93-1.85 (m, 2H), 1.83-1.74 (m, 2H), 1.72-1.65 (m, 1H), 1.45-1.32 (m, 6H).

LCMS (m/z [M+H]⁺): 285.3

### Step 5. 4-(Bromomethyl)-3-cyclohexylthiazol-2(3H)-imine (46.7)

A solution of ethyl (Z)-ethyl (3-cyclohexyl-4-(hydroxymethyl)thiazol-2(3H)-ylidene) carbamate (41.6) (0.500 g, 1.76 mmol) in hydrobromic acid (8 mL, 40% in water) was stirred at 100° C. for 12 hours. The mixture was concentrated under reduced pressure to afford 0.60 g of the crude title compound (46.7) hydrobromide as a solid, which was used without further purification.

¹H NMR (400 MHz, DMSO-d₆) δ 9.41 (br. s, 2H), 7.27 (s, 1H), 4.92 (s, 2H), 4.29-4.15 (m, 1H), 2.27-2.14 (m, 2H), 1.89-1.77 (m, 4H), 1.53-1.32 (m, 4H)

### Step 6. 1-(3-Cyclohexyl-2-imino-2,3-dihydrothiazol-4-yl)-N,N-dimethylmethanamine (E13)

To a mixture of 4-(bromomethyl)-3-cyclohexylthiazol-2(3H)-imine (46.7) hydrobromide (0.600 g, 1.68 mmol) and Et₃N (0.170 g, 1.68 mmol) in DCM (10 mL) was added dimethylamine hydrochloride (0.274 g, 3.37 mmol) at 25° C. and the mixture stirred at 25° C. for 2 hours. The reaction was quenched with water (50 mL) and extracted with EtOAc (3×50 mL). The combined organic layers were dried over anhydrous Na₂SO₄, filtered, and concentrated. The residue was purified by reversed-phase flash chromatography (0.1% NH₃·H₂O/MeCN condition) to afford 0.15 g of 1-(3-cyclohexyl-2-imino-2,3-dihydrothiazol-4-yl)-N,N-dimethylmethanamine (E13) as an oil.

### Preparation of ethyl 3-benzyl-2-imino-2,3-dihydrothiazole-4-carboxylate (E14)

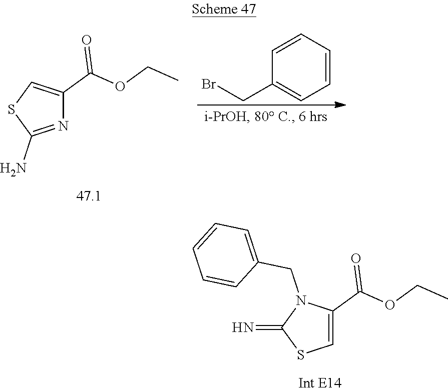

To a solution of ethyl 2-aminothiazole-4-carboxylate (47.1) (2.00 g, 11.6 mmol) in i-PrOH (20 mL) was added benzyl bromide (1.38 mL, 11.6 mmol) at 25° C. The reaction mixture was heated to 80° C. and stirred for 6 hours then cooled to room temperature and allowed to stand for 3 days. The resulting precipitate was collected by filtration, and the filter cake was dried under vacuum to afford 0.80 g of ethyl 3-benzyl-2-imino-2,3-dihydrothiazole-4-carboxylate (E14) as a solid.

### Preparation of 3-(2-chlorophenyl)-2-imino-N, N-dimethyl-thiazole-4-carboxamide (E15)

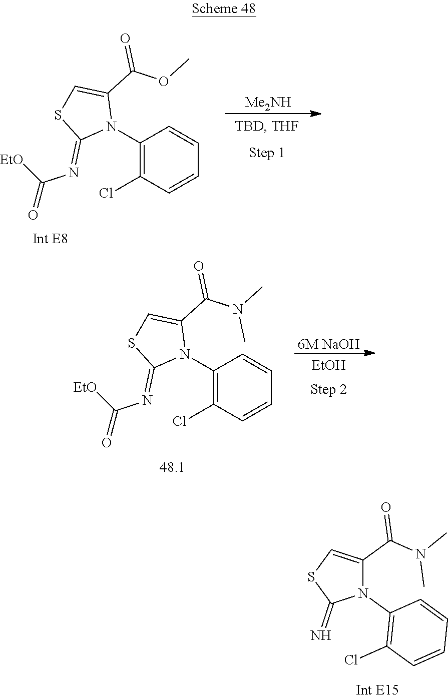

### Step 1. Ethyl (Z)-(3-(2-chlorophenyl)-4-(dimethylcarbamoyl)thiazol-2(3H)-ylidene)carbamate (48.1)

To a mixture of methyl (Z)-3-(2-chlorophenyl)-2-((ethoxycarbonyl)imino)-2,3-dihydrothiazole-4-carboxylate (E8) (0.380 g, 1.12 mmol) and dimethylamine (2 M, 5.6 mL) in THF (1 mL) was added TBD (0.078 g, 0.560 mmol) in one portion at 25° C. under nitrogen atmosphere. The reaction mixture was stirred at 60° C. for 2 hours and was then poured slowly into 100 mL of ice-water. The aqueous phase was extracted with EtOAc (3×50 mL). The combined organic phases were washed with brine (10 mL), dried over anhydrous Na₂SO₄, filtrated, and concentrated. The residue was purified by reversed-phase flash chromatography (0.1% TFA/MeCN/water) to afford 0.140 g of the title compound (48.1) as a solid.

¹H NMR (DMSO-d₆, 400 MHz) δ 7.63-7.61 (m, 1H), 7.52-7.43 (m, 3H), 7.36 (s, 1H), 4.04 (m, 2H), 3.11 (s, 3H), 2.76 (s, 3H), 1.14 (t, J=7.2 Hz, 3H).]

LCMS (m/z [M+H]⁺): 353.9

### Step 2. 3-(2-Chlorophenyl)-2-imino-N, N-dimethyl-thiazole-4-carboxamide (E15)

To a solution of ethyl (Z)-[3-(2-chlorophenyl)-4-(dimethylcarbamoyl)thiazol-2-ylidene]carbamate (48.1) (0.390 g, 1.10 mmol) in EtOH (4 mL) was added solid NaOH (0.960 g, 24.0 mmol). The reaction mixture was stirred at 50° C. for 2 hours. The crude product was purified by reversed-phase flash chromatography directly without any workup (0.1% ammonium hydroxide/MeCN/water) to afford 0.090 g of 3-(2-Chlorophenyl)-2-imino-N, N-dimethyl-thiazole-4-carboxamide (E15) as a solid.

### Preparation of 3-(2-Chlorophenyl)-2-imino-N-methyl-2,3-dihydrothiazole4-carboxamide (E16)

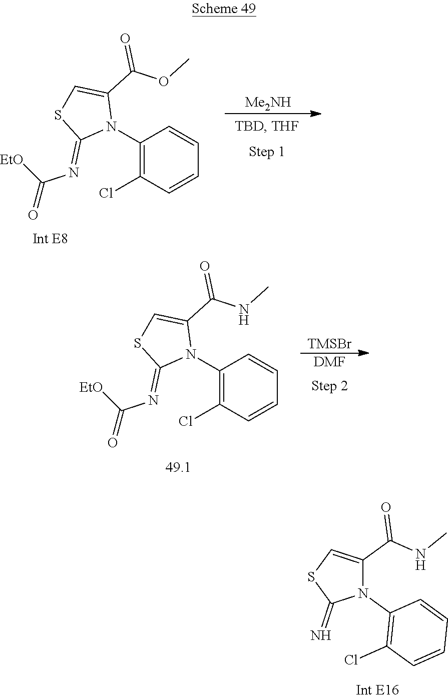

### Step 1. (Z)-Ethyl (3-(2-chlorophenyl)-4-(methylcarbamoyl)thiazol-2(3H)-ylidene)carbamate (49.1)

To a mixture of (Z)-methyl 3-(2-chlorophenyl)-2-((ethoxycarbonyl)imino)-2,3-dihydro thiazole-4-carboxylate (E8) (1.5 g, 4.40 mmol) in THF (15 mL) was added methylamine (2 M, 22 mL) dropwise and TBD (0.306 g, 2.20 mmol) at 25° C. The reaction mixture was stirred at 60° C. for 2 hours under nitrogen atmosphere then slowly poured into ice water (50 mL) and extracted with EtOAc (3×100 mL). The combined organic layers were washed with brine (2×200 mL), dried over anhydrous Na₂SO₄, filtered, and concentrated to afford 1.55 g of the title compound (49.1) as a solid.

¹H NMR (DMSO-d₆, 400 MHz) δ 8.63 (br. d, J=4.8 Hz, 1H), 7.59 (d, J=7.6 Hz, 1H), 7.54 (s, 1H), 7.49-7.43 (m, 3H), 4.02 (q, J=7.2 Hz, 2H), 2.57 (d, J=4.8 Hz, 3H), 1.19-1.04 (t, J=7.2 Hz, 3H)

LCMS (m/z [M+H]⁺): 339.9

### Step 2. 3-(2-Chlorophenyl)-2-imino-N-methyl-2,3-dihydrothiazole-4-carboxamide (E16)

To a solution of (Z)-ethyl (3-(2-chlorophenyl)-4-(methylcarbamoyl)thiazol-2(3H)-ylidene) carbamate (49.1) (0.360 g, 1.06 mmol) in DMF (7 mL) was added bromotrimethylsilane (0.687 mL, 5.30 mmol) at 25° C. The reaction mixture was stirred at 80° C. for 1 hour, cooled and poured slowly into ice water (20 mL). The mixture was neutralized with a sat. solution of sodium bicarbonate. The mixture was purified by reversed-phase flash chromatography (0.1% NH₃·H₂O/MeCN/water) to afford 0.080 g of 3-(2-chlorophenyl)-2-imino-N-methyl-2,3-dihydrothiazole-4-carboxamide (E16) as a solid.

### Preparation of 3-benzylthiazol-2(3H)-imine (E17)

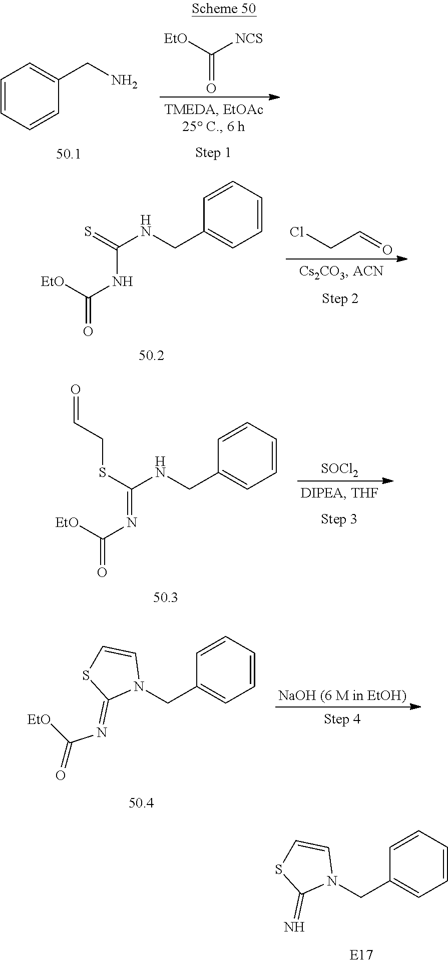

### Step 1. Ethyl N-(benzylcarbamothioyl)carbamate (50.2)

To a solution of benzylamine (50.1) (5.00 g, 46.7 mmol) and O-ethyl carbonisothiocyanatidate (6.43 g, 49.0 mmol) in EtOAc (40 mL) was added TMEDA (0.54 g, 4.67 mmol) at 25° C. under nitrogen. The reaction mixture was stirred at 25° C. for 6 hours and then the solvent was evaporated. The residue was tritrated using a mixture of 10% EtOAc in petroleum ether (55 mL) twice to afford 10.0 g of the the title compound (50.2) as a solid.

¹H NMR (400 MHz, DMSO-d₆) δ 11.04 (br. s, 1H), 10.22 (br. t, J=5.6 Hz, 1H), 7.39-7.23 (m, 5H), 4.81 (d, J=5.6 Hz, 2H), 4.14 (q, J=7.2 Hz, 2H), 1.21 (t, J=7.2 Hz, 3H)

### Step 2. (Z)-Ethyl ((benzylamino)((2-oxoethyl)thio)methylene)carbamate (50.3)

To a mixture of ethyl N-(benzylcarbamothioyl)carbamate (50.2) (4.00 g, 16.8 mmol) and cesium carbonate (13.7 g, 42.0 mmol) in acetonitrile (50 mL) was added 2-chloroacetaldehyde (13.2 g, 67.2 mmol). The reaction mixture was stirred at 25° C. for 12 hours. The mixture was poured into ice-water (20 mL) and extracted with MTBE (2×200 mL). The combined organic phases were washed with brine (2×20 mL), dried over anhydrous Na₂SO₄, filtered and concentrated. The residue was purified by silica gel column chromatography, eluting with 1 to 30% EtOAc in petroleum ether to afford 5.00 g of the title compound (50.3) as a solid.

LCMS (m/z [M+H]⁺): 280.9

### Step 3. (Z)-Ethyl (3-benzylthiazol-2(3H)-ylidene)carbamate (50.4)

To a mixture of (Z)-ethyl ((benzylamino)((2-oxoethyl)thio)methylene)carbamate (50.3) (5.00 g, 12.4 mmol) and DIPEA (4.81 g, 37.2 mmol) in THF (50 mL) was added thionyl chloride (1.48 g, 12.4 mmol) and the mixture stirred at 25° C. for 16 hours then poured into ice-water (20 mL) and extracted with EtOAc (3×100 mL). The combined organic phases were washed with brine (20 mL), dried over anhydrous Na₂SO₄, filtered, and concentrated. The residue was purified by silica gel column chromatography, eluting with 1 to 25% EtOAc in petroleum ether to afford 2.80 g of the title compound (50.4) as a solid.

¹H NMR (400 MHz, CD₃OD) δ 7.36-7.28 (m, 5H), 7.22 (d, J=4.8 Hz, 1H), 6.84 (d, J=4.8 Hz, 1H), 5.31 (s, 2H), 4.20 (q, J=7.2 Hz, 2H), 1.30 (t, J=7.2 Hz, 3H).

LCMS (m/z [M+H]⁺): 262.9

### Step 4. 3-Benzylthiazol-2(3H)-imine (E17)

A mixture (Z)-ethyl (3-benzylthiazol-2(3H)-ylidene)carbamate (50.4) (2.80 g, 9.06 mmol) and NaOH (7.25 g, 181 mmol) in EtOH (20 mL) was stirred at 50° C. for 1.5 hours. The mixture was poured into ice-water (20 mL) and extracted with EtOAc (5×100 mL). The combined organic phases were washed with brine (20 mL), dried over anhydrous Na₂SO₄, filtered and concentrated to afford 1.60 g of 3-benzylthiazol-2(3H)-imine (E17) as a gum.

### Preparation of (Z)—N-(3-benzylthiazolidin-2-ylidene)-1H-pyrrolo[2,3-b]pyridine-3-carboxamide (Example 1)

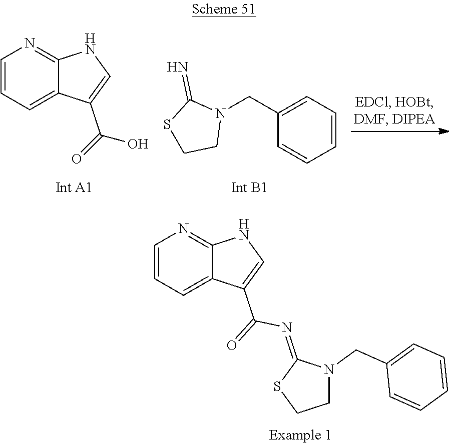

To a mixture of 1H-pyrrolo[2,3-b] pyridine-3-carboxylic acid (A1) (0.084 g, 0.520 mmol), EDCI (0.180 g, 0.939 mmol), DIPEA (0.700 mL 4.02 mmol) and HOBt (0.035 g, 0.260 mmol) in DMF (1 mL) was added 3-benzylthiazolidin-2-imine (B1) (0.100 g, 0.520 mmol) in DMF (1 mL). The mixture was stirred at 50° C. for 12 hours then diluted with water (20 mL) and extracted with EtOAc (3×20 mL). The extracts were washed with brine (3×30 mL), dried over anhydrous Na₂SO₄, filtered, and the filtrate was concentrated under reduced pressure. The residue was triturated with water (5 mL), followed by acetonitrile (2 mL) twice, and then lyophilized to afford 0.056 g of (Z)—N-(3-benzylthiazolidin-2-ylidene)-1H-pyrrolo[2,3-b] pyridine-3-carboxamide (Example 1) as a solid.

¹H NMR (400 MHz, DMSO-d₆) δ 12.23 (br. s, 1H), 8.47 (dd, J=1.6, 8.0 Hz, 1H), 8.26 (dd, J=1.6 Hz, 4.4 Hz, 1H), 8.20 (d, J=2.0 Hz, 1H), 7.41-7.35 (m, 4H), 7.33-7.28 (m, 1H), 7.15 (dd, J=4.8 Hz, 8.0 Hz, 1H), 4.98 (s, 2H), 3.62 (t, J=8.0 Hz, 2H), 3.17 (t, J=8.0 Hz, 2H)

LCMS (m/z [M+H]⁺): 337.0

Examples in Table F were prepared in a similar fashion to that shown above in Scheme 51 using intermediates from Table A-E.

TABLE F

| Ex. | Structure | Int. | LCMS | 1H NMR |
|---|---|---|---|---|
| 2 | 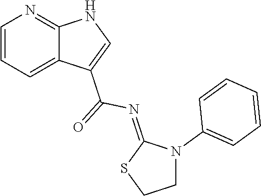 [SMILES: O=C(/N=C1\SCCN1c1ccccc1)c1c[nH]c2ncccc12] | A1 C1 | 323.0 | (DMSO-d₆ + D₂O, 400 MHz) δ 8.22 − 8.17 (m, 1H), 8.13 ( d, J = 7.6 Hz, 1H), 7.91 (s, 1H), 7.57 − 7.48 (m, 4 H), 7.35 (t, J = 7.2 Hz 1H), 7.03 (dd, J =4.8 Hz, 7.6 Hz, 1H), 4.12 (br. t, J = 7.6 Hz, 2H), 3.29 (t, J = 7.6 Hz, 2H). |
| 3 | 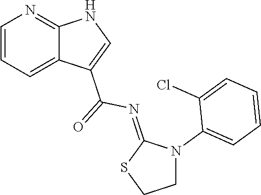 [SMILES: O=C(/N=C1\SCCN1c1ccccc1Cl)c1c[nH]c2ncccc12] | A1 C12 | 356.8 | (DMSO-d₆, 400 MHz) δ 12.16 (s, 1H), 8.17 (dd, J = 1.6 Hz, 4.4 Hz, 1H), 7.82 (d, J = 2.8 Hz, 1H), 7.77 (d, J = 8.0 Hz, 1H), 7.74 − 7.69 (m, 1H), 7.65 − 7.61 (m, 1H), 7.57 − 7.52 (m, 2H), 6.90 (dd, J = 4.4, 8.0 Hz, 1H), 4.04 − 3.97 (m, 2H), 3.48 − 3.38 (m, 2H). |
| 4 | 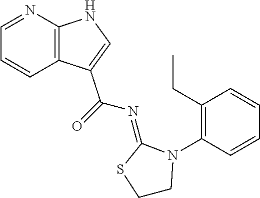 [SMILES: CCc1ccccc1N1CCS/C1=N\C(=O)c1c[nH]c2ncccc12] | A1 C2 | 351.1 | (DMSO-d₆, 400 MHz) δ 12.13 (br. s, 1H), 8.14 (d, J = 4.4 Hz, 1H), 7.81 (s, 1H), 7.70 (d, J = 7.6 Hz, 1H), 7.52 − 7.43 (m, 2H), 7.40 − 7.33 (m, 2H), 6.84 (dd, J = 4.8 Hz, 8.0 Hz, 1H), 4.11 − 4.01 (m, 1H), 3.99 − 3.88 (m, 1H), 3.41 (t, J = 8.0 Hz, 2H), 2.61 − 2.53 (m, 2H), 1.12 (t, J = 7.2 Hz, 3H). |
| 5 | 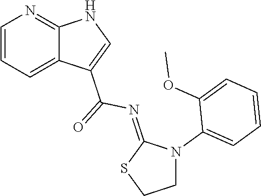 [SMILES: COc1ccccc1N1CCS/C1=N\C(=O)c1c[nH]c2ncccc12] | A1 C3 | 353.1 | (DMSO-d₆, 400 MHz) δ 12.11 (br s, 1H), 8.16 (dd, J = 1.6 Hz, 4.4 Hz, 1H), 7.85 (dd, J = 1.2 Hz, 8.0 Hz, 1H), 7.81 (s, 1H), 7.47 − 7.44 (m, 1H), 7.41 (dd, J = 1.6 Hz, 7.6 Hz, 1H), 7.24 (dd, J = 1.2 Hz, 8.4 Hz, 1H), 7.14 − 7.09 (m, 1H), 6.90 (dd, J = 4.8 Hz, 8.0 Hz, 1H), 3.96 (t, J = 7.6 Hz, 2H), 3.78 (s, 3H), 3.36 (t, J = 7.6 Hz, 2H). |
| 6 | 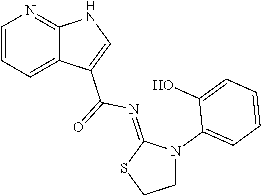 [SMILES: O=C(/N=C1\SCCN1c1ccccc1O)c1c[nH]c2ncccc12] | A1 C4 | 339.0 | (DMSO-d₆ + D₂O, 400 MHz) δ 8.16 (dd, J = 1.6, 4.8 Hz, 1H), 7.90 (d, J = 6.8 Hz, 1H), 7.84 (s, 1H), 7.33 − 7.31 (m, 2H), 7.03 (d, J = 8.0 Hz, 1H), 6.96 (dt, J = 1.6 Hz, 7.6 Hz, 1H), 6.92 − 6.87 (m, 1H), 3.97 (t, J = 7.6 Hz, 2H), 3.36 (t, J = 7.6 Hz, 2H). |
| 7 | 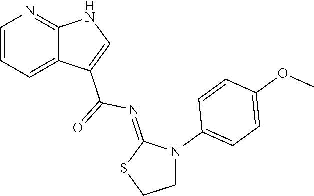 [SMILES: COc1ccc(N2CCS/C2=N\C(=O)c2c[nH]c3ncccc23)cc1] | A1 C5 | 353.1 | (DMSO-d₆ + D₂O, 400 MHz) δ 8.20 (dd, J = 1.6, 4.8 Hz, 1H), 8.11 (dd, J = 1.2, 8.0 Hz, 1H), 7.90 (s, 1H), 7.47 − 7.44 (m, 2H), 7.08 − 7.06 (m, 2H), 7.04 − 6.98 (m, 1H), 4.07 (t, J = 7.6 Hz, 2H), 3.81 (s, 3H), 3.30 (t, J = 7.6 Hz, 2H) |
| 8 | 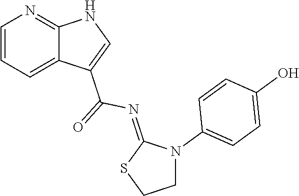 [SMILES: O=C(/N=C1\SCCN1c1ccc(O)cc1)c1c[nH]c2ncccc12] | T07 | 339.3 | (DMSO-d₆, 400 MHz) δ 12.15 (br. s, 1H), 9.65 (br. s, 1H), 8.22 − 8.16 (m, 2H), 7.89 (s, 1H), 7.36 − 7.34 (m, 2H), 7.02 (dd, J = 4.8, 8.0 Hz, 1H), 6.90 − 6.88 (m, 2H), 4.06 (t, J = 7.6 Hz, 2H), 3.29 (t, J = 7.6 Hz, 2H) |
| 9 | 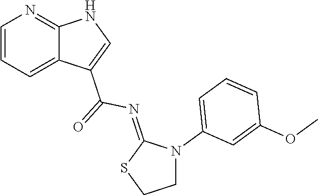 [SMILES: COc1cccc(N2CCS/C2=N\C(=O)c2c[nH]c3ncccc23)c1] | A1 C7 | 353.0 | (DMSO-d₆ + D₂O, 400 MHz) δ 8.23 − 8.20 (m, 2H), 7.93 (s, 1H), 7.43 (t, J = 8.4 Hz, 1H), 7.20 (t, J = 2.0 Hz, 1H), 7.16 − 7.14 (m, 1H), 7.05 (dd, J = 4.8 Hz, 7.6 Hz, 1H), 6.97 − 6.94 (m, 1H), 4.14 (t, J = 7.6 Hz, 2H), 3.79 (s, 3H), 3.30 (t, J = 8.0 Hz, 2H) |
| 10 | 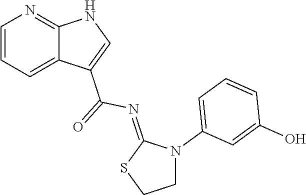 [SMILES: O=C(/N=C1\SCCN1c1cccc(O)c1)c1c[nH]c2ncccc12] | T09 | 339.0 | (DMSO-d₆ + D₂O, 400 MHz) δ 8.26 − 8.22 (m, 2H), 7.95 (s, 1H), 7.31 (t, J = 8.4 Hz, 1H), 7.08 − 7.05 (m, 2H), 6.98 (d, J = 8.0 Hz, 1H), 6.77 (d, J = 8.0 Hz, 1H), 4.10 (t, J = 7.6 Hz, 2H), 3.29 (t, J = 7.6 Hz, 2H) |
| 11 | 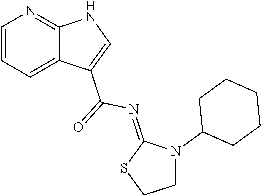 [SMILES: O=C(/N=C1\SCCN1C1CCCCC1)c1c[nH]c2ncccc12] | A1 C9 | 329.1 | (CD₃OD, 400 MHz) δ 8.82 (dd, J = 1.2, 8.0 Hz, IH), 8.34 − 8.29 (m, 1H), 8.19 (s, 1H), 7.36 (dd, J = 8.0, 5.2 Hz, IH), 4.71 − 4.61 (m, 1H), 3.81 (t, J = 8.0 Hz, 2H), 3.19 (t, J = 8.0 Hz, 2H), 1.99 − 1.89 (m, 4H), 1.82 − 1.73 (m, 1H), 1.71 − 1.60 (m, 2H), 1.58 − 1.47 (m, 2H), 1.35 − 1.20 (m, 1H). |
| 12 | 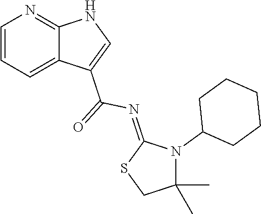 [SMILES: CC1(C)CS/C(=N\C(=O)c2c[nH]c3ncccc23)N1C1CCCCC1] | A1 C10 | 357.3 | (DMSO-d₆, 400 MHz) δ 12.35 (br. s, 1H), 8.63 (dd, J = 1.6, 8.0 Hz, 1H), 8.34 (dd, J = 1.2, 4.8 Hz, 1H), 8.03 (s, 1H), 7.29 (dd, J = 4.8, 8.0 Hz, 1H), 3.42 − 3.24 (m, 1H), 3.03 (s, 2H), 2.93 − 2.74 (m, 2H), 1.88 − 1.76 (m, 2H), 1.73 − 1.64 (m, 1H), 1.61 − 1.52 (m, 2H), 1.44 − 1.31 (m, 8H), 1.30 − 1.18 (m, 1H). |
| 13 | 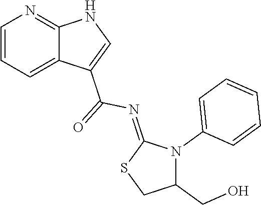 [SMILES: O=C(/N=C1\SCC(CO)N1c1ccccc1)c1c[nH]c2ncccc12] | A1 C11 | 335.1 | (DMSO-d₆, 400 MHz) δ 12.16 (br. s, 1H), 8.17 (d, J = 4.0 Hz, 1H), 7.91 (d, J = 7.6 Hz, 1H), 7.82 (s, 1H), 7.61 − 7.48 (m, 4H), 7.47 − 7.39 (m, 1H), 6.92 (dd, J = 4.4, 7.6 Hz, 1H), 5.10 (t, J = 4.8 Hz, 1H), 4.49 − 4.38 (m, 1H), 3.57 − 3.49 (m, 2H), 3.48 − 3.42 (m, 2H). |
| 16 | 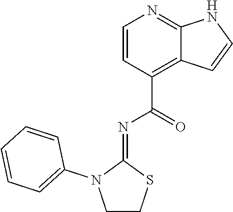 [SMILES: O=C(/N=C1\SCCN1c1ccccc1)c1ccnc2[nH]ccc12] | A2 C1 | 323.1 | (CD₃OD, 400 MHz) δ 6.65 (d, J = 4.8 Hz, 1H), 6.11 (d, J = 5.2 Hz, 1H), 6.03 − 5.96 (m, 4H), 5.88 − 5.84 (m, 1H), 5.79 (d, J = 3.2 Hz, 1H), 5.27 (d, J = 3.6 Hz, 1H), 2.70 (t, J = 8.0 Hz, 2H), 1.89 (t, J = 8.0 Hz, 2H) |
| 17 | 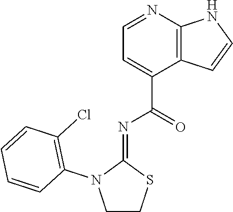 [SMILES: O=C(/N=C1\SCCN1c1ccccc1Cl)c1ccnc2[nH]ccc12] | A2 C12 | 357.0 | (DMSO-d₆, 400 MHz) δ 11.71 (br. s, 1H), 8.21 (d, J = 4.8 Hz, 1H), 7.74 − 7.62 (m, 2H), 7.58 − 7.50 (m, 2H), 7.43 (d, J = 4.8 Hz, 1H), 7.39 − 7.33 (m, 1H), 6.44 − 6.42 (m, 1H), 4.07- 4.06 (m, 2H), 3.55 − 3.44 (m, 2H). |
| 18 | 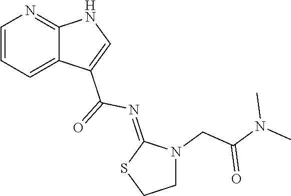 [SMILES: CN(C)C(=O)CN1CCS/C1=N\C(=O)c1c[nH]c2ncccc12] | A1 B2 | 332.0 | (DMSO-d₆, 400 MHz) δ 12.22 (br. s, 1H), 8.44 (dd, J = 1.2, 8.0 Hz, 1H), 8.26 (dd, J = 1.2, 4.4 Hz, 1H), 8.16 (s, 1H), 7.18 (dd, J = 4.8, 8.0 Hz, 1H), 4.65 (s, 2H), 3.74 (t, J = 8.0 Hz, 2H), 3.19 (t, J = 8.0 Hz, 2H), 3.10 (s, 3H), 2.86 (s, 3H). |
| 21 | 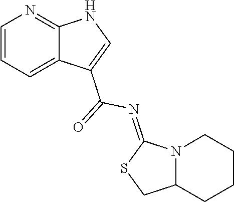 [SMILES: O=C(/N=C1\SCC2CCCCN12)c1c[nH]c2ncccc12] | A1 C13 | 300.9 | (DMSO-d₆, 400 MHz) δ 12.23 (br. s, 1H), 8.50 (dd, J = 2.0, 8.0 Hz, 1H), 8.26 (dd, J = 2.0, 4.4 Hz, 1H), 8.17 (s, 1H), 7.19 (dd, J = 4.4, 8.0 Hz, 1H), 4.68 − 4.64 (m, 1H), 3.72 − 3.64 (m, 1H), 3.35 − 3.33 (m, 1H), 2.99 − 2.92 (m, 1H), 2.82 − 2.77 (m, 1H), 2.07 − 1.76 (m, 3H), 1.53 − 1.35 (m, 3H). (DMSO- d6 + D₂O, 400 MHz) δ 8.51(dd, J = 2.0, 8.0 Hz, 1H), 8.25 (dd, J = 2.0, 4.8 Hz, 1H), 8.15 (s, 1H), 7.21 (dd, J = 4.8, 8.0 Hz, 1H), 4.65 − 4.61 (m, 1H), 3.72 − 3.68 (m, 1H), 3.35 − 3.30 (m, 1H), 2.98 − 2.91 (m, 1H), 2.81 − 2.76 (m, 1H), 1.95 − 1.74 (m, 3H), 1.51 − |
| | | | | 1.33 (m, 3H). |
| 23 | 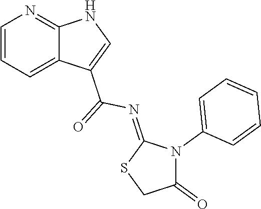 [SMILES: O=C(/N=C1\SCC(=O)N1c1ccccc1)c1c[nH]c2ncccc12] | A1 C15 | 337.1 | (DMSO-d₆, 400 MHz) δ 12.42 (br. s, 1H), 8.31 − 8.14 (m, 1H), 7.96 − 7.88 (m, 1H), 7.86 − 7.76 (m, 1H), 7.66 − 7.55 (m, 3H), 7.49 − 7.42 (m, 2H), 7.03 − 6.93 (m, 1H), 4.15 (s, 2H) |
| 25 | 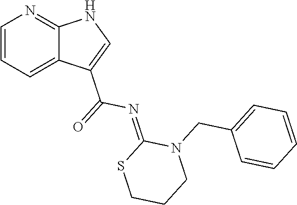 [SMILES: O=C(/N=C1\SCCCN1Cc1ccccc1)c1c[nH]c2ncccc12] | A1 C16 | 351.0 | (DMSO-d₆, 400 MHz) δ 12.05 (br. s, 1H), 8.31 (dd, J = 1.6, 8.0 Hz, 1H), 8.20 (dd, J = 2.0, 7.2 Hz, 1H), 7.90 (s, 1H), 7.41 − 7.36 (m, 4H), 7.31 − 7.29 (m, 1H), 7.06 (dd, J = 3.6, 7.6 Hz, 1H), 5.09 (s, 2H), 3.56 − 3.50 (m, 2H), 3.02 − 2.94 (m, 2H), 2.16 − 2.07 (m, 2H). |
| 26 | 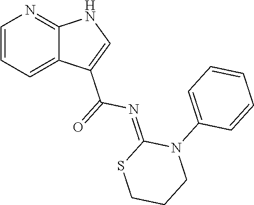 [SMILES: O=C(/N=C1\SCCCN1c1ccccc1)c1c[nH]c2ncccc12] | A1 C17 | 336.9 | (DMSO-d₆, 400 MHz) δ 11.98 (br. s, 1H), 8.21 − 8.02 (m, 1H), 7.63 (s, 1H), 7.56 − 7.50 (m, 2H), 7.48 − 7.33 (m, 4H), 6.84 − 6.70 (m, 1H), 3.85 − 3.74 (m, 2H), 3.14 − 3.06 (m, 2H), 2.35 − 2.27 (m, 2H). |
| 27 | 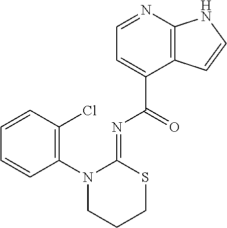 [SMILES: O=C(/N=C1\SCCCN1c1ccccc1Cl)c1ccnc2[nH]ccc12] | A2 C18 | 371.0 | (DMSO-d₆, 400 MHz) δ 11.74 (br. s, 1H), 8.14 (d, J = 5.2 Hz, 1H), 7.71 − 7.57 (m, 2H), 7.56 − 7.43 (m, 2H), 7.35 − 7.29 (m, 1H), 7.23 (d, J = 5.2 Hz, 1H), 6.22 − 6.15 (m, 1H), 3.88 − 3.82 (m, 1H), 3.70 − 3.63 (m, 1H), 3.25 − 3.08 (m, 2H), 2.39 − 2.30 (m, 2H) |
| 28 | 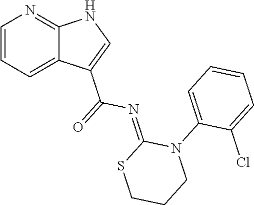 [SMILES: O=C(/N=C1\SCCCN1c1ccccc1Cl)c1c[nH]c2ncccc12] | A1 C18 | 371.1 | (DMSO-d₆, 400 MHz) δ 12.01 (br. s, 1H), 8.12 (dd, J = 1.6, 4.4 Hz, 1H), 7.70 − 7.67 (m, 1H), 7.61 − 7.58 (m, 2H), 7.56 − 7.49 (m, 2H), 7.32 (d, J = 5.6 Hz, 1H), 6.82 (dd, J = 4.8, 7.6 Hz, 1H), 3.84 − 3.78 (m, 1H), 3.66 − 3.60 (m, 1H), 3.16 − 3.05 (m, 2H), 2.35 − 2.28 (m, 2H). |
| 29 | 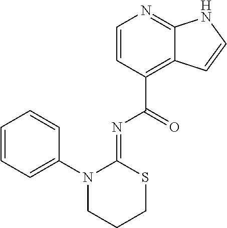 [SMILES: O=C(/N=C1\SCCCN1c1ccccc1)c1ccnc2[nH]ccc12] | A2 C17 | 337.1 | (CD₃OD-d₄,400 MHz) δ 8.09 − 8.04 (m, 1H), 7.56 − 7.49 (m, 2H), 7.46 − 7.33 (m, 4H), 7.20 (d, J = 3.2 Hz, 1H), 6.31 (d, J = 3.6 Hz, 1H), 3.97 − 3.91 (m, 2H), 3.25 − 3.19 (m, 2H), 2.49 − 2.38 (m, 2H) |
| 30 | 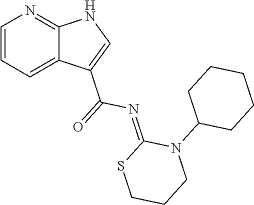 [SMILES: O=C(/N=C1\SCCCN1C1CCCCC1)c1c[nH]c2ncccc12] | A1 C19 | 343.1 | (CDCl₃, 400 MHz) δ 10.43 (br. s, 1H), 8.78 (dd, J = 1.2, 8.0 Hz, 1H), 8.33 (d, J = 3.6 Hz, 1H), 8.07 (s, 1H), 7.24 (dd, J = 4.8, 8.0 Hz, 1H), 5.19 (s, 1H), 3.49 − 3.40 (m, 2H), 2.95 (t, J = 6.4 Hz, 2H), 2.22 − 2.16 (m, 2H), 1.93 − 1.91 (m, 4H), 1.77 − 1.74 (m, 1H), 1.54 − 1.43 (m, 4H), 1.23 − 1.12 (m, 1H). |
| 31 | 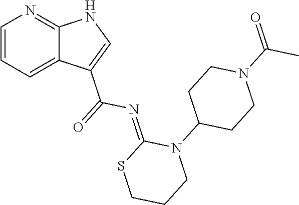 [SMILES: CC(=O)N1CCC(N2CCCS/C2=N\C(=O)c2c[nH]c3ncccc23)CC1] | A1 C20 | 386.1 | (CD₃OD, 400 MHz) δ 8.57 (dd, J = 1.6, 8.0 Hz, 1H), 8.25 (dd, J = 1.6, 4.8 Hz, 1H), 8.04 (s, 1H), 7.24 (dd, J = 4.4, 8.0 Hz, 1H), 5.45 − 5.32 (m, 1H), 4.78 − 4.68 (m, 1H), 4.15 − 4.03 (m, 1H), 3.53 − 3.47 (m, 2H), 3.28 − 3.23 (m, 1H), 3.01 (t, J = 6.4 Hz, 2H), 2.80 − 2.71 (m, 1H), 2.23 − 2.16 (m, 2H), 2.14 (s, 3H), 1.99 − 1.72 (m, 4H). |
| 32 | 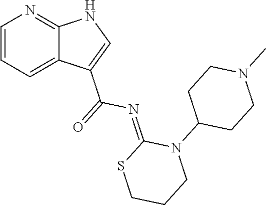 [SMILES: CN1CCC(N2CCCS/C2=N\C(=O)c2c[nH]c3ncccc23)CC1] | A1 C21 | 358.1 | (CD₃OD, 400 MHz) δ 8.57 (dd, J = 1.6, 8.0 Hz, 1H), 8.25 (dd, J = 1.6, 4.8 Hz, 1H), 8.01 (s, 1H), 7.23 (dd, J = 4.8, 8.0 Hz, 1H), 5.26 − 5.17 (m, 1H), 3.54 − 3.49 (m, 2H), 3.09 − 3.03 (m, 2H), 3.01 (t, J = 6.4 Hz, 2H), 2.35 (s, 3H), 2.28 − 2.15 (m, 4H), 2.01 − 1.90 (m, 2H), 1.89 − 1.82 (m, 2H). |
| 33 | 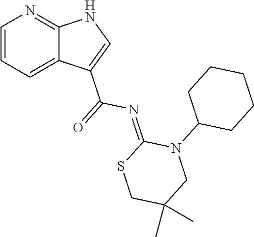 [SMILES: CC1(C)CS/C(=N\C(=O)c2c[nH]c3ncccc23)N(C2CCCCC2)C1] | A1 C22 | 371.1 | (DMSO-d₆, 400 MHz) δ 12.15 (br. s, 1H), 8.25 (dd, J = 1.6, 4.8 Hz, 1H), 8.11 (dd, J = 1.6, 8.0 Hz, 1H), 7.76 (s, 1H), 7.18 (dd, J = 4.8, 8.0 Hz, 1H), 3.56 (s, 2H), 3.22 − 3.16 (m, 1H), 2.96 (s, 2H), 1.44 − 1.36 (m, 3H), 1.27 − 1.21 (m, 2H), 1.15 − 1.07 (m, 8H), 1.01 − 0.93 (m, 1H), 0.85 − 0.75 (m, 2H). |
| 38 | 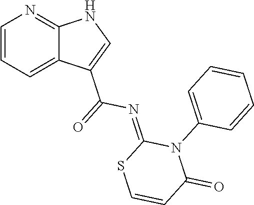 [SMILES: O=C(/N=c1\sccc(=O)n1-c1ccccc1)c1c[nH]c2ncccc12] | A1 B3 | 349.2 | (DMSO-d₆, 400 MHz) δ 12.41(br.s, 1H), 8.21(dd, J = 1.2, 4.8 Hz, 1H), 8.03 (d, J = 10.8 Hz, 1H), 7.77 (s, 1H), 7.62 − 7.52 (m, 4H), 7.42 − 7.39 (m, 2H), 6.96 (s, 1H), 6.75 (d, J =10.4 Hz, 1H). |
| 41 | 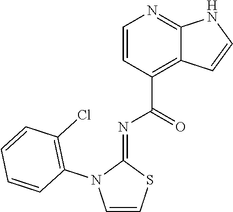 [SMILES: O=C(/N=c1\sccn1-c1ccccc1Cl)c1ccnc2[nH]ccc12] | A2 D1 | 355.0 | (CD₃OD, 400 MHz) δ 8.15 − 8.14 (m, 1H), 7.95 − 7.94 (m, 2H), 7.78 (d, J = 8.0 Hz, 1H), 7.70 − 7.64 (m, 3H), 7.40 (d, J = 4.8 Hz, 1H), 7.07 (d, J = 4.8 Hz, 1H), 6.95 − 6.92 (m, 1H). |
| 42 | 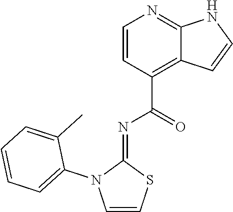 [SMILES: Cc1ccccc1-n1ccs/c1=N\C(=O)c1ccnc2[nH]ccc12] | A2 D2 | 335.0 | (DMSO-d₆, 400 MHz) δ 11.76 (br. s, 1H), 8.26 (d, J = 5.2 Hz, 1H), 7.70 (d, J = 4.8 Hz, 1H), 7.57 − 7.54 (m, 3H), 7.48 − 7.47 (m, 2H), 7.38 − 7.36 (m, 1H), 7.31 (d, J = 4.8 Hz, 1H), 6.52 (dd, J = 2.0, 3.6 Hz, 1H), 2.10 (s, 3H). ¹⁹F NMR (DMSO-d₆, 400 MHz) δ − 74.77 (s, 3F). |
| T43 | 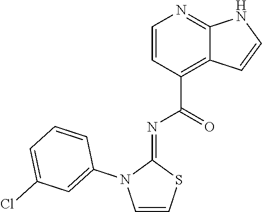 [SMILES: O=C(/N=c1\sccn1-c1cccc(Cl)c1)c1ccnc2[nH]ccc12] | A2 D3 | 355.0 | (DMSO-d₆, 400 MHz) δ 11.76 (br s, 1H), 8.27 (d, J = 4.8 Hz, 1H), 7.94 (s, 1H), 7.84 (d, J = 4.8 Hz, 1H), 7.72 − 7.67 (m, 3H), 7.62 (d, J = 4.8 Hz, 1H), 7.48 − 7.47 (m, 1H), 7.28 (d, J = 4.8 Hz, 1H), 6.83 (d, J = 0.8 Hz, 1H). |
| 44 | 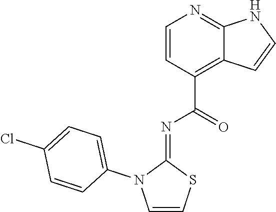 [SMILES: O=C(/N=c1\sccn1-c1ccc(Cl)cc1)c1ccnc2[nH]ccc12] | A2 D4 | 355.0 | (DMSO-d₆, 400 MHz) δ 11.76 (br. s, 1H), 8.27 (d, J = 4.8 Hz, 1H), 7.81 − 7.76 (m, 3H), 7.75 − 7.70 (m, 2H), 7.61 (d, J = 5.2 Hz, 1H), 7.51 − 7.47 (m, 1H), 7.27 (d, J = 4.8 Hz, 1H), 6.85 − 6.82 (m, 1H) |
| 45 | 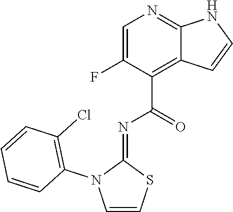 [SMILES: O=C(/N=c1\sccn1-c1ccccc1Cl)c1c(F)cnc2[nH]ccc12] | A3 D1 | 373.3 | (CDCl₃, 400 MHz) δ 9.14 (br. s, 1H), 8.19 (d, J = 3.6 Hz, 1H), 7.66 − 7.64 (m, 1H), 7.58 − 7.47 (m, 3H), 7.22 (t, J = 2.8 Hz, 1H), 7.13 (d, J = 4.8 Hz, 1H), 6.90 (d, J = 4.8 Hz, 1H), 6.62 (dd, J = 2.4, 3.6 Hz, 1H) ¹⁹F NMR (CDCl₃, 400 MHz) δ − 139.779 |
| 46 | 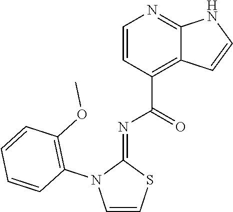 [SMILES: COc1ccccc1-n1ccs/c1=N\C(=O)c1ccnc2[nH]ccc12] | A2 D5 | 351.3 | (DMSO-d₆, 400 MHz) δ 11.67 (br. s, 1H), 8.23 (d, J = 4.8 Hz, 1H), 7.62 − 7.58 (m, 2H), 7.55 − 7.53 (m, 2H), 7.37 − 7.34 (m, 2H), 7.21 − 7.17 (m, 2H), 6.61 − 6.59 (m, 1H), 3.77 (s, 3H). |
| 47 | 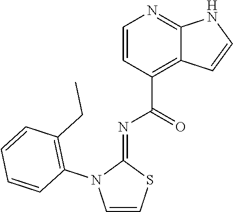 [SMILES: CCc1ccccc1-n1ccs/c1=N\C(=O)c1ccnc2[nH]ccc12] | A2 E1 | 348.9 | (Methanol-d₄, 400 MHz) δ 8.18 (d, J = 5.2 Hz, 1H), 7.66 (d, J = 4.8 Hz, 1H), 7.63 − 7.54 (m, 2H), 7.51 − 7.44 (m, 2H), 7.40 − 7.35 (m, 1H), 7.23 (d, J = 3.2 Hz, 1H), 7.19 (d, J = 4.8 Hz, 1H), 6.54 (d, J = 3.2 Hz, 1H), 2.48 (q, J = 7.6 Hz, 2H), 1.10 (t, J = 7.6 Hz, 3H). |
| 48 | 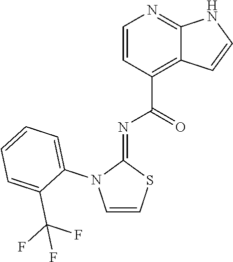 [SMILES: O=C(/N=c1\sccn1-c1ccccc1C(F)(F)F)c1ccnc2[nH]ccc12] | A2 E2 | 389.0 | (DMSO-d₆, 400 MHz) δ 11.67 (br. s, 1H), 8.22 (d, J = 4.8 Hz, 1H), 8.08 (d, J = 7.2 Hz, 1H), 7.99 (t, J = 7.2 Hz, 1H), 7.90 (t, J = 7.6 Hz, 1H), 7.81 (d, J = 7.6 Hz, 1H), 7.75 (d, J = 4.8 Hz, 1H), 7.48 (d, J = 4.8 Hz, 1H), 7.34 (t, J = 3.2 Hz, 1H), 7.28 (d, J = 4.8 Hz, 1H), 6.38 (dd, J = 2.0, 3.6 Hz, 1H). ¹⁹F NMR (DMSO-d₆, 400 MHz) δ −59.48 (s, 3F). |
| 49 | 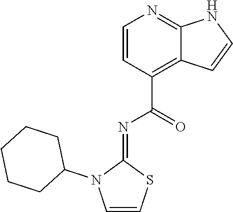 [SMILES: O=C(/N=c1\sccn1C1CCCCC1)c1ccnc2[nH]ccc12] | A2 E3 | 327.4 | (DMSO-d₆, 400 MHz) δ 11.79 (br. s, 1H), 8.33 (d, J = 4.8 Hz, 1H), 7.79 (d, J = 4.8 Hz, 1H), 7.77 (d, J = 4.8 Hz, 1H), 7.62 (d, J = 3.2 Hz, 1H), 7.17 (d, J = 3.2 Hz, 1H), 7.13 (d, J = 4.8 Hz, 1H), 5.09 − 4.93 (m, 1H), 2.07 − 2.00 (m, 2H), 1.96 − 1.88 (m, 2H), 1.86 − 1.73 (m, 3H), 1.57 − 1.46 (m, 2H), 1.35 − 1.25 (m, 1H). |
| 50 | 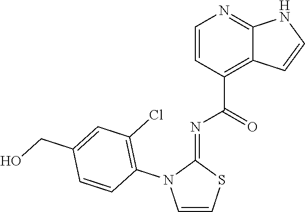 [SMILES: O=C(/N=c1\sccn1-c1ccc(CO)cc1Cl)c1ccnc2[nH]ccc12] | A2 E4 | 385.2 | (DMSO-d₆, 400 MHz) δ 11.69 (br. s, IH), 8.23 (d, J = 4.8 Hz, 1H), 7.77 (d, J = 8.0 Hz, 1H), 7.73 − 7.71 (m, 1H), 7.65 − 7.59 (m, 2H), 7.52 (dd, J = 0.8, 4.8 Hz, IH), 7.39 − 7.36 (m, 1H), 7.52 (dd, J = 0.8, 4.8 Hz, 1H), 6.57 − 6.56 (m, 1H), 5.48 (t, J = 5.2 Hz, IH), 4.62 (d, J = 5.6 Hz, 1H). |
| 51 | 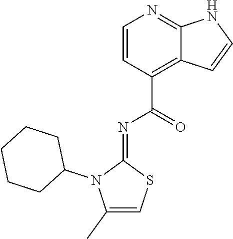 [SMILES: Cc1cs/c(=N\C(=O)c2ccnc3[nH]ccc23)n1C1CCCCC1] | A2 D6 | 341.0 | (DMSO-d₆, 400 MHz) δ 11.79 (br. s, 1H), 8.35 (d, J = 4.8 Hz, 1H), 7.80 (d, J = 4.8 Hz, 1H), 7.61 (t, J = 2.8 Hz, 1H), 7.19 − 7.18 (m, 1H), 6.74 (s, 1H), 4.50 − 3.92 (m, 1H), 3.25 − 2.75 (m, 2H), 2.41 (s, 3H), 1.93 − 1.90 (m, 2H), 1.81 − 1.75 (m, 3H), 1.47 − 1.41 (m, 3H). |
| 52 | 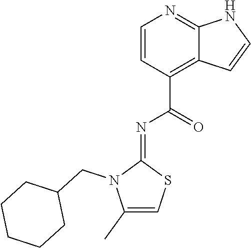 [SMILES: Cc1cs/c(=N\C(=O)c2ccnc3[nH]ccc23)n1CC1CCCCC1] | A2 D7 | 355.1 | (DMSO-d₆, 400 MHz) δ 12.02 (br. s, 1H), 8.40 (d, J = 5.2 Hz, 1H), 7.88 (d, J = 5.2 Hz, 1H), 7.67 − 7.65 (m, 1H), 7.26 − 7.24 (m, 1H), 6.82 (d, J = 0.8 Hz, 1H), 4.20 (d, J = 7.6 Hz, 2H), 2.54 (s, 1H), 2.38 (d, J = 0.8 Hz, 3H), 2.18 − 1.97 (m, 1H), 1.75 − 1.65 (m, 2H), 1.65 − 1.5 (m, 2H), 1.20 − 1.05 (m, 5H). |
| 53 | 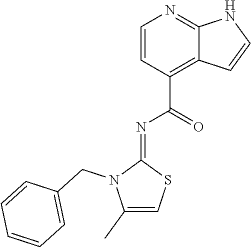 [SMILES: Cc1cs/c(=N\C(=O)c2ccnc3[nH]ccc23)n1Cc1ccccc1] | A2 D8 | 349.1 | (DMSO-d₆, 400 MHz) δ 11.76 (br. s, 1H), 8.30 (d, J = 4.8 Hz, 1H), 7.78 (d, J = 4.8 Hz, 1H), 7.53 − 7.52 (m, 1H), 7.39 − 7.35 (m, 2H), 7.31 − 7.26 (m, 3H), 7.06 − 7.05 (m, 1H) 6.84 (d, J = 1.2 Hz, 1H), 5.68 (s, 2H), 2.28 (d, J = 0.8 Hz, 3H) |
| 55 | 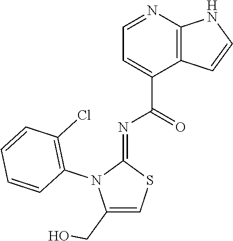 [SMILES: O=C(/N=c1\scc(CO)n1-c1ccccc1Cl)c1ccnc2[nH]ccc12] | A2 E5 | 385.0 | (DMSO-d₆, 400 MHz) δ 11.66 (br. s, 1H), 8.21 (d, J = 5.2 Hz, 1H), 7.84 − 7.78 (m, 1H), 7.75 − 7.61 (m, 3H), 7.49 (d, J = 4.8 Hz, 1H), 7.32 (t, J = 4.8 Hz, 1H), 7.09 (s, 1H), 6.38 − 6.34 (m, 1H), 5.47 (t, J = 5.6 Hz, 1H), 4.17 (d, J = 5.6 Hz, 2H). |
| 56 | 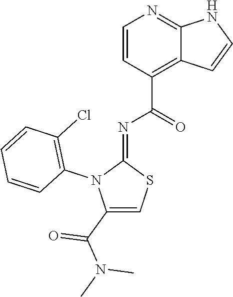 [SMILES: CN(C)C(=O)c1cs/c(=N\C(=O)c2ccnc3[nH]ccc23)n1-c1ccccc1Cl] | A2 D10 | 426.0 | (DMSO-d₆, 400 MHz) δ 11.72 (br s, 1H), 8.24 (d, J = 4.8 Hz, 1H), 7.76 − 7.73 (m, 1H), 7.67 − 7.66 (m, 1H), 7.62 − 7.57 (m, 2H), 7.55 (s, 1H), 7.51 (d, J = 5.2 Hz, 1H), 7.37 (t, J = 2.8 Hz, 1H), 6.43 (dd, J = 1.6, 3.2 Hz, 1H), 3.17 (s, 3H), 2.83 (s, 3H). |
| 58 | 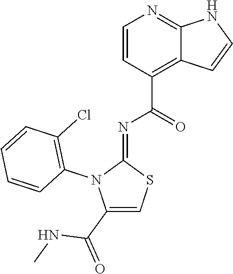 [SMILES: CNC(=O)c1cs/c(=N\C(=O)c2ccnc3[nH]ccc23)n1-c1ccccc1Cl] | A2 D11 | 412.0 | (DMSO-d₆, 400 MHz) δ 11.70 (br s, 1H), 8.77 (br. d, J = 4.8 Hz, 1H), 8.23 (d, J = 4.8 Hz, 1H), 7.73 − 7.69 (m, 2H), 7.62 − 7.54 (m, 3H), 7.49 (d, J = 5.2 Hz, 1H), 7.34 (t, J = 2.8 Hz, 1H), 6.35 (s, 1H), 2.63 (d, J = 4.4 Hz, 3H). |
| 61 | 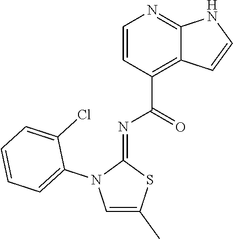 [SMILES: Cc1cn(-c2ccccc2Cl)/c(=N/C(=O)c2ccnc3[nH]ccc23)s1] | A2 E6 | 369.0 | (DMSO-d₆, 400 MHz) δ 11.69 (br. s, 1H), 8.22 (d, J = 4.8 Hz, 1H), 7.88 − 7.80 (m, 1H), 7.77 − 7.71 (m, 1H), 7.71 − 7.59 (m, 2H), 7.19 (d, J = 4.2 Hz, 1H), 7.47 − 7.42 (m, 1H), 7.38 − 7.30 (m, 1H), 6.54 − 6.41 (m, 1H), 2.42 − 2.35 (m, 3H). |
| 67 | 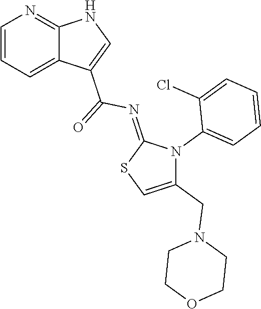 [SMILES: O=C(/N=c1\scc(CN2CCOCC2)n1-c1ccccc1Cl)c1c[nH]c2ncccc12] | A1 E11 | 454.1 | (DMSO-d₆, 400 MHz) δ 12. 10 (br. s, IH), 8.16 (dd, J = 1.6, 4.8 Hz, 1H), 7.86 (d, J = 1.6 Hz, 1H), 7.79 (dd, J = 1.2, 8.0 Hz, 1H), 7.72 (dd, J = 2.0, 7.6 Hz, 1H), 7.71 − 7.68 (m, 1H), 7.66 (dd, J = 2.0, 7.6 Hz, 1H), 7.62 (td, J = 1.2, 7.6 Hz, IH), 6.98 (s, 1H), 6.85 (dd, J = 4.8, 8.0 Hz, 1H), 3.46 − 3.39 (m, 4H), 3.28 − 3.27 (m, 1H), 3.19 − 3.14 (m, 1H), 2.25 − 2.15 (m, 2H), 2.14 − 2.06 (m, 2H). |
| 68 | 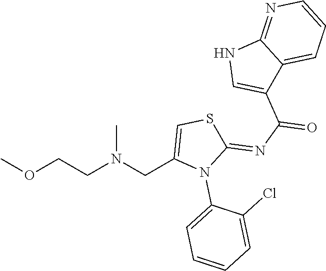 [SMILES: COCCN(C)Cc1cs/c(=N\C(=O)c2c[nH]c3ncccc23)n1-c1ccccc1Cl] | A1 E12 | 456.2 | (CD₃OD, 400 MHz) 6 8.13 (dd, J = 1.6, 4.8 Hz, 1H), 7.92 (s, IH), 7.80 − 7.74 (m, 2H), 7.73 − 7.66 (m, 1H), 7.65 − 7.60 (m, 2H), 6.92 (s, 1H), 6.89 (dd, J = 4.8, 8.0 Hz, 1H), 3.37 − 3.33 (m, 2H), 3.23 (s, 3H), 3.22 − 3.19 (m, 2H), 2.51 − 2.38 (m, 2H), 2.18 (s, 3H) |
| 69 | 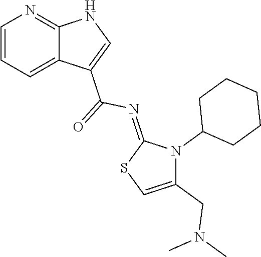 [SMILES: CN(C)Cc1cs/c(=N\C(=O)c2c[nH]c3ncccc23)n1C1CCCCC1] | A1 E13 | 384.2 | (DMSO-d₆, 400 MHz) δ 12.15 (br. s, 1 H), 8.66 (d, J = 7.6 Hz, 1 H), 8.29 (dd, J = 1.2, 4.4 Hz, 1 H), 8.07 (d, J = 2.4 Hz, 1 H), 7.20 (dd, J = 4.4, 7.6 Hz, 1 H), 6.76 (s, 1 H), 4.19 − 4.60 (m, 1 H), 3.42 (s, 2 H), 2.94 − 3.09 (m, 2 H), 2.19 (s, 6 H), 1.87 − 1.94 (m, 2 H), 1.57 − 1.86 (m, 4 H), 1.35 − 1.40 (m, 2 H). |
| 70 | 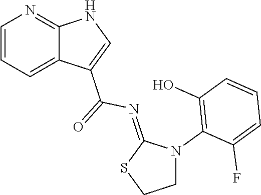 [SMILES: O=C(/N=C1\SCCN1c1c(O)cccc1F)c1c[nH]c2ncccc12] | A1 C27 | 357.1 | (CD₃OD, 400 MHz) δ 8.14 (dd, J = 1.6, 4.8 Hz, 1H), 8.01 (dd, J =1.6, 8.0 Hz, 1H), 7.92 (s, 1H), 7.37 − 7.31 (m, 1H), 6.95 (dd, J = 4.8, 8.0 Hz, 1H), 6.86 (d, J = 8.4 Hz, 1H), 6.81 − 6.77 (m, 1H), 4.16 − 4.10 (m, 1H), 3.95 − 3.88 (m, 1H), 3.52 − 3.41 (m, 2 |
| 71 | 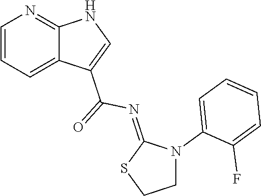 [SMILES: O=C(/N=C1\SCCN1c1ccccc1F)c1c[nH]c2ncccc12] | A1 C28 | 341.1 | (DMSO-d₆, 400 MHz) δ 8.16 (dd, J = 2.1, 4.8 Hz, 1H), 8.03 (d, J = 8.0 Hz, 1H), 7.90 (s, 1H), 7.58 − 7.51 (m, 2H), 7.40 − 7.38 (m, 2H), 6.98 (dd, J = 4.8, 8.0 Hz, 1H), 4.10 (t, J = 7.6 Hz, 2H), 3.45 (t, J = 7.6 Hz, 2H). |
| 72 | 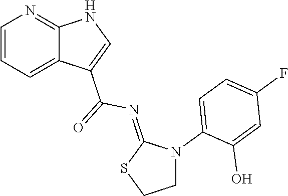 [SMILES: O=C(/N=C1\SCCN1c1ccc(F)cc1O)c1c[nH]c2ncccc12] | A1 C29 | 357.2 | (DMSO-d₆, 400 MHz) δ 11.46 (br s, 1H), 8.16 − 8.15 (m, 1H), 8.10 (dd, J = 1.2, 8.0 Hz, 1H), 7.92 (s, 1H), 7.33 (dd, J = 6.0, 8.8 Hz, 1H), 6.99 (dd, J = 4.8, 8.0 Hz, 1H), 6.80 − 6.72 (m, 2H), 4.06 (t, J = 8.0 Hz, 2H), 3.42 (t, J = 8.0 Hz, 2H). |
| 73 | 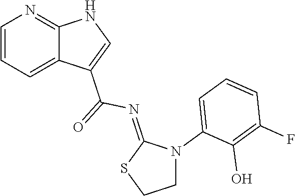 [SMILES: O=C(/N=C1\SCCN1c1cccc(F)c1O)c1c[nH]c2ncccc12] | A1 C30 | 357.1 | (DMSO-d₆ + D₂O, 400 MHz) δ 8.18 (d, J = 4.0 Hz, 1H), 7.92 (d, J = 7.6 Hz, 1H), 7.86 (s, 1H), 7.30 (t, J = 9.2 Hz, 1H), 7.17 (br d, J = 8.0 Hz, 1H), 6.97 − 6.93 (m, 2H), 3.98 (t, J = 8.0 Hz, 2H), 3.37 (t, J = 7.6 Hz, 2H). |
| 74 | 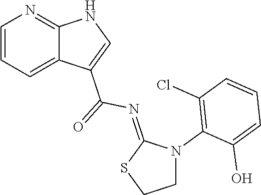 [SMILES: O=C(/N=C1\SCCN1c1c(O)cccc1Cl)c1c[nH]c2ncccc12] | A1 C31 | 373.1 | (CD₃OD, 400 MHz) δ 8.13 (dd, J = 1.2, 4.8 Hz, 1H), 7.93-7.91 (m, 2H), 7.32 (t, J = 8.0 Hz, 1H), 7.10-7.08 (dd, J = 1.2, 8.0 Hz, 1H), 6.99-6.97 (dd, J = 1.2, 8.4 Hz, 1H), 6.93-6.90 (dd, J = 5.2, 8.4 Hz, 1H), 4.09-4.06 (m, 1H), 3.97-3.95 (m, 1H), 3.48 (t, J = 7.6 Hz, 2H). |
| 75 | 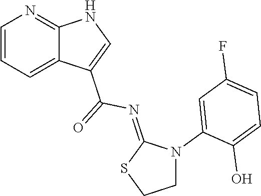 [SMILES: O=C(/N=C1\SCCN1c1cc(F)ccc1O)c1c[nH]c2ncccc12] | A1 C32 | 357.0 | (DMSO-d₆ + D₂O, 400 MHz,) δ 8.15 (s, 1H), 7.88 (s, 2H), 7.28 − 7.10 (m, 2H), 7.04 − 6.89 (m, 2H), 3.94 (t, J = 7.2 Hz, 2H), 3.33 (t, J = 7.2 Hz, 2H) |
| 76 | 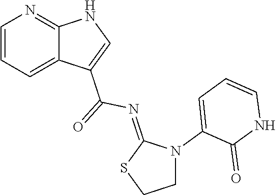 [SMILES: O=C(/N=C1\SCCN1c1ccc[nH]c1=O)c1c[nH]c2ncccc12] | A1 C33 | 340.0 | (DMSO-d₆, 400 MHz) δ 8.19 (d, J = 4.4 Hz, 1H), 8.12 (d, J = 8.0 Hz, 1H), 7.89 (s, 1H), 7.70 (d, J = 6.8 Hz, 1H), 7.53 (d, J = 6.0 Hz, 1H), 7.04 (dd, J = 4.8, 7.6 Hz) 6.42 (t, J = 6.8 Hz, 1H), 3.92 (t, J = 7.2 Hz, 2H), 3.31 (t, J = 7.6 Hz, 2H). |
| 77 | 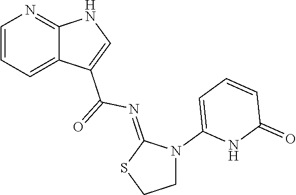 [SMILES: O=C(/N=C1\SCCN1c1cccc(=O)[nH]1)c1c[nH]c2ncccc12] | A1 C34 | 340.1 | (DMSO-d₆, 400 MHz) δ 12.36 (br s, 1H), 8.43 (dd, J = 1.6 Hz, 8.0 Hz, 1H), 8.29 (dd, J = 1.6, 4.4 Hz, 1H), 8.11 (s, 1H), 7.78 (s, 2H), 7.18 (dd, J = 4.4, 8.0 Hz, 1H), 6.48 (d, J = 7.2 Hz, 1H), 4.26 (t, J = 7.6 Hz, 2H), 3.27 (t, J = 7.6 Hz, 2H). |
| 78 | 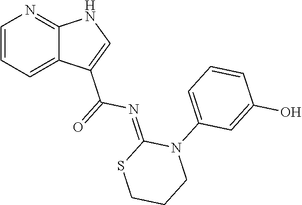 [SMILES: O=C(/N=C1\SCCCN1c1cccc(O)c1)c1c[nH]c2ncccc12] | A1 C35 | 353.1 | (CD₃OD, 400 MHz) δ 8.09 (dd, J = 1.6, 4.8 Hz, 1H), 7.78 (s, 1H), 7.57 (dd, J = 1.6, 8.0 Hz, 1H), 7.35 (t, J = 8.0 Hz, 1H), 6.91-6.80 (m, 4H), 3.88 (t, J = 5.6 Hz, 2H), 3.16 (t, J = 6.0 Hz, 2H), 2.43-2.37 (m, 2H). |
| 80 | 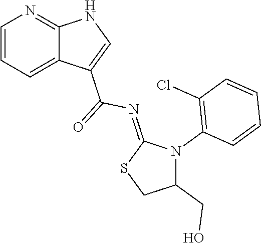 [SMILES: O=C(/N=C1\SCC(CO)N1c1ccccc1Cl)c1c[nH]c2ncccc12] | A1 C36 | 387.0 | (DMSO-d₆, 400 MHz) δ 12.12 (s, 1H), 8.17 (d, J = 8.4 Hz, 1H), 7.79 − 7.50 (m, 6H), 6.88-6.84 (m, 1H), 5.16 − 5.04 (m, 1H), 4.44 − 4.18 (m, 1H), 3.60-3.46 (m, 3H), 3.43 − 3.39 (m, 1H). |
| 82 | 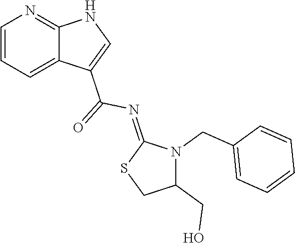 [SMILES: O=C(/N=C1\SCC(CO)N1Cc1ccccc1)c1c[nH]c2ncccc12] | A1 C37 | 367.1 | (DMSO-d₆, 400 MHz) δ 12.18 (s, 1H), 8.40 (dd, J = 1.6, 7.6 Hz, 1H), 8.24 (dd, J = 1.6 Hz, J = 3.6 Hz, 1H), 8.12 (d, J = 2.0 Hz, 1H), 7.39 − 7.34 (m, 4H), 7.31 − 7.27 (m, 1H), 7.12 (dd, J = 4.8 Hz, J = 8.0 Hz, 1H), 5.47 (d, J = 14.4 Hz, 1H), 5.14 (t, J = 5.6 Hz, 1H), 4.62 (d, J = 15.2 Hz, 1H), 3.88 − 3.84 (m, 1H), 3.60 − 3.56 (m, 2H), 3.29 − 3.26 (m, 1H), 3.16 − 3.12 (m, 1H). |

### Preparation of (Z)—N-(3-benzyl-1,3-thiazepan-2-ylidene)-1H-pyrrolo[2,3-b]pyridine-3-carboxamide (Example 39)

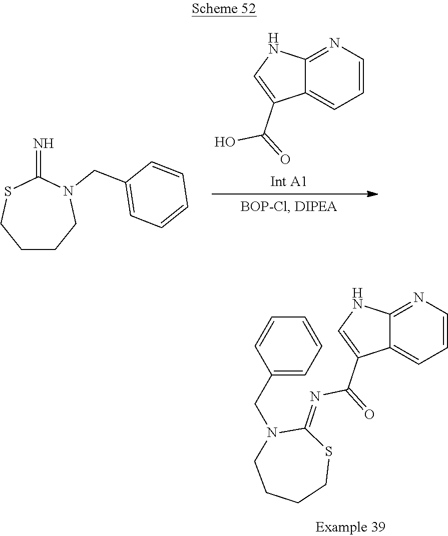 [SMILES: N=C1SCCCCN1Cc1ccccc1 ; O=C(O)c1c[nH]c2ncccc12 ; O=C(/N=C1\SCCCCN1Cc1ccccc1)c1c[nH]c2ncccc12]

To a mixture of 3-benzyl-1,3-thiazepan-2-imine (C25) (0.100 g, 0.454 mmol) and 1H-pyrrolo[2,3-b]pyridine-3-carboxylic acid (A1) (0.088 g, 0.545 mmol) in DCM (1 mL) was added DIPEA (0.237 mL, 1.36 mmol) and a solution of BOP—Cl (0.139 g, 0.545 mmol) in DCM (1 mL) at 25° C. The mixture was stirred at 25° C. for 12 hours., quenched with ice-water (20 mL) and extracted with EtOAc (3×20 mL). The combined organic layers were washed with brine (2×20 mL), dried over anhydrous Na₂SO₄, filtered and the filtrate concentrated under reduced pressure. The residue was purified by reversed-phase flash (0.1% NH₃·H₂O/MeCN condition), followed by column chromatography (SiO₂, petroleum ether/EtOAc=10/1 to 1/1) to afford 0.053 g of (Z)—N-(3-benzyl-1,3-thiazepan-2-ylidene)-1H-pyrrolo[2,3-b] pyridine-3-carboxamide (Example 39) as a solid.

¹H NMR (400 MHz, DMSO-d₆) δ 12.18 (br. s, 1H), 8.35 (dd, J=1.2, 7.6 Hz, 1H), 8.26 (dd, J=1.6, 4.8 Hz, 1H), 7.86 (s, 1H), 7.42-7.35 (m, 4H), 7.31-7.27 (m, 1H), 7.16 (dd, J=4.4, 7.6 Hz, 1H), 4.89 (s, 2H), 3.62-3.60 (m, 2H), 2.86-2.84 (m, 2H), 1.99-1.87 (m, 2H), 1.64-1.63 (m, 2H) LCMS (m/z [M+H]⁺): 365.1

### Preparation of (Z)—N-(3-phenyl-1,3-thiazepan-2-ylidene)-1H-pyrrolo[2,3-b]pyridine-3-carboxamide (Example 40)

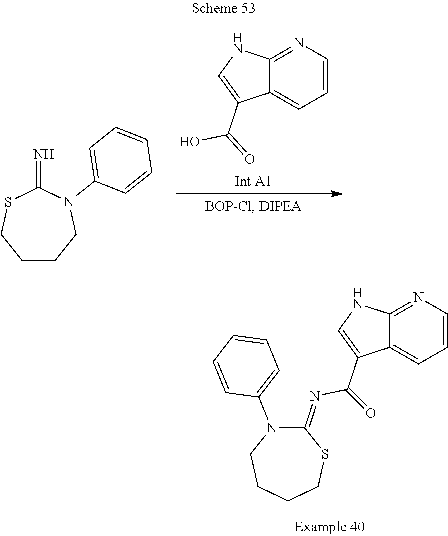 [SMILES: N=C1SCCCCN1c1ccccc1 ; O=C(O)c1c[nH]c2ncccc12 ; O=C(/N=C1\SCCCCN1c1ccccc1)c1c[nH]c2ncccc12]

To a mixture of 3-phenyl-1,3-thiazepan-2-imine (C26) as the hydrobromide salt (0.100 g, 0.348 mmol) and 1H-pyrrolo[2,3-b]pyridine-3-carboxylic acid (A1) (0.085 g, 0.522 mmol) in dichloromethane (5 mL) was added diisopropylethylamine (0.212 mL, 1.22 mmol), followed by a solution of BOP—Cl (0.133 g, 0.522 mmol) in dichloromethane (1 mL). The mixture was stirred at 20° C. for 1 hour then water (50 mL) was added and the mixture extracted with ethyl acetate (2×50 mL). The combined organic layers were dried over anhydrous sodium sulfate, filtered, and the filtrate was concentrated under reduced pressure. The residue was purified by preparative TLC (ethyl acetate mobile phase) to afford 0.037 g of (Z)—N-(3-phenyl-1,3-thiazepan-2-ylidene)-1H-pyrrolo[2,3-b]pyridine-3-carboxamide (Example 40) as a solid.

¹H NMR (CD₃OD, 400 MHz) δ 8.17 (dd, J=1.6, 4.8 Hz, 1H), 7.88 (d, J=8.0 Hz, 1H), 7.76 (s, 1H), 7.56-7.48 (m, 2H), 7.43-7.37 (m, 3H), 7.02 (dd, J₁=4.8, 8.0 Hz, 1H), 4.16-4.10 (m, 2H), 3.11-3.06 (m, 2H), 2.15-2.08 (m, 2H), 1.88-1.82 (m, 2H)

LCMS (m/z [M+H]⁺): 351.1

### Preparation of (Z)—N-(4-((dimethylamino)methyl)-3-phenylthiazolidin-2-ylidene)-1H-pyrrolo[2,3-b]pyridine-3-carboxamide (Example 14)

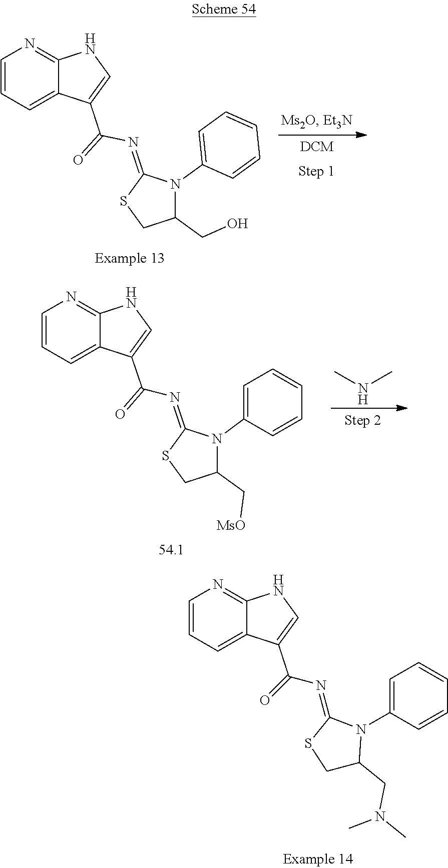

### Step 1. (Z)-(2-((1H-Pyrrolo[2,3-b]pyridine-3-carbonyl)imino)-3-phenylthiazolidin-4-yl)methyl methanesulfonate (54.1)

To a solution of (Z)—N-(4-(hydroxymethyl)-3-phenylthiazolidin-2-ylidene)-1H-pyrrolo[2,3-b]pyridine-3-carboxamide (Example 13) (0.120 g, 0.325 mmol) and methanesulfonic anhydride (0.227 g, 1.30 mmol) in DCM (3 mL) and THF (3 mL) was added Et₃N (0.136 mL, 0.976 mmol) at 25° C. The mixture was stirred at 25° C. for 12 hours then diluted with water (50 mL) and extracted with EtOAc (3×50 mL). The combined organic layers were washed with brine (80 mL), dried over anhydrous Na₂SO₄, filtered, and concentrated to afford 0.16 g of the title compound (54.1) as a solid.

¹H NMR (400 MHz, DMSO-d₆) δ 12.18 (br. s, 1H), 8.18 (dd, J=1.2, 4.8 Hz, 1H), 7.88 (d, J=7.6 Hz, 1H), 7.84 (d, J=2.4 Hz, 1H), 7.62-7.52 (m, 4H), 7.51-7.45 (m, 1H), 6.93 (dd, J=4.4, 7.6 Hz, 1H), 4.88-4.80 (m, 1H), 4.33-4.21 (m, 2H), 3.73-3.62 (m, 2H), 3.16 (s, 3H) LCMS (m/z [M+H]⁺): 431.0

### Step 2. (Z)—N-(4-((Dimethylamino)methyl)-3-phenylthiazolidin-2-ylidene)-1H-pyrrolo[2,3-b]pyridine-3-carboxamide (Example 14)

A solution of (Z)-(2-((1H-pyrrolo[2,3-b]pyridine-3-carbonyl)imino)-3-phenylthiazolidin-4-yl)methyl methanesulfonate (54.1) (0.160 g, 0.249 mmol) in dimethylamine (2 M in THF, 4 mL) was stirred at 100° C. for 12 hours in a sealed tube. The cooled mixture was concentrated under reduced pressure and the residue was dissolved in a mixture of MeOH (3 mL) and THF (3 mL). To this was added potassium carbonate (0.103 g, 0.746 mmol) and the mixture was stirred at 25° C. for 1 hour then filtered and the filtrate concentrated. The residue was purified by preparative HPLC (Waters Xbridge 25×150 mm, 5 μm; mobile phase A: water/10 mM NH₄HCO₃, mobile phase B: ACN]; gradient: 31% B to 61% B over 8 min) to afford 27 mg of (Z)—N-(4-((dimethylamino)methyl)-3-phenylthiazolidin-2-ylidene)-1H-pyrrolo[2,3-b]pyridine-3-carboxamide (Example 14) as a solid.

¹H NMR (400 MHz, DMSO-d₆) δ 12.14 (br. s, 1H), 8.18 (dd, J=1.6, 4.4 Hz, 1H), 7.95 (d, J=7.2 Hz, 1H), 7.83 (s, 1H), 7.57-7.52 (m, 4H), 7.47-7.40 (m, 1H), 6.94 (dd, J=4.8, 8.0 Hz, 1H), 4.65-4.60 (m, 1H), 3.54 (dd, J 1=8.0, 11.2 Hz, 1H), 3.30-3.26 (m, 1H), 2.61 (dd, J=10.4, 12.0 Hz, 1H), 2.17 (dd, J=4.0, 12.4 Hz, 1H), 2.11 (s, 6H)

LCMS (m/z [M+H]⁺): 380.1

### Preparation of (Z)-2-((1H-pyrrolo[2,3-b]pyridine-3-carbonyl)imino)-N-methyl-3-phenylthiazolidine-4-carboxamide (example 15)

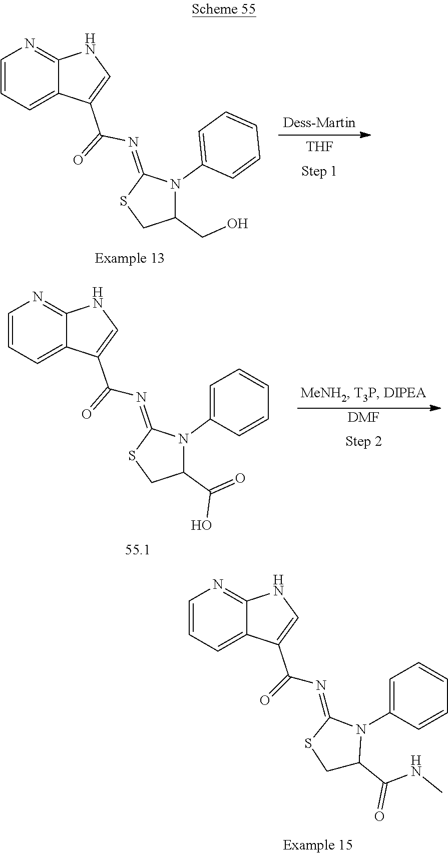

### Step 1. (Z)-2-((1H-Pyrrolo[2,3-b]pyridine-3-carbonyl)imino)-3-phenylthiazolidine-4-carboxylic acid (55.1)

To a solution of (Z)—N-(4-(hydroxymethyl)-3-phenylthiazolidin-2-ylidene)-1H-pyrrolo [2,3-b]pyridine-3-carboxamide (Example 13) (0.300 g, 0.692 mmol) in THF (10 mL) was added Dess-Martin periodinane (1.47 g, 3.46 mmol) at 25° C. The resulting mixture was stirred at 25° C. for 12 hours, diluted with water (80 mL) and extracted with THF (3×40 mL). The combined organic layers were dried over anhydrous Na₂SO₄, filtered and concentrated. The residue was triturated with water (3 mL) to afford 0.30 g of the title compound (55.1) as a solid.

¹H NMR (400 MHz, DMSO-d₆) δ 12.25 (br. s, 1H), 8.25-8.20 (m, 1H), 8.14-8.07 (m, 1H), 7.93-7.89 (m, 1H), 7.62-7.57 (m, 2H), 7.55-7.49 (m, 2H), 7.42-7.35 (m, 1H), 7.05-6.99 (m, 1H), 5.19 (dd, J=2.4, 8.8 Hz, 1H), 3.78-3.71 (m, 2H)

LCMS (m/z [M+H]⁺): 367.0

### Step 2. (Z)-2-((1H-Pyrrolo[2,3-b]pyridine-3-carbonyl)imino)-N-methyl-3-phenylthiazolidine-4-carboxamide (Example 15)

To a solution of methylamine hydrochloride (0.008 g, 0.117 mmol) and (Z)-2-((1H-pyrrolo[2,3-b]pyridine-3-carbonyl)imino)-3-phenylthiazolidine-4-carboxylic acid (55.1) (0.060 g, 0.117 mmol) in DMF (1 mL) at 0° C. was added T₃P (0.105 mL, 0.176 mmol, 50% in EtOAc) and DIPEA (0.102 mL, 0.586 mmol). The mixture was stirred at 25° C. for 1 hour, diluted with water (50 mL) and extracted with EtOAc (3×30 mL). The combined organic layers were washed with brine (80 mL), dried with anhydrous Na₂SO₄, filtered, and concentrated. The residue was triturated with MeOH (3 mL) to afford 0.013 g of (Z)-2-((1H-pyrrolo[2,3-b]pyridine-3-carbonyl)imino)-N-methyl-3-phenylthiazolidine-4-carboxamide (Example 15) as a solid.

¹H NMR (400 MHz, DMSO-d₆) δ 12.19 (br. s, 1H), 8.27-8.18 (m, 2H), 8.01 (d, J=8.0 Hz, 1H), 7.87 (d, J=2.8 Hz, 1H), 7.54-7.48 (m, 4H), 7.42-7.36 (m, 1H), 6.97 (dd, J=4.8, 8.0 Hz, 1H), 4.92 (dd, J=4.8, 8.8 Hz, 1H), 3.69 (dd, J=9.2, 11.6 Hz, 1H), 3.30 (dd, J=4.8, 11.6 Hz, 1H), 2.57 (d, J=4.8 Hz, 3H) LCMS (m/z [M+H]⁺): 380.0

### Preparation of (Z)—N-(3-(2-hydroxypropyl)thiazolidin-2-ylidene)-1H-pyrrolo[2,3-b]pyridine-3-carboxamide (Example 19)

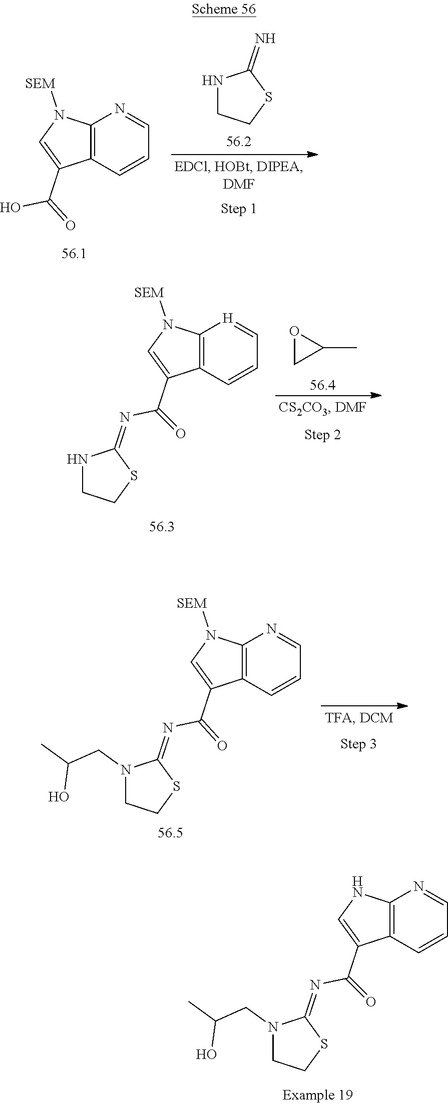

### Step 1. (ZN-(Thiazolidin-2-ylidene)-1-((2-(trimethylsilyl)ethoxy)methyl)-1H-pyrrolo[2,3-b]pyridine-3-carboxamide (56.3)

To a mixture of 1-((2-(trimethylsilyl)ethoxy)methyl)-1H-pyrrolo[2,3-b]pyridine-3-carboxylic acid (56.1) (0.500 g, 1.71 mmol) and thiazolidin-2-imine (56.2) (0.170 g 1.71 mmol) in DMF (20 mL) was added DIPEA (0.330 g, 2.56 mmol), HOBt (0.280 g, 2.05 mmol) and EDCI (0.390 g, 2.05 mmol). The reaction mixture was stirred at 20° C. for 12 hours, filtered, and the filtrate purified by reversed-phase flash chromatography (0.1% FA/MeCN condition) to afford 0.34 g of the title compound (56.3) as a solid.

LCMS (m/z [M+H]*): 377.3

### Step 2. (Z)—N-(3-(2-Hydroxypropyl)thiazolidin-2-ylidene)-1-((2-(trimethylsilyl)ethoxy)methyl)-1H-pyrrolo[2,3-b]pyridine-3-carboxamide (56.5)

To a solution of 2-methyloxirane (56.4) (0.100 g 1.79 mmol) in DMF (3 mL) was added cesium carbonate (0.580 g, 1.79 mmol) and (Z)—N-(thiazolidin-2-ylidene)-1-((2-(trimethylsilyl)ethoxy)methyl)-1H-pyrrolo[2,3-b]pyridine-3-carboxamide (51.3) (0.450 g, 1.20 mmol). The reaction mixture was stirred at 20° C. for 36 hours, filtered, and the filtrate purified by reversed-phase flash chromatography (0.1% FA/MeCN condition) to afford 0.13 g of the title compound (56.5) as a solid.

¹H NMR (400 MHz, CDCl₃) δ 8.69 (d, J=7.6 Hz, 1H), 8.49-8.42 (m, 1H), 8.40-8.38 (m, 1H), 7.30-7.23 (m, 1H), 5.79-5.72 (m, 2H), 4.28-4.25 (m, 1H), 3.93-3.89 (m, 3H), 3.80-3.76 (m, 1H), 3.63-3.58 (m, 3H), 3.28 (t, J=8.0 Hz, 2H), 1.33 (d, J=2.4 Hz, 3H), 0.96-0.91 (m, 2H), 0.06 (s, 9H)

### Step 3. (Z)—N-(3-(2-Hydroxypropyl)thiazolidin-2-ylidene)-1H-pyrrolo[2,3-b]pyridine-3-carboxamide (Example 19)

To a solution of (Z)—N-(3-(2-hydroxypropyl)thiazolidin-2-ylidene)-1-((2-(trimethylsilyl) ethoxy)methyl)-1H-pyrrolo[2,3-b]pyridine-3-carboxamide (56.5) (0.120 g, 0.280 mmol) in DCM (1 mL) was added TFA (0.2 mL). The reaction mixture was stirred at 20° C. for 2 hours and then concentrated under reduced pressure. The residue was purified by preparative HPLC (Waters xbridge 25×150 mm, 10 μm; mobile phase A: water/10 mM NH₄HCO₃, mobile phase B: ACN; gradient: 5% B to 35% B over 11 min) to afford 0.036 g of (Z)—N-(3-(2-hydroxypropyl)thiazolidin-2-ylidene)-1H-pyrrolo[2,3-b]pyridine-3-carboxamide (Example 19) as a solid.

¹H NMR (400 MHz, CDCl₃) δ 10.14 (br. s, 1H), 8.74 (dd, J=1.6, 8.0 Hz, 1H), 8.36 (dd, J=1.6, 4.8 Hz, 1H), 8.15 (s, 1H), 7.23 (dd, J=4.8, 8.0 Hz, 1H), 4.26-4.23 (m, 1H), 3.87-3.80 (m, 3H), 3.72 (dd, J=2.4, 14.4 Hz, 1H), 3.31-3.21 (t, J=7.6 Hz, 2H), 1.31 (d, J=6.4 Hz, 3H) LCMS (m/z [M+H]⁺): 305.1

### Preparation of (Z)—N-(hexahydro-3H-thiazolo[3,4-a]pyrazin-3-ylidene)-1H-pyrrolo[2,3-b]pyridine-3-carboxamide (Example 22)

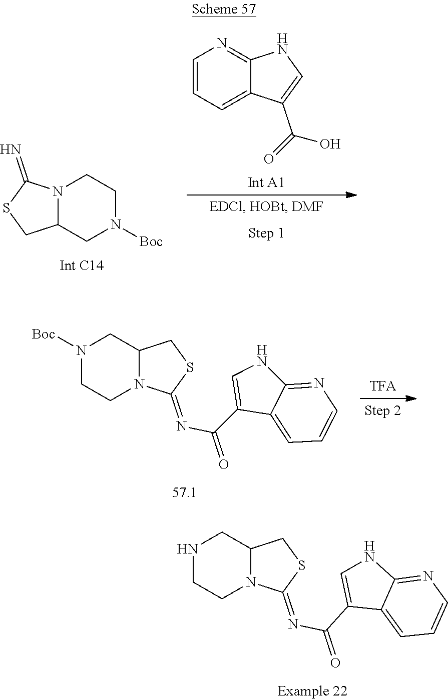 [SMILES: CC(C)(C)OC(=O)N1CCN2C(=N)SCC2C1 ; O=C(O)c1c[nH]c2ncccc12 ; CC(C)(C)OC(=O)N1CCN2/C(=N/C(=O)c3c[nH]c4ncccc34)SCC2C1 ; O=C(/N=C1\SCC2CNCCN12)c1c[nH]c2ncccc12]

### Step 1. tert-Butyl (Z)-3-((1H-pyrrolo[2,3-b]pyridine-3-carbonyl)imino)tetrahydro-3H-thiazolo[3,4-a]pyrazine-7(1H)-carboxylate (57.1)

To a solution of tert-butyl 3-iminotetrahydro-1H-thiazolo[3,4-a]pyrazine-7(3H)-carboxylate (C14) (1.05 g, 4.08 mmol) and 1H-pyrrolo[2,3-b]pyridine-3-carboxylic acid (A1) (0.663 g, 4.08 mmol) in DMF (10 mL) was added DIPEA (2.64 g, 20.4 mmol), EDCI (1.17 g, 6.12 mmol) and HOBt (0.440 g, 3.2 mmol) at 25° C. The reaction mixture was stirred at 25° C. for 12 hours then purified by reversed phase flash chromatography (0.1% NH₃·H₂O/MeCN condition) to afford 0.65 g of the title compound (57.1) as a solid.

¹H NMR (400 MHz, DMSO-d₆) δ 12.25 (br. s, 1H), 8.51 (d, J=6.4 Hz, 1H), 8.27 (d, J=4.8 Hz, 1H), 8.21 (s, 1H), 7.20 (dd, J=4.8, 8.0 Hz, 1H), 4.59 (d, J=11.2 Hz, 1H), 4.28-4.13 (m, 1H), 4.12-4.00 (m, 1H), 3.78-3.68 (m, 1H), 3.27-3.23 (m, 1H), 3.05-3.00 (m, 1H), 2.97-2.73 (m, 3H), 1.44 (s, 9H) LCMS (m/z [M+H]⁺): 402.1

### Step 2. (Z)—N-(Hexahydro-3H-thiazolo[3,4-a]pyrazin-3-ylidene)-1H-pyrrolo[2,3-b]pyridine-3-carboxamide (Example 22)

To a solution of (Z)-tert-butyl 3-((1H-pyrrolo[2,3-b]pyridine-3-carbonyl)imino)tetrahydro-1H-thiazolo[3,4-a]pyrazine-7(3H)-carboxylate (57.1) (0.350 g, 0.871 mmol) in DCM (3 mL) was added TFA (1.54 g, 13.5 mmol) at 25° C. The reaction mixture was stirred at 25° C. for 0.5 hour, neutralized to pH=7 with trimethylamine and the solvent was evaporated. The residue was purified by preparative HPLC (Waters xbridge, 25×150 mm, 10 μm; mobile phase A: water/10 mM NH₄HCO₃, mobile phase B: MeCN]; gradient: 5% B to 35% B over 11 min) to afford 0.185 g of (Z)—N-(hexahydro-3H-thiazolo[3,4-a]pyrazin-3-ylidene)-1H-pyrrolo[2,3-b]pyridine-3-carboxamide (Example 22) as a solid.

¹H NMR (400 MHz, DMSO-d₆) δ 12.23 (br. s, 1H), 8.51 (dd, J=1.6, 7.6 Hz, 1H), 8.26 (dd, J=1.6, 4.4 Hz, 1H), 8.17 (d, J=2.4 Hz, 1H), 7.24-7.16 (m, 1H), 4.49 (dd, J=2.4, 12.0 Hz, 1H), 3.68-3.57 (m, 1H), 3.30-3.27 (m, 1H), 3.17 (dd, J=3.2, 12.4 Hz, 1H), 3.04-2.94 (m, 2H), 2.77 (t, J=10.8 Hz, 1H), 2.59-2.52 (m, 1H), 2.46-2.43 (m, 1H)

LCMS (m/z [M+H]⁺): 302.1

### Preparation of (Z)-2-((1H-pyrrolo[2, 3-b]pyridine-3-carbonyl)imino)-N-methyl-3-phenyl-1,3-thiazinane-5-carboxamide (Example 34)

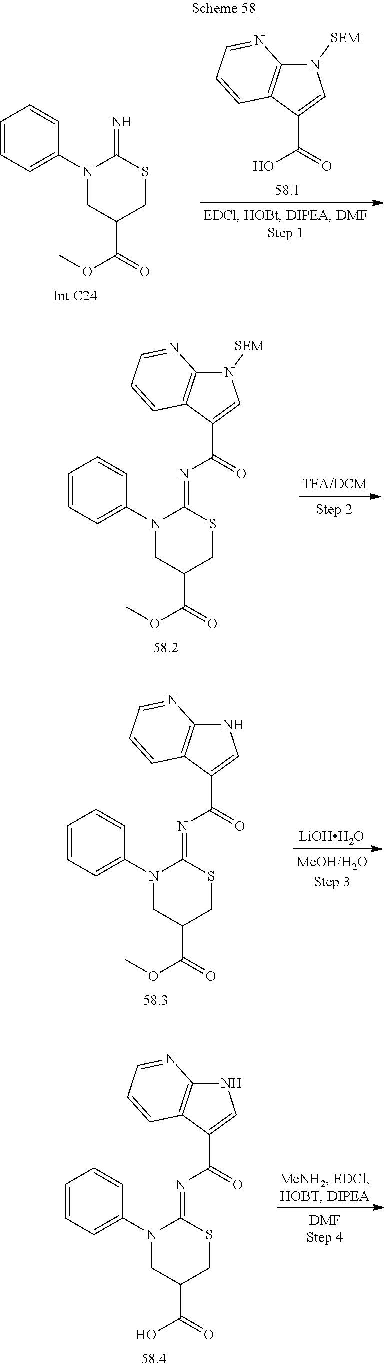

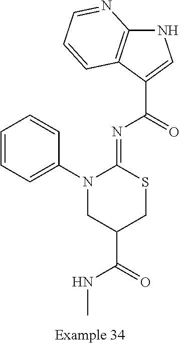 [SMILES: CNC(=O)C1CS/C(=N\C(=O)c2c[nH]c3ncccc23)N(c2ccccc2)C1]

### Step 1. Methyl (Z)-3-phenyl-2-((1-((2-(trimethylsilyl)ethoxy)methyl)-1H-pyrrolo[2,3-b] pyridine-3-carbonyl)imino)-1, 3-thiazinane-5-carboxylate (58.2)

To a mixture of methyl 2-imino-3-phenyl-1, 3-thiazinane-5-carboxylate (C24) (1.24 g, 4.32 mmol) in DMF (10 mL) was added 1-((2-(trimethylsilyl)ethoxy)methyl)-1H-pyrrolo[2,3-b]pyridine-3-carboxylic acid (58.1) (1.26 g, 4.32 mmol), EDCI (1.66 g, 8.65 mmol), HOBt (1.17 g, 8.65 mmol) and DIPEA (2.26 mL, 12.9 mmol) at 20° C. The reaction mixture was stirred at 20° C. for 2 hours, then concentrated under reduced pressure. The residue was purified by reversed phase flash chromatography (0.1% FA/MeCN condition) to afford 1.50 g of the title compound (58.2) as a solid.

¹H NMR (400 MHz, DMSO-d₆) δ 8.20-8.15 (m, 1H), 7.91-7.87 (m, 1H), 7.59-7.53 (m, 2H), 7.50-7.45 (m, 1H), 7.44-7.39 (m, 2H), 7.25 (d, J=7.6 Hz, 1H), 6.86-6.81 (m, 1H), 5.57 (s, 2H), 4.04-3.95 (m, 2H), 3.72 (s, 3H), 3.54-3.52 (m, 2H), 3.42-3.37 (m, 1H), 3.30-3.25 (m, 1H), 3.19-3.14 (m, 1H), 0.81-0.75 (m, 2H), −0.13 (s, 9H)

### Step 2. Methyl (Z)-2-((1H-pyrrolo[2,3-b]pyridine-3-carbonyl)imino)-3-phenyl-1, 3-thiazin ane-5-carboxylate (58.3)

To a mixture of methyl (Z)-3-phenyl-2-((1-((2-(trimethylsilyl)ethoxy)methyl)-1H-pyrrolo[2,3-b]pyridine-3-carbonyl)imino)-1,3-thiazinane-5-carboxylate (58.2) (1.50 g, 2.86 mmol) in DCM (10 mL) was added TFA (5.00 mL, 67.5 mmol) at 20° C. The reaction mixture was stirred at 20° C. for 1 hour then concentrated under reduced pressure. The residue was purified by reversed phase flash chromatography (0.1% FA/MeCN condition) to afford 0.80 g of the title compound (58.3) as a solid.

¹H NMR (400 MHz, DMSO-d₆) δ 12.02 (br. s, 1H), 8.12 (s, 1H), 7.64-7.63 (m, 1H), 7.53-7.52 (m, 2H), 7.45-7.41 (m, 4H), 6.82-6.80 (m, 1H), 4.04-3.95 (m, 2H), 3.71 (s, 3H), 3.59-3.58 (m, 1H), 3.39-3.38 (m, 1H), 3.37-3.36 (m, 1H)

### Step 3. (Z)-2-((1H-Pyrrolo[2,3-b]pyridine-3-carbonyl)imino)-3-phenyl-1,3-thiazinane-5-carboxylic acid (58.4)

To a mixture of methyl (Z)-2-((1H-pyrrolo[2,3-b]pyridine-3-carbonyl)imino)-3-phenyl-1, 3-thiazinane-5-carboxylate (58.3) (0.150 g, 0.380 mmol) in MeOH (4 mL) was added lithium hydroxide monohydrate (0.319 g, 0.761 mmol) and water (1 mL) at 20° C. The mixture was stirred at 20° C. for 2 hours then concentrated under reduced pressure. The residue was adjusted to pH=6 with 1 M hydrochloric acid. The resulting precipitate was collected by filtration to afford 0.102 g of the title compound (58.4) as a solid.

¹H NMR (400 MHz, DMSO-d₆) δ 13.03 (br. s, 1H), 12.05 (br. s, 1H), 8.13 (s, 1H), 7.63 (s, 1H), 7.53-7.52 (m, 2H), 7.45-7.43 (m, 1H), 7.43-7.41 (m, 3H), 6.82-6.80 (m, 1H), 3.98-3.97 (m, 2H), 3.48-3.47 (m, 1H), 3.29-3.26 (m, 1H), 3.26-3.25 (m, 1H)

### Step 4. (Z)-2-((1H-Pyrrolo[2, 3-b]pyridine-3-carbonyl)imino)-N-methyl-3-phenyl-1,3-thiazinane-5-carboxamide (Example 34)

To a mixture of (Z)-2-((1H-pyrrolo[2,3-b]pyridine-3-carbonyl)imino)-3-phenyl-1, 3-thiazinane-5-carboxylic acid (58.4) (0.102 g, 0.268 mmol) in DMF (2 mL) was added methylamine hydrochloride (0.018 g, 0.268 mmol), HOBt (0.073 g, 0.536 mmol), EDCI (0.102 g, 0.536 mmol) and DIPEA (0.140 mL, 0.804 mmol) at 20° C. The reaction mixture was stirred at 20° C. for 2 hours then purified by preparative HPLC (Waters xbridge 25×150 mm, 10 μm; mobile phase A: water/NH₄HCO₃, mobile phase B: ACN; gradient: 12% B to 42% B over 11 min) to afford 0.038 g of (Z)-2-((1H-pyrrolo[2, 3-b]pyridine-3-carbonyl)imino)-N-methyl-3-phenyl-1,3-thiazinane-5-carboxamide (Example 34) as a solid.

¹H NMR (DMSO-d₆, 400 MHz) δ 12.01 (br. s, 1H), 8.12 (dd, J=1.2, 4.4 Hz, 2H), 7.65 (d, J=1.6 Hz, 1H), 7.56-7.52 (m, 2H), 7.46-7.39 (m, 4H), 6.81 (dd, J=4.8, 8.0 Hz, 1H), 3.99-3.91 (m, 1H), 3.89-3.84 (m, 1H), 3.31-3.21 (m, 2H), 3.17-3.15 (m, 1H), 2.62 (d, J=4.8 Hz, 3H). LCMS (m/z [M+H]⁺): 394.3

### Preparation of (Z)—N-(5-(hydroxymethyl)-3-phenyl-1,3-thiazinan-2-ylidene)-1H-pyrrolo[2,3-b]pyridine-3-carboxamide (Example 35)

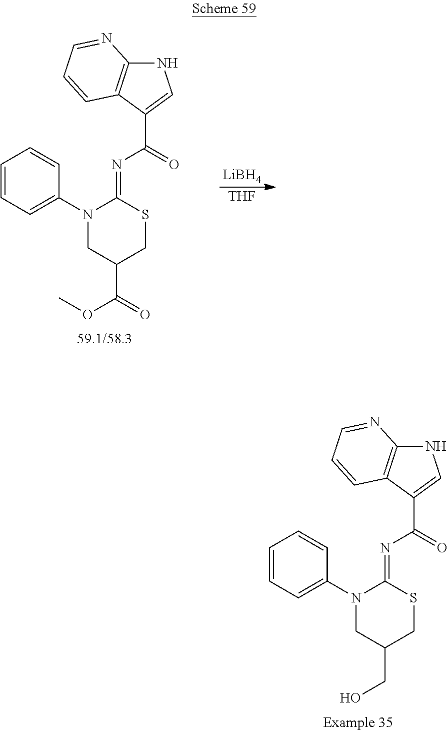 [SMILES: COC(=O)C1CS/C(=N\C(=O)c2c[nH]c3ncccc23)N(c2ccccc2)C1 ; "LiBH4" ; O=C(/N=C1\SCC(CO)CN1c1ccccc1)c1c[nH]c2ncccc12 ; "THF"]

To a mixture of methyl (Z)-2-((1H-pyrrolo[2,3-b]pyridine-3-carbonyl)imino)-3-phenyl-1,3-thiazinane-5-carboxylate (59.1/58.3) (0.200 g, 0.507 mmol) in THF (5 mL) was added lithium borohydride (0.110 g, 5.07 mmol) at −20° C. under nitrogen atmosphere. The mixture was stirred at −20° C. for 0.5 hour under nitrogen atmosphere. The reaction mixture was quenched by addition of saturated aqueous solution of ammonium chloride (40 mL), extracted with ethyl acetate (3×40 mL). The combined organic layers were dried over anhydrous Na₂SO₄, filtered and the filtrate was concentrated under reduced pressure. The residue was purified by preparative TLC (petroleum ether:ethyl acetate=1:4), followed by preparative HPLC (Phenomenex Luna Cis 30×100 mm, 5 μm; mobile phase A:water/FA, mobile phase B: ACN; gradient:10% B to 40% B over 8 min) to afford 0.022 g of (Z)—N-(5-(hydroxymethyl)-3-phenyl-1,3-thiazinan-2-ylidene)-1H-pyrrolo[2,3-b]pyridine-3-carboxamide (Example 35) as a white solid.

¹H NMR (400 MHz, DMSO-d₆) δ11.99 (br. s, 1H), 8.11 (d, J=4.8 Hz, 1H), 7.64 (s, 1H), 7.55-7.52 (m, 2H), 7.45-7.39 (m, 4H), 6.81 (dd, J=4.6 Hz, 8.0 Hz, 1H), 4.91 (br. t, J=5.2 Hz, 1H), 3.82 (dd, J=4.0 Hz, 13.2 Hz, 1H), 3.64 (dd, J=8.8 Hz, 12.8 Hz, 1H), 3.53-3.48 (m, 2H), 3.29 (s, 1H), 3.07 (dd, J=4.0 Hz, 12.4 Hz, 1H), 2.93 (dd, J=9.2 Hz, 12.0 Hz, 1H) LCMS (m/z [M+H]⁺): 367.1

### Preparation of (Z)—N-(3-(2-(Dimethylamino)-2-oxoethyl)-1,3-thiazinan-2-ylidene)-1H-pyrrolo[2,3-b]pyridine-3-carboxamide (Example 37)

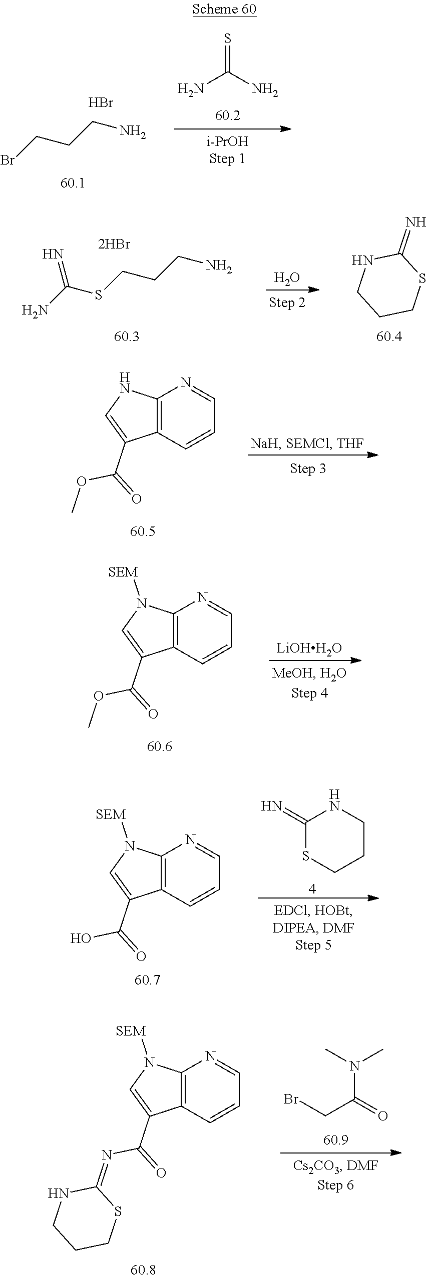

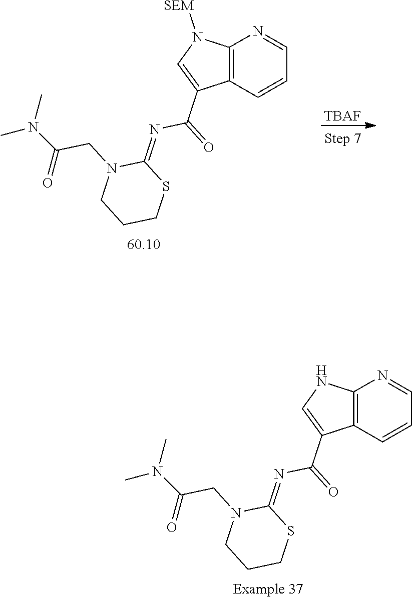

### Step 1. 3-Aminopropylcarbamimidothioate (60.3)

A mixture of 3-bromopropan-1-amine hydrobromic salt (60.1) (5.00 g, 22.8 mmol) and thiourea (60.2) (1.74 g, 22.8 mmol) in isopropanol (20 mL) was stirred at 80° C. for 3 hours. The cooled mixture was filtered, and the filter cake washed with isopropanol (30 mL). The filter cake was dried under reduced pressure to afford 4.00 g of the title compound (60.3) as the di-hydrobromide salt as a solid.

¹H NMR (400 MHz, DMSO-d₆) δ 9.08 (br. s, 4H), 7.94 (br. s, 3H), 3.28 (t, J=7.2 Hz, 2H), 2.88 (t, J=7.2 Hz, 2H), 1.98-1.86 (m, 2H)

### Step 2. 1,3-thiazinan-2-imine (60.4)

A solution of 3-aminopropylcarbamimidothioate di-hydrobromide (60.3) (4.00 g, 13.6 mmol) in water (40 mL) was stirred at 100° C. for 12 hours. The mixture was concentrated under reduced pressure and the residue was diluted with isopropanol (150 mL) and filtered. The filtrate was concentrated under reduced pressure to 80 mL. The resulting precipitate was collected by filtration and the filter cake dried under reduced pressure to afford 1.20 g of 1,3-thiazinan-2-imine (60.4) hydrobromde salt as a solid.

¹H NMR (400 MHz, DMSO-d₆) δ 9.63 (br. s, 1H), 8.71 (br. s, 2H), 3.39 (t, J=5.6 Hz, 2H), 3.19 (t, J=5.6 Hz, 2H), 2.03-1.98 (m, 2H)

### Step 3. Methyl 1-((2-(trimethylsilyl)ethoxy)methyl)-1H-pyrrolo [2,3-b] pyridine-3-carboxylate (60.6)

To a solution of methyl 1H-pyrrolo[2,3-b]pyridine-3-carboxylate (60.5) (2.00 g, 11.3 mmol) in THF (30 mL) was added NaH (0.544 g, 13.6 mmol, 60% in mineral oil) at 0° C. The mixture was stirred at 20° C. for 30 minutes, and then SEM-Cl (2.84 g, 17.0 mmol) was added to the mixture at 0° C. The resulting mixture was stirred at 20° C. for 12 hours. The reaction was quenched with a saturated aqueous solution of ammonium chloride (100 mL) and extracted with EtOAc (3×50 mL). The combined organic layers were dried over anhydrous Na₂SO₄, filtered, and concentrated. The residue was purified by silica gel column chromatography, eluting with 2 to 10% EtOAc in petroleum ether to afford 3.20 g of the title compound (60.6) as an oil.

¹H NMR (400 MHz, CDCl₃) δ 8.45 (dd, J=1.6, 8.0 Hz, 1H), 8.41 (dd, J=1.6 Hz, 4.8 Hz, 1H), 8.07 (s, 1H), 7.29-7.24 (m, 1H), 5.72 (s, 2H), 3.94 (s, 3H), 3.58 (t, J=8.0 Hz, 2H), 0.92 (t, J=8.0 Hz, 2H), 0.06 (s, 9H).

### Step 4. 1-((2-(Trimethylsilyl)ethoxy)methyl)-1H-pyrrolo[2,3-b]pyridine-3-carboxylic acid (60.7)

A mixture of methyl 1-((2-(trimethylsilyl)ethoxy)methyl)-1H-pyrrolo[2,3-b]pyridine-3-carboxylate (60.6) (3.20 g, 10.4 mmol) and NaH (2.09 g, 52.2 mmol) in MeOH (15 mL) was stirred at 20° C. for 12 hours and then concentrated under reduced pressure. The residue was diluted with water (100 mL) and adjusted pH to 5 with 1M HCl. The precipitate formed was collected by filtration and dried under reduced pressure to afford 3.00 g of the crude title compound (60.7) as a solid.

LCMS (m/z [M+H]⁺): 293.0

### Step 5. (Z)—N-(1,3-Thiazina-2-ylidene)-1-((2-(trimethylsilyl)ethoxy)methyl)-1H-pyrrolo[2,3-b]pyridine-3-carboxamide (60.8)

A mixture of 1-((2-(trimethylsilyl)ethoxy)methyl)-1H-pyrrolo[2,3-b]pyridine-3-carboxylic acid (60.7) (0.050 g, 0.17 mmol), 1,3-thiazinan-2-imine (60.4) hydrobromide (0.034 g, 0.17 mmol), EDCI (0.049 g, 0.26 mmol), HOBt (0.035 g, 0.26 mmol) and DIPEA (0.044 g, 0.34 mmol) in DMF (2 mL) was stirred at 20° C. for 12 hours. The mixture was concentrated under reduced pressure and the residue purified by preparative HPLC (Phenomenex luna Cis 25×150 mm, 10 μm; mobile phase A: water/0.225% FA, mobile phase B: ACN/0.225% FA; gradient: 22% B to 52% B over 10 min) to afford 0.020 g of the title compound (60.8) as a solid.

¹H NMR (400 MHz, DMSO-d₆) δ 8.56 (dd, J=1.6 Hz, 8.0 Hz, 1H), 8.31 (dd, J=1.6 Hz, 4.8 Hz, 1H), 8.18 (s, 1H), 7.25 (dd, J=4.6 Hz, 8.0 Hz, 1H), 5.66 (s, 2H), 3.53 (t, J=8.0 Hz, 2H), 3.49-3.44 (m, 2H), 3.13-3.02 (m, 2H), 2.12-1.95 (m, 2H), 0.82 (t, J=8.0 Hz, 2H), −0.11 (s, 9H)

### Step 6. (Z)—N-(3-(2-(Dimethylamino)-2-oxoethyl)-1,3-thiazinan-2-ylidene)-1-((2-(trimethylsilyl)ethoxy)methyl)-1H-pyrrolo[2,3-b]pyridine-3-carboxamide (60.10)

A mixture of (Z)—N-(1,3-thiazinan-2-ylidene)-1-((2-(trimethylsilyl)ethoxy)methyl)-1H-pyrrolo[2,3-b]pyridine-3-carboxamide (60.8) (0.050 g, 0.13 mmol), cesium carbonate (0.083 g, 0.26 mmol) and 2-bromo-N,N-dimethyl-acetamide (60.9) (0.043 g, 0.26 mmol) in DMF (2 mL) was stirred at 20° C. for 2 hours. The mixture was filtered and the filtrate concentrated under reduced pressure. The residue was purified by reversed-phase flash chromatography (0.1% FA/MeCN condition) to afford 0.035 g of the title compound (60.10) as a oil.

¹H NMR (400 MHz, DMSO-d₆) δ 8.35 (dd, J=1.6 Hz, 7.6 Hz, 1H), 8.31 (dd, J=1.6 Hz, 7.6 Hz, 1H), 8.06 (s, 1H), 7.23 (dd, J=4.8 Hz, 8.0 Hz, 1H), 5.66 (s, 2H), 4.66 (s, 2H), 3.57-3.49 (m, 4H), 3.06 (s, 3H), 3.02-2.94 (m, 2H), 2.91 (s, 3H), 2.22-2.12 (m, 2H), 0.85-0.78 (m, 2H), 0.11 (s, 9H) LCMS (m/z [M+H]⁺): 476.3

### Step 7. (Z)—N-(3-(2-(Dimethylamino)-2-oxoethyl)-1,3-thiazinan-2-ylidene)-1H-pyrrolo[2,3-b]pyridine-3-carboxamide (Example 37)

A solution of (Z)—N-(3-(2-(dimethylamino)-2-oxoethyl)-1,3-thiazinan-2-ylidene)-1-((2-(trimethylsilyl)ethoxy)methyl)-1H-pyrrolo[2,3-b]pyridine-3-carboxamide (60.10) (0.120 g, 0.252 mmol) in tetrabutylammonium fluoride (2 mL, 1 M in tetrahydrofuran) was stirred at 25° C. for 12 hours. The mixture was concentrated under reduced pressure and the residue purified by prep-HPLC (column: Waters xbridge, 25×150 mm, 10 μm; mobile phase A: water/10 mM NH₄HCO₃, mobile phase B: MeCN; gradient: 0% B to 30% B over 11 min) to afford 0.024 g of (Z)—N-(3-(2-(dimethylamino)-2-oxoethyl)-1,3-thiazinan-2-ylidene)-1H-pyrrolo[2,3-b]pyridine-3-carboxamide (Example 37) as a solid.

¹H NMR (400 MHz, DMSO-d₆) δ 12.07 (br. s, 1H), 8.37-8.28 (m, 1H), 8.27-8.17 (m, 1H), 7.89 (s, 1H), 7.23-7.00 (m, 1H), 4.63 (s, 2H), 3.52 (t, J=6.0 Hz, 2H), 3.01 (s, 3H), 2.96 (t, J=5.6 Hz, 2H), 2.89 (s, 3H), 2.19-2.08 (m, 2H) LCMS (m/z [M+H]⁺): 346.1

### Preparation of (Z)—N-(3-benzyl-4-(hydroxymethyl)thiazol-2(3H)-ylidene)-1H-pyrrolo[2,3-b]pyridine-4-carboxamide (Example 54)

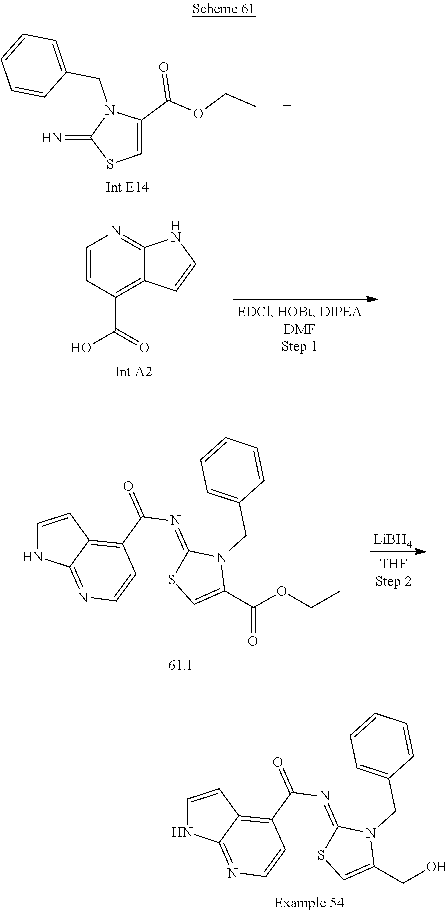 [SMILES: CCOC(=O)c1csc(=N)n1Cc1ccccc1 ; O=C(O)c1ccnc2[nH]ccc12 ; CCOC(=O)c1cs/c(=N\C(=O)c2ccnc3[nH]ccc23)n1Cc1ccccc1 ; "LiBH4" ; O=C(/N=c1\scc(CO)n1Cc1ccccc1)c1ccnc2[nH]ccc12]

### Step 1. Ethyl (Z)-2-((1H-pyrrolo[2,3-b]pyridine-4-carbonyl)imino)-3-benzyl-2,3-dihydrothiazole-4-carboxylate (61.1)

To a solution of 1H-pyrrolo[2,3-b]pyridine-4-carboxylic acid (A2) (0.163 g, 1.01 mmol) in DMF (100 mL) were added HOBt (0.099 g, 0.732 mmol) and DIPEA (0.478 mL, 2.74 mmol), EDCI (0.263 g, 1.37 mmol) and ethyl 3-benzyl-2-imino-2, 3-dihydrothiazole-4-carboxylate (E14) (0.240 g, 0.915 mmol) at 25° C. The resulting mixture was stirred at 25° C. for 2 hours, and then poured into ice-water (200 mL). The resulting precipitate was collected by filtration and triturated with MeOH (6 mL) to afford 0.10 g of the title compound (61.1) as a solid, which was used without further purification.

### Step 2. (Z)—N-(3-Benzyl-4-(hydroxymethyl)thiazol-2(3H)-ylidene)-1H-pyrrolo[2,3-b]pyridine-4-carboxamide (Example 54)

To a suspension of (Z)-ethyl 2-((1H-pyrrolo[2,3-b]pyridine-4-carbonyl)imino)-3-benzyl-2, 3-dihydrothiazole-4-carboxylate (61.1) (0.070 g, 0.17 mmol) in THF (5 mL) at 0° C. under nitrogen was added lithium borohydride (1.8 mL, 9.3 mmol, 2 M in THF) dropwise. The resulting mixture was stirred at 40° C. for 4 hours, and then poured into a saturated aqueous solution of ammonium chloride (50 mL). The aqueous phase was extracted with EtOAc (3×30 mL) and the combined organic phases were washed with brine (2×50 mL) then dried over anhydrous Na₂SO₄, filtered, and concentrated. The residue was dissolved in MeOH (5.0 mL) and stirred at 75° C. for 12 hours. The solvent was evaporated and the residue purified by preparative HPLC (Waters Xbridge 25×150 mm, 5 μm; mobile phase A: water/10 mM NH₄HCO₃, mobile phase B: ACN; gradient: 19% B to 49% B over min) to afford 0.315 g of (Z)—N-(3-benzyl-4-(hydroxymethyl)thiazol-2(3H)-ylidene)-1H-pyrrolo[2,3-b]pyridine-4-carboxamide (Example 54) as a solid.

¹H NMR (400 MHz, DMSO-d₆) δ 611.76 (br. s, 1H), 8.29 (d, J=4.8 Hz, 1H), 7.76 (d, J=5.2 Hz, 1H), 7.52 (d, J=3.2 Hz, 1H), 7.38-7.34 (m, 2H), 7.30-7.26 (m, 3H), 7.03-7.02 (m, 2H), 5.71 (s, 3H), 4.44 (s, 2H) LCMS (m/z [M+H]⁺): 365.1

### Preparation of (Z)-2-((1H-pyrrolo[2,3-b]pyridine-4-carbonyl)imino)-3-benzyl-N-(2-(dimethylamino)ethyl)-2,3-dihydrothiazole-4-carboxamide (Example 57)

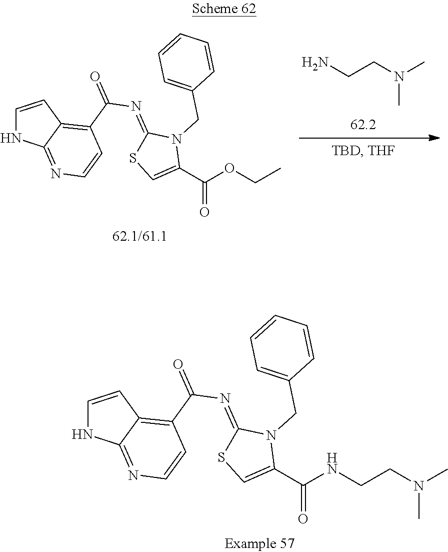

To a solution of (Z)-ethyl 2-((1H-pyrrolo[2,3-b]pyridine-4-carbonyl)imino)-3-benzyl-2,3-dihydrothiazole-4-carboxylate (62.1/61.1) (0.070 g, 0.172 mmol) and N,N-dimethylethylenediamine (62.2) (0.152 g, 1.72 mmol) in THF (2 mL) was added TBD (0.012 g, 0.086 mmol) and the mixture stirred at 60° C. for 1.5 hours. The cooled reaction mixture was concentrated, and the residue was triturated with MeOH (3.0 mL) at 25° C. to afford 0.027 g of (Z)-2-((1H-pyrrolo[2,3-b]pyridine-4-carbonyl)imino)-3-benzyl-N-(2-(dimethylamino)ethyl)-2,3-dihydrothiazole-4-carboxamide (Example 57) as a solid.

¹H NMR (400 MHz, DMSO-d₆) δ 11.82 (br. s, 1H), 8.81 (t, J=6.0 Hz, 1H), 8.33 (d, J=4.8 Hz, 1H), 7.84 (d, J=5.2 Hz, 1H), 7.57 (t, J=2.8 Hz, 1H), 7.51 (s, 1H), 7.31-7.25 (m, 5H), 7.10-7.08 (m, 1H), 5.94 (s, 2H), 3.27-3.24 (m, 2H), 2.28 (t, J=6.8 Hz, 2H), 2.13 (s, 6H) LCMS (m/z [M+H]⁺): 449.0

### Preparation of (Z)—N-(3-(2-chlorophenyl)-4-((dimethylamino)methyl)thiazol-2(3H)-ylidene)-1H-pyrrolo[2,3-b]pyridine-4-carboxamide (Example 59)

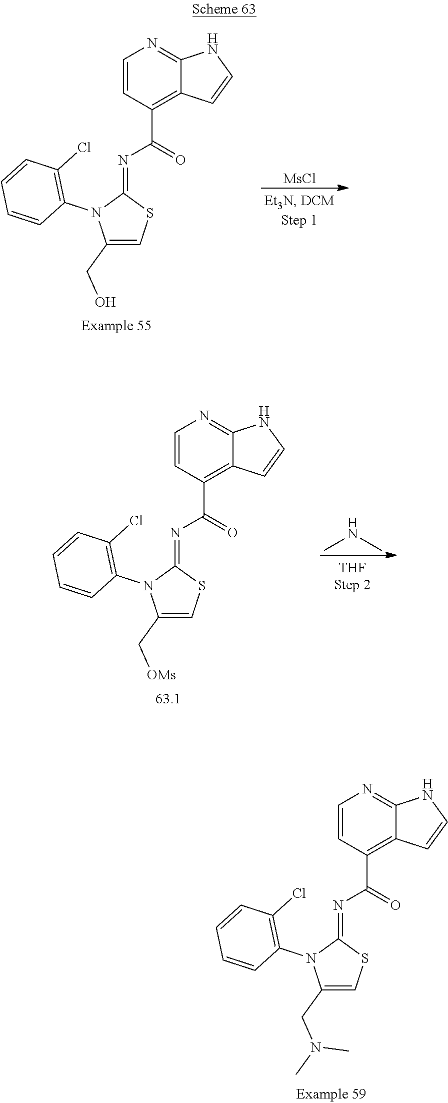

### Step 1. (Z)-(2-((1H-Pyrrolo[2,3-b]pyridine-4-carbonyl)imino)-3-(2-chlorophenyl)-2,3-dihydrothiazol-4-yl)methyl methanesulfonate (63.1)

To a mixture of (Z)—N-(3-(2-chlorophenyl)-4-(hydroxymethyl)thiazol-2(3H)-ylidene)-1H-pyrrolo[2,3-b]pyridine-4-carboxamide (Example 55) (0.030 g, 0.077 mmol), Et₃N (0.023 g, 0.23 mmol) in DCM (1 mL) at 0° C. under nitrogen was added MsCl (0.014 g, 0.12 mmol) in one portion. The reaction mixture was stirred at 25° C. for 1 hour then poured into ice-water (w/w=1/1) (10 mL) and stirred for 1 minute. The aqueous phase was extracted with DCM (3×10 mL). The combined organic phases were washed with brine (10 mL), dried over anhydrous Na₂SO₄, filtered, and concentrated to afford 40 mg of the crude title compound (63.1) as a solid. LCMS (m/z [M+H]⁺): 462.9

### Step 2. (Z)—N-(3-(2-Chlorophenyl)-4-((dimethylamino)methyl)thiazol-2(3H)-ylidene)-1H-pyrrolo[2,3-b]pyridine-4-carboxamide (Example 59)

A mixture of (Z)-(2-((1H-pyrrolo[2,3-b]pyridine-4-carbonyl)imino)-3-(2-chlorophenyl)-2, 3-dihydrothiazol-4-yl)methyl methanesulfonate (63.1) (0.040 g, 0.086 mmol) and dimethylamine (2 M, 0.216 mL, 0.430 mL) was stirred at 50° C. for 2 hours. The mixture was poured into ice-water (w/w=1/1) (10 mL) and stirred for 1 minute. The aqueous phase was extracted with EtOAc (2×20 mL). The combined organic phases were washed with brine (2×10 mL), dried over anhydrous Na₂SO₄, filtered, and concentrated. The residue was purified by reversed phase HPLC (0.1% NH₃·H₂O/MeCN condition) to afford 0.009 g of (Z)—N-(3-(2-chlorophenyl)-4-((dimethylamino)methyl)thiazol-2(3H)-ylidene)-1H-pyrrolo[2,3-b]pyridine-4-carboxamide (Example 59) as a solid.

¹H NMR (400 MHz, CD₃OD) δ 8.17 (d, J=5.2 Hz, 1H), 7.75-7.74 (m, 1H), 7.67-7.60 (m, 4H), 7.23 (d, J=3.2 Hz, 1H), 7.04 (s, 1H), 6.47 (d, J=3.6 Hz, 1H), 3.26 (d, J=6.4 Hz, 2H), 2.15 (s, 6H) LCMS (m/z [M+H]⁺): 412.1

### Preparation of (Z)—N-(4-(acetamidomethyl)-3-(2-chlorophenyl)thiazol-2(3H)-ylidene)-1H-pyrrolo[2,3-b]pyridine-4-carboxamide (Example 60)

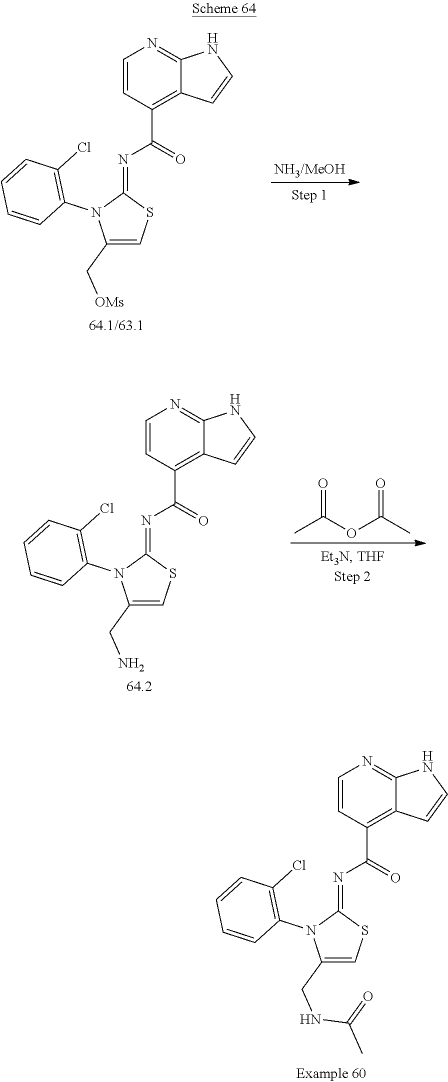

### Step 1. (Z)—N-(4-(Aminomethyl)-3-(2-chlorophenyl)thiazol-2(3H)-ylidene)-1H-pyrrolo[2,3-b]pyridine-4-carboxamide (64.2)

A mixture of (Z)-(2-((1H-pyrrolo[2,3-b]pyridine-4-carbonyl)imino)-3-(2-chlorophenyl)-2,3-dihydrothiazol-4-yl)methyl methanesulfonate (64.1/63.1) (0.120 g, 0.259 mmol) and ammonia (7 M solution in MeOH, 1.00 mL, 7.00 mmol) was stirred for 16 hours at 75° C. The solvent was evaporated and the residue purified by reversed flash chromatography (0.1% NH₃·H₂O/MeCN condition) to afford 0.070 g of the title compound (64.2) as a solid.

¹H NMR (400 MHz, DMSO-d₆) δ 11.68 (br. s, 1H), 8.21 (d, J=4.8 Hz, 1H), 7.84 (dd, J=1.6 Hz, 8.0 Hz, 1H), 7.79-7.76 (m, 1H), 7.72-7.67 (m, 2H), 7.47 (d, J=5.2 Hz, 1H), 7.33-7.32 (m, 1H), 7.09 (s, 1H), 6.36-6.33 (m, 1H), 3.49-3.46 (m, 2H) LCMS (m/z [M+H]⁺): 384.2

### Step 2. (Z)—N-(4-(Acetamidomethyl)-3-(2-chlorophenyl)thiazol-2(3H)-ylidene)-1H-pyrrolo[2,3-b]pyridine-4-carboxamide (Example 60)

To a mixture of (Z)—N-(4-(aminomethyl)-3-(2-chlorophenyl)thiazol-2(3H)-ylidene)-1H-pyrrolo[2,3-b]pyridine-4-carboxamide (64.2) (0.060 g, 0.16 mmol) and acetic anhydride (0.047 g, 0.47 mmol) in THF (1 mL) was added Et₃N (0.047 g, 0.47 mmol). The reaction mixture was stirred at 25° C. for 2 hours under nitrogen. The mixture was filtered, the filtrate concentrated, and the residue purified by preparative HPLC (Phenomenex Gemini-NX Cis 30×75 mm, 3 μm; mobile phase A: water/10 mM NH₄HCO₃, mobile phase B: ACN; gradient: 12% B to 42% B over 8 min) to afford 0.022 g of (Z)—N-(4-(acetamidomethyl)-3-(2-chlorophenyl)thiazol-2(3H)-ylidene)-1H-pyrrolo[2,3-b]pyridine-4-carboxamide (Example 60) as a solid.

¹H NMR (400 MHz, DMSO-d₆) δ 11.67 (br. s, 1H), 8.26-8.21 (m, 2H), 7.81 (dd, J=1.6 Hz, 8.0 Hz, 1H), 7.74-7.62 (m, 3H), 7.48 (d, J=4.8 Hz, 1H), 7.32 (d, J=3.6 Hz, 1H), 7.06 (s, 1H), 6.35 (d, J=3.6 Hz, 1H), 4.03-3.98 (m, 2H), 1.77 (s, 3H) LCMS (m/z [M+H]⁺): 426.1

### Preparation of (Z)—N-(3-benzyl-5-(hydroxymethyl)thiazol-2(3H)-ylidene)-1H-pyrrolo[2,3-b]pyridine-4-carboxamide (Example 62)

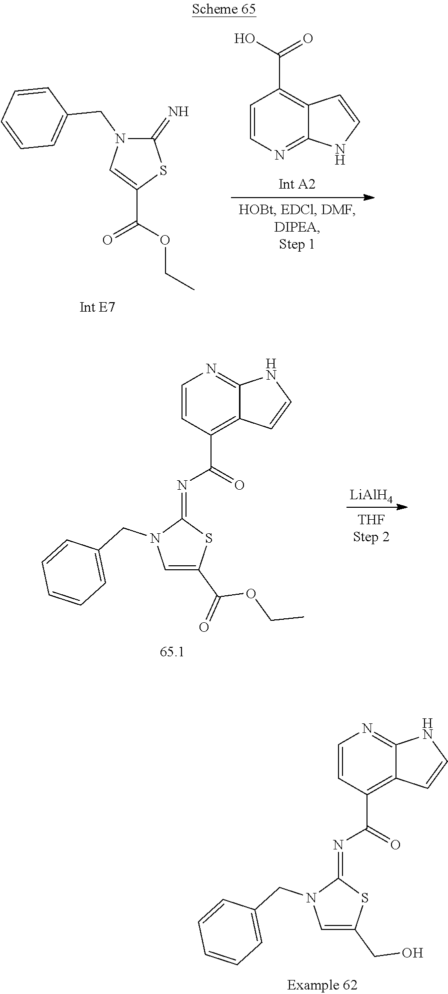 [SMILES: CCOC(=O)c1cn(Cc2ccccc2)c(=N)s1 ; O=C(O)c1ccnc2[nH]ccc12 ; CCOC(=O)c1cn(Cc2ccccc2)/c(=N/C(=O)c2ccnc3[nH]ccc23)s1 ; "LiAlH4" ; O=C(/N=c1\sc(CO)cn1Cc1ccccc1)c1ccnc2[nH]ccc12]

### Step 1. Ethyl (Z)-2-((1H-pyrrolo[2,3-b]pyridine-4-carbonyl)imino)-3-benzyl-2,3-dihydrothiazole-5-carboxylate (65.1)

To a solution of 1H-pyrrolo[2,3-b]pyridine-4-carboxylic acid (A2) (0.120 g, 0.741 mmol) in DMF (3 mL) at 0° C. was added EDCI (0.426 g, 2.22 mmol), HOBt (0.080 g 0.59 mmol), DIPEA (0.194 mL, 1.11 mmol), and ethyl 3-benzyl-2-imino-2,3-dihydrothiazole-5-carboxylate hydrobromide (E7) (0.200 g, 0.741 mmol). The mixture was stirred at 25° C. for 1 hour then poured into ice-water (20 mL). The resulting precipitate was collected by filtration and the filter cake was triturated with ethanol (6 mL) to afford 0.20 g of the title compound (65.1) as a solid.

¹H NMR (400 MHz, DMSO-d₆) δ 11.88 (br. s, 1H), 8.69 (s, 1H), 8.37 (d, J=4.8 Hz, 1H), 7.88 (d, J=4.8 Hz, 1H), 7.62 (t, J=2.8 Hz, 1H), 7.50-7.45 (m, 2H), 7.40-7.34 (m, 2H), 7.33-7.28 (m, 1H), 7.10 (dd, J=2.0, 2.8 Hz, 1H), 5.63 (s, 2H), 4.32 (q, J=7.2 Hz, 2H), 1.32 (t, J=7.2 Hz, 3H) LCMS (m/z [M+H]⁺): 407.0

### Step 2. (Z)—N-(3-Benzyl-5-(hydroxymethyl)thiazol-2(3H)-ylidene)-1H-pyrrolo[2,3-b]pyridine-4-carboxamide (Example 62)

To a suspension of (Z)-ethyl 2-((1H-pyrrolo[2,3-b]pyridine-4-carbonyl)imino)-3-benzyl-2, 3-dihydrothiazole-5-carboxylate (65.1) (0.200 g, 0.488 mmol) in THF (3 mL) at 0° C. under nitrogen was added lithium borohydride (0.540 mL, 1.08 mmol, 2 M in THF) dropwise. The mixture was stirred at 25° C. for 12 hours then quenched with an ice-saturated aqueous solution of ammonium chloride (50 mL) and extracted with EtOAc (3×50 mL). The combined organic phases were washed with brine (3×50 mL), dried over anhydrous Na₂SO₄, filtered, and concentrated. The residue was dissolved in a solution of HCl in MeOH (4 M, 3 mL, 12 mmol) and stirred at 25° C. for 1 hour. The reaction mixture was adjusted to pH˜8 with a saturated aqueous solution of sodium bicarbonate. The resulting precipitate was collected by filtration and the filter cake was triturated with MeOH (3 mL) to afford 0.034 g of (Z)—N-(3-Benzyl-5-(hydroxymethyl)thiazol-2(3H)-ylidene)-1H-pyrrolo[2,3-b]pyridine-4-carboxamide (Example 62) as a solid.

¹H NMR (400 MHz, DMSO-d₆) δ 11.78 (br. s, 1H), 8.36-8.30 (m, 1H), 7.88-7.82 (m, 1H), 7.61-7.53 (m, 2H), 7.46-7.41 (m, 2H), 7.40-7.27 (m, 3H), 7.13 (s, 1H), 5.56 (s, 3H), 4.53 (s, 2H) LCMS (m/z [M+H]⁺): 365.

### Preparation of (Z)—N-(3-(2-chlorophenyl)-5-(hydroxymethyl)thiazol-2(3H)-ylidene)-1H-pyrrolo[2,3-b]pyridine-4-carboxamide (Example 63)

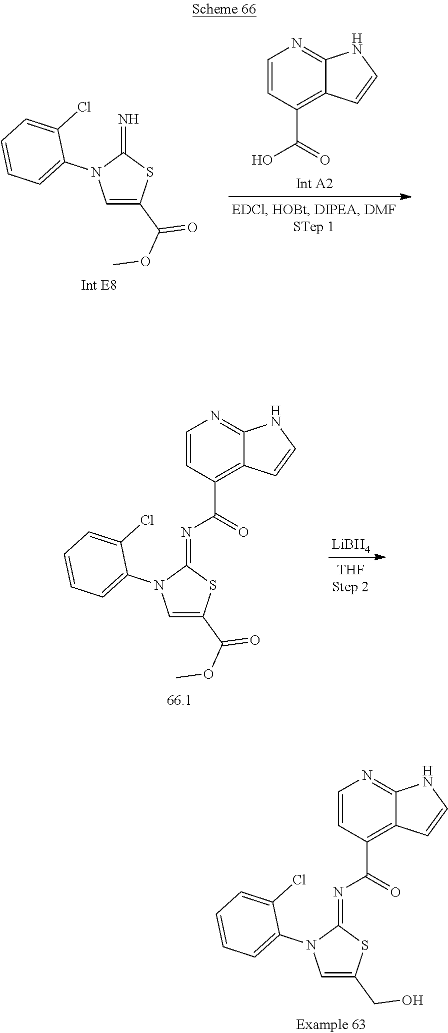 [SMILES: COC(=O)c1cn(-c2ccccc2Cl)c(=N)s1 ; O=C(O)c1ccnc2[nH]ccc12 ; COC(=O)c1cn(-c2ccccc2Cl)/c(=N/C(=O)c2ccnc3[nH]ccc23)s1 ; "LiBH4" ; O=C(/N=c1\sc(CO)cn1-c1ccccc1Cl)c1ccnc2[nH]ccc12]

### Step 1. Methyl (Z)-2-((1H-pyrrolo[2,3-b]pyridine-4-carbonyl)imino)-3-(2-chlorophenyl)-2,3-dihydrothiazole-5-carboxylate (66.1)

To a solution of 1H-pyrrolo[2,3-b]pyridine-4-carboxylic acid (A2) (0.084 g, 0.52 mmol) in DMF (3 mL) at 0° C. was added EDCI (0.300 g, 1.56 mmol), HOBt (0.056 g, 0.42 mmol), DIPEA (0.202 g, 1.56 mmol) and methyl 3-(2-chlorophenyl)-2-imino-2,3-dihydrothiazole-5-carboxylate (E8) (0.140 g, 0.521 mmol). The mixture was stirred at 25° C. for 1 hour and poured into water (30 mL). The resulting precipitate was collected by filtration to afford 0.22 g of the title compound (66.1) as a solid. ¹H NMR (400 MHz, DMSO-d₆) δ 11.77 (br. s, 1H), 8.71 (s, 1H), 8.26 (d, J=4.8 Hz, 1H), 7.87-7.81 (m, 2H), 7.71 (td, J=1.6 Hz, 8.0 Hz, 1H), 7.65 (td, J=1.6 Hz, 8.0 Hz, 1H), 7.53 (d, J=5.2 Hz, 1H), 7.42 (t, J=3.2 Hz, 1H), 6.49 (dd, J=2.0 Hz, 3.2 Hz, 1H), 3.89 (s, 3H) LCMS (m/z [M+H]⁺): 412.9

### Step 2. (Z)—N-(3-(2-Chlorophenyl)-5-(hydroxymethyl)thiazol-2(3H)-ylidene)-1H-pyrrolo[2,3-b]pyridine-4-carboxamide (Example 63)

To a solution of (Z)-methyl 2-((1H-pyrrolo[2,3-b]pyridine-4-carbonyl)imino)-3-(2-chlorophenyl)-2,3-dihydrothiazole-5-carboxylate (66.1) (0.150 g, 0.360 mmol) in THF (5 mL) at 0° C. under nitrogen was added lithium borohydride (0.025 g, 1.1 mmol). The reaction mixture was stirred at 25° C. for 12 hours, poured into a cold saturated aqueous solution of ammonium chloride (50 mL) and adjusted to pH˜9 with a saturated aqueous solution of sodium bicarbonate. The aqueous phase was extracted with EtOAc (3×30 mL). The combined organic layers were washed with brine (3×50 mL), dried over anhydrous Na₂SO₄, filtered, and concentrated. The residue was added into a solution of HCl in MeOH (4 M, 3.0 mL, 12.0 mmol) and stirred at 25° C. for 1 hour then concentrated. The residue was purified by preparative HPLC (Phenomenex Gemini-NX C₁₈ 30×75 mm, 3 μm; mobile phase A: water/0.225% FA, mobile phase B:CAN/0.225% FA); gradient: 20% B to 50% B over 7 min) to afford 0.038 g of (Z)—N-(3-(2-Chlorophenyl)-5-(hydroxymethyl)thiazol-2(3H)-ylidene)-1H-pyrrolo[2,3-b]pyridine-4-carboxamide (Example 63) as a solid.

¹H NMR (400 MHz, CD₃OD) δ 8.19 (d, J=4.8 Hz, 1H), 7.78-7.73 (m, 1H), 7.66-7.60 (m, 4H), 7.40 (t, J=1.2 Hz, 1H), 7.27 (d, J=3.2 Hz, 1H), 6.62 (d, J=3.2 Hz, 1H), 4.74 (d, J=0.4 Hz, 2H) LCMS (m/z [M+H]⁺): 385.1

### Preparation of (Z)—N-(3-benzyl-4-((dimethylamino)methyl)thiazol-2(3H)-ylidene)-1H-pyrrolo[2,3-b]pyridine-3-carboxamide (Example 65)

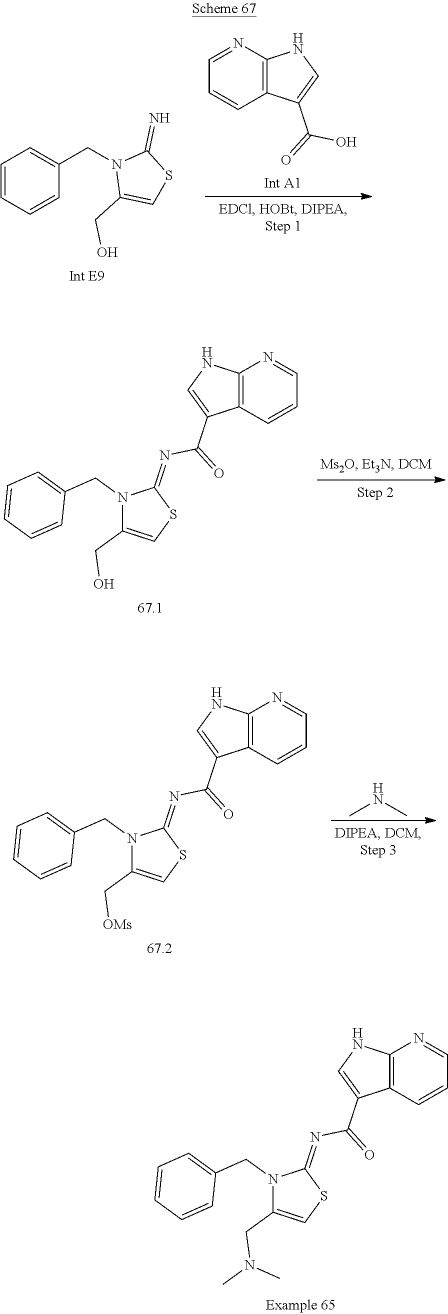

### Step 1. (Z)—N-(3-Benzyl-4-(hydroxymethyl)thiazol-2(3H)-ylidene)-1H-pyrrolo[2,3-b]pyridine-3-carboxamide (67.1)

To a mixture of (3-benzyl-2-imino-2,3-dihydrothiazol-4-yl)methanol (E9) (0.300 g, 1.36 mmol) and 1H-pyrrolo[2,3-b]pyridine-3-carboxylic acid (A1) (0.220 g, 1.36 mmol) in DMF (5 mL) was added DIPEA (0.530 g, 4.09 mmol), HOBt (0.110 g, 0.820 mmol) and EDCI (0.390 g, 2.04 mmol) at 25° C. The reaction mixture was stirred at 25° C. for 3 hours and purified by reversed-phase flash chromatography (0.1% FA/MeCN condition) to afford 0.12 g of the title compound (67.1) as a solid.

¹H NMR (400 MHz, DMSO-d₆) δ 12.16 (br. s, 1H), 8.44 (dd, J=1.6 Hz, 8.0 Hz, 1H), 8.23 (dd, J=1.6 Hz, 4.4 Hz, 1H), 8.11 (d, J=2.8 Hz, 1H), 7.40-7.23 (m, 5H), 7.12 (dd, J=4.8 Hz, 8.0 Hz, 1H), 6.86 (s, 1H), 5.66-5.60 (m, 3H), 4.42 (d, J=5.6 Hz, 2H)

LCMS (m/z [M+H]⁺): 365.3

### Step 2. (Z)-(2-((1H-Pyrrolo[2,3-b]pyridine-3-carbonyl)imino)-3-benzyl-2,3-dihydrothiazol-4-yl)methyl methanesulfonate (67.2)

To a solution of (Z)—N-(3-benzyl-4-(hydroxymethyl)thiazol-2(3H)-ylidene)-1H-pyrrolo [2,3-b]pyridine-3-carboxamide (63.1) (0.050 g, 0.137 mmol) in DCM (1 mL) was added Et₃N (0.027 g, 0.27 mmol) and methanesulfonic anhydride (0.026 g, 0.015 mmol) at 0° C. The reaction mixture was stirred at 25° C. for 1 hour and concentrated under reduced pressure to afford 0.050 g of the crude title compound (67.2) as a solid which was used without further purification. LCMS (m/z [M+H]⁺): 443.1

### Step 3. (Z)—N-(3-Benzyl-4-((dimethylamino)methyl)thiazol-2(3H)-ylidene)-1H-pyrrolo[2,3-b]pyridine-3-carboxamide (Example 65)

To a mixture of (Z)-(2-((1H-pyrrolo[2,3-b]pyridine-3-carbonyl)imino)-3-benzyl-2,3-dihydrothiazol-4-yl)methyl methanesulfonate (63.2) (0.050 g, 0.11 mmol) and dimethylamine hydrochloride (0.027 g, 0.39 mmol) in DCM (1 mL) was added Et₃N (0.011 g, 0.11 mmol) at 25° C. The mixture wa 7tirred for 30 minutes then quenched with water (5 mL) and extracted with DCM (3×10 mL). The combined organic layers were washed with brine (3×10 mL), dried over anhydrous Na₂SO₄, filtered, and concentrated. The residue was purified by preparative HPLC (Phenomenex Gemini, 25×150 mm, 10 μm; mobile phase A: water/10 mM NH₄HCO₃), mobile phase B: MeCN; gradient: 30% B to 60% B over 5 min) to afford 0.031 g of (Z)—N-(3-benzyl-4-((dimethylamino)methyl)thiazol-2(3H)-ylidene)-1H-pyrrolo[2,3-b]pyridine-3-carboxamide (Example 65) as a solid.

¹H NMR (400 MHz, CD₃OD) δ 8.53 (dd, J=1.6 Hz, 8.0 Hz, 1H), 8.20 (dd, J=1.6 Hz, 4.8 Hz, 1H), 8.09 (s, 1H), 7.38-7.33 (m, 2H), 7.30-7.24 (m, 3H), 7.11 (dd, J=4.8 Hz, 8.0 Hz, 1H), 6.81 (s, 1H), 5.88 (s, 2H), 3.34 (s, 2H), 2.25 (s, 6H) LCMS (m/z [M+H]⁺): 392.1

### Preparation of (Z)—N-(3-(2-chlorophenyl)-4-((dimethylamino)methyl)thiazol-2(3H)-ylidene)-1H-pyrrolo[2,3-b]pyridine-3-carboxamide (Example 66)

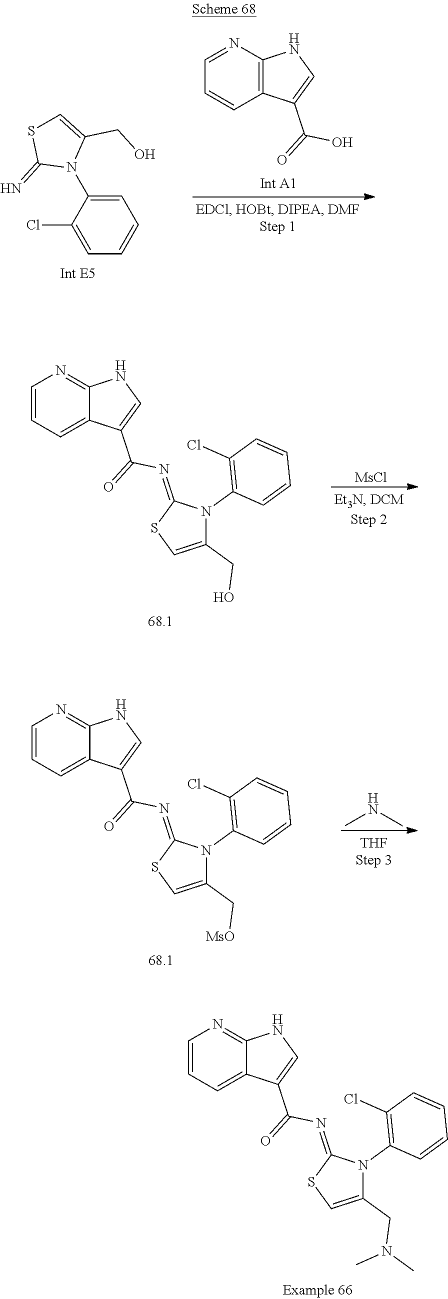

### Step 1. (Z)—N-(3-(2-Chlorophenyl)-4-(hydroxymethyl)thiazol-2(3H)-ylidene)-1H-pyrrolo[2,3-b]pyridine-3-carboxamide (68.1)

To a mixture of (3-(2-chlorophenyl)-2-imino-2,3-dihydrothiazol-4-yl)methanol (E5) (0.150 g, 0.623 mmol), 1H-pyrrolo[2,3-b]pyridine-3-carboxylic acid (A1) (0.101 g, 0.623 mmol), HOBt (0.151 g, 1.12 mmol), DIPEA (0.241 g, 1.87 mmol) in DMF (5 mL) was added EDCI (0.215 g, 1.12 mmol). The reaction mixture was stirred at 25° C. for 3 hours. The mixture was poured into ice-water (w/w=1/1) (20 mL) and stirred for 5 minutes. The resulting precipitate was collected by filtration and dried under reduced pressure. The crude product was purified by reversed phase flash chromatography (0.1% TFA/MeCN condition) to afford 0.080 g of the title compound (68.1) as a solid.

¹H NMR (400 MHz, DMSO-d₆) δ 12.15 (br. s, 1H), 8.16 (dd, J=1.6 Hz, 4.8 Hz, 1H), 7.86 (d, J=2.4 Hz, 1H), 7.84-7.79 (m, 1H), 7.73-7.61 (m, 4H), 6.93 (s, 1H), 6.87-6.84 (m, 1H), 4.19-4.14 (m, 2H) LCMS (m/z [M+H]⁺): 385.0

### Step 2. (Z)-(2-((1H-Pyrrolo[2,3-b]pyridine-3-carbonyl)imino)-3-(2-chlorophenyl)-2,3-dihydrothiazol-4-yl)methyl methanesulfonate (68.2)

To a mixture of (Z)—N-(3-(2-chlorophenyl)-4-(hydroxymethyl)thiazol-2(3H)-ylidene)-1H-pyrrolo[2,3-b]pyridine-3-carboxamide (68.1) (0.060 g, 0.13 mmol) and Et₃N (0.025 g, 0.25 mmol) in DCM (1 mL) was added MsCl (0.021 g, 0.189 mmol) at 0° C. under nitrogen atmosphere. The mixture was stirred at 25° C. for 1 hour. The mixture was poured into ice-water (w/w=1/1, 10 mL) and stirred for 1 minutes. The aqueous phase was extracted with DCM (3×10 mL). The combined organic phases were washed with brine (10 mL), dried over anhydrous Na₂SO₄, filtered, and concentrated to afford 0.05 g of the crude title compound (68.2) as a solid. LCMS (m/z [M+H]⁺): 462.9

### Step 3. (Z)—N-(3-(2-Chlorophenyl)-4-((dimethylamino)methyl)thiazol-2(3H)-ylidene)-1H-pyrrolo[2,3-b]pyridine-3-carboxamide (Example 66)

A mixture of (Z)-(2-((1H-pyrrolo[2,3-b]pyridine-3-carbonyl)imino)-3-(2-chlorophenyl)-2, 3-dihydrothiazol-4-yl)methyl methanesulfonate (68.2) (0.050 g, 0.11 mmol) and N-methylmethanamine (2 M, 1.50 mL, 3.00 mmol) was stirred at 65° C. for 16 hours under nitrogen. The mixture was poured into ice-water (w/w=1/1, 10 mL) and stirred for 1 minute then the aqueous phase extracted with EtOAc (2×20 mL). The combined organic phases were washed with brine (2×10 mL), dried over anhydrous Na₂SO₄, filtered, and concentrated. The residue was purified by preparative HPLC (Phenomenex Gemini-NX Cis 30×75 mm, 3 μm; mobile phase A: water/10 mM NH₄HCO₃, mobile phase B: ACN; gradient: 25% B to 55% B over 8 min) to give 0.015 g of (Z)—N-(3-(2-chlorophenyl)-4-((dimethylamino)methyl)thiazol-2(3H)-ylidene)-1H-pyrrolo[2,3-b]pyridine-3-carboxamide (Example 66) as a solid.

¹H NMR (400 MHz, CD₃OD) δ 8.13 (dd, J=0.8 Hz, 4.8 Hz, 1H), 7.92 (s, 1H), 7.77-7.74 (m, 2H), 7.69-7.63 (m, 1H), 7.65-7.59 (m, 2H), 6.91-6.87 (m, 2H), 3.20 (d, J=3.2 Hz, 2H), 2.12 (s, 6H) LCMS (m/z [M+H]⁺): 412.1

### Preparation of (NZ)—N-[3-(2-chloro-6-hydroxy-phenyl)thiazolidin-2-ylidene]-1H-pyrrolo[2,3-b]pyridine-3-carboxamide (Example 74)

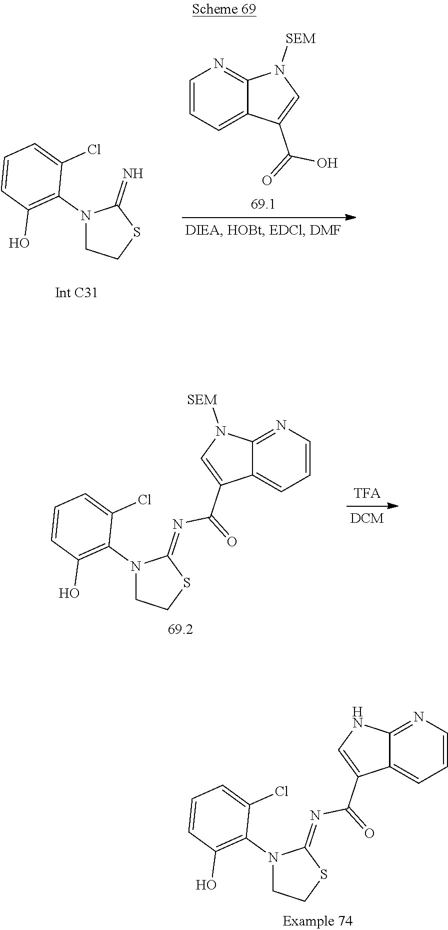 [SMILES: N=C1SCCN1c1c(O)cccc1Cl ; C[Si](C)(C)CCOCn1cc(C(=O)O)c2cccnc21 ; C[Si](C)(C)CCOCn1cc(C(=O)/N=C2\SCCN2c2c(O)cccc2Cl)c2cccnc21 ; O=C(/N=C1\SCCN1c1c(O)cccc1Cl)c1c[nH]c2ncccc12]

### Step 1. (NZ)—N-[3-(2-chloro-6-hydroxy-phenyl)thiazolidin-2-ylidene]-1-(2-trimethylsilylethoxymethyl)pyrrolo[2,3-b]pyridine-3-carboxamide

To a solution of 1-(2-trimethylsilylethoxymethyl)pyrrolo[2,3-b]pyridine-3-carboxylic acid (69.1) (0.100 g, 0.342 mmol) in dimethyl formamide (1.5 mL) was added diisopropylethylamine (0.132 g 1.03 mmol), HOBt (0.069 g 0.513 mmol) and EDCI (0.098 g, 0.513 mmol). After 30 minutes, 3-chloro-2-(2-iminothiazolidin-3-yl)phenol (Int C31) (0.156 g, 0.684 mmol) was added at 25° C. and the mixture was stirred for 3 hours. The reaction mixture was diluted with water (5 mL) and extracted with ethyl acetate (4×8 mL). The combined organic phases were washed with brine (2×8 mL), dried over anhydrous sodium sulfate and filtered. The filtrate was concentrated under reduced pressure. The residue was purified by prep-TLC (silica gel, petroleum ether: ethyl acetate=3:1) to afford 0.085 g of (NZ)—N-[3-(2-chloro-6-hydroxy-phenyl)thiazolidin-2-ylidene]-1-(2-trimethylsilylethoxymethyl)pyrrolo[2,3-b]pyridine-3-carboxamide(69.2) as yellow oil.

¹H NMR: (400 MHz, CDCl₃) δ 8.34 (d, J=1.2 Hz, 1H), 8.33 (d, J=1.2 Hz, 1H), 7.97 (s, 1H), 7.32 (t, J=8.4 Hz, 1H), 7.27 (s, 3H), 5.66 (s, 2H), 4.36-4.19 (m, 1H), 4.10-3.54 (m, 1H), 3.56-3.53 (m, 3H), 3.52-3.32 (m, 1H), 0.92-0.87 (m, 2H), −0.07 (s, 9H).

### Step 2. (NZ)—N-[3-(2-chloro-6-hydroxy-phenyl)thiazolidin-2-ylidene]-1H-pyrrolo[2,3-b]pyridine-3-carboxamide (Example 74)

To a solution of (NZ)—N-[3-(2-chloro-6-hydroxy-phenyl)thiazolidin-2-ylidene]-1-(2-trimethylsilylethoxymethyl)pyrrolo[2,3-b]pyridine-3-carboxamide (69.2) (0.065 g, 0.129 mmol) in dichloromethane (1 mL) was added trifluoroacetic acid (1 mL) at 25° C. The resulting mixture was stirred for 1 hour and then concentrated under reduced pressure. The crude was dissolved in methanol (8 mL) and treated with potassium carbonate (0.045 g) at 25° C. for 1 hour. The reaction mixture was filtered, and the filtrate was concentrated under reduced pressure. The residue was diluted with water (5 mL) and extracted with ethyl acetate (4×8 mL). The combined organic phases were washed with brine (2×8 mL), dried over anhydrous sodium sulfate and filtered. The filtrate was concentrated under reduced pressure. The residue was triturated with acetonitrile (10 mL) at 25° C. to afford 0.015 g of (NZ)—N-[3-(2-chloro-6-hydroxy-phenyl)thiazolidin-2-ylidene]-1H-pyrrolo[2,3-b]pyridine-3-carboxamide (Example 74) as a white solid.

### Preparation of (NZ)—N-[3-(2-hydroxycyclopentyl)thiazolidin-2-ylidene]-1H-pyrrolo[2,3-b]pyridine-3-carboxamide (Example 79)

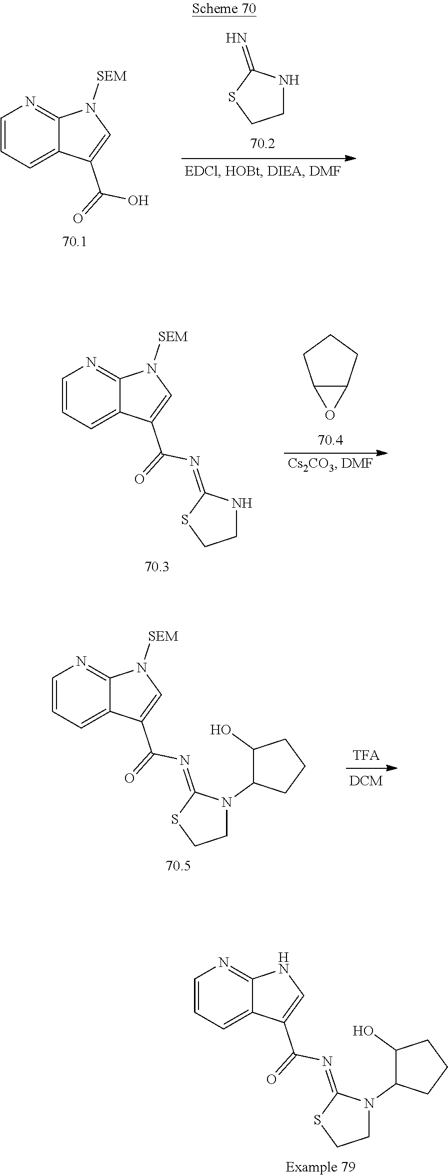

### Step 1. (NZ)—N-thiazolidin-2-ylidene-1-(2-trimethylsilylethoxymethyl)pyrrolo[2,3-b]pyridine-3-carboxamide (70.3)

To a solution of thiazolidin-2-imine (0.650 g, 6.36 mmol) (70.2) and 1-(2-trimethylsilylethoxymethyl)pyrrolo[2,3-b]pyridine-3-carboxylic acid (70.1) (1.49 g, 5.09 mmol) in N,N-dimethylformamide (15 mL) was added N,N-diisopropylethylamine (2.47 g, 19.09 mmol), 1-hydroxybenzotriazole (1.29 g, 9.54 mmol) and 3-(ethyliminomethylideneamino)propyl-dimethylazanium chloride (1.83 g, 9.54 mmol). The resulting mixture was stirred at 25° C. for 12 hours. The reaction mixture was quenched with water (50 mL) and extracted with ethyl acetate (3×30 mL). The combined organic phases were washed with brine (2×50 mL), dried over anhydrous sodium sulfate, and filtered. The filtrate was concentrated under reduced pressure to give (NZ)—N-thiazolidin-2-ylidene-1-(2-trimethylsilylethoxymethyl)pyrrolo[2,3-b]pyridine-3-carboxamide (70.3).

¹H NMR: (400 MHz, DMSO-d₆) δ 8.60 (dd, J=1.6 Hz, 8.0 Hz, 1H), 8.33 (dd, J=1.6 Hz, 4.8 Hz, 1H), 8.25 (br s, 1H), 7.27 (dd, J=4.8 Hz, 8.0 Hz 1H), 5.68 (s, 2H), 3.62 (t, J=7.6 Hz, 2H), 3.54 (t, J=8.0 Hz, 2H), 3.23 (t, J=7.6 Hz, 2H), 0.82 (t, J=8.0 Hz, 2H), −0.12 (s, 9H).

### Step 2. (NZ)—N-[3-(2-hydroxycyclopentyl)thiazolidin-2-ylidene]-1-(2-trimethylsilylethoxymethyl)pyrrolo[2,3-b]pyridine-3-carboxamide (70.5)

To a solution of (NZ)—N-thiazolidin-2-ylidene-1-(2-trimethylsilylethoxymethyl)pyrrolo[2,3-b]pyridine-3-carboxamide (70.3) (0.200 g, 0.531 mmol) in dimethylformamide (5 mL) was added caesium carbonate (0.692 g, 2.12 mmol) and 6-oxabicyclo[3.1.0]hexane (70.4) (0.134 g, 1.59 mmol) at 25° C. The resulting mixture was stirred at 100° C. for 12 hours. The reaction mixture was diluted water (30 mL) and extracted with ethyl acetate (3×30 mL). The combined organic phases were washed with brine (2×30 mL), dried over anhydrous sodium sulfate and filtered. The filtrate was concentrated under reduced pressure. The residue was purified by reversed phase flash chromatography (0.1% TFA/MeCN condition) to afford 0.162 g of (NZ)—N-[3-(2-hydroxycyclopentyl)thiazolidin-2-ylidene]-1-(2-trimethylsilylethoxymethyl)pyrrolo[2,3-b]pyridine-3-carboxamide (70.5) as a yellow oil.

¹H NMR (400 MHz, CDCl₃): δ 8.70 (d, J=8.0 Hz, 1H), 8.49 (d, J=3.2 Hz, 1H), 8.41 (s, 1H), 7.33 (dd, J=4.8 Hz, 7.6 Hz, 1H), 6.57 (br s, 1H), 5.73 (dd, J=10.8 Hz, 24.0 Hz, 2H), 4.76-4.70 (m, 1H), 4.39 (d, J=5.6 Hz, 1H), 4.08-3.90 (m, 2H), 3.61 (t, J=8.0 Hz, 2H), 3.38-3.31 (m, 2H), 2.21-2.16 (m, 2H), 1.96-1.85 (m, 4H), 0.93 (dd, J=9.2 Hz, 7.6 Hz, 1H), −0.06 (s, 9H). LCMS (m/z [M+H]⁺): 461.2

### Step 3. (NZ)—N-[3-(2-hydroxycyclopentyl)thiazolidin-2-ylidene]-1H-pyrrolo[2,3-b]pyridine-3-carboxamide (Example 79)

To a solution of (NZ)—N-[3-(2-hydroxycyclopentyl)thiazolidin-2-ylidene]-1-(2-trimethylsilylethoxymethyl)pyrrolo[2,3-b]pyridine-3-carboxamide (70.5) (0.152 g, 0.330 mmol) in dichloromethane (3 mL) was added trifluoroacetic acid (0.304 g, 2.67 mmol) at 0° C. Then the mixture was stirred at 25° C. for 12 hours. The reaction solution was concentrated under reduced pressure. The residue was purified by preparative HPLC (column: Waters xbridge 25×150 mm 10 μm; mobile phase A: water/10 mM NH₄HCO₃, mobile phase B: ACN; gradient: 11% B to 41% B over 8 min) to afford 0.034 g of (NZ)—N-[3-(2-hydroxycyclopentyl)thiazolidin-2-ylidene]-1H-pyrrolo[2,3-b]pyridine-3-carboxamide (Example 79) as a white solid.

¹H NMR (400 MHz, CDCl₃) δ 9.78 (br. s, 1H), 8.74 (d, J=8.0 Hz, 1H), 8.37 (d, J=4.0 Hz, 1H), 8.14 (d, J=1.6 Hz, 1H), 7.25-7.23 (m, 1H), 5.40 (s, 1H), 4.66-4.60 (m, 1H), 4.31-4.26 (m, 1H), 3.75 (t, J=7.6 Hz, 2H), 3.20 (t, J=7.6 Hz, 2H), 2.17-2.11 (m, 2H), 1.93-1.79 (m, 4H). LCMS (m/z [M+H]⁺): 331.2

### Preparation of (Z)—N-(3-(3-hydroxycyclopentyl)thiazolidin-2-ylidene)-1H-pyrrolo[2,3-b]pyridine-3-carboxamide (Example 81)

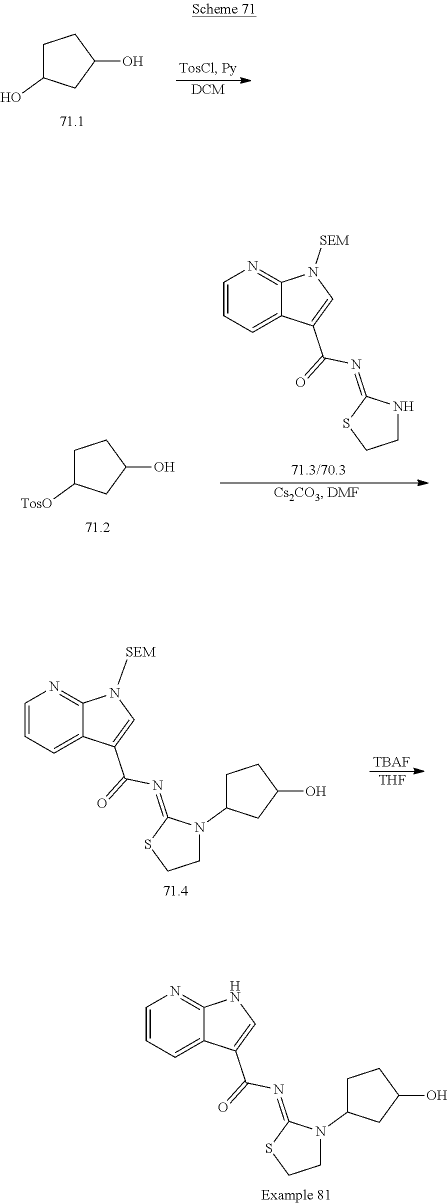

### Step 1. 3-hydroxycyclopentyl 4-methylbenzenesulfonate (71.2)

To a solution of p-toluenesulfonyl chloride (3.36 g, 17.6 mmol) in dichloromethane (10 mL) was added dropwise to an ice-cooled solution of cyclopentane-1,3-diol (71.1) (2.00 g, 19.6 mmol) in dichloromethane (10 mL) and pyridine (20 mL, 247 mmol) at 0° C. The resulting mixture was stirred at 20° C. for 2 hours. The reaction mixture was diluted with dichloromethane (20 mL), washed with diluted hydrochloric acid (1.2 mol/L in water, 3×30 mL). The organic layer was dried over anhydrous sodium sulfate, filtered and the filtrate was concentrated under reduced pressure to afford 0.180 g of 3-hydroxycyclopentyl 4-methylbenzenesulfonate (71.2) as a colorless oil.

¹H NMR (400 MHz, CDCl₃) δ 7.78 (d, J=8.4 Hz, 2H), 7.35 (d, J=8.0 Hz, 2H), 5.08-5.05 (m, 1H), 4.48-4.45 (m, 1H), 2.46 (s, 3H), 2.10-2.02 (m, 6H).

### Step 2. (Z)—N-(3-(3-hydroxycyclopentyl)thiazolidin-2-ylidene)-1-((2-(trimethylsilyl)ethoxy)methyl)-1H-pyrrolo[2,3-b]pyridine-3-carboxamide (71.4)

To a solution of 3-hydroxycyclopentyl 4-methylbenzenesulfonate (71.2) (0.170 g, 0.663 mmol) and (Z)—N-(thiazolidin-2-ylidene)-1-((2-(trimethylsilyl)ethoxy)methyl)-1H-pyrrolo[2,3-b]pyridine-3-carboxamidee (71.3) (0.249 g, 0.663 mmol) in dimethylformamide (3 mL) was added cesium carbonate (0.648 g, 1.99 mmol). The resulting mixture was stirred at 20° C. for 12 hours. The reaction mixture was poured into water (25 mL) and extracted with ethyl acetate (3×25 mL). The combined organic phases were dried over anhydrous sodium sulfate, filtered and the filtrate was concentrated under reduced pressure. The residue was purified by reversed-phase flash chromatography (0.1% FA/MeCN condition) to afford 0.140 g of (Z)—N-(3-(3-hydroxycyclopentyl)thiazolidin-2-ylidene)-1-((2-(trimethylsilyl)ethoxy)methyl)-1H-pyrrolo[2,3-b]pyridine-3-carboxamide as a formate (71.4) as yellow oil.

¹H NMR (400 MHz, DMSO-d₆) δ 8.69 (dd, J=1.2 Hz, 8.0 Hz, 1H), 8.37 (dd, J=1.2 Hz, 4.8 Hz, 1H), 8.21 (s, 1H), 8.05 (s, 1H), 7.24-7.21 (m, 1H), 5.73 (s, 2H), 5.21-5.09 (m, 1H), 5.10-5.07 (m, 1H), 4.46-4.43 (m, 1H), 3.85-3.78 (m, 2H), 3.58 (t, J=8.0 Hz, 2H), 3.14 (t, J=8.0 Hz, 2H), 2.43-2.35 (m, 1H), 2.23-2.16 (m, 1H), 1.91-1.77 (m, 4H), 0.93 (t, J=8.0 Hz, 2H), 0.06 (s, 9H).

### Step 3. (Z)—N-(3-(3-hydroxycyclopentyl)thiazolidin-2-ylidene)-1H-pyrrolo[2,3-b]pyridine-3-carboxamide (Example 81)

To a solution of (Z)—N-(3-(3-hydroxycyclopentyl)thiazolidin-2-ylidene)-1-((2-(trimethylsilyl)ethoxy)methyl)-1H-pyrrolo[2,3-b]pyridine-3-carboxamide (71.4) (0.120 g, 0.260 mmol) in tetrahydrofuran (2 mL) was added tetrabutylammonium fluoride (1 mol/L in tetrahydrofuran, 0.521 mL). Then the mixture was stirred at 20° C. for 5 hours. The mixture was concentrated under pressure. The residue was purified by reversed-phase flash (0.1% ammon/MCCN condition) and then purified by prep-HPLC (column: Welch Xtimate C18 25×150 mm, 5 μm; mobile phase A: water/10 mM NH₄HCO₃, mobile phase B: ACN; gradient: 7% B to 37% B over 8 min) to afford 0.023 g of (Z)—N-(3-(3-hydroxycyclopentyl)thiazolidin-2-ylidene)-1H-pyrrolo[2,3-b]pyridine-3-carboxamide (Example 81) as a white solid.

¹H NMR (400 MHz, CD₃OD) δ 8.63 (d, J=8 Hz, 1H), 8.25 (d, J=4.8 Hz, 1H), 8.17 (s, 1H), 7.24 (dd, J=4.8 Hz, 7.6 Hz, 1H), 5.30-5.26 (m, 1H), 4.35-4.33 (m, 1H), 3.88-3.84 (m, 2H), 3.19 (t, J=8.0 Hz, 2H), 2.40-2.36 (m, 1H), 2.06-2.04 (m, 2H), 1.88-1.85 (m, 2H), 1.84-1.83 (m, 1H). LCMS (m/z [M+H]⁺): 331.2

### Preparation of (Z)—N-(3-phenylthiazolidin-2-ylidene)-1H-pyrrolo[3,2-b]pyridine-1-carboxamide (Example 83)

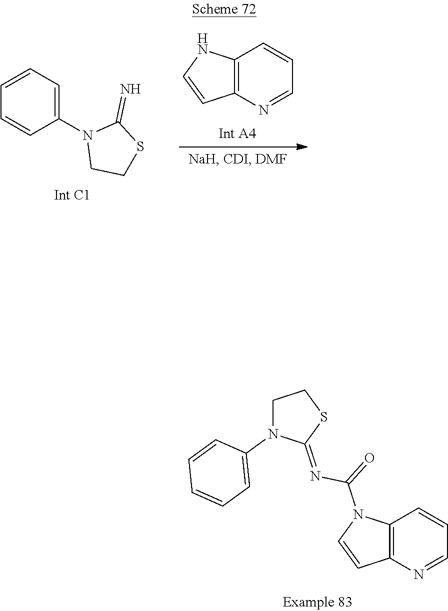 [SMILES: N=C1SCCN1c1ccccc1 ; c1cnc2cc[nH]c2c1 ; O=C(/N=C1\SCCN1c1ccccc1)n1ccc2ncccc21]

A solution of 3-phenylthiazol-2-imine (C1) (0.220 g, 1.25 mmol) and 1,1-carbonyldiimidazole (0.243 g, 1.50 mmol) in THF (5 mL) was stirred at 25° C. for 1 hour. Sodium hydride (0.100 g, 2.50 mmol, 60% in mineral oil) was added to a solution of 1H-pyrrolo[3,2-b]pyridine A4) (0.221 g, 1.87 mmol) in THF (5 mL) at 0° C. under nitrogen and the mixture was stirred at 25° C. for 15 minutes, which was added dropwise to the solution with C1. The resulting mixture was stirred for 2 hours at 25° C. under nitrogen and added dropwise to ice-water (30 mL) at 25° C. and then extracted with EtOAc (3×100 mL). The combined organic layers were washed with brine (2×100 mL), dried over anhydrous Na₂SO₄, filtered, and evaporated. The residue was purified by silica gel column chromatography, eluting with 0 to 50% EtOAc in petroleum ether followed by trituration with acetonitrile (5 mL) to afford 0.050 g of (Z)—N-(3-phenylthiazolidin-2-ylidene)-1H-pyrrolo[3,2-b]pyridine-1-carboxamide (Example 83) as a solid.

¹H NMR (400 MHz, DMSO-d₆) δ 8.37 (dd, J=1.2 Hz, 4.8 Hz, 1H), 8.23 (s, 1H), 8.01 (d, J=3.2 Hz, 1H), 7.81 (d, J=4.8 Hz, 1H), 7.72-7.62 (m, 5H), 7.33 (d, J=4.8 Hz, 1H), 7.03 (s, 1H), 6.73 (d, J=3.6 Hz, 1H).

LCMS (m/z [M+H]⁺): 321.0

Examples 84-88 in Table G were prepared in a similar fashion to that shown above in Scheme 72 using intermediates from Table C-D and the appropriate azaindoles from Table A.

TABLE G

| Ex. | Structure | Int. | LCMS | 1H NMR |
|---|---|---|---|---|
| 84 | 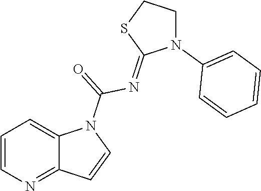 [SMILES: O=C(/N=C1\SCCN1c1ccccc1)n1ccc2ncccc21] | A4 C1 | 323.0 | (DMSO-d₆, 400 MHz) δ 8.36 (d, J = 3.6 Hz, 1H), 8.31 − 8.01 (m, 1H), 7.92 (d, J = 2.4 Hz, 1H), 7.64 − 7.52 (m, 4H), 7.46 − 7.44 (m, 1H), 7.02 (s, 1H), 6.71 (d, J = 3.2 Hz, 1H), 4.27 (t, J = 7.6 Hz, 2H), 3.47 (t, J = 8.0 Hz, 2H) |
| 85 | 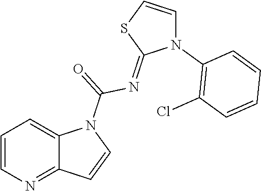 [SMILES: O=C(/N=c1\sccn1-c1ccccc1Cl)n1ccc2ncccc21] | A4 D1 | 355.0 | (DMSO-d₆, 400 MHz) δ 8.37 (dd, J = 1.6, 4.8 Hz, 1 H), 7.83 − 7.97 (m, 3 H), 7.77 − 7.82 (m, 2 H), 7.74 (dt, J = 1.6, 7.6 Hz, 1 H), 7.68 (dt, J = 1.6, 7.6 Hz, 1 H), 7.35 (d, J = 4.8 Hz, 1 H), 6.88 − 7.04 (m, 1 H), 6.73 (d, J = 3.6 Hz, 1 H). |
| 86 | 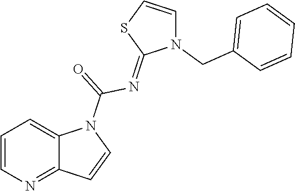 [SMILES: O=C(/N=c1\sccn1Cc1ccccc1)n1ccc2ncccc21] | A4 D12 | 335.1 | (DMSO-d₆, 400 MHz) δ 8.62 (d, J = 8.0 Hz, 1H), 8.42 (dd, J = 1.2, 4.4 Hz, 1H), 8.34 (d, J = 3.6 Hz, 1H), 7.74 (d, J = 4.8 Hz, 1H), 7.41 − 7.36 (m, 4H), 7.33 − 7.29 (m, 1H), 7.25 − 7.19 (m, 2H), 6.76 (d, J = 3.6 Hz, 1H), 5.55 (s, 2H) |
| 87 | 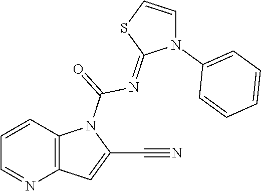 [SMILES: N#Cc1cc2ncccc2n1C(=O)/N=c1\sccn1-c1ccccc1] | A5 D13 | 367.9 | (CDCl₃, 400 MHz) δ 8.56 (d, J = 4.8 Hz, 1H), 8.35 (d, J = 8.4 Hz, 1H), 7.72 − 7.61 (m, 3H), 7.60 − 7.54 (m, 2H), 7.53 (s, 1H), 7.28 − 7.26 (m, 1H), 7.12 (dd, J = 4.8, 8.4 Hz, 1H), 6.99 (d, J = 4.8 Hz, 1H). |
| 88 | 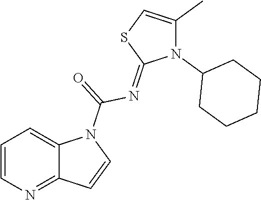 [SMILES: Cc1cs/c(=N\C(=O)n2ccc3ncccc32)n1C1CCCCC1] | A4 D6 | 367.9 | (CDCl₃, 400 MHz) δ 8.56 (d, J = 4.8 Hz, 1H), 8.35 (d, J = 8.4 Hz, 1H), 7.72 − 7.61 (m, 3H), 7.60 − 7.54 (m, 2H), 7.53 (s, 1H), 7.28 − 7.26 (m, 1H), 7.12 (dd, J = 4.8, 8.4 Hz, 1H), 6.99 (d, J = 4.8 Hz, 1H). |

### General Methods

LCMS conditions

| Instrument | | Agilent 1200\G6110A | | |
|---|---|---|---|---|
| Software | | Agilent ChemStation Rev. B. 04.03[54] | | |
| HPLC | Column | Kinetex@ 5 um EVO C18 30*2.1 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.01 | 5 | 0.8 |
| | | 3.00 | 95 | 0.8 |
| | | 3.5 | 95 | 0.8 |
| | | 3.51 | 5 | 0.8 |
| | | 4.00 | 5 | 0.8 |

| | | |
|---|---|---|
| | Column Temp | 50° C. |
| | Detector | DAD (220&254 nm) |
| MS | Ionization source | ESI |
| | Drying Gas | N2 |
| | Drying Gas Flow | 11(L/min) |
| | Nebulizer Pressure | 60 (psig) |
| | Drying Gas Temp | 350(° C.) |
| | Capillary Voltage | 3500(V) |
| | MS Polarity | Positive |
| | MS Mode | Scan |
| | Mass range | 100-1000 |

### LATS1 Biochemical HTRF Assay

In a 384-well white small volume plate (Greiner 784075) add 3.5 μL of 1× Enzymatic buffer with [5 mM] MgCl₂ and [1 mM] DTT. 1× Enzymatic buffer diluted from 5× Enzymatic buffer*. Add 0.5 μL of 20× compound in 100% DMSO. Add 2 μL±[2.8 nM] LATS1 kinase (Carnabio #01-123) in 5× Enzyme Resuspension Buffer (ERB) with [5 mM] MgCl₂, [1 mM] DTIT and [5 mg/mL] BSA. 5×ERR prepared from 5× Enzymatic buffer with [25 mg/mL] BSA. Mix plate on plate shaker set at 1,350 rpm for 30 seconds, then centrifuge plate at 1,000 rpm for 1 minute. Incubate for 30 minutes at 25° C. on plate shaker set to 500 rpm. Add 2 μL [12.5 μM] STK Substrate 1-biotin* then add 2 μL [10 mM] ATP. Mix plate on plate shaker set at 1,350 rpm for 30 seconds, then centrifuge plate at 1,000 rpm for 1 minute. Incubate for 40 minutes at 25° C. on plate shaker set to 500 rpm. Stop reaction by adding 10 μL of a 1:1 mix of [625 nM] Streptavidin-XL665 with 1×STK Antibody-Cryptate*. Mix plate on plate shaker set at 1,350 rpm for 30 seconds, then centrifuge plate at 1,000 rpm for 1 minute. Incubate for 60 minutes at 25° C. on plate shaker set to 500 rpm, covered from light. Read plate: ex 330 nm; em₁ 620 nm and em₂ 665 nm. Calculate the ratio of the acceptor (665 nm) and donor (620 nm) emission signals for each well. Ratio is equal to signal 665 nm/signal 620 nm×10,000.

### *(Included in Cisbio KinEASE-STK Si HTRF Kit: 62ST1PEC)

TABLE H
Inhibitory Activity against LATS1

| | LATS1_HTRF_2 mM |
|---|---|
| | ATP |
| Ex. | IC₅₀ (nM) |
| 1 | 11.2 |
| 2 | 0.829 |
| 3 | 0.297 |
| 4 | 4.2 |
| 5 | 32 |
| 6 | 0.59 |
| 7 | 12 |
| 8 | 13 |
| 9 | 56 |
| 10 | 1.6 |
| 11 | 1.1 |
| 12 | 1.1 |
| 13 | 0.44 |
| 14 | 4.1 |
| 15 | 7.9 |
| 16 | 570 |
| 17 | 82 |
| 18 | 500 |
| 19 | 210 |
| 21 | 3780 |
| 22 | 2130 |
| 23 | 224 |
| 25 | 2.5 |
| 26 | 1.8 |
| 27 | 200 |
| 28 | 1.6 |
| 29 | 1380 |
| 30 | 2.9 |
| 31 | 2750 |
| 32 | 470 |
| 33 | 4440 |
| 34 | 23 |
| 35 | 3.9 |
| 37 | 1680 |
| 38 | 650 |
| 39 | 270 |
| 40 | 220 |
| 41 | 0.93 |
| 42 | 33 |
| 43 | 1670 |
| 44 | 360 |
| 45 | 145 |
| 46 | 1770 |
| 47 | 765 |
| 48 | 1230 |
| 49 | 505 |
| 50 | 2060 |
| 51 | 31 |
| 52 | 12 |
| 53 | 220 |
| 54 | 180 |
| 55 | 68 |
| 56 | 2780 |
| 57 | 460 |
| 58 | 48 |
| 59 | 215 |
| 60 | 330 |
| 61 | 1230 |
| 62 | 4040 |
| 63 | 850 |
| 65 | 15 |
| 66 | 1.3 |
| 67 | 1.1 |
| 68 | 2.8 |
| 69 | 1.8 |
| 70 | 0.6 |
| 71 | 0.3 |
| 72 | 1.1 |
| 73 | 1.4 |
| 74 | 1.8 |
| 75 | 1.5 |
| 76 | 191 |
| 77 | 4825 |
| 78 | 18 |
| 79 | 221 |
| 80 | 1.6 |
| 81 | 50 |
| 82 | 17 |
| 83 | 140 |
| 84 | 150 |
| 85 | 63 |
| 86 | 900 |
| 87 | 2140 |
| 88 | 7.3 |

pYAP HTRF Assay (HEK 293A Cells)
Seed HEK 293A cells in a 96-well plate (Greiner 655098) at a density of 50,000 cells/well in 100 μL of complete medium, incubate overnight (at 37° C., 5% CO2)
Master DMSO-Compound plate: Microplate, small volume 384 well (Greiner 784075)—Follow the compound serial dilution in 100% DMSO at a starting concentration of 10 mM in column 2, then diluted 10× into column 3. Use 3× dilution scheme in DMSO through column 10, lastly diluting further by 10× into column 11. Add 100% DMSO in serum-free and complete medium in columns 1 and 12 as a positive and negative control, respectively. To achieve a dilution of 1:500, use two 0.8 ml 96-well deep-well plates (ThermoFisher AB0765) pre-filled with the medium as intermediate (1:20) and final dilution plate (1:25). (Transfer row-wise)
Flick the cell plate carefully, three to four times or until all wells are clear of any residual liquid. Dispense 50 μL of the compound in medium into respective wells row-wise, incubate at 37° C. for 30 min
After a 30-minute incubation, flick the cell plate carefully, three to four times or until all wells are clear of any residual liquid. Dilute stock Lysis Buffer #4 (4×) in deionized water, complemented with blocking solution, the stock of which is 100×*. Add 75 μL 1× lysis buffer+blocking reagent mix into each well. Mix plate on a shaker for 30 min at room temperature
Dilute stock solution of total/phospho-YAP Eu cryptate & d2 antibody (each) in the ratio of 1:20 with detection buffer. Mix antibody solutions Eu3+ cryptate and d2 (each for total-Yap and phospho-YAP) in the ratio of 1:1*. Transfer 4 μL of the antibody mixture into two separate small-volume 384-well plates assigned for total-YAP and phospho-YAP. Give a short spin to the plates in a benchtop centrifuge set at 1000 rpm for 30 seconds
Row-wise, homogenize the lysate gently by pipetting it up and down, swirling the tip in the well to help scrape off any adherent cells (avoid bubble formation). Row-wise transfer 16 μL cell lysate into the low-volume 384-well plates with 4 μL total-YAP or phospho-YAP antibody reaction mix for a final volume of 20 μL. Spin down the plates in a benchtop centrifuge set at 1000 rpm for 30 seconds
Seal the plates using a vacuum sealer, incubate overnight at room temperature in the dark on a shaker; read the next day
Use a fluorescence emission read using compatible HTRF® reader—BioTek Synergy NEO. Calculate the ratio of the acceptor (665 nm) and donor (620 nm) emission signals for each well. Ratio is equal to signal 665 nm/signal 620 nm×10,000.
   Included in Cisbio HTRF YAP total kit (#64YATPEH) & YAP phospho-S127 kit (#64YAPPEH).

TABLE I
Inhibitory Activity of phosphorylation of YAP

| | LATS1_ICKA_HEK293A |
|---|---|
| Example | IC₅₀ (nM) |
| 1 | 19.7 |
| 2 | 23 |
| 3 | 5.15 |
| 4 | 205 |
| 5 | 1698 |
| 6 | 1.3 |
| 7 | 1854 |
| 8 | 687 |
| 9 | 1735 |
| 10 | 39.2 |
| 11 | 53 |
| 12 | 37 |
| 13 | 45 |
| 14 | 71 |
| 17 | 1485 |
| 19 | 1715 |
| 25 | 77 |
| 26 | 69 |
| 27 | 5560 |
| 28 | 109 |
| 30 | 39 |
| 34 | 1706 |
| 35 | 392.4 |
| 41 | 4.75 |
| 58 | 526.9 |
| 65 | 640 |
| 66 | 327 |
| 67 | 147 |
| 68 | 270 |
| 69 | 68 |
| 84 | 2290 |
| 85 | 31610 |
| 88 | 3100 |

1. A compound of formula I, II, or III: 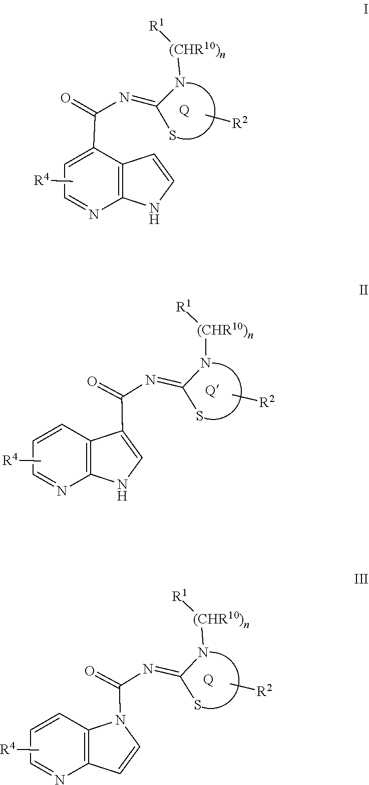
wherein:
the ring designated Q is a five-, six-, or seven-membered heterocycle containing one sulfur and one nitrogen,
the ring designated Q′ is a thiazolidine, a six-, or seven-membered heterocycle containing one sulfur and one nitrogen, or, when R² is —(CH₂)ₘNR³⁰R³¹ or a divalent three or four carbon residue that forms a fused ring, Q′ may additionally be a thiazole;
R¹ is selected from the group consisting of (C₁-C₆)alkyl, —COOH, (C₃-C₇)carbomonocyclyl, (C₉-C₁₁)carbobicyclyl, heteromonocyclyl other than 3-piperidinyl, and heterobicyclyl,
wherein said (C₁-C₆)alkyl, (C₃-C₇)carbomonocyclyl, (C₉-C₁₁)carbobicyclyl, heteromonocyclyl, and heterobicyclyl may be optionally substituted with from one to three substituents selected independently from the group consisting of halogen, cyano, hydroxy, nitro, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, heteroaryl, benzenesulfonyl, (C₁-C₃)alkoxycarbonyl, aminocarbonyl, (C₁-C₃)alkylamino, di(C₁-C₃)alkylamino, amino(C₁-C₃)alkyl, (C₁-C₃)alkylamino(C₁-C₃)alkyl (C₁-C₃)dialkylamino(C₁-C₃)alkyl, (C₁-C₃)alkylthio, (C₁-C₃)alkylsulfonylamino, (C₁-C₃)alkylsulfinyl, (C₁-C₃)alkylsulfonyl, phenoxy, and benzyloxy; or, when R² is a divalent three or four carbon residue that forms a fused ring, R¹ may provide a point of attachment for the ring;
R² is
(a) one or two monovalent substituents selected independently from the group consisting of hydrogen, halogen, (C₁-C₇)hydrocarbyl, halo(C₁-C₆)alkyl, (C₁-C₆)acyl, hydroxy(C₁-C₃)alkyl, —C(═O)O(C₁-C₆)alkyl, —C(═O)NR²⁰R²¹, (C₁-C₆)oxaalkyl, and —(CH₂)ₘNR³⁰R³¹, or
(b) divalent ═O, or
(c) a divalent three or four carbon residue that forms a fused ring, with the proviso that, when R² is methyl, it is not at the 5-position of a thiazole;
R⁴ is selected from the group consisting of hydrogen, halogen, (C₁-C₆)hydrocarbyl, halo(C₁-C₆)alkyl, (C₁-C₆)acyl, and (C₁-C₃)alkoxy;
R¹⁰ is selected independently in each instance from the group consisting of hydrogen and methyl;
R²⁰ is selected from the group consisting of hydrogen and (C₁-C₆)hydrocarbyl;
R²¹ is selected from the group consisting of hydrogen, (C₁-C₆)hydrocarbyl, (C₁-C₆)oxaalkyl, amino(C₁-C₆)alkyl, (C₁-C₃)alkylamino(C₁-C₆)alkyl, di(C₁-C₃)alkylamino(C₁-C₆)alkyl, and —(CH₂)ₘ-Het, wherein Het is an aliphatic mono- or bicyclic heterocycle, optionally substituted with a substituent selected from the group consisting hydroxy, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, aminocarbonyl, (C₁-C₃)alkylaminocarbonyl, di(C₁-C₃)alkylaminocarbonyl, (C₁-C₃)alkylamino, and di(C₁-C₃)alkylamino;
or, taken together with the nitrogen to which they are attached, R²⁰ and R²¹ form an aliphatic heterocyle;
R³⁰ is selected from the group consisting of hydrogen and (C₁-C₆)hydrocarbyl;
R³¹ is selected from the group consisting of hydrogen, (C₁-C₆)hydrocarbyl, (C₁-C₆)oxaalkyl, amino(C₁-C₆)alkyl, (C₁-C₃)alkylamino(C₁-C₆)alkyl, di(C₁-C₃)alkylamino(C₁-C₆)alkyl, and (C₁-C₆)acyl;
or, taken together with the nitrogen to which they are attached, R³⁰ and R³¹ form an aliphatic heterocyle;
n is zero, one or two; and
m is zero, one or two.

2. A compound according to claim 1 of formula II:

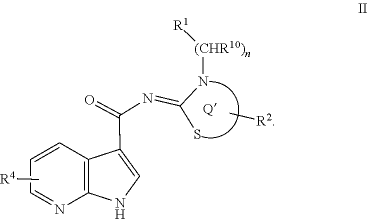

3. A compound according to claim 1 of formula I:

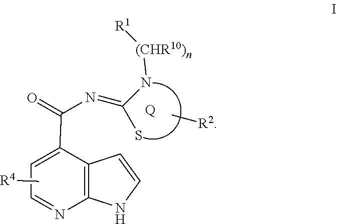

4. A compound according to claim 1 of formula III:

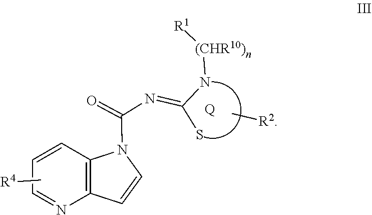

5. A compound according to claim 1 wherein Q or Q′ is thiazolidine:

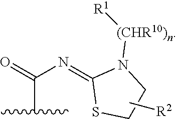

6. A compound according to claim 2 wherein Q′ is thiazolidine: 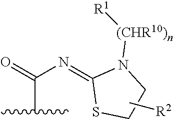
n is zero or one, R¹ is chosen from phenyl, substituted phenyl, and cyclohexyl, and R² is chosen from hydrogen, hydroxymethyl, and —CH₂NR³⁰R³¹.

7. A compound according claim 1 wherein Q is thiazole:

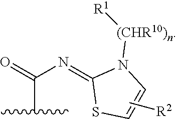

8. A compound according to claim 2 wherein Q′ is thiazole: 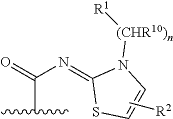
and R² is:
(1) —(CH₂)ₘNR³⁰R³¹, or
(2) a divalent three or four carbon residue that forms a fused ring.

9. A compound according to claim 1 wherein Q or Q′ is thiazine or dihydrothiazine:

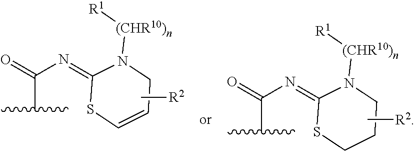

10. A compound according to claim 1 wherein Q or Q′ is thiazepane:

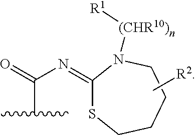

11. A compound according to claim 1 wherein R² is one or two monovalent substituents selected independently from the group consisting of hydrogen, (C₁-C₃)alkyl, halo(C₁-C₆)alkyl, (C₁-C₆)acyl, hydroxy(C₁-C₃)alkyl, —C(═O)O(C₁-C₆)alkyl, —C(═O)NR²⁰R²¹, (C₁-C₆)oxaalkyl, and —CH₂NR³⁰R³¹, or R² is divalent ═O.

12. A compound according to claim 1 wherein R² is a divalent three or four carbon residue that forms a fused ring.

13. A compound according to claim 11 wherein R²⁰ is chosen from hydrogen and methyl, and and R²¹ is chosen from hydrogen, methyl, (C₁-C₆)oxaalkyl, dimethylamino(C₁-C₆)alkyl, and —(CH₂)ₘ-Het or R²⁰ and R²¹ taken together with the nitrogen to which they are attached form a 4-7-membered aliphatic heterocycle.

14. A compound according to claim 1 wherein n is zero.

15. A compound according to claim 1 wherein n is one.

16. A compound according to claim 15 wherein R¹⁰ is hydrogen.

17. A compound according to claim 1 wherein R¹ is chosen from hydroxyalkyl, optionally substituted phenyl, cyclohexyl, and optionally substituted 4-piperidinyl.

18. A compound according to claim 17 wherein R¹ is phenyl or phenyl substituted with one or two substituents selected independently from the group consisting of halogen, cyano, hydroxy, amino, carboxy, (C₁-C₆)hydrocarbyl, trifluoromethyl, methoxy, acetyl, formyl, hydroxy(C₁-C₃)alkyl, methoxycarbonyl, carboxamido, methanesulfonylamino, amino(C₁-C₃)alkyl, phenoxy, and benzyloxy.

19. A compound according to claim 13 wherein R¹ is optionally substituted heterocyclyl selected from the group consisting of pyridinyl, pyrazolyl, piperidinyl, tetrahydropyranyl, tetrahydrofuranyl, and tetrahydroisoquinolinyl, each optionally substituted.

20. A compound according to claim 1 wherein R¹ is cyclohexyl or phenyl wherein said cyclohexyl or phenyl are optionally substituted with one or two substituents chosen from hydroxy and halogen.

21. A compound according to claim 1 wherein R⁴ is hydrogen or halogen.

22. (canceled)

23. A pharmaceutical composition comprising a pharmaceutically acceptable carrier and a compound according to claim 1.

24. A method for activating YAP in a cell expressing YAP comprising exposing the cell to a compound of formula I, II, or III: 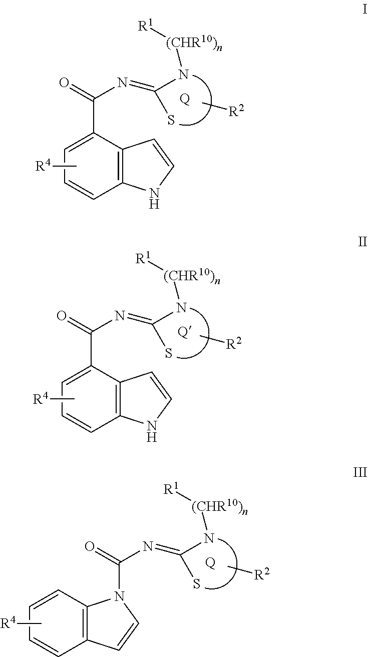
wherein the substituents are as defined in claim 1.

25. A method of LATS inhibition in a cell expressing LATS comprising exposing said cell to a compound of formula I, II, or III: 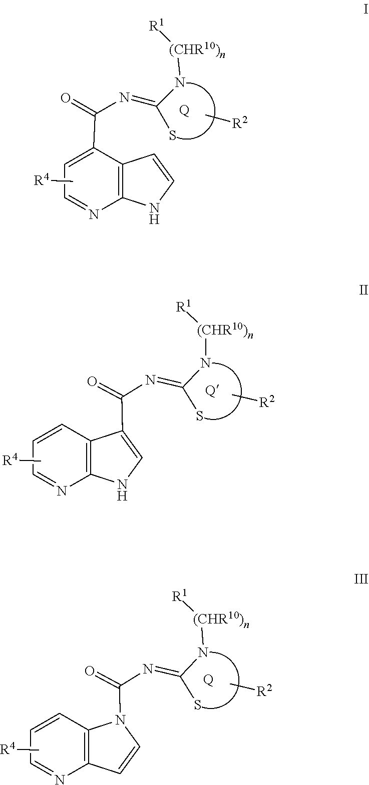
wherein the substituents are as defined in claim 1.

26. A method for stimulating hair cell regeneration comprising exposing a supporting-cell population to a compound of formula I, II, or III. 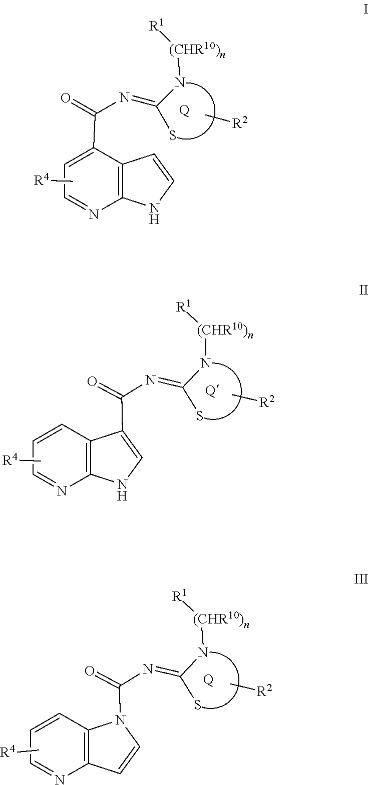
wherein the substituents are as defined in claim 1.

27.-33. (canceled)
